(19) United States
(12) Patent Application Publication
(10) Pub. No.: US 2017/0355708 A1
(43) Pub. Date: Dec. 14, 2017

(54) POTASSIUM CHANNEL MODULATORS

(71) Applicant: Cadent Therapeutics, Inc., Cambridge, MA (US)

(72) Inventors: Martin R. Jefson, Stonington, CT (US); Gregg F. Keaney, Lexington, MA (US); Janus Schreiber Larsen, Holbæk (DK); John A. Lowe, III, Stonington, CT (US); John M. McCall, Boca Grande, FL (US)

(21) Appl. No.: 15/617,091

(22) Filed: Jun. 8, 2017

Related U.S. Application Data
(60) Provisional application No. 62/347,762, filed on Jun. 9, 2016.

(51) Int. Cl. C07D 491/107; C07D 401/14; C07D 403/04; C07D 413/14; C07D 401/04; C07D 487/04; C07D 403/14; C07D 487/08; C07D 471/04; C07D 405/14; C07D 417/14; C07D 417/04

(52) U.S. Cl. CPC C07D 491/107; C07D 417/14; C07D 401/14; C07D 403/04; C07D 413/14; C07D 417/04; C07D 487/04; C07D 403/14; C07D 487/08; C07D 471/04; C07D 405/14; C07D 401/04

(57) ABSTRACT

Provided are novel compounds of Formula (I):

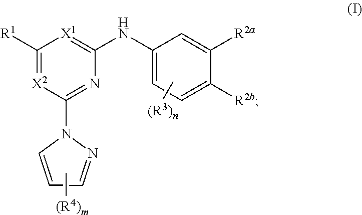

and pharmaceutically acceptable salts thereof, which are useful for treating a variety of diseases, disorders or conditions, associated with potassium channels. Also provided are pharmaceutical compositions comprising the novel compounds of Formula (I), pharmaceutically acceptable salts thereof, and methods for their use in treating one or more diseases, disorders or conditions, associated with potassium channels.

### RELATED APPLICATIONS

This application claims priority to U.S. Provisional Application No. 62/347,762, filed Jun. 9, 2016, the contents of which are incorporated herein by reference.

### BACKGROUND OF THE INVENTION

Among the ion channels, potassium channels are the largest and most diverse, being found in a variety of animal cells such as nervous, muscular, glandular, immune, reproductive, and epithelial tissue. These channels allow the flow of potassium in and/or out of the cell under certain conditions. These channels are regulated, e.g., by calcium sensitivity, voltage-gating, second messengers, extracellular ligands, and ATP-sensitivity.

Dysfunction of potassium channels, as well as other ion channels, generates loss of cellular control and results in altered physiological functioning and disease conditions. Because of their ability to modulate ion channel function and/or regain ion channel activity in acquired or inherited channelopathies, potassium channel modulators are being used in the pharmacological treatment of a wide range of pathological diseases and have the potential to address an even wider variety of therapeutic indications.

The small conductance calcium-activated potassium channels (SK channel) are a subfamily of Ca²⁺-activated K⁺ channels and the SK channel family contains 4 members—SK1, SK2, SK3, and SK4. The physiological roles of the SK channels has been especially studied in the nervous system, where e.g., they are key regulators of neuronal excitability and of neurotransmitter release, and in smooth muscle, where they are crucial in modulating the tone of vascular, broncho-tracheal, urethral, uterine or gastro-intestinal musculature.

Given these implications, small molecule modulators of potassium ion channels could have potentially powerful influence in the modulation and control of numerous consequences of a variety of conditions.

### SUMMARY OF THE INVENTION

Disclosed are compounds and pharmaceutically acceptable salts thereof, and pharmaceutical compositions thereof, which are useful in the treatment of diseases associated with the modulation of ion channels, such as potassium ion channels. (See e.g., Table 2). Such compounds include those of structural Formula I:

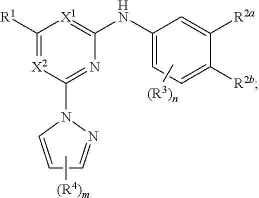

or a pharmaceutically acceptable salt thereof, wherein each of R¹, R^{2a}, R^{2b}, R³, R⁴, X¹, X², m and n are as defined and described herein.

Compounds described herein, and pharmaceutically acceptable compositions thereof, are useful for treating a variety of diseases, disorders or conditions, associated with the modulation of potassium channels. Such diseases, disorders, or conditions include those described herein.

### DETAILED DESCRIPTION OF CERTAIN EMBODIMENTS

### 1. General Description of Compounds of the Invention

In certain embodiments, provided herein is a compound of Formula I:

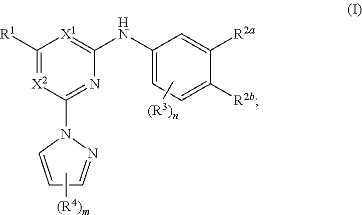

or a pharmaceutically acceptable salt thereof, wherein:

each of X¹ and X² is independently N or C(R⁵), wherein X¹ and X² are not simultaneously N;

R¹ is selected from —CN, —C₁-C₄ alkyl, -heterocyclyl, -heteroaryl, —NH-heterocyclyl, —NH—C₁-C₄ alkyl, —O-heterocyclyl, —O-heteroaryl, —O—C₁-C₄ alkyl, —S—C₁-C₄ alkyl, —S(O)—C₁-C₄ alkyl, —S(O)₂—C₁-C₄ alkyl, and —S(O)₂-heterocyclyl; or

when X¹ or X² is C(R⁵), R¹ is optionally taken together with X¹ or X² and their intervening atoms to form a 5- or 6-membered heteroaryl or heterocyclyl ring comprising 1 or 2 nitrogen atoms;

each of R^{2a} and R^{2b} is independently selected from hydrogen, fluoro, chloro, —CN, —CF₃, —CHF₂, and C₁-C₄ alkyl, wherein at least one of R^{2a} or R^{2b} is fluoro, chloro, —CN, —CF₃, or —CHF₂;

each R³ is independently selected from fluoro, chloro and C₁-C₄ alkyl;

each R⁴ is independently selected from cyano and C₁-C₄ alkyl;

each R⁵ is independently selected from hydrogen and C₁-C₄ alkyl;

n is 0, 1, 2, or 3;

m is 1, 2 or 3;

wherein any alkyl portion of R¹, R^{2a}, R^{2b}, R³, R⁴ or R⁵ and any heterocyclyl or heteroaryl portion of R¹ or the ring formed by taking R¹ together with X¹ or X² is optionally substituted with one or more substituents independently selected from R⁶;

R⁶ is selected from halogen, —CN, —OR^{c}, —NR^{d}R^{e}, —S(O)ₖR^{c}, —NR^{c}S(O)₂R^{c}, —S(O)₂NR^{d}R^{e}, —C(═O)OR^{c}, —OC(═O)OR^{c}, —OC(═O)R^{c}, —OC(═S)OR^{c}, —C(═S)OR^{c}, —OC(═S)R^{c}, —C(═O)NR^{d}R^{e}, —NR^{c}C(═O)R^{c}, —C(═S)NR^{d}R^{e}, —NR^{c}C(═S)R^{c}, —NR^{c}C(═O)OR^{c}, —OC(═O)NR^{d}R^{e}, —NR^{c}(C═S)OR^{c}, —OC(═S)NR^{d}R^{e}, —NR^{c}C(═O)NR^{d}R^{e}, —NR^{c}(C═S)NR^{d}R^{e}, —C(═S)R^{c}, —C(═O)R^{c}, (C₁-C₆)alkyl, cycloalkyl, —(CH₂)₁₋₄-cycloalkyl, heterocyclyl, —(CH₂)₁₋₄-heterocyclyl, aryl, —NHC(═O)-heterocyclyl, —NHC(═O)-cycloalkyl, —(CH₂)₁₋₄-aryl, heteroaryl and —(CH₂)₁₋₄-heteroaryl,

wherein the alkyl, cycloalkyl, heterocyclyl, aryl or heteroaryl portion present in each of said (C₁-C₆)alkyl, cycloalkyl, —(CH₂)₁₋₄-cycloalkyl, heterocyclyl, —(CH₂)₁₋₄-heterocyclyl, aryl, —(CH₂)₁₋₄-aryl, heteroaryl and —(CH₂)₁₋₄-heteroaryl substituent for R⁶ are further optionally substituted with halogen, OR^{c}, —NO₂, —CN, —NR^{c}C(═O)R^{c}, —NR^{d}R^{e}, —S(O)ₖR^{c}, —C(═O)OR^{c}, —C(═O)NR^{d}R^{e}, —C(═O)R^{c}, (C₁-C₃)alkyl, halo(C₁-C₃)alkyl, (C₁-C₃)alkoxy(C₁-C₃)alkyl, (C₁-C₃)alkoxy, or halo(C₁-C₃)alkoxy;

each R^{c} is independently selected from hydrogen and (C₁-C₆)alkyl optionally substituted with 1 to 3 halogen;

each R^{d} and R^{e} is independently selected from hydrogen and (C₁-C₆)alkyl;

k is 0, 1 or 2; and

any alkyl portion of any of R¹, R^{2a}, R^{2b}, R³, or R⁵ and any heterocyclyl or heteroaryl portion of R¹ or the ring formed by taking R¹ together with X¹ or X² is further optionally substituted with ═O;

provided that:

when X² is N; X¹ and R¹ are taken together to form:

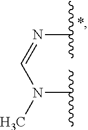 [SMILES: */N=C\N(*)C]

wherein “*” represents a portion of the moiety bound to X¹; n is 0 or 1; and R³, when present, is halo, then the portion of the molecule represented by

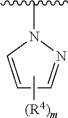

is other than

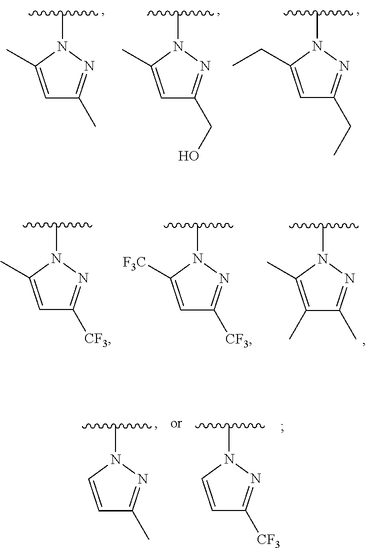

when X² is N; X¹ and R¹ are taken together to form:

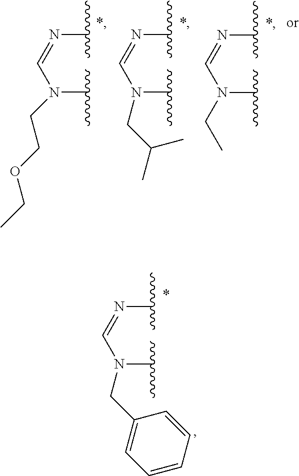

wherein “*” represents a portion of the moiety bound to X¹; and n is 0, then the portion of the molecule represented by

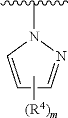

is other than

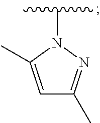

when R¹ is methyl, the portion of the molecule represented by

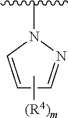

is other than

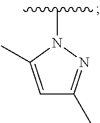

and

the compound is other than

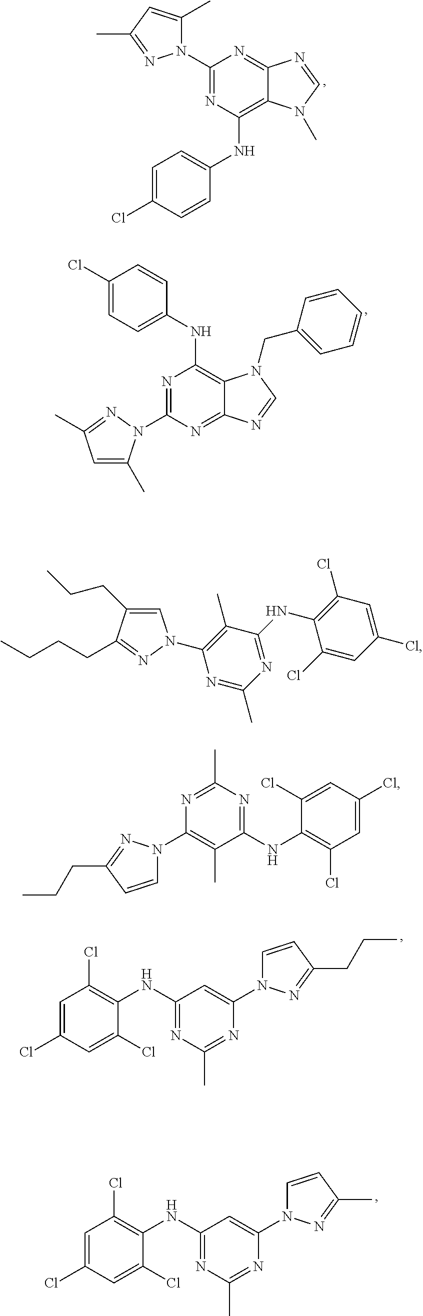

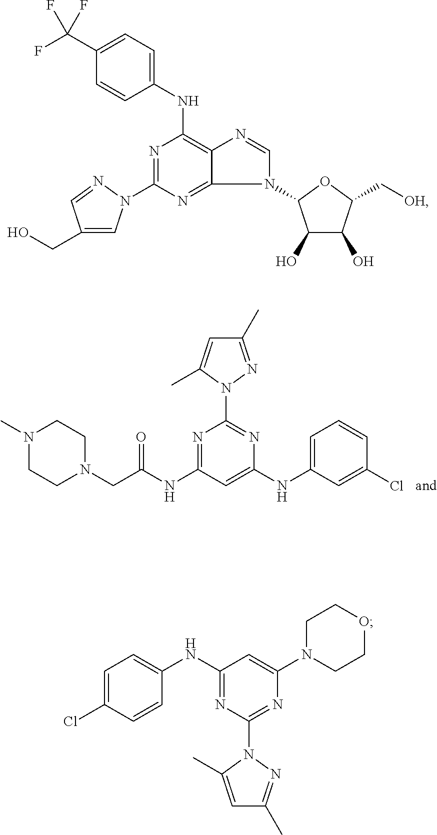 [SMILES: OCc1cnn(-c2nc(Nc3ccc(C(F)(F)F)cc3)c3ncn([C@@H]4O[C@H](CO)[C@@H](O)[C@H]4O)c3n2)c1 ; Cc1cc(C)n(-c2nc(NC(=O)CN3CCN(C)CC3)cc(Nc3cccc(Cl)c3)n2)n1 ; Cc1cc(C)n(-c2nc(Nc3ccc(Cl)cc3)cc(N3CCOCC3)n2)n1]

or a pharmaceutically acceptable salt thereof.

Also provided herein is a compound of Formula I:

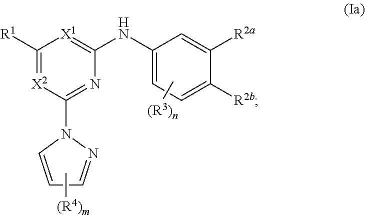

or a pharmaceutically acceptable salt thereof, wherein:

each of X¹ and X² is independently N or C(R⁵), wherein X¹ and X² are not simultaneously N;

R¹ is selected from —CN, —(C₀-C₄ alkylene)-heterocyclyl, —(C₀-C₄ alkylene)-heteroaryl, —C(O)—C₁-C₄ alkyl, —C(O)—O—C₁-C₄ alkyl, —C(O)—NH₂, —C(O)—NH—C₁-C₄ alkyl, —C(O)—N(C₁-C₄ alkyl)₂, —(C₀-C₄ alkylene)-NH-heterocyclyl, —(C₀-C₄ alkylene)-NH—C₁-C₄ alkyl, —(C₀-C₄ alkylene)-NH₂, —(C₀-C₄ alkylene)-O-heterocyclyl, —(C₀-C₄ alkylene)-O-heteroaryl, —(C₀-C₄ alkylene)-O—C₁-C₄ alkyl, —(C₀-C₄ alkylene)-S—C₁-C₄ alkyl, —(C₀-C₄ alkylene)-S(O)—C₁-C₄ alkyl, —(C₀-C₄ alkylene)-S(O)₂—C₁-C₄ alkyl, and —(C₀-C₄ alkylene)-S(O)₂-heterocyclyl;

each of R^{2a} and R^{2b} is independently selected from hydrogen, fluoro, chloro, —CN, —CF₃, —CHF₂, and C₁-C₄ alkyl, wherein at least one of R^{2a} or R^{2b} is fluoro, chloro, —CN, —CF₃, or —CHF₂

each R³ is independently selected from fluoro, chloro and C₁-C₄ alkyl;

each R⁴ is independently selected from cyano and C₁-C₄ alkyl;

each R⁵ is independently selected from hydrogen and C₁-C₄ alkyl;

n is 0, 1, 2, or 3;

m is 1, 2 or 3;

wherein any alkyl portion of R^{2a}, R^{2b}, R³, R⁴ or R⁵ and any alkyl, alkylene, heterocyclyl or heteroaryl portion of R¹ is optionally substituted with one or more substituents independently selected from R⁶;

R⁶ is selected from halogen, —CN, —OR^{c}, —NR^{d}R^{e}, —S(O)ₖR^{c}, —NR^{c}S(O)₂R^{c}, —S(O)₂NR^{d}R^{e}, —C(═O)OR^{c}, —OC(═O)OR^{c}, —OC(═O)R^{c}, —OC(═S)OR^{c}, —C(═S)OR^{c}, —OC(═S)R^{c}, —C(═O)NR^{d}R^{e}, —NR^{c}C(═O)R^{c}, —C(═S)NR^{d}R^{e}, —NR^{c}C(═S)R^{c}, —NR^{c}C(═O)OR^{c}, —OC(═O)NR^{d}R^{e}, —NR^{c}(C═S)OR^{c}, —OC(═S)NR^{d}R^{e}, —NR^{c}C(═O)NR^{d}R^{e}, —NR^{c}(C═S)NR^{d}R^{e}, —C(═S)R^{c}, —C(═O)R^{c}, (C₁-C₆)alkyl, cycloalkyl, —(CH₂)₁₋₄-cycloalkyl, heterocyclyl, —(CH₂)₁₋₄-heterocyclyl, aryl, —NHC(═O)-heterocyclyl, —NHC(═O)-cycloalkyl, —(CH₂)₁₋₄-aryl, heteroaryl and —(CH₂)₁₋₄-heteroaryl,

wherein the alkyl, cycloalkyl, heterocyclyl, aryl or heteroaryl portion present in each of said (C₁-C₆)alkyl, cycloalkyl, —(CH₂)₁₋₄-cycloalkyl, heterocyclyl, —(CH₂)₁₄-heterocyclyl, aryl, —(CH₂)₁₋₄-aryl, heteroaryl and —(CH₂)₁₋₄-heteroaryl substituent for R⁶ are further optionally substituted with halogen, OR^{c}, —NO₂, —CN, —NR^{c}C(═O)R^{c}, —NR^{d}R^{e}, —S(O)ₖR^{c}, —C(═O)OR^{c}, —C(═O)NR^{d}R^{e}, —C(═O)R^{c}, (C₁-C₃)alkyl, halo(C₁-C₃)alkyl, (C₁-C₃)alkoxy(C₁-C₃)alkyl, (C₁-C₃)alkoxy, or halo(C₁-C₃)alkoxy;

each R^{c} is independently selected from hydrogen and (C₁-C₆)alkyl optionally substituted with 1 to 3 halogen;

each R^{d} and R^{e} is independently selected from hydrogen and (C₁-C₆)alkyl;

k is 0, 1 or 2; and

any alkyl portion of any of R^{2a}, R^{2b}, R³, or R⁵ and any alkyl, alkylene, heterocyclyl or heteroaryl portion of R¹ is further optionally substituted with ═O;

provided that the compound is other than

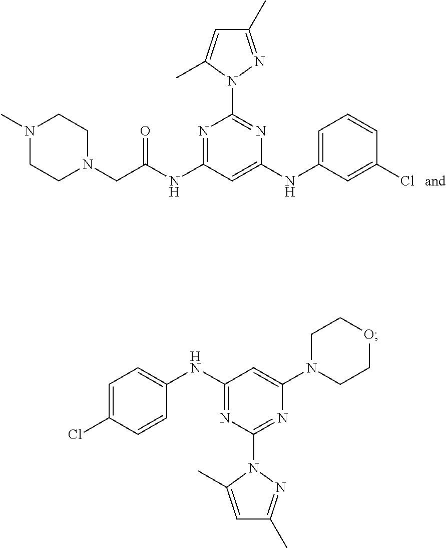 [SMILES: Cc1cc(C)n(-c2nc(NC(=O)CN3CCN(C)CC3)cc(Nc3cccc(Cl)c3)n2)n1 ; Cc1cc(C)n(-c2nc(Nc3ccc(Cl)cc3)cc(N3CCOCC3)n2)n1]

or a pharmaceutically acceptable salt thereof.

### 2. Compounds and Definitions

The terms “halo” and “halogen” as used herein refer to an atom selected from fluorine (fluoro, —F), chlorine (chloro, —Cl), bromine (bromo, —Br), and iodine (iodo, —I).

The term “alkyl” used alone or as part of a larger moiety, such as “alkoxy”, “haloalkyl”, “aralkyl”, “heteroaralkyl” and the like, means saturated straight-chain or branched monovalent hydrocarbon radical. Unless otherwise specified, an alkyl group typically has 1-6 carbon atoms, i.e., (C₁-C₆)alkyl. As used herein, a “(C₁-C₆)alkyl” group is means a radical having from 1 to 6 carbon atoms in a linear or branched arrangement.

The term “haloalkyl” includes mono, poly, and perhaloalkyl groups where the halogens are independently selected from fluorine, chlorine, bromine, and iodine.

“Alkoxy means an alkyl radical attached through an oxygen linking atom, represented by —O-alkyl. For example, “(C₁-C₄)alkoxy” includes methoxy, ethoxy, proproxy, and butoxy.

The term “aryl” used alone or as part of a larger moiety as in “aralkyl”, “aralkoxy”, or “aryloxyalkyl”, refers to an aromatic monocyclic or bicyclic carbon ring system having, unless otherwise specified, a total of 6 to 14 ring members. The term “aryl” may be used interchangeably with the term “aryl ring”, “aryl group”, “aryl moiety,” or “aryl radical”. Also included within the scope of the term “aryl”, as it is used herein, is a group in which an aromatic carbon ring is fused to one or more carbocyclyl rings, e.g., tetrahydronaphthalenyl. In certain embodiments of the present disclosure, “aryl” refers to an aromatic ring system which includes, but is not limited to, phenyl (abbreviated as “Ph”), naphthyl and the like. It will be understood that when specified, optional substituents on an aryl group (e.g., in the case of an optionally substituted aryl or aryl which is optionally substituted) may be present on any substitutable position, i.e., any ring carbon substituted with hydrogen.

The term “carbocyclyl” (also referred to herein as “carbocycle” or “cycloaliphatic”, as used herein, means a monocyclic, bicyclic (e.g., a bridged or spiro bicyclic ring), polycyclic (e.g., tricyclic), or fused hydrocarbon ring system that is completely saturated or that contains one or more units of partial unsaturation, but where there is no aromatic ring. Cycloalkyl is a completely saturated carbocycle. Monocyclic carbocyclyl groups include, without limitation, cyclopropyl, cyclobutyl, cyclopentyl, cyclopentenyl, cyclohexyl, cyclohexenyl, cycloheptyl, cycloheptenyl, and cyclooctyl. Bridged bicyclic carbocyclyl groups include, without limitation, bicyclo[3.2.1]octane, bicyclo[2.2.1]heptane, bicyclo[3.1.0]hexane, and the like. Spiro bicyclic carbocyclyl groups include, e.g., spiro[3.6]decane, spiro[4.5]decane, and the like. Fused carbocyclyl rings include, e.g., decahydronaphthalene, octahydropentalene, and the like. It will be understood that when specified, optional substituents on a carbocyclyl (e.g., in the case of an optionally substituted carbocyclyl or carbocyclyl which is optionally substituted) may be present on any substitutable position and, include, e.g., the position at which the carbocyclyl group is attached.

The term “heteroaryl” used alone or as part of a larger moiety as in “heteroarylalkyl”, “heteroarylalkoxy”, or “heteroarylaminoalkyl”, refers to a 5-10-membered aromatic monocyclic or bicyclic radical containing 1-4 heteroatoms selected from N, quaternary ammonium cation, O, and S, and includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, indazolyl, benzimidazolyl, benzthiazolyl, pyrrolopyridinyl, quinolyl, quinazolinyl, and quinoxalinyl. The term “heteroaryl” may be used interchangeably with the terms “heteroaryl ring”, “heteroaryl group”, or “heteroaromatic”. It will be understood that when specified, optional substituents on a heteroaryl group may be present on any substitutable position (carbon and nitrogen).

The term “heterocyclyl” means a 3-12 membered (e.g., a 4-, 5-, 6- and 7-membered) saturated or partially unsaturated heterocyclic ring containing 1 to 4 heteroatoms independently selected from N, O, and S. It can be mononcyclic, bicyclic (e.g., a bridged, fused, or spiro bicyclic ring), or tricyclic. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, 3-azabicyclo[3.1.0]hexanyl, 2-oxa-6-azaspiro[3.3]heptanyl, 1-azaspiro[4.5]decane, and tetrahydropyrimidinyl. The term “heterocyclyl” also includes, e.g., unsaturated heterocyclic radicals fused to another heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that share one ring atom (e.g., carbon).

The term “fused” refers to two rings that share two adjacent ring atoms.

The term “bridged” refers to two rings that share at least three ring atoms.

As described herein, compounds herein may contain “optionally substituted” moieties. Unless otherwise indicated, an “optionally substituted” group may have a suitable substituent that results in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

Certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. Diastereomers are stereoisomers that contain two or more asymmetrically substituted carbon atoms. “Geometric isomer” are stereoisomers that differ in the orientation of substituent atoms in relationship to a carbon-carbon double bond, to a carbocyclyl ring, or to a bridged bicyclic system.

“Racemate” or “racemic mixture” means a compound of equimolar quantities of two enantiomers, wherein such mixtures exhibit no optical activity, i.e., they do not rotate the plane of polarized light.

The compounds of the invention may be prepared as individual enantiomers by either enantio-specific synthesis or resolved from an enantiomerically enriched mixture. Conventional resolution techniques include forming the salt of a free base of each isomer of an enantiomeric pair using an optically active acid (followed by fractional crystallization and regeneration of the free base), forming the salt of the acid form of each enantiomer of an enantiomeric pair using an optically active amine (followed by fractional crystallization and regeneration of the free acid), forming an ester or amide of each of the enantiomers of an enantiomeric pair using an optically pure acid, amine or alcohol (followed by chromatographic separation and removal of the chiral auxiliary), or resolving an enantiomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisiomer over the weight of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight pure relative to all of the other stereoisomers. The stereoisomeric purity in this case is determined by dividing the total weight in the mixture of the stereoisomers encompassed by the name or structure by the total weight in the mixture of all of the stereoisomers.

When a disclosed compound is named or depicted by structure without indicating the stereochemistry, and the compound has one chiral center, it is to be understood that the name or structure encompasses one enantiomer of compound free from the corresponding optical and geometric isomer, a racemic mixture of the compound, and mixtures enriched in one enantiomer relative to its corresponding optical isomer.

When a disclosed compound is named or depicted by structure without indicating the stereochemistry and e.g, the compound has at least two chiral centers, it is to be understood that the name or structure encompasses one stereoisomer free of other stereoisomers, mixtures of stereoisomers, and mixtures of stereoisomers in which one or more stereoisomers is enriched relative to the other stereoisomer(s). For example, the name or structure may encompass one stereoisomer free of other diastereomers, mixtures of stereoisomers, and mixtures of stereoisomers in which one or more diastereomers is enriched relative to the other diastereomer(s).

With respect to the compound defined by generic Formula I and Ia, unless otherwise specified, one or more hydrogens can be replaced by deuterium. Isotopic enrichments include e.g., at least 10%, 25%, 50%, 75%, 80%, 85%, 90&, 95%, 87%, 98%, 99.0%, 99.5% and 99.8%”. In one embodiment, all hydrogen atoms represented in Formula I and Ia are present in natural abundance. With respect to specific compounds disclosed herein, such as those in Table 1 and in the Exemplification section, all hydrogen atoms are present in natural abundance unless otherwise specified.

The compounds described herein may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

### 3. Description of Exemplary Compounds

In a first embodiment, the present disclosure provides a compound of Formula I:

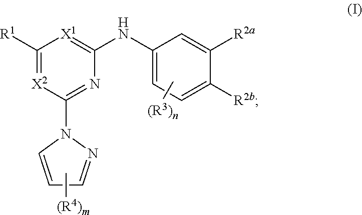

or a pharmaceutically acceptable salt thereof, wherein the variables are as described above.

In a second embodiment, the present disclosure provides a compound of Formula Ia:

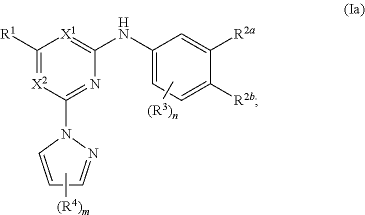

or a pharmaceutically acceptable salt thereof, wherein the variables are as described above.

In a third embodiment, R¹ in Formula I or Ia is selected from —CN, —C(O)NH₂, heterocyclyl, heteroaryl, —NH-heterocyclyl, —NH—(C₁-C₄ alkylene)-heteroaryl, —O—(C₁-C₄ alkylene)-heteroaryl, —O-heterocyclyl, —O-heteroaryl, —(C₁-C₄ alkylene)-NH—C(O)—C₁-C₄ alkyl, —NH—(C₁-C₄ alkylene)-C(O)—NH₂, —(C₁-C₄ alkylene)-heteroaryl, and —S(O)₂—C₁-C₄ alkyl, wherein each alkyl, alkylene, heterocyclyl and heteroaryl portion of R¹ is optionally substituted with one or more substituents independently selected from R⁶, wherein the remaining variables are as described in Formula I or Ia.

In a fourth embodiment, R¹ in Formula I or Ia is selected from —CN, —C(O)NH₂, heterocyclyl, heteroaryl, —NH-heterocyclyl, —NH—(C₁-C₂ alkylene)-heteroaryl, —O—(C₁-C₂ alkylene)-heteroaryl, —O-heterocyclyl, —O-heteroaryl, —(C₁-C₂ alkylene)-NH—C(O)—C₁-C₄ alkyl, —NH—(C₁-C₂ alkylene)-C(O)—NH₂, —(C₁-C₂ alkylene)-heteroaryl, and —S(O)₂—C₁-C₂ alkyl, wherein each heterocyclyl and heteroaryl portion of R¹ is optionally substituted with one or more substituents independently selected from R⁶, wherein the remaining variables are as described in Formula I or Ia, or the third embodiment.

In a fifth embodiment, R¹ in Formula I or Ia is selected from piperazinyl, 2,5-diazabicyclo[2.2.1]heptan-2-yl, 2-oxa-6-azaspiro[3.3]heptan-6-yl, morpholinyl, —NH-3-azabicyclo[3.1.0]hexan-6-ylamino, O-oxetanyl, O-pyridazinyl, —(C₁-C₂ alkylene)-pyrazolyl, 0-pyridazinyl, —NH—(C₁-C₂ alkylene)-thiazolyl, —NH—(C₁-C₂ alkylene)-oxazolyl, —NH-pyrrolidin-2-one-3-yl, —NH-piperidin-2-one-3-yl, —O—(C₁-C₂ alkylene)-triazolyl, —CN, —C(O)NH₂, —(C₁-C₂ alkylene)-NH—C(O)—C₁-C₄ alkyl, —NH—(C₁-C₂ alkylene)-C(O)—NH₂, and —S(O)₂—C₁-C₂ alkyl, wherein each of said piperazinyl 2,5-diazabicyclo[2.2.1]heptan-2-yl, 2-oxa-6-azaspiro[3.3]heptan-6-yl, morpholinyl, 3-azabicyclo[3.1.0]hexan-6-ylamino, oxetanyl, pyridazinyl, pyrazolyl, thiazolyl, oxazolyl, triazolyl, pyrrolidin-2-one-3-yl, and piperidin-2-one-3-yl are optionally substituted with one or more substituents independently selected from R⁶ wherein the remaining variables are as described in Formula I or Ia, or the third or fourth embodiment.

In a sixth embodiment, R⁶ in Formula I or Ia is selected from —C(═O)R^{c}, —OR^{c}, —NR^{d}R^{e}, —C(═O)NR^{d}R^{e}, —(C₁-C₄)alkyl, —(C₁-C₄)alkyl-OR^{c}, and —(C₁-C₄)alkyl-phenyl; R^{d} and R^{e} are each independently hydrogen or (C₁-C₄)alkyl; and R^{c} is hydrogen or (C₁-C₃)alkyl, wherein the remaining variables are as described in Formula I or Ia, or the third, fourth, or fifth embodiment.

In a seventh embodiment, R⁶ in Formula I or Ia is selected from —C(═O)R^{c}, —C(═O)NR^{d}R^{e}, —(C₁-C₄)alkyl, —(C₁-C₄)alkyl-OR^{c}, and —(C₁-C₄)alkyl-phenyl; R^{d} and R^{e} are each hydrogen; and R^{c} is hydrogen or (C₁-C₃)alkyl, wherein the remaining variables are as described in Formula I or Ia, or the third, fourth, fifth, or sixth embodiment.

In an eighth embodiment, R¹ in Formula I or Ia is selected from —CN, —C(O)NH₂, —CH₂NH₂, —CH₂NHCOCH₃, —S(O)₂CH₃, —NHCH(C(O)NH₂)CH(CH₃)₂,

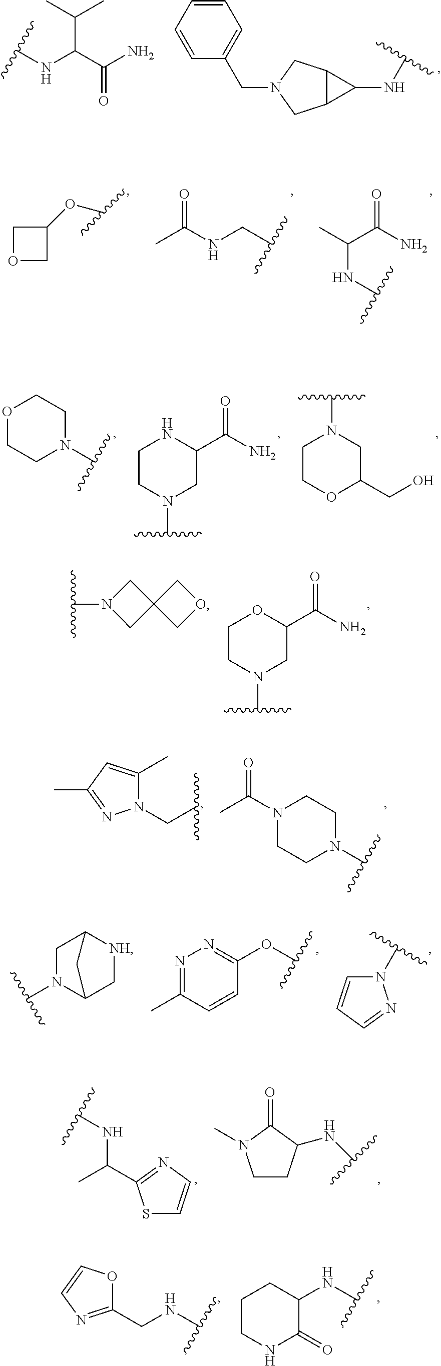

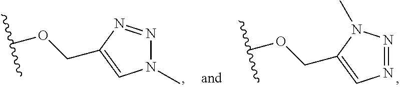

wherein the remaining variables are as described in Formula I or Ia, or the third, fourth, fifth, sixth, or seventh embodiment.

In a ninth embodiment, R^{2b} in Formula I or Ia is selected from hydrogen, fluoro and chloro; and R^{2a} is selected from hydrogen and —CF₃, wherein the remaining variables are as described in Formula I or Ia, or the third, fourth, fifth, sixth, seventh, or eighth embodiment.

In a tenth embodiment, n is 0 or 1 in Formula I or Ia, wherein the remaining variables are as described in Formula I or Ia, or the third, fourth, fifth, sixth, seventh, eighth, or ninth embodiment.

In an eleventh embodiment, R³ in Formula I or Ia is selected from fluoro and CF₃, wherein the remaining variables are as described in Formula I or Ia, or the third, fourth, fifth, sixth, seventh, eighth, ninth, or tenth embodiment.

In a twelfth embodiment, R⁴ in Formula I or Ia is independently selected from —CN and C₁-C₄ alkyl optionally substituted hydroxy or one or more with halo, wherein the remaining variables are as described in Formula I or Ia, or the third, fourth, fifth, sixth, seventh, eighth, ninth, tenth, or eleventh embodiment.

In a thirteenth embodiment, R⁴ in Formula I or Ia is independently selected from —CN, —CH₃, —CH₂F, —CHF₂, —CH₂OH, —CH(OH)CH₃, —C(CH₃)₂OH, and —CH(CH₃)F, wherein the remaining variables are as described in Formula I or Ia, or the third, fourth, fifth, sixth, seventh, eighth, ninth, tenth, eleventh, or twelfth embodiment.

In a fourteenth embodiment, R⁴ in Formula I or Ia is —CH₃, wherein the remaining variables are as described in Formula I or Ia, or the third, fourth, fifth, sixth, seventh, eighth, ninth, tenth, eleventh, twelfth, or thirteenth embodiment.

In a fifteenth embodiment, each R⁵ in Formula I or Ia, if present, is independently selected from CH₃ and CH(OH)CH₃, wherein the remaining variables are as described in Formula I or Ia, or the third, fourth, fifth, sixth, seventh, eighth, ninth, tenth, eleventh, twelfth, thirteenth, or fourteenth embodiment.

In a sixteenth embodiment, R¹ in Formula I or Ia is taken together with X² to form a ring selected from:

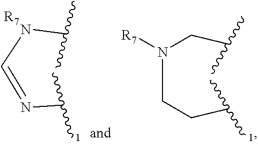

wherein “1” represents a point of attachment to X², and R⁷ is selected from hydrogen and C₁-C₄ alkyl optionally substituted with one or more substituents independently selected from R⁶, wherein the remaining variables are as described in Formula I or Ia, or the ninth, tenth, eleventh, twelfth, thirteenth, fourteenth, or fifteenth embodiment.

In a seventeenth embodiment, R¹ in Formula I or Ia is taken together with X² to form a ring selected from:

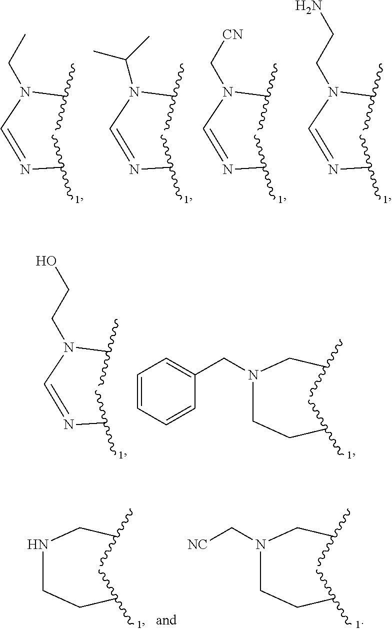

wherein the remaining variables are as described in Formula I or Ia, or the ninth, tenth, eleventh, twelfth, thirteenth, fourteenth, fifteenth, or sixteenth embodiment.

In an eighteenth embodiment, R¹ in Formula I or Ia is C₁-C₄ alkyl optionally substituted with halo, wherein the remaining variables are as described in Formula I or Ia, or the ninth, tenth, eleventh, twelfth, thirteenth, fourteenth, or fifteenth embodiment.

In a nineteenth embodiment, R¹ in Formula I or Ia is methyl, wherein the remaining variables are as described in Formula I or Ia, or the ninth, tenth, eleventh, twelfth, thirteenth, fourteenth, fifteenth, or eighteenth embodiment

Specific examples of compounds are provided in Table 1 and the EXEMPLIFICATION section and are included as part of a twenty-second embodiment herein. Pharmaceutically acceptable salts as well as the neutral forms of the compounds in Table 1 and Table 2 as well as the EXEMPLIFICATION are also included.

TABLE 1

| Com- | |
|---|---|
| pound | |
| # | Structure |
| 100 | 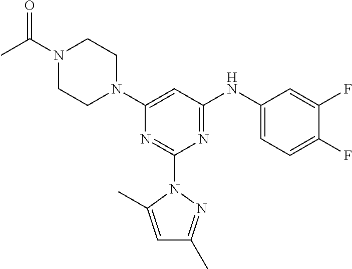 [SMILES: CC(=O)N1CCN(c2cc(Nc3ccc(F)c(F)c3)nc(-n3nc(C)cc3C)n2)CC1] |
| 101 | 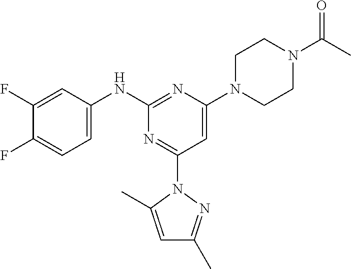 [SMILES: CC(=O)N1CCN(c2cc(-n3nc(C)cc3C)nc(Nc3ccc(F)c(F)c3)n2)CC1] |
| 102 | 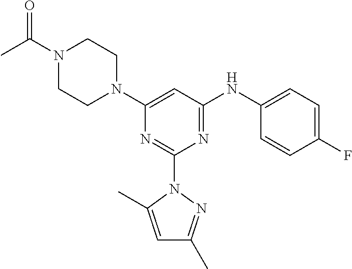 [SMILES: CC(=O)N1CCN(c2cc(Nc3ccc(F)cc3)nc(-n3nc(C)cc3C)n2)CC1] |
| 103 | 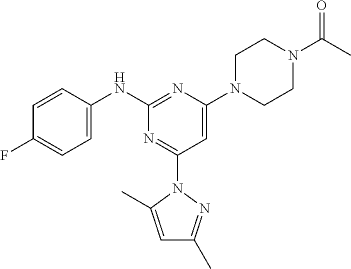 [SMILES: CC(=O)N1CCN(c2cc(-n3nc(C)cc3C)nc(Nc3ccc(F)cc3)n2)CC1] |
| 104 | 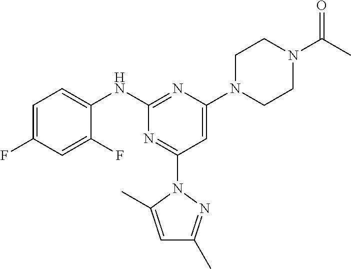 [SMILES: CC(=O)N1CCN(c2cc(-n3nc(C)cc3C)nc(Nc3ccc(F)cc3F)n2)CC1] |
| 105 | 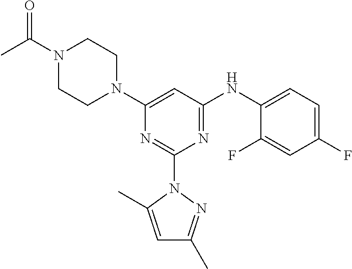 [SMILES: CC(=O)N1CCN(c2cc(Nc3ccc(F)cc3F)nc(-n3nc(C)cc3C)n2)CC1] |
| 106 | 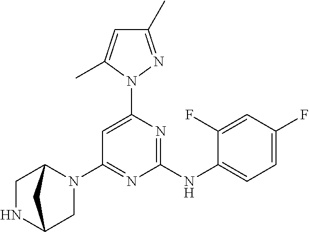 [SMILES: Cc1cc(C)n(-c2cc(N3C[C@H]4C[C@@H]3CN4)nc(Nc3ccc(F)cc3F)n2)n1] |
| 107 | 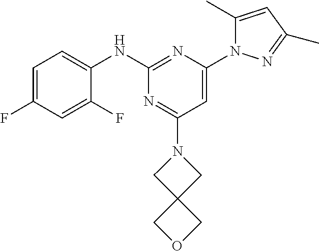 [SMILES: Cc1cc(C)n(-c2cc(N3CC4(COC4)C3)nc(Nc3ccc(F)cc3F)n2)n1] |
| 108 | 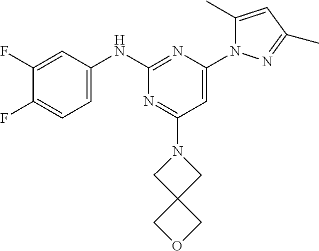 [SMILES: Cc1cc(C)n(-c2cc(N3CC4(COC4)C3)nc(Nc3ccc(F)c(F)c3)n2)n1] |
| 109 | 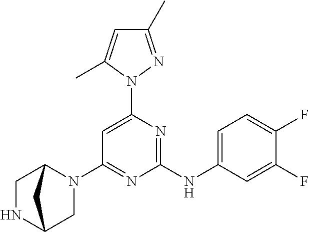 [SMILES: Cc1cc(C)n(-c2cc(N3C[C@H]4C[C@@H]3CN4)nc(Nc3ccc(F)c(F)c3)n2)n1] |
| 110 | 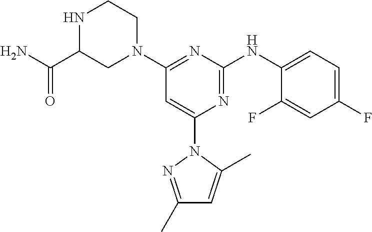 [SMILES: Cc1cc(C)n(-c2cc(N3CCNC(C(N)=O)C3)nc(Nc3ccc(F)cc3F)n2)n1] |
| 111 | 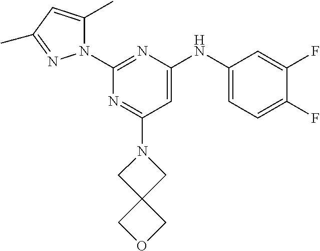 [SMILES: Cc1cc(C)n(-c2nc(Nc3ccc(F)c(F)c3)cc(N3CC4(COC4)C3)n2)n1] |
| 112 | 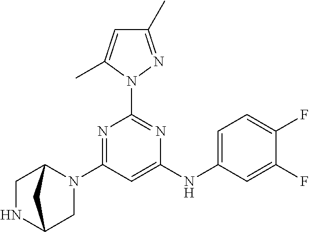 [SMILES: Cc1cc(C)n(-c2nc(Nc3ccc(F)c(F)c3)cc(N3C[C@H]4C[C@@H]3CN4)n2)n1] |
| 113 | 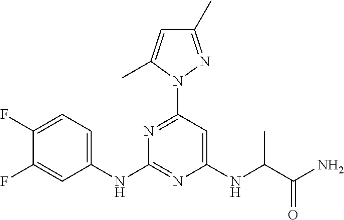 [SMILES: Cc1cc(C)n(-c2cc(NC(C)C(N)=O)nc(Nc3ccc(F)c(F)c3)n2)n1] |
| 114 | 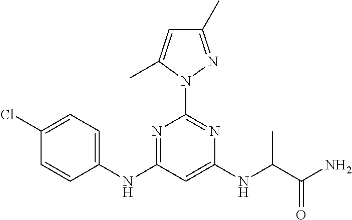 [SMILES: Cc1cc(C)n(-c2nc(Nc3ccc(Cl)cc3)cc(NC(C)C(N)=O)n2)n1] |
| 115 | 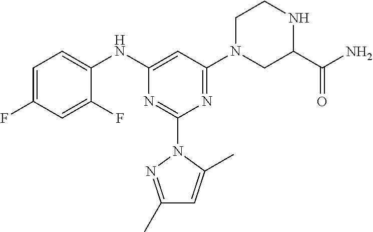 [SMILES: Cc1cc(C)n(-c2nc(Nc3ccc(F)cc3F)cc(N3CCNC(C(N)=O)C3)n2)n1] |
| 116 | 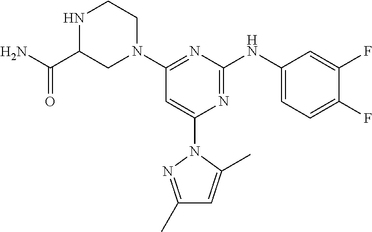 [SMILES: Cc1cc(C)n(-c2cc(N3CCNC(C(N)=O)C3)nc(Nc3ccc(F)c(F)c3)n2)n1] |
| 117 | 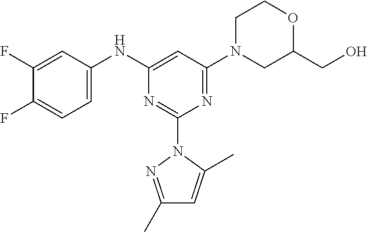 [SMILES: Cc1cc(C)n(-c2nc(Nc3ccc(F)c(F)c3)cc(N3CCOC(CO)C3)n2)n1] |
| 118 | 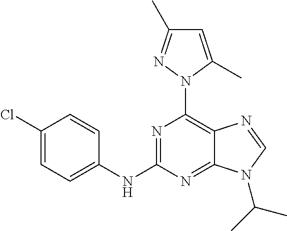 [SMILES: Cc1cc(C)n(-c2nc(Nc3ccc(Cl)cc3)nc3c2ncn3C(C)C)n1] |
| 119 | 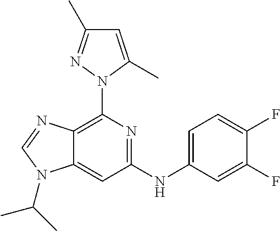 [SMILES: Cc1cc(C)n(-c2nc(Nc3ccc(F)c(F)c3)cc3c2ncn3C(C)C)n1] |
| 120 | 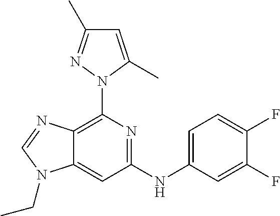 [SMILES: CCn1cnc2c(-n3nc(C)cc3C)nc(Nc3ccc(F)c(F)c3)cc21] |
| 121 | 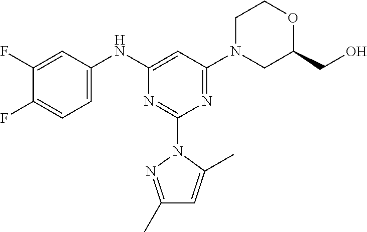 [SMILES: Cc1cc(C)n(-c2nc(Nc3ccc(F)c(F)c3)cc(N3CCO[C@@H](CO)C3)n2)n1] |
| 122 | 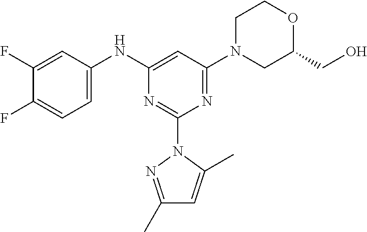 [SMILES: Cc1cc(C)n(-c2nc(Nc3ccc(F)c(F)c3)cc(N3CCO[C@H](CO)C3)n2)n1] |
| 123 | 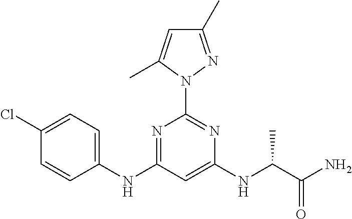 [SMILES: Cc1cc(C)n(-c2nc(Nc3ccc(Cl)cc3)cc(N[C@H](C)C(N)=O)n2)n1] |
| 124 | 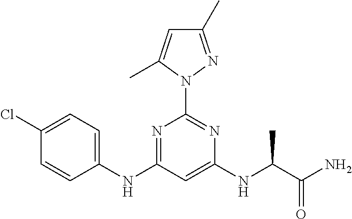 [SMILES: Cc1cc(C)n(-c2nc(Nc3ccc(Cl)cc3)cc(N[C@@H](C)C(N)=O)n2)n1] |
| 125 | 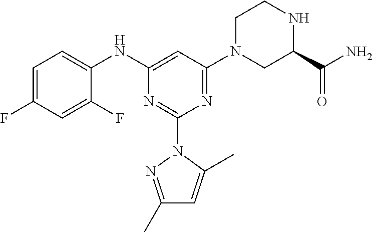 [SMILES: Cc1cc(C)n(-c2nc(Nc3ccc(F)cc3F)cc(N3CCN[C@@H](C(N)=O)C3)n2)n1] |
| 126 | 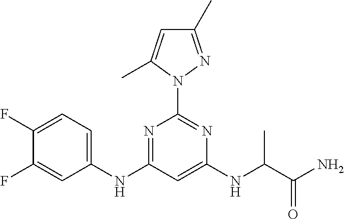 [SMILES: Cc1cc(C)n(-c2nc(Nc3ccc(F)c(F)c3)cc(NC(C)C(N)=O)n2)n1] |
| 127 | 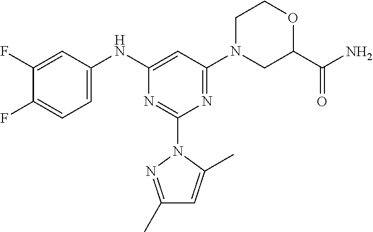 [SMILES: Cc1cc(C)n(-c2nc(Nc3ccc(F)c(F)c3)cc(N3CCOC(C(N)=O)C3)n2)n1] |
| 128 | 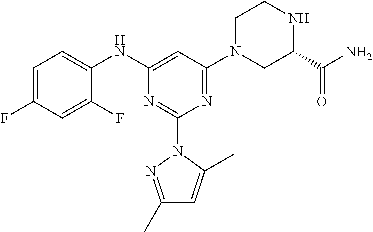 [SMILES: Cc1cc(C)n(-c2nc(Nc3ccc(F)cc3F)cc(N3CCN[C@H](C(N)=O)C3)n2)n1] |
| 129 | 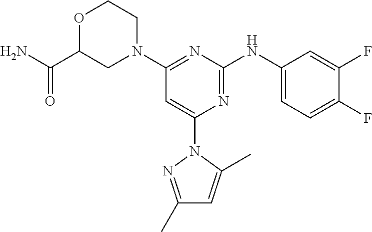 [SMILES: Cc1cc(C)n(-c2cc(N3CCOC(C(N)=O)C3)nc(Nc3ccc(F)c(F)c3)n2)n1] |
| 130 | 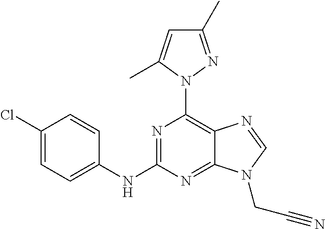 [SMILES: Cc1cc(C)n(-c2nc(Nc3ccc(Cl)cc3)nc3c2ncn3CC#N)n1] |
| 131 | 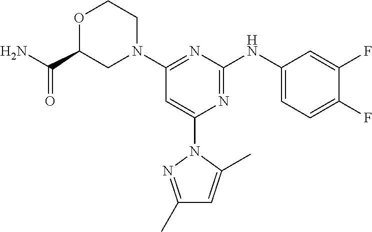 [SMILES: Cc1cc(C)n(-c2cc(N3CCO[C@H](C(N)=O)C3)nc(Nc3ccc(F)c(F)c3)n2)n1] |
| 132 | 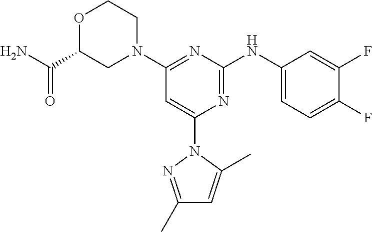 [SMILES: Cc1cc(C)n(-c2cc(N3CCO[C@@H](C(N)=O)C3)nc(Nc3ccc(F)c(F)c3)n2)n1] |
| 133 | 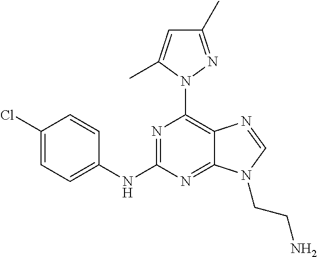 [SMILES: Cc1cc(C)n(-c2nc(Nc3ccc(Cl)cc3)nc3c2ncn3CCN)n1] |
| 134 | 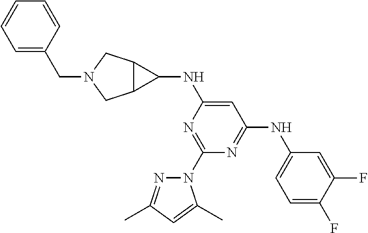 [SMILES: Cc1cc(C)n(-c2nc(Nc3ccc(F)c(F)c3)cc(NC3C4CN(Cc5ccccc5)CC43)n2)n1] |
| 135 | 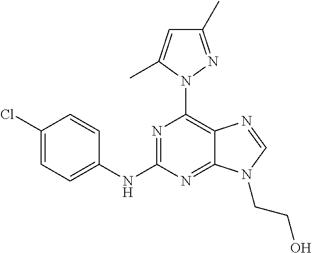 [SMILES: Cc1cc(C)n(-c2nc(Nc3ccc(Cl)cc3)nc3c2ncn3CCO)n1] |
| 136 | 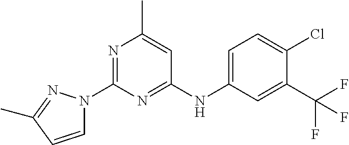 [SMILES: Cc1cc(Nc2ccc(Cl)c(C(F)(F)F)c2)nc(-n2ccc(C)n2)n1] |
| 137 | 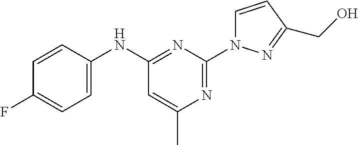 [SMILES: Cc1cc(Nc2ccc(F)cc2)nc(-n2ccc(CO)n2)n1] |
| 138 | 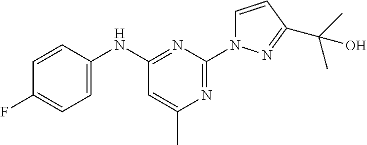 [SMILES: Cc1cc(Nc2ccc(F)cc2)nc(-n2ccc(C(C)(C)O)n2)n1] |
| 139 | 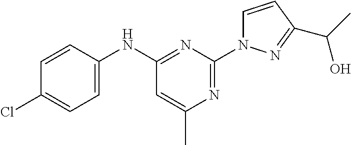 [SMILES: Cc1cc(Nc2ccc(Cl)cc2)nc(-n2ccc(C(C)O)n2)n1] |
| 140 | 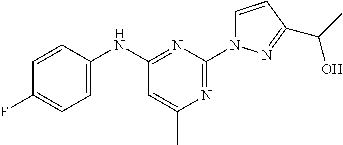 [SMILES: Cc1cc(Nc2ccc(F)cc2)nc(-n2ccc(C(C)O)n2)n1] |
| 141 | 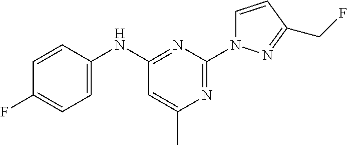 [SMILES: Cc1cc(Nc2ccc(F)cc2)nc(-n2ccc(CF)n2)n1] |
| 142 | 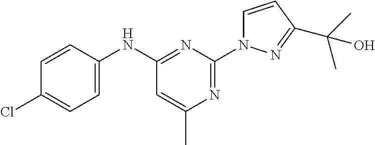 [SMILES: Cc1cc(Nc2ccc(Cl)cc2)nc(-n2ccc(C(C)(C)O)n2)n1] |
| 143 | 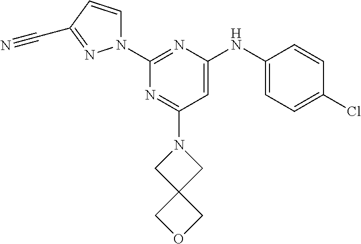 [SMILES: N#Cc1ccn(-c2nc(Nc3ccc(Cl)cc3)cc(N3CC4(COC4)C3)n2)n1] |
| 144 | 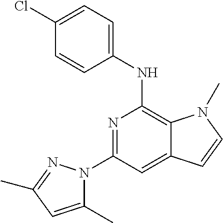 [SMILES: Cc1cc(C)n(-c2cc3ccn(C)c3c(Nc3ccc(Cl)cc3)n2)n1] |
| 145 | 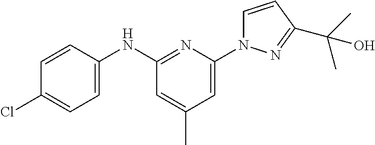 [SMILES: Cc1cc(Nc2ccc(Cl)cc2)nc(-n2ccc(C(C)(C)O)n2)c1] |
| 146 | 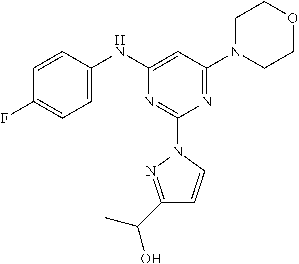 [SMILES: CC(O)c1ccn(-c2nc(Nc3ccc(F)cc3)cc(N3CCOCC3)n2)n1] |
| 147 | 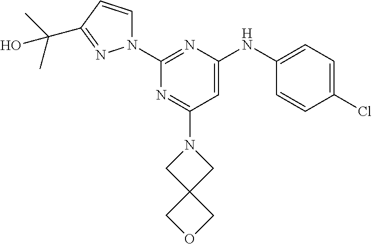 [SMILES: CC(C)(O)c1ccn(-c2nc(Nc3ccc(Cl)cc3)cc(N3CC4(COC4)C3)n2)n1] |
| 148 | 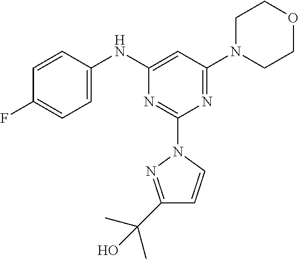 [SMILES: CC(C)(O)c1ccn(-c2nc(Nc3ccc(F)cc3)cc(N3CCOCC3)n2)n1] |
| 149 | 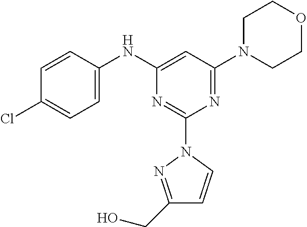 [SMILES: OCc1ccn(-c2nc(Nc3ccc(Cl)cc3)cc(N3CCOCC3)n2)n1] |
| 150 | 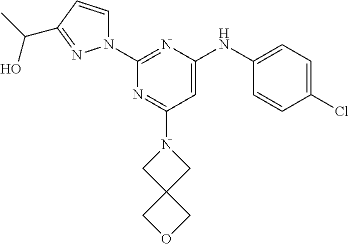 [SMILES: CC(O)c1ccn(-c2nc(Nc3ccc(Cl)cc3)cc(N3CC4(COC4)C3)n2)n1] |
| 151 | 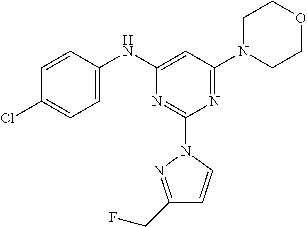 [SMILES: FCc1ccn(-c2nc(Nc3ccc(Cl)cc3)cc(N3CCOCC3)n2)n1] |
| 152 | 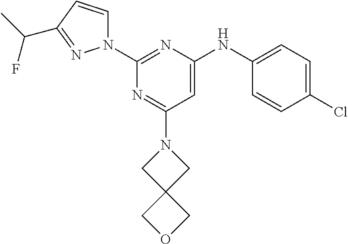 [SMILES: CC(F)c1ccn(-c2nc(Nc3ccc(Cl)cc3)cc(N3CC4(COC4)C3)n2)n1] |
| 153 | 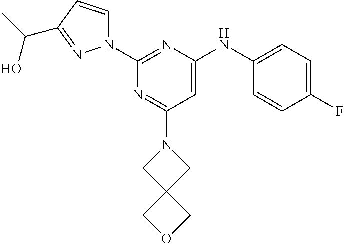 [SMILES: CC(O)c1ccn(-c2nc(Nc3ccc(F)cc3)cc(N3CC4(COC4)C3)n2)n1] |
| 154 | 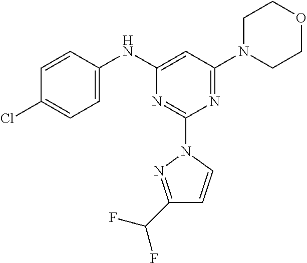 [SMILES: FC(F)c1ccn(-c2nc(Nc3ccc(Cl)cc3)cc(N3CCOCC3)n2)n1] |
| 155 | 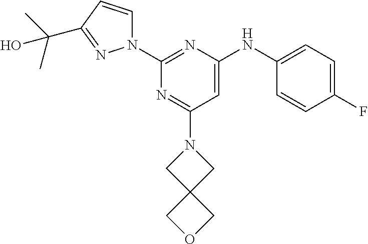 [SMILES: CC(C)(O)c1ccn(-c2nc(Nc3ccc(F)cc3)cc(N3CC4(COC4)C3)n2)n1] |
| 156 | 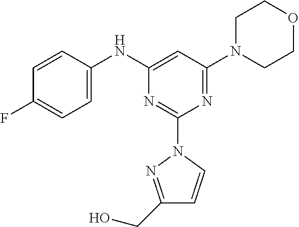 [SMILES: OCc1ccn(-c2nc(Nc3ccc(F)cc3)cc(N3CCOCC3)n2)n1] |
| 157 | 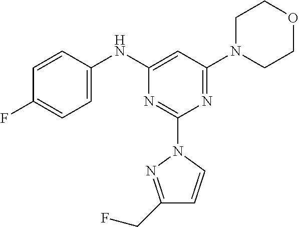 [SMILES: FCc1ccn(-c2nc(Nc3ccc(F)cc3)cc(N3CCOCC3)n2)n1] |
| 158 | 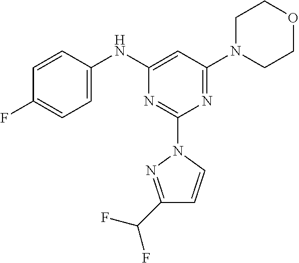 [SMILES: Fc1ccc(Nc2cc(N3CCOCC3)nc(-n3ccc(C(F)F)n3)n2)cc1] |
| 159 | 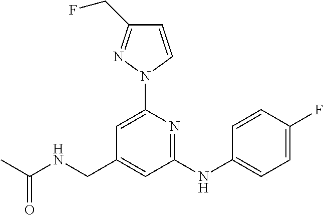 [SMILES: CC(=O)NCc1cc(Nc2ccc(F)cc2)nc(-n2ccc(CF)n2)c1] |
| 160 | 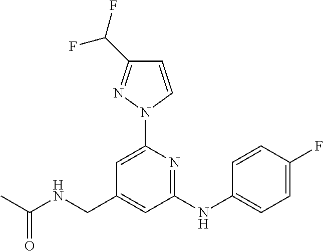 [SMILES: CC(=O)NCc1cc(Nc2ccc(F)cc2)nc(-n2ccc(C(F)F)n2)c1] |
| 161 | 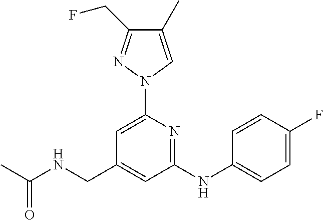 [SMILES: CC(=O)NCc1cc(Nc2ccc(F)cc2)nc(-n2cc(C)c(CF)n2)c1] |
| 162 | 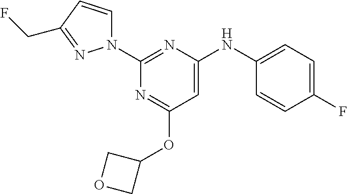 [SMILES: FCc1ccn(-c2nc(Nc3ccc(F)cc3)cc(OC3COC3)n2)n1] |
| 163 | 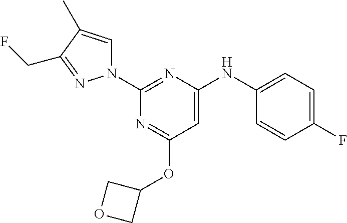 [SMILES: Cc1cn(-c2nc(Nc3ccc(F)cc3)cc(OC3COC3)n2)nc1CF] |
| 164 | 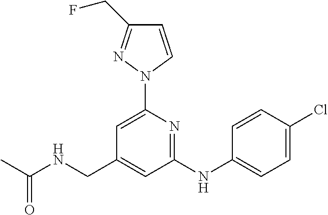 [SMILES: CC(=O)NCc1cc(Nc2ccc(Cl)cc2)nc(-n2ccc(CF)n2)c1] |
| 165 | 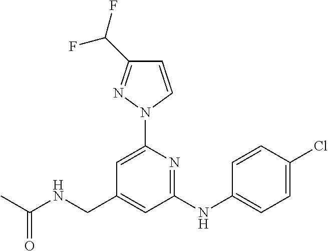 [SMILES: CC(=O)NCc1cc(Nc2ccc(Cl)cc2)nc(-n2ccc(C(F)F)n2)c1] |
| 166 | 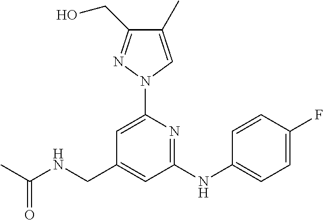 [SMILES: CC(=O)NCc1cc(Nc2ccc(F)cc2)nc(-n2cc(C)c(CO)n2)c1] |
| 167 | 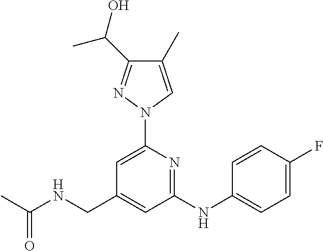 [SMILES: CC(=O)NCc1cc(Nc2ccc(F)cc2)nc(-n2cc(C)c(C(C)O)n2)c1] |
| 168 | 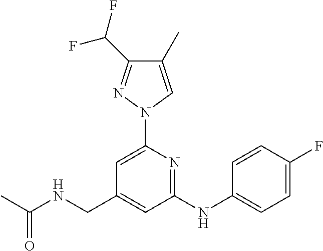 [SMILES: CC(=O)NCc1cc(Nc2ccc(F)cc2)nc(-n2cc(C)c(C(F)F)n2)c1] |
| 169 | 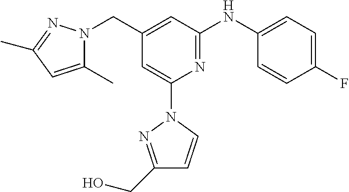 [SMILES: Cc1cc(C)n(Cc2cc(Nc3ccc(F)cc3)nc(-n3ccc(CO)n3)c2)n1] |
| 170 | 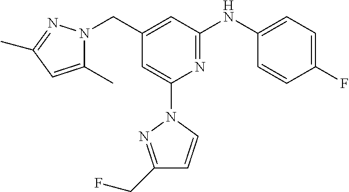 [SMILES: Cc1cc(C)n(Cc2cc(Nc3ccc(F)cc3)nc(-n3ccc(CF)n3)c2)n1] |
| 171 | 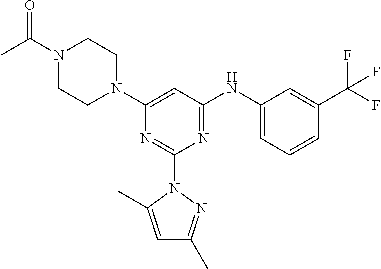 [SMILES: CC(=O)N1CCN(c2cc(Nc3cccc(C(F)(F)F)c3)nc(-n3nc(C)cc3C)n2)CC1] |
| 172 | 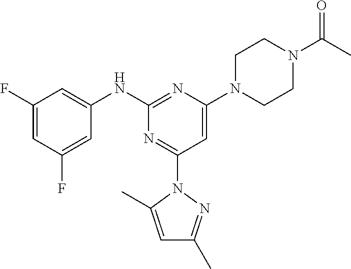 [SMILES: CC(=O)N1CCN(c2cc(-n3nc(C)cc3C)nc(Nc3cc(F)cc(F)c3)n2)CC1] |
| 173 | 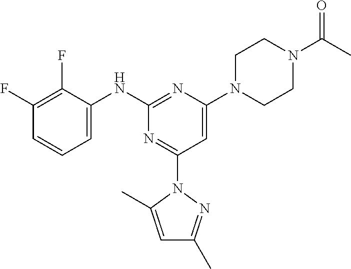 [SMILES: CC(=O)N1CCN(c2cc(-n3nc(C)cc3C)nc(Nc3cccc(F)c3F)n2)CC1] |
| 174 | 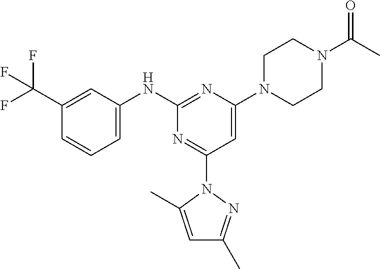 [SMILES: CC(=O)N1CCN(c2cc(-n3nc(C)cc3C)nc(Nc3cccc(C(F)(F)F)c3)n2)CC1] |
| 175 | 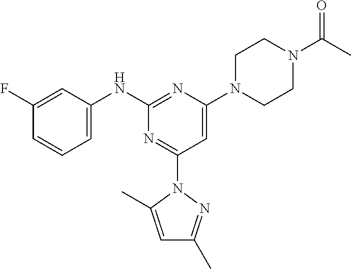 [SMILES: CC(=O)N1CCN(c2cc(-n3nc(C)cc3C)nc(Nc3cccc(F)c3)n2)CC1] |
| 176 | 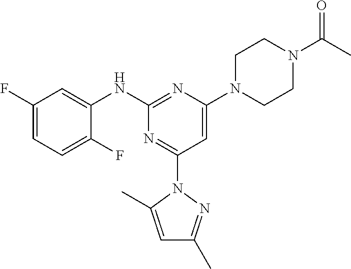 [SMILES: CC(=O)N1CCN(c2cc(-n3nc(C)cc3C)nc(Nc3cc(F)ccc3F)n2)CC1] |
| 177 | 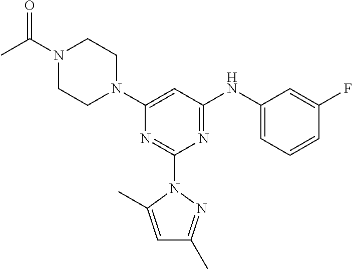 [SMILES: CC(=O)N1CCN(c2cc(Nc3cccc(F)c3)nc(-n3nc(C)cc3C)n2)CC1] |
| 178 | 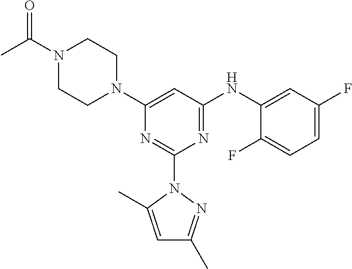 [SMILES: CC(=O)N1CCN(c2cc(Nc3cc(F)ccc3F)nc(-n3nc(C)cc3C)n2)CC1] |
| 179 | 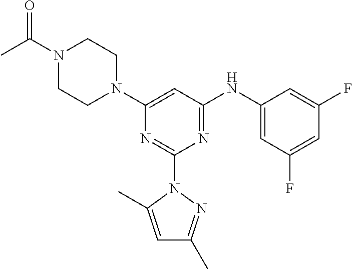 [SMILES: CC(=O)N1CCN(c2cc(Nc3cc(F)cc(F)c3)nc(-n3nc(C)cc3C)n2)CC1] |
| 180 | 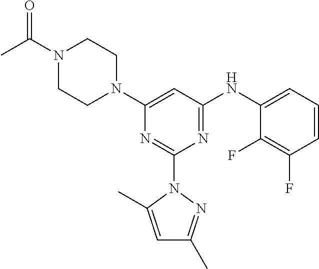 [SMILES: CC(=O)N1CCN(c2cc(Nc3cccc(F)c3F)nc(-n3nc(C)cc3C)n2)CC1] |
| 181 | 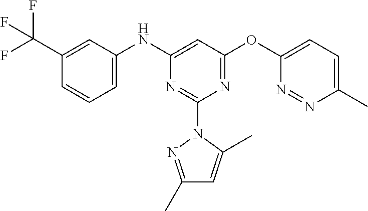 [SMILES: Cc1ccc(Oc2cc(Nc3cccc(C(F)(F)F)c3)nc(-n3nc(C)cc3C)n2)nn1] |
| 182 | 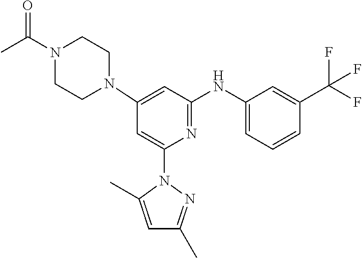 [SMILES: CC(=O)N1CCN(c2cc(Nc3cccc(C(F)(F)F)c3)nc(-n3nc(C)cc3C)c2)CC1] |
| 183 | 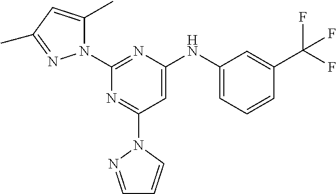 [SMILES: Cc1cc(C)n(-c2nc(Nc3cccc(C(F)(F)F)c3)cc(-n3cccn3)n2)n1] |
| 184 | 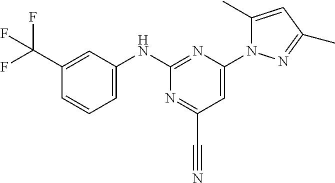 [SMILES: Cc1cc(C)n(-c2cc(C#N)nc(Nc3cccc(C(F)(F)F)c3)n2)n1] |
| 185 | 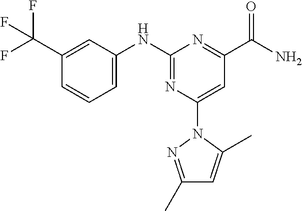 [SMILES: Cc1cc(C)n(-c2cc(C(N)=O)nc(Nc3cccc(C(F)(F)F)c3)n2)n1] |
| 186 | 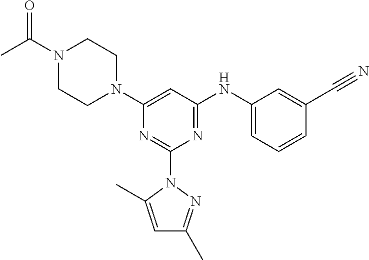 [SMILES: CC(=O)N1CCN(c2cc(Nc3cccc(C#N)c3)nc(-n3nc(C)cc3C)n2)CC1] |
| 187 | 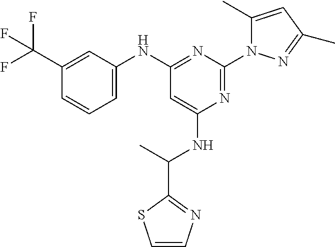 [SMILES: Cc1cc(C)n(-c2nc(Nc3cccc(C(F)(F)F)c3)cc(NC(C)c3nccs3)n2)n1] |
| 188 | 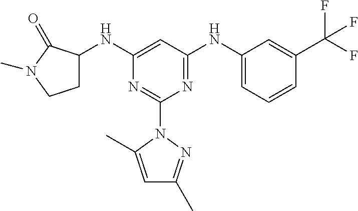 [SMILES: Cc1cc(C)n(-c2nc(Nc3cccc(C(F)(F)F)c3)cc(NC3CCN(C)C3=O)n2)n1] |
| 189 | 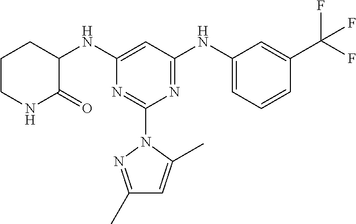 [SMILES: Cc1cc(C)n(-c2nc(Nc3cccc(C(F)(F)F)c3)cc(NC3CCCNC3=O)n2)n1] |
| 190 | 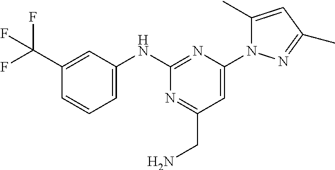 [SMILES: Cc1cc(C)n(-c2cc(CN)nc(Nc3cccc(C(F)(F)F)c3)n2)n1] |
| 191 | 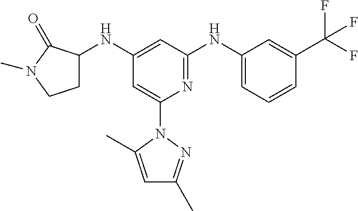 [SMILES: Cc1cc(C)n(-c2cc(NC3CCN(C)C3=O)cc(Nc3cccc(C(F)(F)F)c3)n2)n1] |
| 192 | 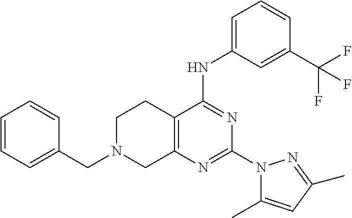 [SMILES: Cc1cc(C)n(-c2nc3c(c(Nc4cccc(C(F)(F)F)c4)n2)CCN(Cc2ccccc2)C3)n1] |
| 193 | 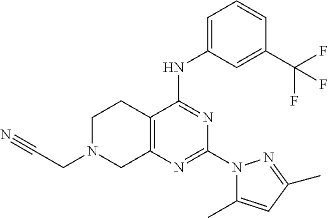 [SMILES: Cc1cc(C)n(-c2nc3c(c(Nc4cccc(C(F)(F)F)c4)n2)CCN(CC#N)C3)n1] |
| 194 | 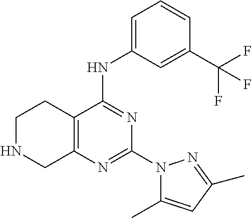 [SMILES: Cc1cc(C)n(-c2nc3c(c(Nc4cccc(C(F)(F)F)c4)n2)CCNC3)n1] |
| 195 | 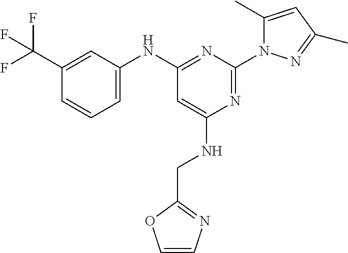 [SMILES: Cc1cc(C)n(-c2nc(NCc3ncco3)cc(Nc3cccc(C(F)(F)F)c3)n2)n1] |
| 196 | 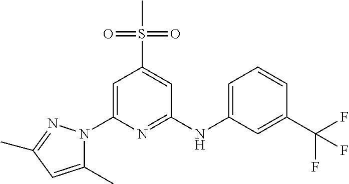 [SMILES: Cc1cc(C)n(-c2cc(S(C)(=O)=O)cc(Nc3cccc(C(F)(F)F)c3)n2)n1] |
| 197 | 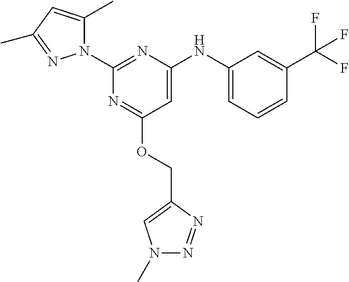 [SMILES: Cc1cc(C)n(-c2nc(Nc3cccc(C(F)(F)F)c3)cc(OCc3cn(C)nn3)n2)n1] |
| 198 | 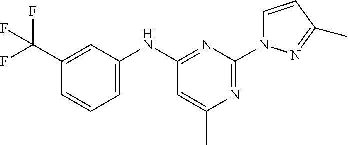 [SMILES: Cc1cc(Nc2cccc(C(F)(F)F)c2)nc(-n2ccc(C)n2)n1] |
| 199 | 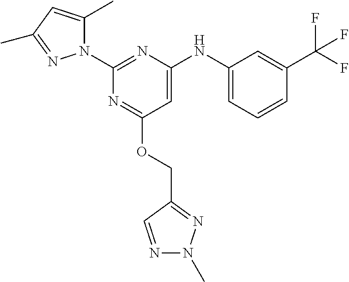 [SMILES: Cc1cc(C)n(-c2nc(Nc3cccc(C(F)(F)F)c3)cc(OCc3cnn(C)n3)n2)n1] |
| 200 | 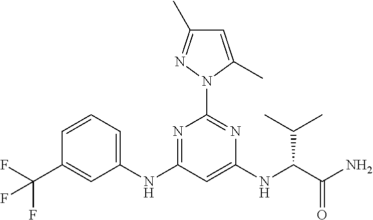 [SMILES: Cc1cc(C)n(-c2nc(Nc3cccc(C(F)(F)F)c3)cc(N[C@@H](C(N)=O)C(C)C)n2)n1] |
| 201 | 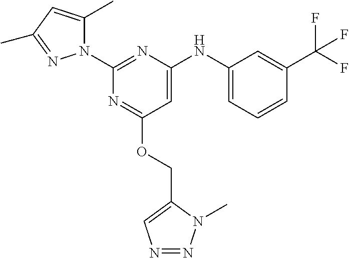 [SMILES: Cc1cc(C)n(-c2nc(Nc3cccc(C(F)(F)F)c3)cc(OCc3cnnn3C)n2)n1] |
| 202 | 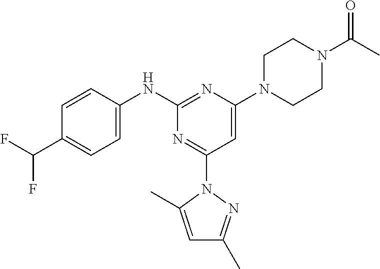 [SMILES: CC(=O)N1CCN(c2cc(-n3nc(C)cc3C)nc(Nc3ccc(C(F)F)cc3)n2)CC1] |
| 203 | 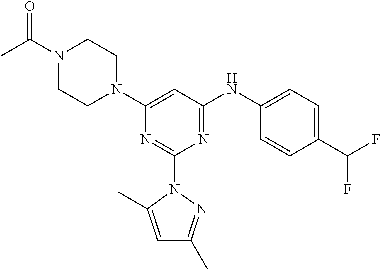 [SMILES: CC(=O)N1CCN(c2cc(Nc3ccc(C(F)F)cc3)nc(-n3nc(C)cc3C)n2)CC1] |

TABLE 2

| Compound # | Structure |
|---|---|
| IN11196-053-P1 | 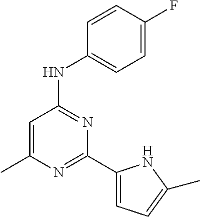 |
| IN11237-001-P1 | 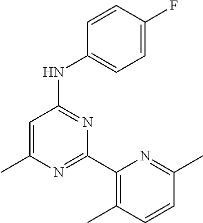 |
| IN11121-037-P1 | 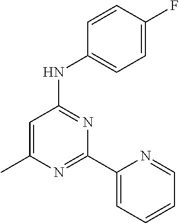 [SMILES: Cc1cc(Nc2ccc(F)cc2)nc(-c2ccccn2)n1] |
| IN11130-036-P1 | 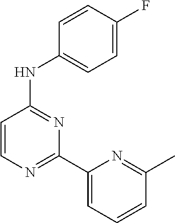 [SMILES: Cc1cccc(-c2nccc(Nc3ccc(F)cc3)n2)n1] |
| IN11166-018-P1 | 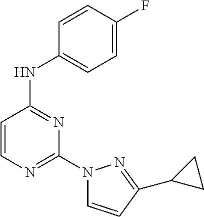 [SMILES: Fc1ccc(Nc2ccnc(-n3ccc(C4CC4)n3)n2)cc1] |
| NSSy6921 | 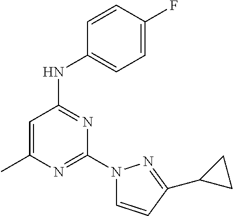 [SMILES: Cc1cc(Nc2ccc(F)cc2)nc(-n2ccc(C3CC3)n2)n1] |
| NSSy6908 | 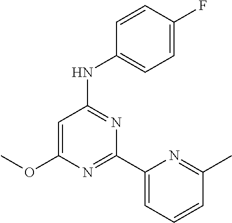 |
| NSSy6907 | 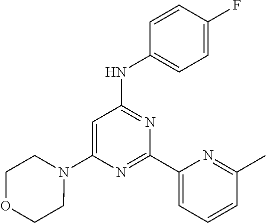 [SMILES: Cc1cccc(-c2nc(Nc3ccc(F)cc3)cc(N3CCOCC3)n2)n1] |
| IN11059-067-P1 | 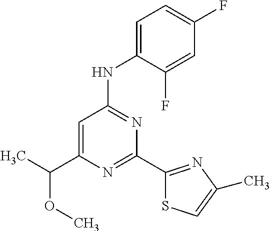 [SMILES: COC(C)c1cc(Nc2ccc(F)cc2F)nc(-c2nc(C)cs2)n1] |
| NSSy6773 | 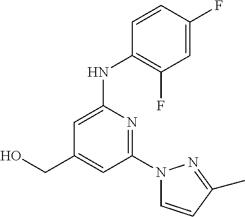 [SMILES: Cc1ccn(-c2cc(CO)cc(Nc3ccc(F)cc3F)n2)n1] |
| IN11059-059-P1 | 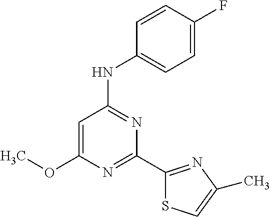 |
| IN11059-023-P1 | 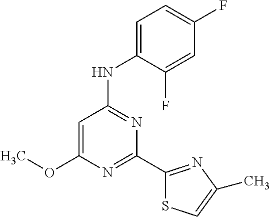 |
| IN10964-084-P1 | 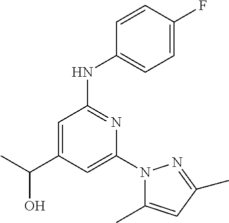 [SMILES: Cc1cc(C)n(-c2cc(C(C)O)cc(Nc3ccc(F)cc3)n2)n1] |
| IN10973-007-P1 | 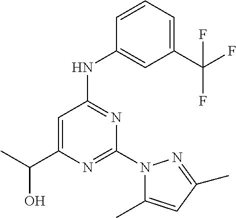 [SMILES: Cc1cc(C)n(-c2nc(Nc3cccc(C(F)(F)F)c3)cc(C(C)O)n2)n1] |
| IN10973-006-P1 | 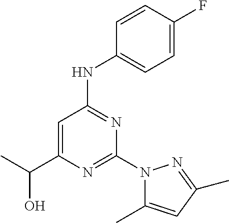 [SMILES: Cc1cc(C)n(-c2nc(Nc3ccc(F)cc3)cc(C(C)O)n2)n1] |
| IN10880-094-P1 | 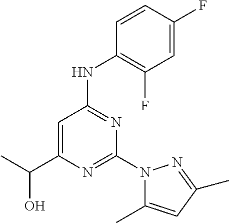 [SMILES: Cc1cc(C)n(-c2nc(Nc3ccc(F)cc3F)cc(C(C)O)n2)n1] |
| IN10880-092-P1 | 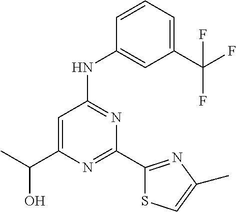 [SMILES: Cc1csc(-c2nc(Nc3cccc(C(F)(F)F)c3)cc(C(C)O)n2)n1] |
| IN10880-091-P1 | 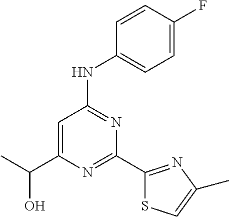 [SMILES: Cc1csc(-c2nc(Nc3ccc(F)cc3)cc(C(C)O)n2)n1] |
| IN10864-077-P1 | 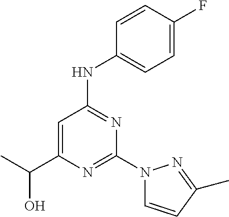 [SMILES: Cc1ccn(-c2nc(Nc3ccc(F)cc3)cc(C(C)O)n2)n1] |
| IN10864-081-P1 | 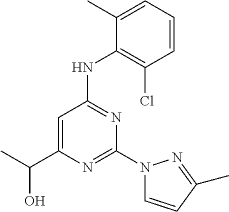 [SMILES: Cc1ccn(-c2nc(Nc3c(C)cccc3Cl)cc(C(C)O)n2)n1] |
| IN10881-055-P1 | 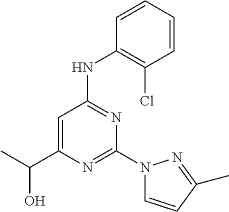 [SMILES: Cc1ccn(-c2nc(Nc3ccccc3Cl)cc(C(C)O)n2)n1] |
| IN10882-054-P1 | 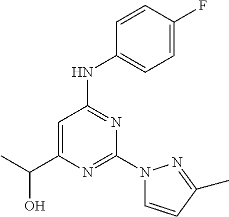 [SMILES: Cc1ccn(-c2nc(Nc3ccc(F)cc3)cc(C(C)O)n2)n1] |
| IN10876-038-P1 | 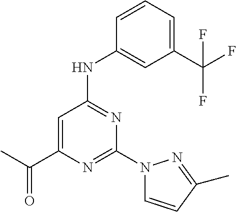 [SMILES: CC(=O)c1cc(Nc2cccc(C(F)(F)F)c2)nc(-n2ccc(C)n2)n1] |
| IN10876-033-P1 | 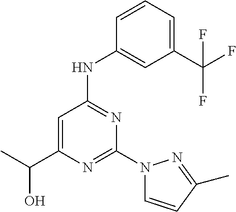 [SMILES: Cc1ccn(-c2nc(Nc3cccc(C(F)(F)F)c3)cc(C(C)O)n2)n1] |
| NSSy5713 | 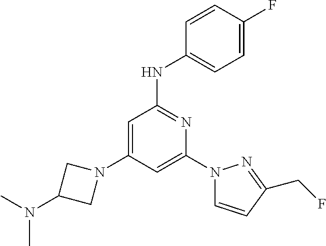 [SMILES: CN(C)C1CN(c2cc(Nc3ccc(F)cc3)nc(-n3ccc(CF)n3)c2)C1] |
| NSSy5701 | 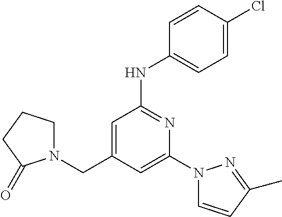 [SMILES: Cc1ccn(-c2cc(CN3CCCC3=O)cc(Nc3ccc(Cl)cc3)n2)n1] |
| NSSy5632 | 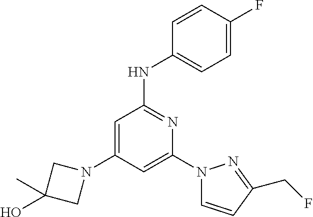 [SMILES: CC1(O)CN(c2cc(Nc3ccc(F)cc3)nc(-n3ccc(CF)n3)c2)C1] |

### 4. Uses, Formulation and Administration

Pharmaceutically Acceptable Compositions

According to another embodiment, this disclosure provides a composition comprising a compound described herein or a pharmaceutically acceptable derivative thereof and a pharmaceutically acceptable carrier, adjuvant, or vehicle. The amount of compound in compositions is such that is effective to measurably modulate potassium channels in a biological sample or in a patient.

In certain embodiments, a composition described herein is formulated for administration to a patient in need of such composition. In some embodiments, a composition described herein is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Pharmaceutically acceptable compositions described herein may also be prepared in injectable form. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

The amount of compounds described herein that may be combined with the carrier materials to produce a composition in a single dosage form will vary depending upon the host treated and the particular mode of administration. In some embodiments, provided compositions should be formulated so that a dosage of between 0.01-100 mg/kg body weight/day of the inhibitor, such as e.g., 0.1-100 mg/kg body weight/day, can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

Uses of Compounds and Pharmaceutically Acceptable Compositions

In some embodiments, compounds and compositions described herein are useful in treating diseases and/or disorders associated with the activity of potassium channels. Such diseases and/or disorders include e.g., neurodegenerative and neurological conditions (e.g., Parkinson's disease, Alzheimer's disease, dementia, amyotrophic lateral sclerosis (ALS) ataxia, anxiety, depression, mood disorders, memory and attention deficits, bipolar disorder, psychosis, schizophrenia, traumatic brain injury, tremors, and narcolepsy), heart disease and realted conditions (e.g., ischaemic heart disease, coronary heart disease, angina pectoris, and coronary artery spasms), metabolic disease and bladder diseases (e.g., bladder spasms, urinary incontinence, bladder outflow obstruction, gastrointestinal dysfunction, irritable bowel syndrome, and diabetes), withdrawal symptoms associated with termination of addiction, and other conditions associated with the modulation of potassium channels such as e.g., respiratory diseases, epilepsy, convulsions, seizures, absence seizures, vascular spasms, renal disorders (e.g., polycystic kidney disease), erectile dysfunction, secretory diarrhoea, ischaemia, cerebral ischaemia, dysmenorrhea, Reynaud's disease, intermittent claudication, Sjorgren's syndrome, arrhythmia, hypertension, myotonic muscle dystrophia, spasticity, xerostomi, hyperinsulinemia, premature labour, baldness, cancer, immune suppression, migraine and pain.

In some embodiments, the present disclosure provides a method of treating a disease or condition selected from a neurodegenerative disease, dementia, heart disease, withdrawal symptoms associated with termination of addiction, metabolic disease, and bladder disease. In other embodiments, the present disclosure provides a method of treating a disease or condition selected from ataxia, dystonia, Parkinson's disease, ischemia, traumatic brain injury, amyotrophic lateral sclerosis, hypertension, atherosclerosis, diabetes, arrhythmia, over-active bladder, and withdrawal symptoms caused by the termination of abuse of alcohol and other drugs of abuse.

In one aspect, the present disclosure provides a method of modulating the activity of a potassium channel in a subject comprising the step of administering a compound of Formula I or Ia, or a composition comprising any of the compounds herein. In another embodiment, the present disclosure provides a method of positively modulating a SK2 channel in a cell comprising the step of contacting the cell with a compound of Formula I or Ia, or a composition comprising any of the compounds herein.

Certain exemplary provided compounds, e.g., having structural formula I and Ia are set forth in the EXEMPLIFICATION section below. In some embodiments, a provided compound is one or more compounds selected from those exemplified in the EXEMPLIFICATION section below, or a pharmaceutically acceptable salt thereof.

### EXEMPLIFICATION

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples that follow and the references to the scientific and patent literature cited herein. It should further be appreciated that the contents of those cited references are incorporated herein by reference to help illustrate the state of the art.

As depicted in the Examples below, in certain exemplary embodiments, compounds are prepared according to the following general procedures. It will be appreciated that, although the synthetic methods and Schemes depict the synthesis of certain compounds of the present invention, the following methods and other methods known to one of ordinary skill in the art can be applied to all compounds and subclasses and species of each of these compounds, as described herein.

General Synthetic Scheme:

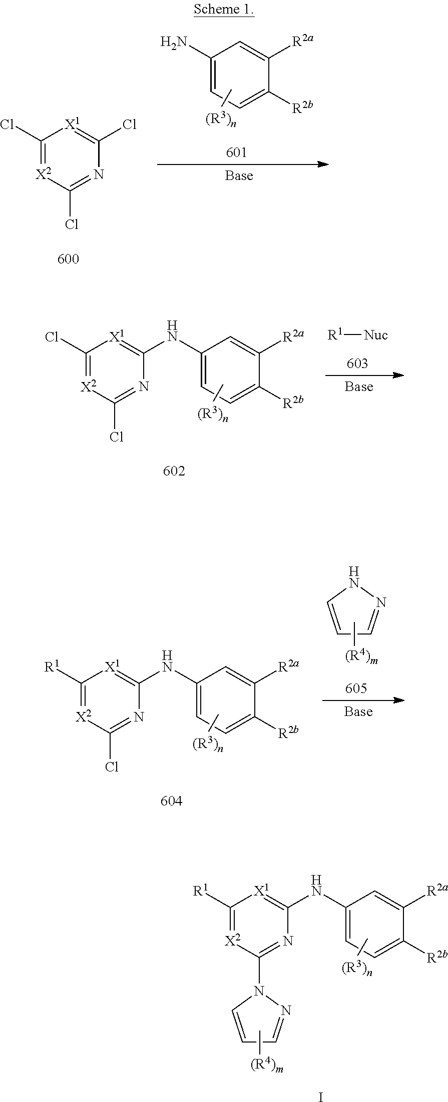

In one aspect, compounds of Formula I can be prepared according to Scheme 1, where the variables R¹, R³, R⁵, R^{4a}, R^{4b}, X¹, X², and A are defined for Formula I. For example, compounds of Formula I can be prepared by reacting a compound of Formula 600 with a compound of Formula 601 in the presence of base, such as, e.g., cesium carbonate to form intermediate 602. Intermediate 602 is then reacted with a compound of Formula 603, where Nuc is a nucleophile, such as, e.g., NH, NH₂, OH, etc., in the presence of base, such as, e.g., diisopropylethylamine to give an intermediate of the Formula 604. Treatment of 604 in the presence of 605 at e.g., elevated temperature with base, such as, e.g., cesium carbonate gives a compound of Formula I. Scheme 1 is in no way limiting and represents only one method by which certain compounds described herein can be made. Other methods of making compounds of Formula I would be apparent to one of skill in the art.

### Preparation of Compounds of Formula I

Compounds of Formula I and Ia were prepared according to the general procedures outlined below.

### Example 1

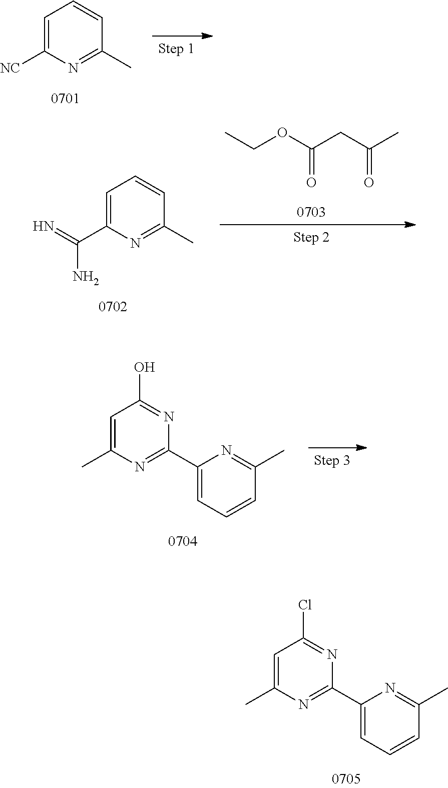

Step 1[0702]: To a pre cooled (−78° C.) solution of 6-methyl-2-pyridinecarbonitrile [0701] (5 g, 42.3 mmol) in tetrahydrofuran 50 mL) was added lithium bis(trimethylsilyl)amide (14.1 g, 84.6 mmol). The reaction mixture was slowly warmed to rt and stirred for 16 h. After the completion of the reaction, the reaction mixture was quenched with 1.5N hydrochloric acid and then washed with ethyl acetate to remove the amide formed. Then the resultant aqueous layer was taken as such taken for next step. MS(M+1)⁺=136.

Step 2[0704]: To the aqueous solution of Step 1 [0702] in ethanol (50 mL), was added sodium hydroxide (3.37 g, 84.33 mmol). Then the reaction mixture was stirred at rt for 16 h. After the completion of the reaction, the reaction mixture was concentrated under reduced pressure. The resulting aqueous layer was extracted with ethyl acetate (2×100 mL), the combined organic layer was dried over sodium sulfate and concentrated to afford 6-methyl-2-(6-methylpyridin-2-yl)pyrimidin-4-ol [0704] as a brownish gum (4 g), MS(M+1)+=202.

Step 3[0705]: To a solution of 6-methyl-2-(6-methylpyridin-2-yl)pyrimidin-4-ol [0704] (4 g, 19.88 mmol) in toluene (25 mL), was added phosphorus oxychloride (6.09 g, 39.7 mmol). Then the reaction mixture was heated at 110° C. for 16 h. The reaction mixture was quenched with saturated bicarbonate solution and extracted with ethyl acetate (2×75 mL), the combined organic layer was dried over sodium sulfate and concentrated to afford 4-chloro-6-methyl-2-(6-methylpyridin-2-yl)pyrimidine [0705] as a brownish gum (2.5 g). MS(M+1)+=220.

### Example 2

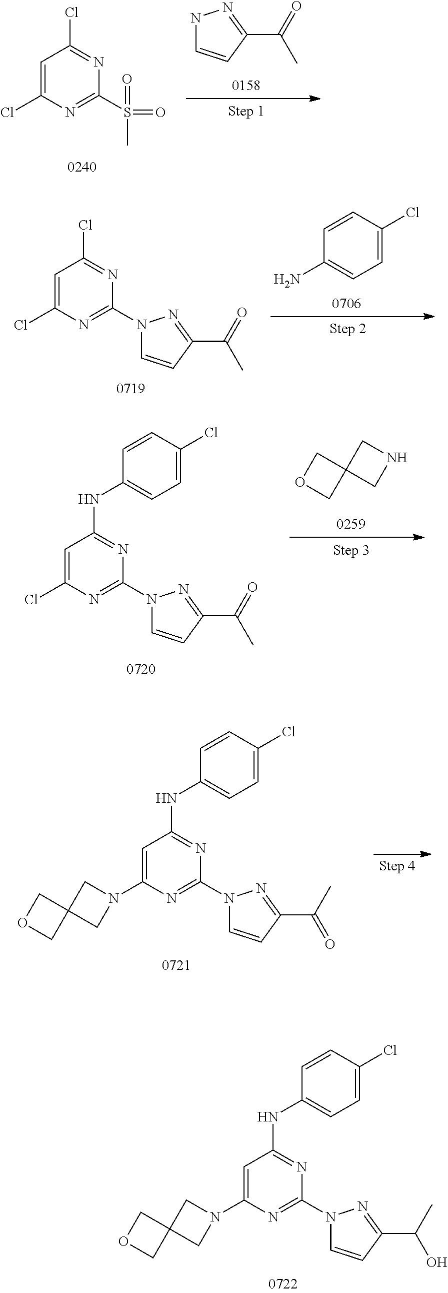

Step 1[0719]: To a suspension of sodium hydride (0.704 g, 17.61 mmol) in dry dichloromethane (20 mL) was added 1-(1h-pyrazol-3-yl)ethan-1-onehydrochloride [0158] (1.29 g, 8.8 mmol) portion wise under N2 atm. The reaction mixture was stirred at rt for 30 min, then cooled to −78° C. and followed by 4,6-dichloro-2-(methylsulfonyl)pyrimidine [0240] (2 g, 8.80 mmol) in dichloromethane was added drop wise. After addition the reaction mixture was stirred at −78° C. for 3 h. The reaction mixture was quenched with ice cold water (25 mL), extracted with dichloromethane (2×50 mL). The combined organic extracts were dried over sodium sulfate, filtered and concentrated under reduced pressure to afford 1.8 g of 1-(1-(4,6-dichloropyrimidin-2-yl)-1H-pyrazol-3-yl)ethan-1-one [0719] as an off-white solid. MS(M+1)+=257.1/259.1.

Step 2[0720]: To a stirred solution of 1-(1-(4,6-dichloropyrimidin-2-yl)-1H-pyrazol-3-yl)ethan-1-one [0719] (1.8 g, 7.00 mmol) in acetonitrile (20 mL), was added 4-chloroaniline [0706] (0.893 g, 7.00 mmol) and N, N-di-isopropyl ethylamine (1.46 mL, 8.4 mmol). The reaction mixture was heated at 75° C. for 16 h. The reaction mixture got almost solidified and the solid was filtered and dried by suction to afford 1.1 g of 1-(1-(4-chloro-6-((4-chlorophenyl) amino) pyrimidin-2-yl)-1H-pyrazol-3-yl) ethan-1-one [0720] as an off-white solid. MS(M+1)+=348.1/349.1.

Step 3[0721]: To a stirred solution of 1-(1-(4-chloro-6-((4-chlorophenyl) amino) pyrimidin-2-yl)-1H-pyrazol-3-yl) ethan-1-one [0720] (0.5 g, 1.436 mmol) in acetonitrile (10 mL), was added 2-oxa-6-azaspiro (3, 3) heptane [0259] (0.142 g, 1.436 mmol) and cesium carbonate (0.701 g, 2.154 mmol). The reaction mixture was irradiated in microwave at 110° C. for 2 h. The reaction mixture was filtered; then the filtrate was concentrated under reduced pressure to afford crude product which was purified by column chromatography using 51% ethyl acetate in pet ether as solvent to afford 0.070 g of 1-(1-(4-((4-chlorophenyl)amino)-6-(2-oxa-6-azaspiro[3.3]heptan-6-yl) pyrimidin-2-yl)-1H-pyrazol-3-yl)ethan-1-one [0721] as an off-white solid. MS(M+1)+=411.0/411.7.

Step 4[0722] To a stirred solution of 1-(1-(4-((4-chlorophenyl)amino)-6-(2-oxa-6-azaspiro[3.3]heptan-6-yl) pyrimidin-2-yl)-1H-pyrazol-3-yl)ethan-1-one [0721] (0.070 g, 0.174 mmol) in methanol (3 mL), was added sodium borohydride (0.013 g, 0.340 mmol) at 0° C. The reaction mixture was allowed to stir at rt for 2 h. The reaction mixture was quenched with ice, and then the reaction mixture was concentrated under reduced pressure to afford crude product which was purified by column chromatography using 6% methanol in chloroform as solvent to afford 0.030 g of 1-(1-(4-((4-chlorophenyl)amino)-6-(2-oxa-6-azaspiro[3.3]heptan-6-yl)pyrimidin-2-yl)-1H-pyrazol-3-yl)ethan-1-ol [0722], Compound 150 as an off-white solid. MS(M+1)+=413.2/416.2. 1H NMR (400 MHz, DMSO-d6) δ 9.49 (s, 1H), 8.39 (d, J=2.7 Hz, 1H), 7.70-7.67 (d, J=8.84 Hz, 2H), 7.36-7.34 (d, J=8.84 Hz, 2H), 6.47 (d, J=2.5 Hz, 1H), 5.48 (s, 1H), 5.23 (d, J=4.8 Hz, 1H), 4.81-4.78 (m, 1H), 4.74 (s, 4H), 4.21 (s, 4H), 1.41 (d, J=6.5 Hz, 3H).

### Example 3

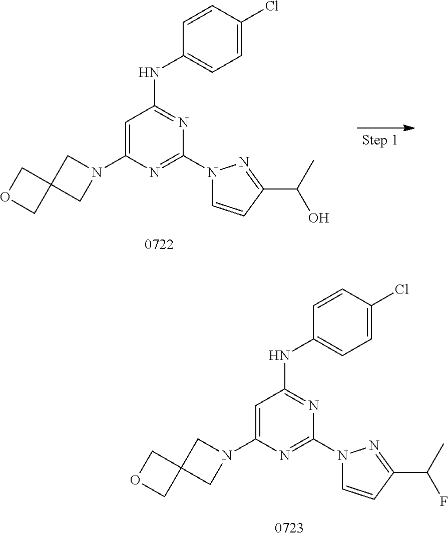 [SMILES: CC(O)c1ccn(-c2nc(Nc3ccc(Cl)cc3)cc(N3CC4(COC4)C3)n2)n1 ; CC(F)c1ccn(-c2nc(Nc3ccc(Cl)cc3)cc(N3CC4(COC4)C3)n2)n1]

Step 1[0723]: To a stirred solution of 1-(1-(4-((4-chlorophenyl)amino)-6-(2-oxa-6-azaspiro[3.3] heptan-6-yl)pyrimidin-2-yl)-1H-pyrazol-3-yl)ethan-1-ol [0722] (0.15 g, 0.363 mmol) in dichloromethane (5 mL), was added diethyl aminosulphurtrifluoride (0.117 g, 0.726 mmol) at 0° C. The reaction mixture was stirred at rt for 2 h, quenched with 10% sodium bicarbonate solution (2 mL) at 0° C. and extracted with dichloromethane (20 mL). The organic extracts was dried over sodium sulfate, filtered and concentrated under reduced pressure to afford crude product which was purified by column chromatography using 25% ethyl acetate in pet ether as solvent to afford 0.055 g of N-(4-chlorophenyl)-2-(3-(1-fluoroethyl)-1H-pyrazol-1-yl)-6-(2-oxa-6-azaspiro[3.3]heptan-6-yl)pyrimidin-4-amine [0723], Compound 152 as an off-white solid. MS(M+1)+=415.0/416.0. 1H-NMR (400 MHz, DMSO-d6): δ 9.53 (s, 1H), 8.47 (d, J=2.56 Hz, 1H), 7.67 (d, J=8.88 Hz, 2H), 7.35 (d, J=1.92 Hz, 2H), 6.64 (d, J=2.4 Hz, 1H), 5.77 (dq, J_{F}=48.0 Hz, J=6.52 Hz, 1H), 5.50 (s, 1H), 4.73 (s, 4H), 4.21 (s, 4H), 1.67 (dd, J=6.44, J_{F}=23.90 Hz, 3H).

### Example 4

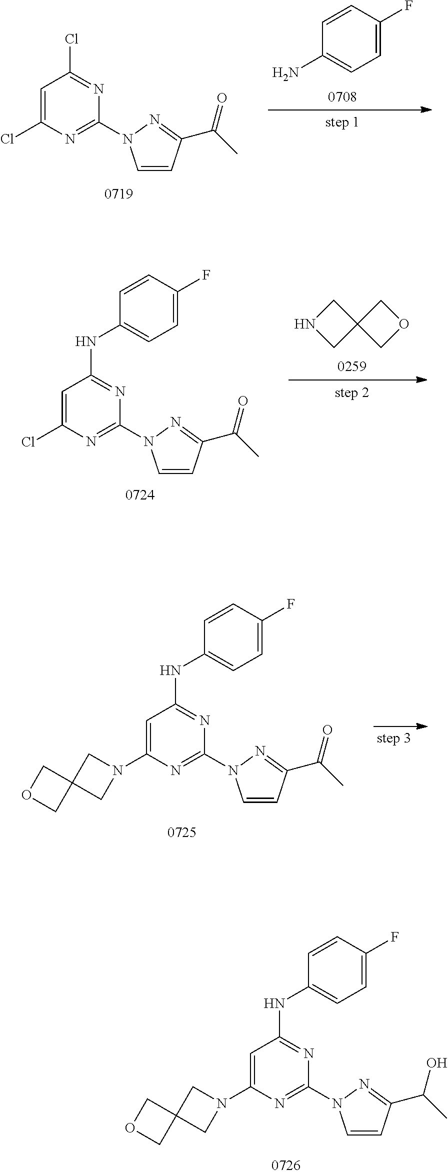 [SMILES: CC(=O)c1ccn(-c2nc(Cl)cc(Cl)n2)n1 ; Nc1ccc(F)cc1 ; CC(=O)c1ccn(-c2nc(Cl)cc(Nc3ccc(F)cc3)n2)n1 ; C1NCC12COC2 ; CC(=O)c1ccn(-c2nc(Nc3ccc(F)cc3)cc(N3CC4(COC4)C3)n2)n1 ; CC(O)c1ccn(-c2nc(Nc3ccc(F)cc3)cc(N3CC4(COC4)C3)n2)n1]

Step 1[0724]: The procedure is similar to step 2[0720] in example 2, 2 g of 1-(1-(4,6-dichloropyrimidin-2-yl)-1H-pyrazol-3-yl)ethan-1-one [0719] and 4-fluoroaniline [0708] (0.87 g, 7.77 mmol) gave 2.2 g of 1-(1-(4-chloro-6-((4-fluorophenyl)amino)pyrimidin-2-yl)-1H-pyrazol-3-yl) ethan-1-one [0724] as an off-white solid. MS(M+1)+=332.1/333.1.

Step 2[0725]: The procedure is similar to step 3 [0721] in example 2. 0.7 g of 1-(1-(4-chloro-6-((4-fluorophenyl)amino)pyrimidin-2-yl)-1H-pyrazol-3-yl) ethan-1-one [0724] and 2-oxa-6-azaspiro(3,3) heptane [0259] (0.25 g, 2.53 mmol) gave 0.180 g of 1-(1-(4-((4-fluorophenyl)amino)-6-(2-oxa-6-azaspiro [3.3]heptan-6-yl)pyrimidin-2-yl)-1H-pyrazol-3-yl)ethan-1-one [0725] as an off-white solid. MS(M+1)+=394.9/395.7.

Step 3[0726]: The procedure is similar to the step 4[0722] in example 2. 0.150 g of 1-(1-(4-((4-fluorophenyl)amino)-6-(2-oxa-6-azaspiro [3.3]heptan-6-yl)pyrimidin-2-yl)-1H-pyrazol-3-yl)ethan-1-one [0725] gave 0.038 g of 1-(1-(4-((4-fluorophenyl)amino)-6-(2-oxa-6-azaspiro[3.3]heptan-6-yl)pyrimidin-2-yl)-1H-pyrazol-3-yl)ethan-1-ol [0726], Compound 153 as a white solid. MS(M+1)+=397.2/398.2. 1H-NMR (400 MHz, DMSO-d6): δ 9.37 (s, 1H), 8.38 (d, J=2.4 Hz, 1H), 7.64-7.61 (m, 2H), 7.17-7.13 (m, 2H), 6.46 (d, J=2.4 Hz, 1H), 5.43 (s, 1H), 5.23 (d, J=5.2 Hz, 1H), 4.82-4.76 (m, 1H), 4.73 (s, 4H), 4.19 (s, 4H), 1.40 (d, J=6.4 Hz, 3H).

### Example 5

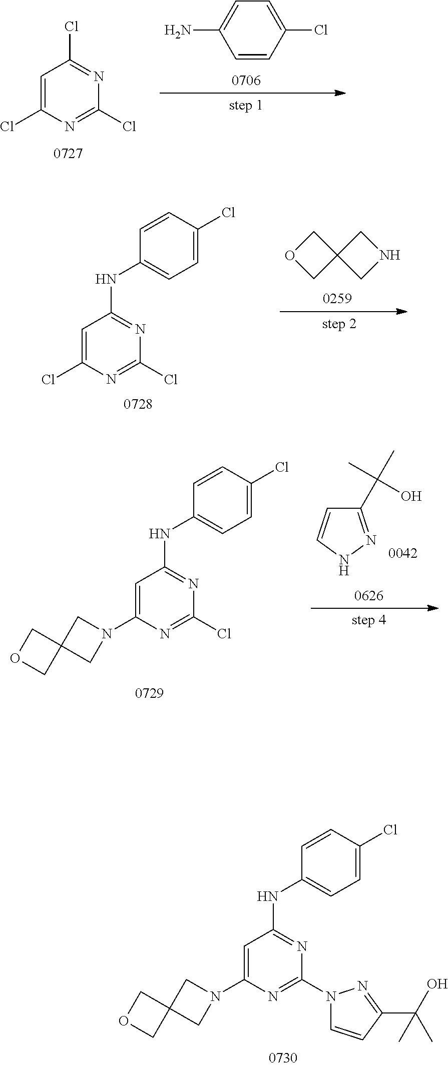

Step 1[0728]: To a stirred solution of 2, 4, 6-trichloropyrimidine [0727] (2 g, 10.903 mmol) in ethanol (20 mL), was added 4-chloroaniline [0706] (1.4 g, 10.903 mmol) and cesium carbonate (3.9 g, 11.994 mmol). The reaction mixture was stirred at rt for 16 h. The reaction mixture was filtered to remove cesium carbonate, then the filtrate was concentrated under reduced pressure to afford crude product which was purified by column chromatography using 6% ethyl acetate in pet ether as solvent to afford 1.3 g of 2,6-dichloro-N-(4-chlorophenyl)pyrimidin-4-amine [0728] as a pale brown solid. MS(M+1)+=274.1.

Step 2[0729]: 0.800 g of 2,6-dichloro-N-(4-chlorophenyl)pyrimidin-4-amine [0728] and 2-oxa-6-azaspiro(3,3) heptane [0259] (0.28 g, 2.914 mmol) gave 0.1 g of 2-chloro-N-(4-chlorophenyl)-6-(2-oxa-6-azaspiro[3.3]heptan-6-yl)pyrimidin-4-amine [0729] as an off-white solid [using DIPEA/ACN, 80° C. for 16 h]. MS(M+1)+=337.2/338.2.

Step 3[0730]: To a stirred solution of 2-chloro-N-(4-chlorophenyl)-6-(2-oxa-6-azaspiro[3.3]heptan-6-yl)pyrimidin-4-amine [0729] (0.090 g, 0.2669 mmol) in acetonitrile (2.5 mL), was added 2-(1h-pyrazol-3-yl)propan-2-ol [0626] (0.040 g, 0.320 mmol) and cesium carbonate (0.130 g, 0.4 mmol). The reaction mixture was irradiated in microwave at 100° C. for 2 h. The reaction mixture was filtered to remove cesium carbonate, then the filtrate was concentrated under reduced pressure to afford crude product which was purified by column chromatography using 85% ethyl acetate in pet ether as solvent to afford 0.031 g of 2-(1-(4-((4-chlorophenyl)amino)-6-(2-oxa-6-azaspiro [3.3]heptan-6-yl)pyrimidin-2-yl)-1H-pyrazol-3-yl)propan-2-ol of [0730], Compound 147 as a pale yellow solid. MS(M+1)+=426.9/427.6. 1H NMR (400 MHz, DMSO-d6) δ 9.48 (s, 1H), 8.35 (t, J=2.1 Hz, 1H), 7.75-7.72 (d, J=8.8 Hz, 2H), 7.32-7.30 (d, J=8.8 Hz, 2H), 6.48-6.48 (d, J=4 Hz, 1H), 5.47 (s, 1H), 5.05 (s, 1H), 4.73 (s, 4H), 4.20 (s, 4H), 1.49 (s, 6H).

### Example 6

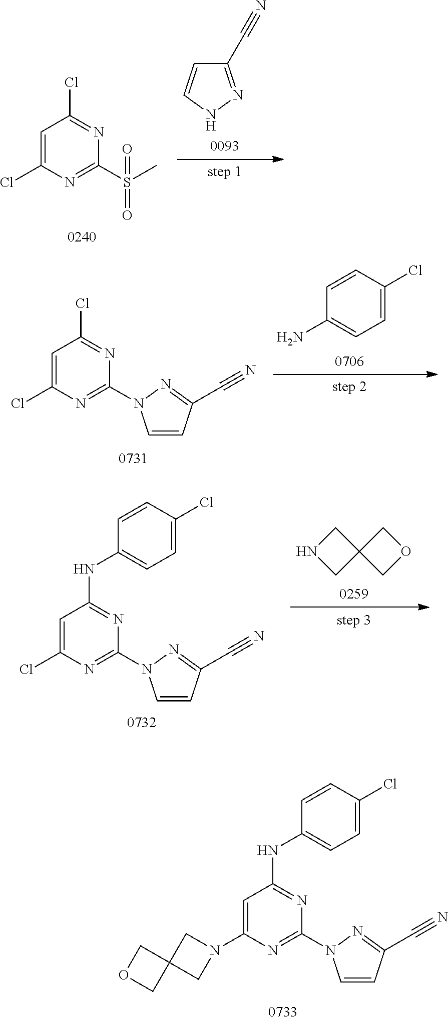

Step 1[0731]: The procedure is similar to step 1[0719] in example 2. 2 g of 4,6-dichloro-2-(methylsulfonyl)pyrimidine [0240] and 1H-pyrazole-3-carbonitrile [0093] (0.819 g, 8.80 mmol) gave 1.9 g of 1-(4,6-dichloropyrimidin-2-yl)-1h-pyrazole-3-carbonitrile [0731] as a white solid. MS(M+1)+=240.0/241.1.

Step 2[0732]: The procedure is similar to step 2[0720] in example 2. 1.9 g of 1-(4,6-dichloropyrimidin-2-yl)-1h-pyrazole-3-carbonitrile [0731] and 4-chloroaniline [0706] (1.0 g, 7.914 mmol) gave 2 g of 1-(4-chloro-6-((4-chlorophenyl)amino)pyrimidin-2-yl)-1h-pyrazole-3-carbonitrile [0732] as a pale yellow solid. MS(M+1)+=332.0/333.0.

Step 3[0733]: The procedure is similar to step 3 [0721] in example 2. 0.5 g of 1-(4-chloro-6-((4-chlorophenyl)amino)pyrimidin-2-yl)-1h-pyrazole-3-carbonitrile [0732] and 2-oxa-6-azaspiro(3,3) heptane (0.14 g, 1.5 mmol) gave 0.280 g of 1-(4-((4-chlorophenyl)amino)-6-(2-oxa-6-azaspiro[3.3]heptan-6-yl)pyrimidin-2-yl)-1h-pyrazole-3-carbonitrile [0733], Compound 143 as an off-white solid. MS(M+1)+=394.3/395.3. 1H NMR (400 MHz, DMSO-d6) δ 9.64 (s, 1H), 8.69 (d, J=2.7 Hz, 1H), 7.63 (d, J=8.76 Hz, 2H), 7.38 (d, J=8.76 Hz, 2H), 7.21 (d, J=2.7 Hz, 1H), 5.57 (s, 1H), 4.74 (s, 4H), 4.24 (s, 4H).

### Example 7

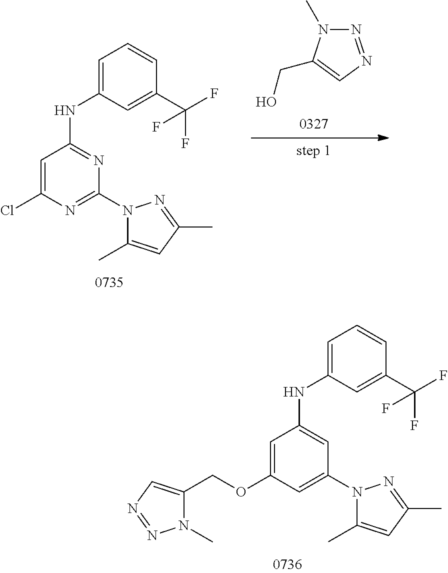

Step 1[0736]: 0.3 g of 6-chloro-2-(3,5-dimethyl-1H-pyrazol-1-yl)-N-(3-(trifluoromethyl)phenyl)pyrimidin-4-amine [0735] and (1-methyl-1H-1,2,3-triazol-5-yl)methanol [0327] (0.184 g, 1.63 mmol) in 50% aq. sodium hydroxide solution (2 mL) was added tetrabutyl ammonium hydrogen Sulfate (0.11 g, 0.35 mmol). The reaction mixture was heated at 75° C. in a closed vial for 16 h. After the completion of the reaction, the reaction mixture was extracted with ethyl acetate (2×40 mL), the combined organic layer was dried over anhydrous sodium sulfate and concentrated to afford crude and which was purified by column of silica gel (60-120 mesh), using 25% ethyl acetate in hexane as eluent to afford 0.3 g of 2-(3,5-dimethyl-1H-pyrazol-1-yl)-6-((1-methyl-1H-1,2,3-triazol-5-yl)methoxy)-N-(3-(trifluoromethyl) phenyl)pyrimidin-4-amine [0736], Compound 201 as a white solid. MS(M+1)+=445.2. 1H NMR (400 MHz, DMSO-d6) δ 10.02 (s, 1H), 8.59 (s, 1H), 7.88 (s, 1H), 7.69 (d, J=8.8 Hz, 1H), 7.53 (t, J=8.0 Hz, 1H), 7.34 (d, J=8.2 Hz, 1H), 6.15 (s, 1H), 6.05 (s, 1H), 5.56 (s, 2H), 4.10 (s, 3H), 2.58 (s, 3H), 2.21 (s, 3H).

### Example 8

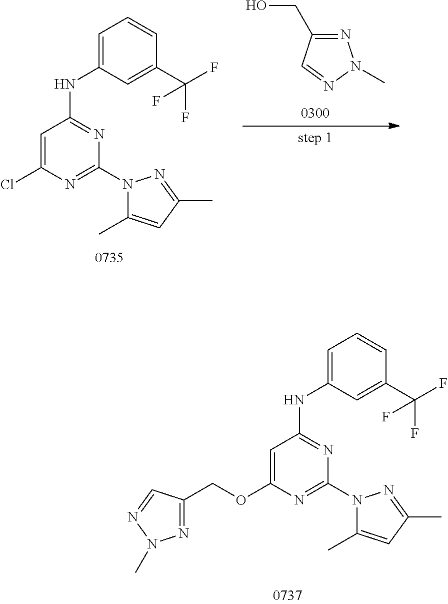

Step 1[0737]: 0.3 g of 6-chloro-2-(3,5-dimethyl-1H-pyrazol-1-yl)-N-(3-(trifluoromethyl)phenyl)pyrimidin-4-amine [0735] and (2-methyl-2H-1,2,3-triazol-4-yl)methanol [0300] (0.184 g, 1.63 mmol) in 50% aq. sodium hydroxide solution (2 mL) was added tetrabutyl ammonium hydrogen Sulfate (0.11 g, 0.35 mmol). The reaction mixture was heated at 75° C. in a closed vial for 16 h. After the completion of the reaction, the reaction mixture was extracted with ethyl acetate (2×40 mL), the combined organic layer was dried over anhydrous sodium sulfate and concentrated to afford crude and which was purified by column of silica gel (60-120 mesh), using 25% ethyl acetate in hexane as eluent to afford 0.072 g of 2-(3,5-dimethyl-1H-pyrazol-1-yl)-6-((2-methyl-2H-1,2,3-triazol-4-yl)methoxy)-N-(3-(trifluoromethyl) phenyl) pyrimidin-4-amine [0737], Compound 199 as white solid. MS(M+1)+=445.2. 1H NMR (400 MHz, DMSO-d6) δ 9.99 (s, 1H), 8.64 (s, 1H), 7.94 (s, 1H), 7.68 (d, J=8.8 Hz, 1H), 7.53 (t, J=8.0 Hz, 1H), 7.33 (d, J=7.8 Hz, 1H), 6.14 (s, 1H), 6.02 (s, 1H), 5.45 (s, 2H), 4.14 (s, 3H), 2.59 (s, 3H), 2.21 (s, 3H).

### Example 9

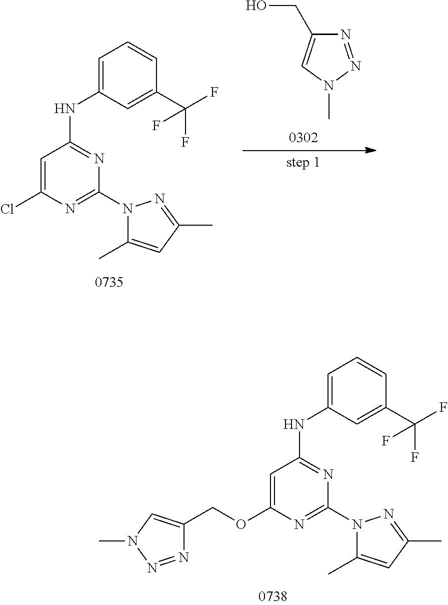

Step 1[0738]: 0.4 g 6-chloro-2-(3,5-dimethyl-1H-pyrazol-1-yl)-N-(3-(trifluoromethyl)phenyl)pyrimidin-4-amine [0735] and (1-methyl-1H-1,2,3-triazol-4-yl)methanol [0302] (0.24 g, 2.175 mmol) in 50% aq. sodium hydroxide solution (2 mL) was added tetrabutyl ammonium hydrogen Sulfate (0.11 g, 0.35 mmol). The reaction mixture was heated at 75° C. in a closed vial for 16 h. After the completion of the reaction, the reaction mixture was extracted with ethyl acetate (2×40 mL), the combined organic layer was dried over anhydrous sodium sulfate and concentrated to afford crude and which was purified by column of silica gel (60-120 mesh), using 25% ethyl acetate in hexane as eluent to afford 0.015 g of 2-(3,5-dimethyl-1H-pyrazol-1-yl)-6-((1-methyl-1H-1,2,3-triazol-4-yl)methoxy)-N-(3-(trifluoromethyl) phenyl) pyrimidin-4-amine [0738], Compound 197 as white solid. MS(M+1)+=445.2. 1H NMR (400 MHz, DMSO-d6) δ 9.99 (s, 1H), 8.65 (s, 1H), 8.33 (s, 1H), 7.74 (d, J=7.7 Hz 1H), 7.54 (t, J=7.9 Hz, 1H), 7.34 (d, J=7.7 Hz, 1H), 6.15 (s, 1H), 6.02 (s, 1H), 5.46 (s, 2H), 4.05 (s, 3H), 2.61 (s, 3H), 2.23 (s, 3H).

### Example 10

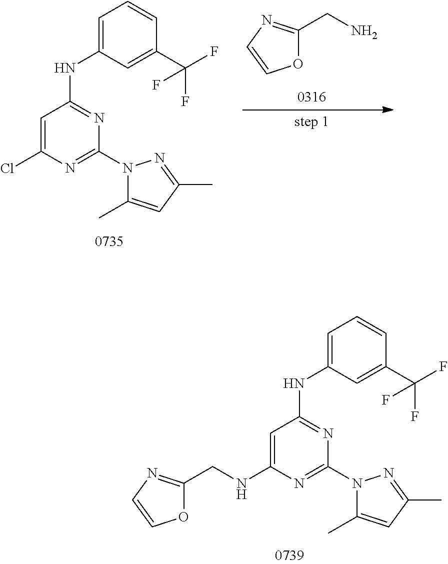

Step1[0739]: To a solution of 6-chloro-2-(3,5-dimethyl-1H-pyrazol-1-yl)-N-(3-(trifluoromethyl)phenyl)pyrimidin-4-amine [0735] (0.2 g, 0.543 mmol) in acetonitrile (8 mL), was added oxazol-2-yl-methylamine [0316] (0.106 g, 1.08 mmol) and N,N-diisopropyl ethylamine (0.14 g, 1.08 mmol). The reaction mixture was heated at 180° C. under MW condition for 4 h. After the completion of the reaction, the reaction mixture was concentrated to afford crude product which was purified by Prep HPLC to afford 2-(3,5-dimethyl-1H-pyrazol-1-yl)-N4-(oxazol-2-ylmethyl)-N6-(3-(trifluoromethyl)phenyl) pyrimidine-4,6-diamine [0739], Compound 195 as an light brown solid (35 g). MS(M+1)+=430, 1H NMR (400 MHz, DMSO-d6) δ 9.61 (s, 1H), 8.62 (s, 1H), 8.05 (s, 1H), 7.93 (s, 1H), 7.68 (d, J=8.3 Hz, 1H), 7.48 (t, J=7.8 Hz, 1H), 7.25 (d, J=7.6 Hz, 1H), 7.16 (s, 1H), 6.04 (s, 1H), 5.85 (s, 1H), 4.64 (s, 2H), 2.44 (s, 3H), 2.17 (s, 3H).

### Example 11

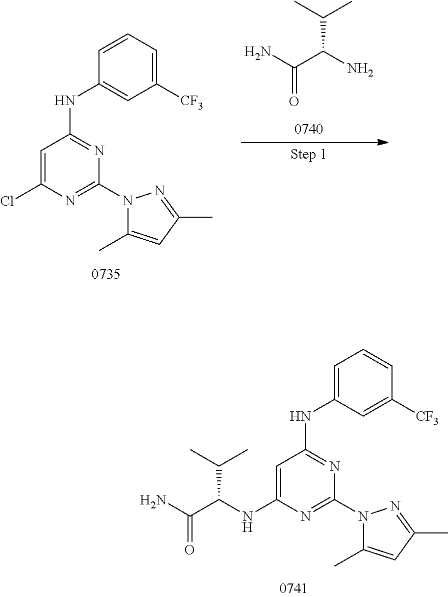

Step 1[0741]: To a solution of 0.4 g of 6-chloro-2-(3,5-dimethyl-1H-pyrazol-1-yl)-N-(3-(trifluoromethyl)phenyl)pyrimidin-4-amine [0735] in acetonitrile was added L-valinamide hydrochloride [0740] (0.332 g, 2.175 mmol) and N,N-diisopropyl ethylamine. The reaction mixture was heated at 180° C. under microwave for 5 h. The reaction mixture was concentrated under reduced pressure to afford crude product which was purified by column chromatography using 40% ethyl acetate in pet ether to afford gave 0.128 g of (S)-2-((2-(3,5-dimethyl-1H-pyrazol-1-yl)-6-((3-(trifluoromethyl)phenyl)amino)pyrimidin-4-yl)amino)-3-methylbutanamide [0741], Compound 200 as white solid. MS(M+1)+=448.2. 1H NMR (400 MHz, DMSO-d6) δ 9.53 (s, 1H), 8.48 (s, 1H), 7.71 (d, J=8.3 Hz, 1H), 7.48 (t, J=8.0 Hz, 1H), 7.39 (s, 1H), 7.24 (d, J=7.7 Hz, 2H), 7.06 (bs, 1H), 6.05 (s, 1H), 5.98 (bs, 1H), 4.40 (bs, 1H), 2.54 (s, 3H), 2.18 (s, 3H), 2.15-2.05 (m, 1H), 0.94 (d, J=6.9 Hz, 6H).

### Example 12

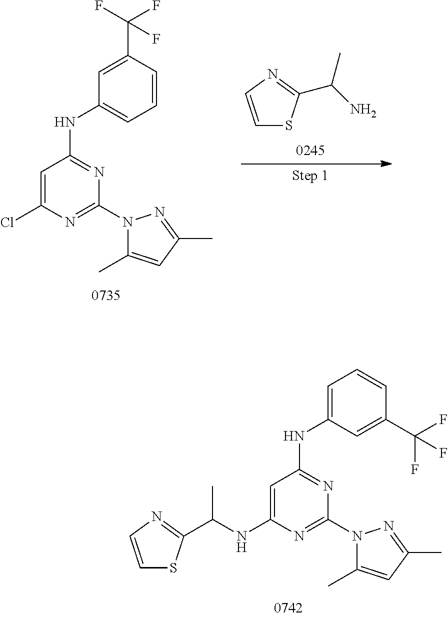

Step 1[0742]: To a solution of 6-chloro-2-(3,5-dimethyl-1H-pyrazol-1-yl)-N-(3-(trifluoromethyl) phenyl)pyrimidin-4-amine [0735] (0.25 g, 0.679 mmol) in acetonitrile (2 mL) was added 1-thiazol-2-yl-ethylamine [0245] (0.174 g, 1.359 mmol) and N,N-diisopropyl ethylamine (0.177 mL, 1.01 mmol). The reaction mixture was heated at 180° C. under microwave condition for 3 h. The reaction mixture was concentrated under reduced pressure to afford crude product which was purified by preparative HPLC to afford 0.1 g of 2-(3,5-dimethyl-1H-pyrazol-1-yl)-N4-(1-(thiazol-2-yl)ethyl)-N6-(3-(trifluoromethyl)phenyl)pyrimidine-4,6-diamine [0742], Compound 187 as a yellow solid. MS(M+1)+=460.2. 1H-NMR (400 MHz, DMSO-d6): δ 9.61 (s, 1H), 8.60 (bs, 1H), 8.03 (d, J=7.20 Hz, 1H), 7.72 (d, J=3.20 Hz, 1H), 7.67 (d, J=8.40 Hz, 1H), 7.55 (d, J=3.12 Hz, 1H), 7.47 (t, J=8.00 Hz, 1H), 7.24 (d, J=7.64 Hz, 1H), 6.00 (s, 1H), 5.84 (bs, 1H), 5.40 (bs, 1H), 2.33 (s, 3H), 2.15 (s, 3H), 1.58 (d, J=6.96 Hz, 3H).

### Example 13

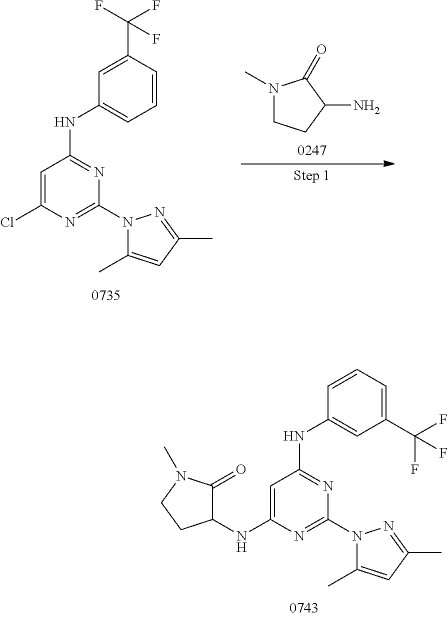

Step 1[0743]: To a solution of 0.25 g of 6-chloro-2-(3,5-dimethyl-1H-pyrazol-1-yl)-N-(3-(trifluoromethyl)phenyl)pyrimidin-4-amine [0735] in acetonitrile was added 3-amino-1-methylpyrrolidin-2-one (0.155 g, 1.35 mmol) and N,N-diisopropyl ethylamine. The reaction mixture was heated at 180° C. under microwave for 5 h. The reaction mixture was concentrated under reduced pressure to afford crude product which was purified by column chromatography using 40% ethyl acetate in pet ether to afford 0.14 g of 3-((2-(3,5-dimethyl-1H-pyrazol-1-yl)-6-((3-(trifluoromethyl)phenyl)amino)pyrimidin-4-yl)amino)-1-methyl pyrrolidin-2-one [0743], Compound 188 as a yellow solid. MS(M+1)+=446.4. 1H NMR (400 MHz, DMSO-d6) δ 9.55 (s, 1H), 8.52 (s, 1H), 7.68 (d, J=8.2 Hz, 1H), 7.58 (bs, 1H), 7.48 (t, J=7.9 Hz, 1H), 7.24 (d, J=7.4 Hz, 1H), 6.05 (s, 1H), 5.82 (s, 1H), 4.61 (bs, 1H), 3.40-3.36 (m, 2H), 2.78 (s, 3H), 2.5 (s, 3H), 2.48-2.35 (m, 1H), 2.18 (s, 3H), 1.89-1.80 (m, 1H).

### Example 14

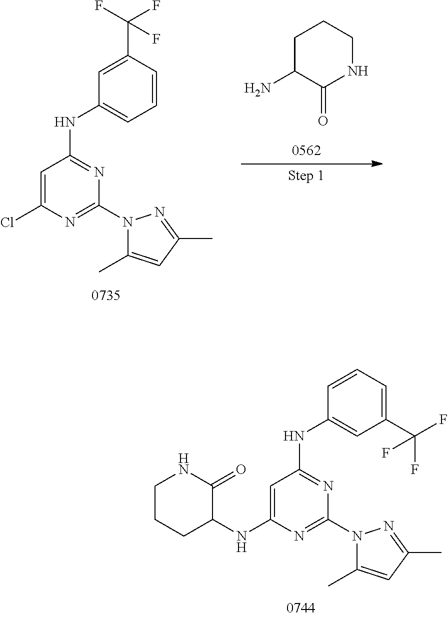 [SMILES: Cc1cc(C)n(-c2nc(Cl)cc(Nc3cccc(C(F)(F)F)c3)n2)n1 ; NC1CCCNC1=O ; Cc1cc(C)n(-c2nc(Nc3cccc(C(F)(F)F)c3)cc(NC3CCCNC3=O)n2)n1]

Step 1[0744]: To a solution of 0.1 g of 6-chloro-2-(3,5-dimethyl-1H-pyrazol-1-yl)-N-(3-(trifluoromethyl) phenyl) pyrimidin-4-amine [0735] in acetonitrile was added 3-amino-2-piperidone [0562] (0.062 g, 0.543 mmol) and N,N-diisopropyl ethylamine. The reaction mixture was heated at 180° C. under microwave for 5 h. The reaction mixture was concentrated under reduced pressure to afford crude product which was purified by column chromatography using 40% ethyl acetate in pet ether to afford 0.038 g of 3-((2-(3,5-dimethyl-1H-pyrazol-1-yl)-6-((3-(trifluoromethyl)phenyl)amino)pyrimidin-4-yl)amino) piperidin-2-one [0744], Compound 189 as a white solid. MS(M+1)+=446.2. 1H NMR (400 MHz, DMSO-d6) δ 9.52 (s, 1H), 8.55 (s, 1H), 7.69-7.66 (m, 2H), 7.47 (t, J=8.0 Hz, 2H), 7.23 (d, J=7.7 Hz, 1H), 6.05 (s, 1H), 5.82 (s, 1H), 4.43 (s, 1H), 3.17 (bs, 2H), 2.52 (s, 3H), 2.18 (s, 3H), 2.15-2.10 (m, 1H), 1.89-1.64 (m, 3H).

### Example 15

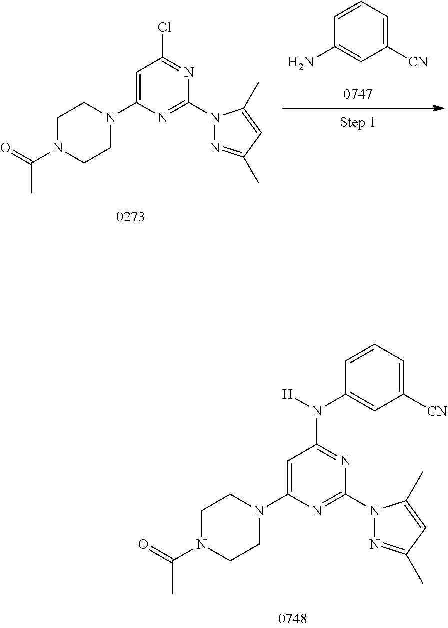 [SMILES: CC(=O)N1CCN(c2cc(Cl)nc(-n3nc(C)cc3C)n2)CC1 ; N#Cc1cccc(N)c1 ; CC(=O)N1CCN(c2cc(Nc3cccc(C#N)c3)nc(-n3nc(C)cc3C)n2)CC1]

Step 1[0748]1: To a stirred solution of 1-(4-(6-chloro-2-(3,5-dimethyl-1H-pyrazol-1-yl)pyrimidin-4-yl)piperazin-1-yl)ethan-1-one [0273] (0.9 g, 2.68 mmol) in dioxane (10 mL) was added 3-aminobenzonitrile [0747] (0.64 g, 5.376 mmol), 4,5-bis(diphenylphosphino)-9,9-dimethylxanthene (0.46 g, 0.806 mmol) and cesium carbonate (1.31 g, 4.03 mmol). The reaction mixture was degassed with nitrogen for 10 min before adding tris(dibenzylideneacetone)dipalladium(0) (0.49 g, 0.53 mmol) and heated at 100° C. for 16 h. The reaction mixture was filtered through celite and filtrate was concentrated under reduced pressure to afford crude mass which was purified by preparative HPLC to afford 3-((6-(4-acetylpiperazin-1-yl)-2-(3,5-dimethyl-1H-pyrazol-1-yl)pyrimidin-4-yl)amino)benzonitrile [0748], Compound 186 as a yellow solid (0.1 g). MS(M+1)⁺=417.2. 1H NMR (400 MHz, DMSO-d6) δ 9.72 (s, 1H), 8.71 (s, 1H), 7.72-7.69 (m, 1H), 7.48 (t, J=8.0 Hz, 1H), 7.39-7.36 (m, 1H), 6.10 (s, 1H), 5.89 (s, 1H), 3.65-3.46 (m, 8H), 2.56 (s, 3H), 2.21 (s, 3H), 2.06 (s, 3H).

### Example 16

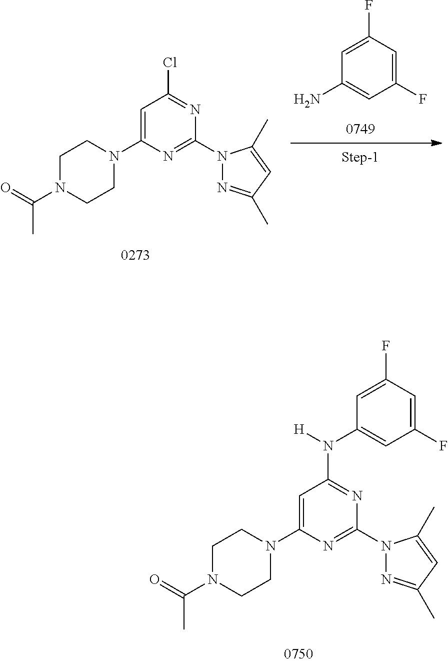 [SMILES: CC(=O)N1CCN(c2cc(Cl)nc(-n3nc(C)cc3C)n2)CC1 ; Nc1cc(F)cc(F)c1 ; CC(=O)N1CCN(c2cc(Nc3cc(F)cc(F)c3)nc(-n3nc(C)cc3C)n2)CC1]

Step 1[0750]: The procedure is similar to step 1[0748] in example 15. 0.3 g of 1-(4-(6-chloro-2-(3,5-dimethyl-1H-pyrazol-1-yl)pyrimidin-4-yl)piperazin-1-yl)ethan-1-one [0750] and 3,5-difluoroaniline [0749] (0.23 g, 1.792 mmol) gave 0.02 g of 1-(4-(6-((3,5-difluorophenyl)amino)-2-(3,5-dimethyl-1H-pyrazol-1-yl)pyrimidin-4-yl)piperazin-1-yl)ethan-1-one [0750], Compound 179 as an off-white solid. MS(M+1)+=428, ¹H NMR (400 MHz, DMSO-d6): δ 9.76 (s, 1H), 7.56-7.53 (m, 2H), 6.75-6.70 (m, 1H), 6.09 (s, 1H), 5.87 (s, 1H), 3.67-3.53 (m, 8H), 2.54 (s, 3H), 2.18 (s, 3H), 2.04 (s, 3H).

### Example 17

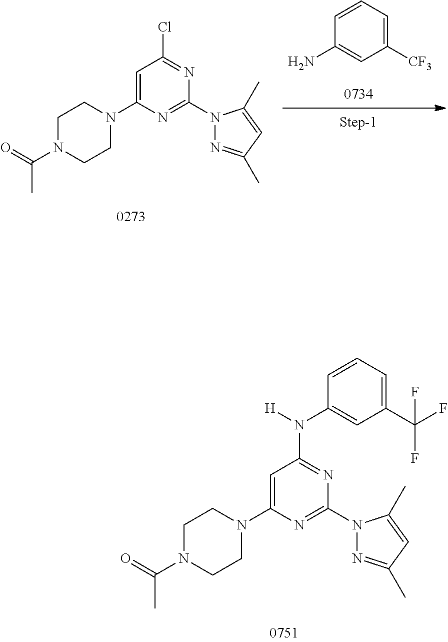 [SMILES: CC(=O)N1CCN(c2cc(Cl)nc(-n3nc(C)cc3C)n2)CC1 ; Nc1cccc(C(F)(F)F)c1 ; CC(=O)N1CCN(c2cc(Nc3cccc(C(F)(F)F)c3)nc(-n3nc(C)cc3C)n2)CC1]

Step-1[0751]: The procedure is similar to step 1[0748] in example 15. 0.3 g of 1-(4-(6-chloro-2-(3,5-dimethyl-1H-pyrazol-1-yl)pyrimidin-4-yl)piperazin-1-yl)ethan-1-one [0273] and 3-(trifluoromethyl)aniline [0734] (0.288 g, 1.792 mmol) gave 0.04 g of 1-(4-(2-(3,5-dimethyl-1H-pyrazol-1-yl)-6-((3-(trifluoromethyl) phenyl)amino) pyrimidin-4-yl)piperazin-1-yl)ethan-1-one [0751], Compound 17 as a yellow solid. MS(M+1)+=460, 1H-NMR (400 MHz, DMSO-d6): δ 9.70 (s, 1H), 8.67 (s, 1H), 7.68 (d, J=8.36 Hz, 1H), 7.50 (t, J=8.04 Hz, 1H), 7.27 (d, J=7.72 Hz, 1H), 6.09 (s, 1H), 5.89 (s, 1H), 3.63-3.55 (m, 8H), 2.55 (s, 3H), 2.19 (s, 3H), 2.06 (s, 3H).

### Example 18

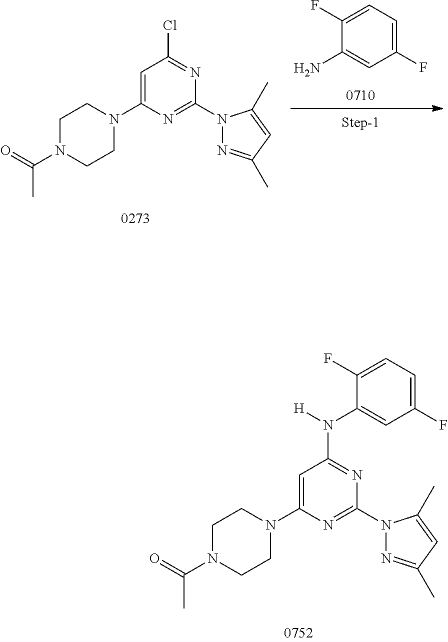 [SMILES: CC(=O)N1CCN(c2cc(Cl)nc(-n3nc(C)cc3C)n2)CC1 ; Nc1cc(F)ccc1F ; CC(=O)N1CCN(c2cc(Nc3cc(F)ccc3F)nc(-n3nc(C)cc3C)n2)CC1]

Step-1[0752]: The procedure is similar to step 1[0748] in example 15. 0.5 g of 1-(4-(6-chloro-2-(3,5-dimethyl-1H-pyrazol-1-yl)pyrimidin-4-yl)piperazin-1-yl)ethan-1-one [0273] and 2,5-difluoroaniline [0710] (0.386 g, 2.986 mmol) gave 0.145 g of 1-(4-(6-((2,5-difluorophenyl)amino)-2-(3,5-dimethyl-1H-pyrazol-1-yl)pyrimidin-4-yl)piperazin-1-yl)ethan-1-one [0752], Compound 178 as an off-white solid. MS(M+1)+=428, 1H NMR (400 MHz, DMSO-d6) δ 9.19 (s, 1H), 8.45 (ddd, J=11.5, 6.7, 3.2 Hz, 1H), 7.28 (ddd, J=11.0, 9.0, 5.3 Hz, 2H), 6.84 (m, 1H), 6.19 (s, 1H), 6.08 (s, 1H), 3.70-3.52 (m, 8H), 2.19 (s, 3H), 2.06 (s, 3H).

### Example 19

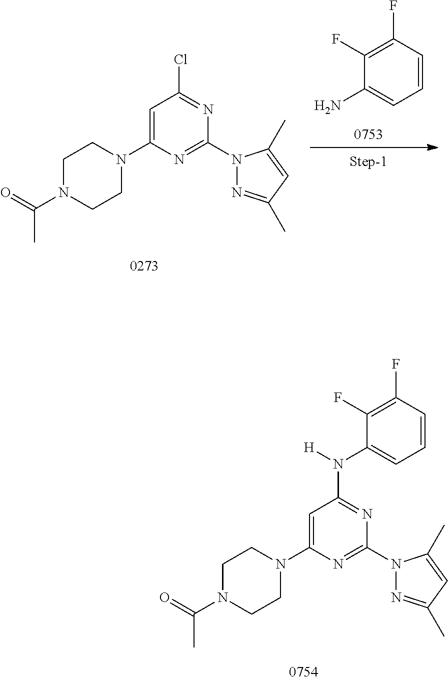 [SMILES: CC(=O)N1CCN(c2cc(Cl)nc(-n3nc(C)cc3C)n2)CC1 ; Nc1cccc(F)c1F ; CC(=O)N1CCN(c2cc(Nc3cccc(F)c3F)nc(-n3nc(C)cc3C)n2)CC1]

Step-1[0754]: The procedure is similar to step 1[0748] in example 15. 0.5 g of 1-(4-(6-chloro-2-(3,5-dimethyl-1H-pyrazol-1-yl)pyrimidin-4-yl)piperazin-1-yl)ethan-1-one [0273] and 2,3-difluoroaniline [0753] (0.192 g, 1.493 mmol) gave 0.045 g of 1-(4-(6-((2,3-difluorophenyl)amino)-2-(3,5-dimethyl-1H-pyrazol-1-yl)pyrimidin-4-yl)piperazin-1-yl)ethan-1-one [0754], Compound 180 as an off-white solid. MS(M+1)+=428, 1H NMR (400 MHz, DMSO-d6) δ 9.23 (s, 1H), 7.82 (t, J=7.6 Hz, 1H), 7.14 (q, J=8.9, 8.2 Hz, 2H), 6.04 (s, 1H), 6.00 (s, 1H), 3.68-3.48 (m, 8H), 2.43 (s, 3H), 2.17 (s, 3H), 2.05 (s, 3H).

### Example 20

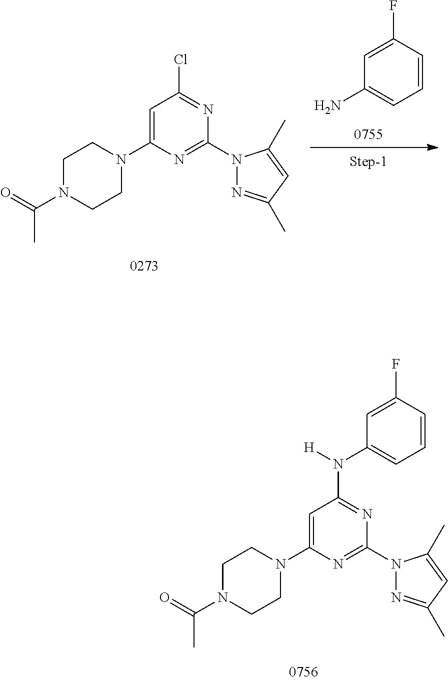 [SMILES: CC(=O)N1CCN(c2cc(Cl)nc(-n3nc(C)cc3C)n2)CC1 ; Nc1cccc(F)c1 ; CC(=O)N1CCN(c2cc(Nc3cccc(F)c3)nc(-n3nc(C)cc3C)n2)CC1]

Step-1[0756]: The procedure is similar to step 1[0748] in example 15. 0.5 g of 1-(4-(6-chloro-2-(3,5-dimethyl-1H-pyrazol-1-yl)pyrimidin-4-yl)piperazin-1-yl)ethan-1-one [0273] and 3-fluoroaniline [0755] (0.33 g, 2.986 mmol) gave 0.040 g of 1-(4-(6-((3-fluorophenyl)amino)-2-(3,5-dimethyl-1H-pyrazol-1-yl)pyrimidin-4-yl)piperazin-1-yl)ethan-1-one [0756], Compound 177 as an off-white solid. MS(M+1)+=410, 1H NMR (400 MHz, DMSO-d6) δ 9.56 (s, 1H), 7.92 (dt, J=12.3, 2.2 Hz, 1H), 7.38-7.12 (m, 2H), 6.82-6.67 (m, 1H), 6.07 (s, 1H), 5.90 (s, 1H), 3.57 (m, 8H), 2.53 (s, 3H), 2.19 (s, 3H), 2.04 (s, 3H).

### Example 21

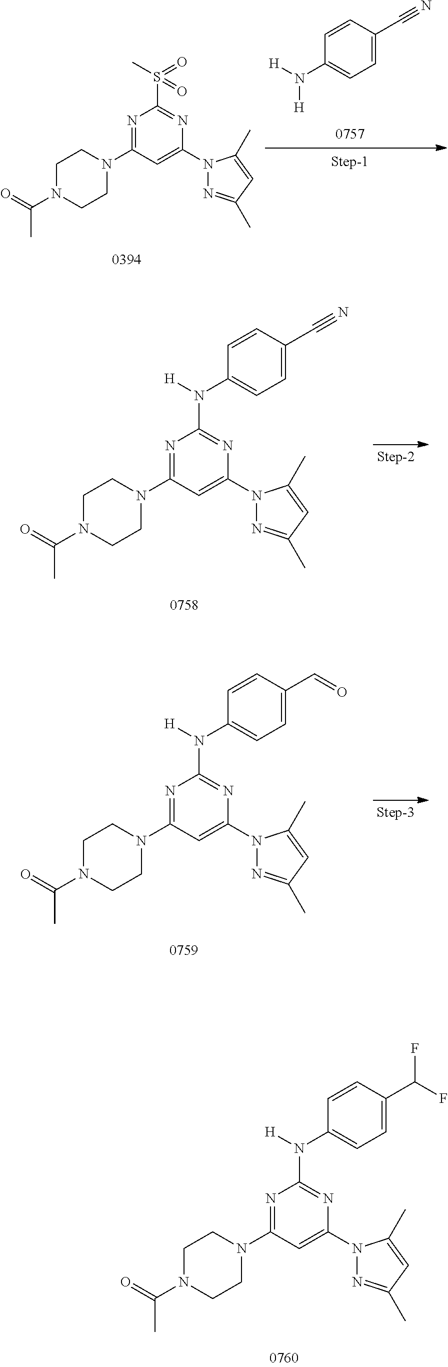 [SMILES: CC(=O)N1CCN(c2cc(-n3nc(C)cc3C)nc(S(C)(=O)=O)n2)CC1 ; N#Cc1ccc(N)cc1 ; CC(=O)N1CCN(c2cc(-n3nc(C)cc3C)nc(Nc3ccc(C#N)cc3)n2)CC1 ; CC(=O)N1CCN(c2cc(-n3nc(C)cc3C)nc(Nc3ccc(C=O)cc3)n2)CC1 ; CC(=O)N1CCN(c2cc(-n3nc(C)cc3C)nc(Nc3ccc(C(F)F)cc3)n2)CC1]

Step-1[0758]: The procedure is similar to step 1 [0748] in example 15. 0.3 g of 1-(4-(6-(3,5-dimethyl-1H-pyrazol-1-yl)-2-(methylsulfonyl)pyrimidin-4-yl)piperazin-1-yl)ethan-1-one [0394] and 4-aminobenzonitrile [0757] (0.187 g, 1.585 mmol) gave 0.150 g of 4-((4-(4-acetylpiperazin-1-yl)-6-(3,5-dimethyl-1H-pyrazol-1-yl)pyrimidin-2-yl)amino)benzonitrile [0758] as an white solid. MS(M+1)+=417, 1H NMR (400 MHz, DMSO-d6) δ 9.70 (s, 1H), 7.87 (d, J=8.5 Hz, 2H), 7.73 (d, J=8.5 Hz, 2H), 6.63 (s, 1H), 6.13 (s, 1H), 3.73 (bs, 2H), 3.64 (m, 2H), 3.58 (m, 4H), 2.62 (s, 3H), 2.20 (s, 3H), 2.06 (s, 3H).

Step-2 [0759]: To a solution of 4-((4-(4-acetylpiperazin-1-yl)-6-(3,5-dimethyl-1H-pyrazol-1-yl)pyrimidin-2-yl)amino)benzonitrile [0758] (0.1 g, 0.24 mmol) in aqueous formic acid (80%, 10 mL) was added platinum (IV)oxide (0.016 g, 0.07 mmol). Then the reaction mixture was heated at 55° C. for 4h. The reaction mixture was filtered through celite bed, filtrate was concentrated to afford 4-((4-(4-acetylpiperazin-1-yl)-6-(3,5-dimethyl-1H-pyrazol-1-yl)pyrimidin-2-yl)amino) benzaldehyde [0759] as an yellow solid (0.1 g). MS(M+1)+=420.

Step-3[0760]: To a stirred solution of 4-((4-(4-acetylpiperazin-1-yl)-6-(3,5-dimethyl-1H-pyrazol-1-yl)pyrimidin-2-yl)amino) benzaldehyde [0759] (0.1 g, 0.23 mmol) in dichloromethane (5 mL) was added diethylaminosulfur trifluoride (0.063 mL, 0.47 mmol) at 0° C. The reaction mixture was stirred at rt for 15 min, quenched with 10% sodium bicarbonate solution (10 mL) and extracted with dichloromethane (2×20 mL). The combined organic layer was dried over anhydrous sodium sulfate, filtered and concentrated under reduced pressure to afford crude which was purified by Prep HPLC to obtain 1-(4-(2-((4-(difluoromethyl)phenyl)amino)-6-(3,5-dimethyl-1H-pyrazol-1-yl)pyrimidin-4-yl)piperazin-1-yl)ethan-1-one [0760], Compound 202 as a white gum (0.035 g). MS(M+1)+=442. 1H NMR (400 MHz, DMSO-d6): δ 9.41 (s, 1H), 7.79 (d, J=8.52 Hz, 2H), 7.47 (d, J=8.56 Hz, 2H), 6.94 (t, J_{F}=56.20 Hz, 1H), 6.57 (s, 1H), 6.10 (s, 1H), 3.71 (m, 2H), 3.57 (m, 2H), 3.57 (m, 4H), 2.61 (s, 3H), 2.19 (s, 3H), 2.05 (s, 3H).

### Example 22

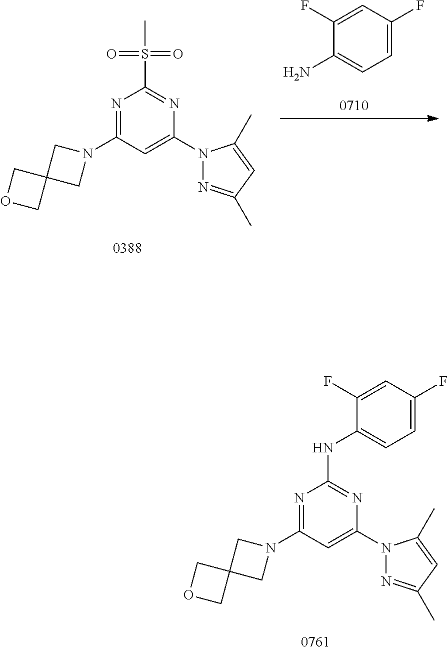 [SMILES: Cc1cc(C)n(-c2cc(N3CC4(COC4)C3)nc(S(C)(=O)=O)n2)n1 ; Nc1ccc(F)cc1F ; Cc1cc(C)n(-c2cc(N3CC4(COC4)C3)nc(Nc3ccc(F)cc3F)n2)n1]

Step 1[0761] To the solution of 6-(6-(3,5-dimethyl-1H-pyrazol-1-yl)-2-(methylsulfonyl)pyrimidin-4-yl)-2-oxa-6-azaspiro[3.3]heptane [0388] (0.25 g, 0.71 mmol) and 2,4-difluoroaniline [0710] (0.184 g, 1.43 mmol) in dimethylsulfoxide was added cesium carbonate (0.46 g, 1.43 mmol) in closed vial and the reaction mixture was heated at 100° C. After 16 h, the reaction mixture was quenched with water and stirred for 10 min. The solid formed was filtered, washed with water and hexane to afford a white solid which was purified in the Reveleris flash system using 65% ethyl acetate in hexane as eluent to afford N-(2,4-difluorophenyl)-4-(3,5-dimethyl-1H-pyrazol-1-yl)-6-(2-oxa-6-azaspiro[3.3]heptan-6-yl)pyrimidin-2-amine [0761], Compound 107 as an off-white solid (0.06 g). MS(M+1)+=399. 1H-NMR (400 MHz, DMSO-d6): δ 8.73 (s, 1H), 7.56-7.50 (m, 1H), 7.27-7.24 (m, 1H), 7.05-7.01 (m, 1H), 6.09 (s, 1H), 5.99 (s, 1H), 4.71 (s, 4H), 4.19 (s, 4H), 2.25 (s, 3H), 2.14 (s, 3H).

### Example 23

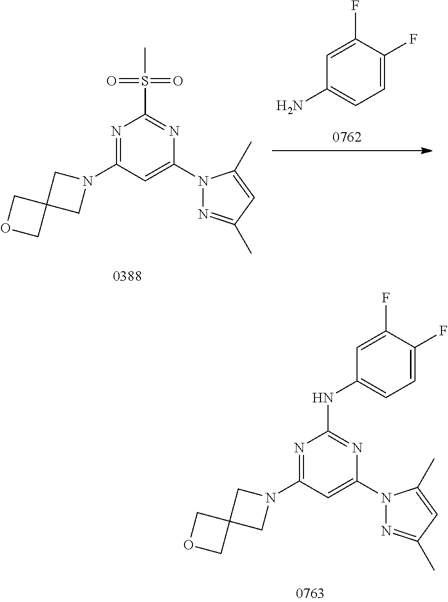 [SMILES: Cc1cc(C)n(-c2cc(N3CC4(COC4)C3)nc(S(C)(=O)=O)n2)n1 ; Nc1ccc(F)c(F)c1 ; Cc1cc(C)n(-c2cc(N3CC4(COC4)C3)nc(Nc3ccc(F)c(F)c3)n2)n1]

Step 4[0763]: The procedure is similar to step-1 [0761] in example 22, 0.25 g of 6-(6-(3,5-dimethyl-1H-pyrazol-1-yl)-2-(methylsulfonyl)pyrimidin-4-yl)-2-oxa-6-azaspiro[3.3]heptane [0388] and 3,4-difluoroaniline [0762] (0.184 g, 1.43 mmol) gave 0.08 g of N-(3,4-difluorophenyl)-4-(3,5-dimethyl-1H-pyrazol-1-yl)-6-(2-oxa-6-azaspiro[3.3]heptan-6-yl)pyrimidin-2-amine [0763], Compound 108 as an off-white solid. MS(M+1)+=399. 1H NMR (400 MHz, DMSO-d6) δ 9.36 (s, 1H), 8.05-7.82 (m, 1H), 7.45-7.39 (m, 1H), 7.38-7.25 (m, 1H), 6.14 (s, 1H), 6.09 (s, 1H), 4.74 (s, 4H), 4.25 (s, 4H), 2.59 (s, 3H), 2.18 (s, 3H).

### Example 24

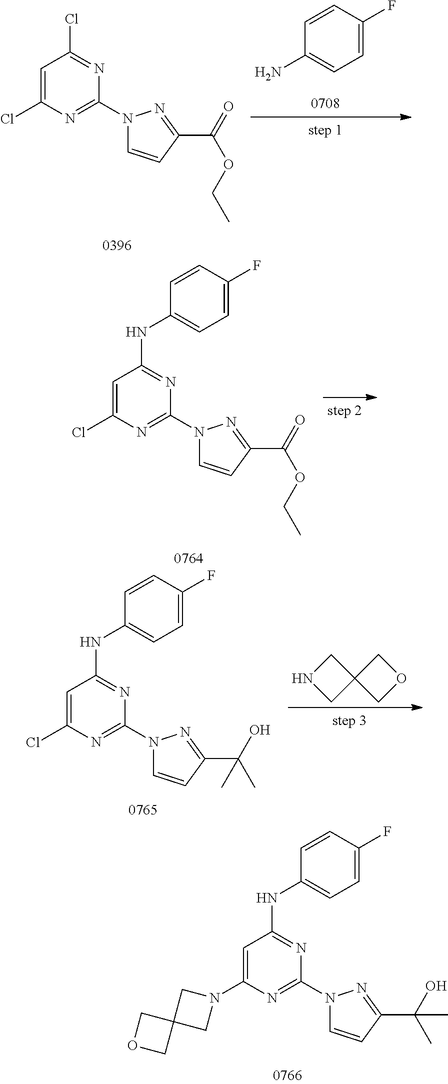 [SMILES: CCOC(=O)c1ccn(-c2nc(Cl)cc(Cl)n2)n1 ; Nc1ccc(F)cc1 ; CCOC(=O)c1ccn(-c2nc(Cl)cc(Nc3ccc(F)cc3)n2)n1 ; CC(C)(O)c1ccn(-c2nc(Cl)cc(Nc3ccc(F)cc3)n2)n1 ; C1NCC12COC2 ; CC(C)(O)c1ccn(-c2nc(Nc3ccc(F)cc3)cc(N3CC4(COC4)C3)n2)n1]

Step 1[0764]: 2 g of ethyl 1-(4,6-dichloropyrimidin-2-yl)-1H-pyrazole-3-carboxylate [0396] and 4-fluoroaniline [0708] gave 2.5 g of ethyl 1-(4-chloro-6-((4-fluorophenyl)amino)pyrimidin-2-yl)-1H-pyrazole-3-carboxylate [0764] as a brown solid (Using DIPEA, ACN, 75° C., 16h). MS(M+1)+=362.2.

Step 2[0765]: To a solution of ethyl 1-(4-chloro-6-((4-fluorophenyl)amino)pyrimidin-2-yl)-1H-pyrazole-3-carboxylate [0764] (1 g, 2.76 mmol) in tetrahydrofuran (5 mL) was added methyl magnesium bromide (0.98 g, 8.29 mmol) drop-wise at 0° C. under inert atm. Resultant reaction mixture was allowed to stir at same 0 OC to rt for 4 h. The reaction mixture was quenched with saturated ammonium chloride solution (10 mL) and product was extracted with dichloromethane (3×30 ml). The combined organic layer were washed with brine (10 mL), dried over anhydrous sodium sulfate and concentrated under reduced pressure to afford crude product, which was purified by column chromatography using 20% ethyl acetate in pet ether as solvent to afford 2-(1-(4-chloro-6-((4-fluorophenyl)amino)pyrimidin-2-yl)-1H-pyrazol-3-yl)propan-2-ol [0765] as a white solid (0.3 g). MS(M+1)+=348.2.

Step 3[0766]: To a solution of 2-(1-(4-chloro-6-((4-fluorophenyl)amino)pyrimidin-2-yl)-1H-pyrazol-3-yl)propan-2-ol [0765] (0.15 g, 0.43 mmol) in acetonitrile (10 mL) was added 2-oxa-6-azaspiro(3,3)heptane [0259] (0.042 g, 0.43 mmol) and cesium carbonate (0.28 g, 0.86 mmol). The reaction mixture was irradiated at 100° C. in MW for 2 h. After the completion, the reaction mixture was filtered to remove cesium carbonate. The filtrate was concentrated to afford brownish gum and which was purified by column chromatography using ethyl acetate as eluent to afford 2-(1-(4-((4-fluorophenyl)amino)-6-(2-oxa-6-azaspiro[3.3]heptan-6-yl)pyrimidin-2-yl)-1H-pyrazol-3-yl)propan-2-ol (0.09 g). [0766], Compound 155 as a white solid. MS(M+1)+=411.2. 1H NMR (400 MHz, DMSO-d6) δ 9.37 (s, 1H), 8.35 (d, J=2.6 Hz, 1H), 7.67 (dd, J=9.0, 4.9 Hz, 2H), 7.13 (t, J=8.8 Hz, 2H), 6.48 (d, J=2.5 Hz, 1H), 5.42 (s, 1H), 5.05 (s, 1H), 4.73 (s, 4H), 4.19 (s, 4H), 1.48 (s, 6H).

### Example 25

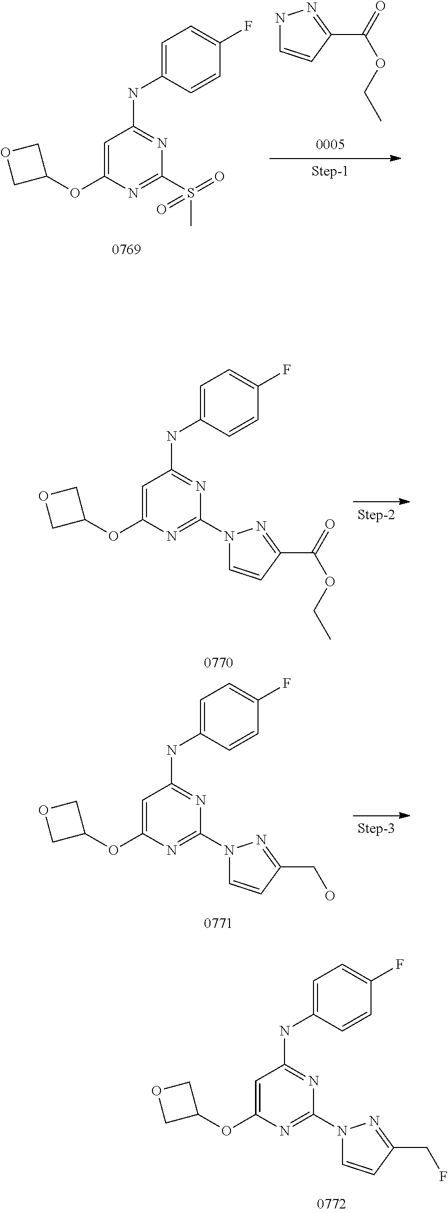

Step-1[0770]: The procedure is similar to step 3 [0766] in example 24 (at 100° C.). 0.65 g of N-(4-fluorophenyl)-2-(methylsulfonyl)-6-(oxetan-3-yloxy)pyrimidin-4-amine [0769] and 0.41 g of ethyl 1h-Pyrazole-3-carboxylate gave 0.65 g of ethyl 1-(4-((4-fluorophenyl)amino)-6-(oxetan-3-yloxy)pyrimidin-2-yl)-1H-pyrazole-3-carboxylate [0770] as an off-white gum. MS(M+1)+=400.

Step-2[0771]: To a stirred solution of ethyl 1-(4-((4-fluorophenyl)amino)-6-(oxetan-3-yloxy)pyrimidin-2-yl)-1H-pyrazole-3-carboxylate [0770] (0.65 g, 1.62 mmol) in tetrahydrofuran (10 mL) was added a solution of lithium aluminium hydride in tetrahydrofuran (1.6 mL, 2 M, 3.25 mmol) at 0° C. The reaction mixture was stirred at rt for 1 h. The reaction mixture was quenched with saturated aqueous ammonium chloride solution (3 mL) and extracted with ethyl acetate (2×25 mL). The combined organic layer was dried over anhydrous sodium sulfate, filtered and concentrated to afford ((1-(4-((4-fluorophenyl)amino)-6-(oxetan-3-yloxy)pyrimidin-2-yl)-1H-pyrazol-3-yl)methanol [0771] as an off-white gum (0.35 g). MS(M+1)+=358.

Step-3[0772]: The procedure is similar to step-3 [0760] in example 21. 0.35 g of ((1-(4-((4-fluorophenyl)amino)-6-(oxetan-3-yloxy)pyrimidin-2-yl)-1H-pyrazol-3-yl)methanol [0771] gave 0.085 g of 2-(3-(fluoromethyl)-1H-pyrazol-1-yl)-N-(4-fluorophenyl)-6-(oxetan-3-yloxy)pyrimidin-4-amine [0772], Compound 162 as an off-white solid. MS(M+1)+=360. 1H-NMR (400 MHz, DMSO-d6): δ 9.85 (s, 1H), 8.54 (d, J=2.40 Hz, 1H), 7.65-7.62 (m, 2H), 7.23-7.18 (m, 2H), 6.69 (d, J=0.80 Hz, 1H), 5.98 (s, 1H), 5.68-5.65 (m, 1H), 5.45 (d, J_{F}=48.5 Hz, 2H), 4.92 (t, J=6.80 Hz, 2H), 4.62-4.59 (m, 2H).

### Example 26

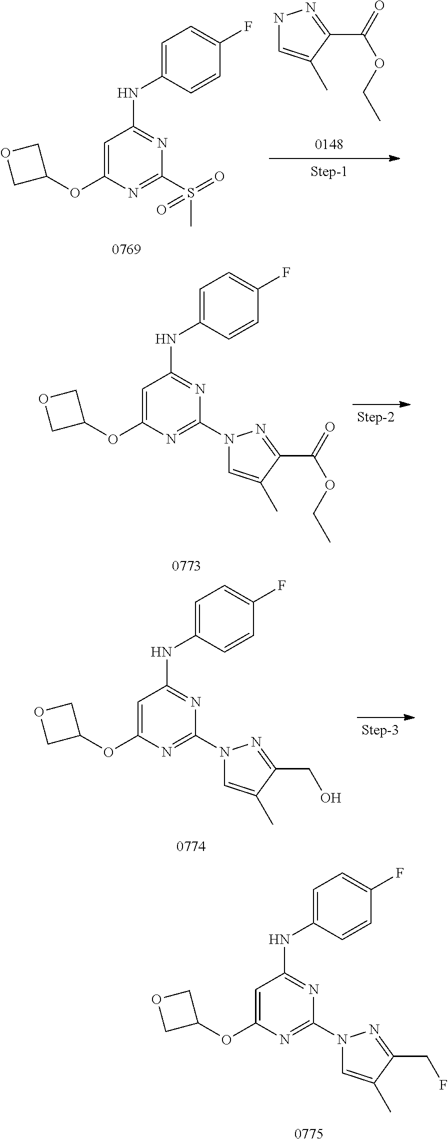

Step 1[0773]: The procedure is similar to step 3 [0766] in example 24 (at 100° C.). 0.3 g of N-(4-fluorophenyl)-2-(methylsulfonyl)-6-(oxetan-3-yloxy)pyrimidin-4-amine [0769] and 0.2 g of ethyl 4-methyl-1H-pyrazole-3-carboxylate gave 0.3 g of 2-(3-(fluoromethyl)-4-methyl-1H-pyrazol-1-yl)-N-(4-fluorophenyl)-6-(oxetan-3-yloxy)pyrimidin-4-amine [0773] as a brownish gum. MS(M+1)+=414.

Step-2[0774]: The procedure is similar to step-2 [0771] in example 25. 0.3 g of ethyl 4-methyl-1H-pyrazole-3-carboxylate gave 0.3 g of 2-(3-(fluoromethyl)-4-methyl-1H-pyrazol-1-yl)-N-(4-fluorophenyl)-6-(oxetan-3-yloxy)pyrimidin-4-amine [0773] gave 0.25 g of (1-(4-((4-fluorophenyl)amino)-6-(oxetan-3-yloxy)pyrimidin-2-yl)-4-methyl-1H-pyrazol-3-yl)methanol [0774] as a brownish gum. MS(M+1)+=372.

Step 3: [0775]: The procedure is similar to step-3 [0760] in example 21. 0.25 g of (1-(4-((4-fluorophenyl)amino)-6-(oxetan-3-yloxy)pyrimidin-2-yl)-4-methyl-1H-pyrazol-3-yl)methanol [0774] gave 0.046 g of 2-(3-(fluoromethyl)-4-methyl-1H-pyrazol-1-yl)-N-(4-fluorophenyl)-6-(oxetan-3-yloxy)pyrimidin-4-amine [0775], Compound 163 as an off-white solid. MS(M+1)+=374. 1H NMR (400 MHz, DMSO-d6) δ 9.83 (s, 1H), 8.35 (s, 1H), 7.63 (t, J=6.4 Hz, 2H), 7.21 (t, J=8.9 Hz, 2H), 5.96 (s, 1H), 5.69-5.62 (m, 1H), 5.52 (d, J_{F}=48.5 Hz, 2H), 4.94 (t, J=6.9 Hz, 2H), 4.61 (dd, J=7.5, 5.3 Hz, 2H), 2.15 (s, 3H).

### Example 27

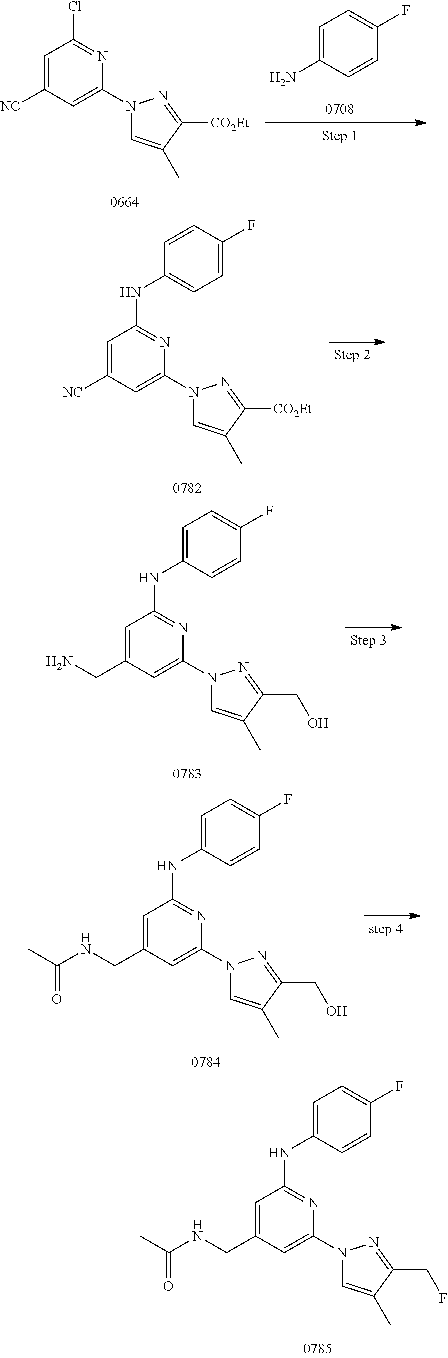 [SMILES: CCOC(=O)c1nn(-c2cc(C#N)cc(Cl)n2)cc1C ; Nc1ccc(F)cc1 ; CCOC(=O)c1nn(-c2cc(C#N)cc(Nc3ccc(F)cc3)n2)cc1C ; Cc1cn(-c2cc(CN)cc(Nc3ccc(F)cc3)n2)nc1CO ; CC(=O)NCc1cc(Nc2ccc(F)cc2)nc(-n2cc(C)c(CO)n2)c1 ; CC(=O)NCc1cc(Nc2ccc(F)cc2)nc(-n2cc(C)c(CF)n2)c1]

Step 1[0782]: This procedure is similar to step 1[0748] in example 15. 4 g of ethyl 1-(6-chloro-4-cyanopyridin-2-yl)-4-methyl-1H-pyrazole-3-carboxylate[0664] and 3 g of 4-fluoroaniline [0708] gave 4 g of ethyl 1-(4-cyano-6-((4-fluorophenyl)amino)pyridin-2-yl)-4-methyl-1H-pyrazole-3-carboxylate [0782] as a yellow solid. MS(M+1)+=366.2.

Step 2[0783]: This procedure is similar to step 2[0771] in example 25. 4 g of ethyl 1-(4-cyano-6-((4-fluorophenyl)amino)pyridin-2-yl)-4-methyl-1H-pyrazole-3-carboxylate [0782] gave 3 g (crude) of (1-(4-(aminomethyl)-6-((4-fluorophenyl)amino)pyridin-2-yl)-4-methyl-1H-pyrazol-3-yl)methanol [0783] as a yellow solid. MS(M+1)+=328.1.

Step 3[0784]: To a solution of ethyl (1-(4-(aminomethyl)-6-((4-fluorophenyl)amino)pyridin-2-yl)-4-methyl-1H-pyrazol-3-yl)methanol [0783] (3 g, 9.164 mmol), in dichloromethane (30 mL) was added acetyl chloride(2.5 g, 22.91 mmol) in drop wise and followed by triethylamine (4.6 g, 45.82 mmol) at 0° C. After addition the reaction mixture was stirred at rt for 1 h. The reaction mixture was diluted with water (10 mL) and extracted with ethyl acetate (2×50 mL). The combined organic layer was washed with brine (10 mL), dried over sodium sulfate, filtered and concentrated under reduced pressure to afford crude product and which was dissolved in methanol:water(1:1) followed by addition of potassium carbonate (0.63 g, 4.58 mmol) and stirred at rt. The reaction mixture was concentrated under reduced pressure to afford crude product and which was purified by column chromatography using 5% methanol in chloroform as solvent to afford N-((2-((4-fluorophenyl)amino)-6-(3-(hydroxymethyl)-4-methyl-1H-pyrazol-1-yl)pyridin-4-yl)methyl)acetamide [0784], Compound 166 as yellow solid (0.07 g). MS(M+1)+=370.2, 1H NMR (400 MHz, DMSO-d6) δ 9.26 (s, 1H), 8.49 (t, J=6.1 Hz, 1H), 8.16 (s, 1H), 7.72-7.59 (m, 2H), 7.24-7.12 (m, 2H), 7.08 (s, 1H), 6.51 (s, 1H), 5.05 (t, J=5.7 Hz, 1H), 4.48 (d, J=5.6 Hz, 2H), 4.24 (d, J=5.9 Hz, 2H), 2.13 (s, 3H), 1.92 (s, 3H).

Step 4[0785]: This procedure is similar to step 3[0760] in example 21. 0.35 g of N-((2-((4-fluorophenyl)amino)-6-(3-(hydroxymethyl)-4-methyl-1H-pyrazol-1-yl)pyridin-4-yl)methyl) acetamide gave 0.03 g of N-((2-(3-(fluoromethyl)-4-methyl-1H-pyrazol-1-yl)-6-((4-fluorophenyl)amino)pyridin-4-yl)methyl)acetamide [0784], Compound 161 as white solid. MS(M+1)+=372.2, 1H NMR (400 MHz, DMSO-d6) δ 9.32 (s, 1H), 8.50 (t, J=6.1 Hz, 1H), 8.27 (s, 1H), 7.69-7.59 (m, 2H), 7.22-7.15 (m, 2H), 7.12 (s, 1H), 6.57 (s, J=1.1 Hz, 1H), 5.50 (d, J_{F}=48.5 Hz, 2H), 4.25 (d, J=6.1 Hz, 2H), 2.17 (s, 3H), 1.92 (s, 3H).

### Example 28

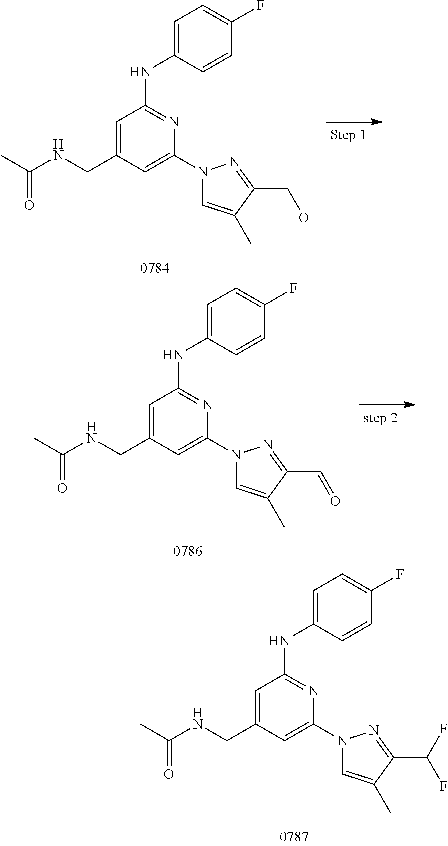

Step 1[0786]: To a solution of N-((2-((4-fluorophenyl)amino)-6-(3-(hydroxymethyl)-4-methyl-1H-pyrazol-1-yl)pyridin-4-yl)methyl) acetamide [0784] (1.5 g, 4.06 mmol) in tetrahydrofuran was added manganese dioxide (3.5 g, 40.06 mmol) under N2 and the reaction mixture was stirred at rt for 24 h. The reaction mixture was filtered through celite bed, washed with tetrahydrofuran. The filtrate was concentrated under reduced pressure and the residue was triturated with ethyl acetate, decanted and dried under vacuum to afford N-((2-((4-fluorophenyl)amino)-6-(3-formyl-4-methyl-1H-pyrazol-1-yl)pyridin-4-yl)methyl)acetamide [0786] as a yellow solid (0.8 g). MS(M+1)+=390.3.

Step 2[0787]: This procedure is similar to step 3 [0760] in example 21. 0.2 g of N-((2-((4-fluorophenyl)amino)-6-(3-formyl-4-methyl-1H-pyrazol-1-yl)pyridin-4-yl)methyl)acetamide [0786] gave 0.03 g of N-((2-(3-(difluoromethyl)-4-methyl-1H-pyrazol-1-yl)-6-((4-fluorophenyl)amino)pyridin-4-yl)methyl)acetamide [0787], Compound 168 as a yellow solid. MS(M+1)+=368.2, 1H NMR (400 MHz, DMSO-d6) δ 9.36 (s, 1H), 8.50 (bs, 1H), 8.33 (s, 1H), 7.66 (dd, J=9.1, 4.8 Hz, 2H), 7.28-7.15 (m, 2H), 7.11 (t, J_{F}=54.4 Hz, 1H), 6.60 (s, 1H), 4.25 (d, J=6.0 Hz, 2H), 2.21 (s, 3H), 1.91 (s, 3H).

### Example 29

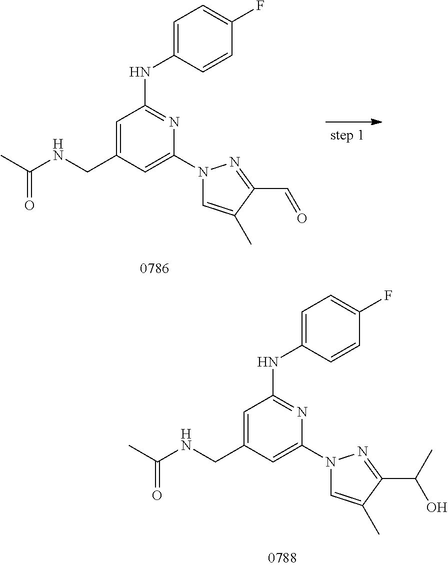 [SMILES: CC(=O)NCc1cc(Nc2ccc(F)cc2)nc(-n2cc(C)c(C=O)n2)c1 ; CC(=O)NCc1cc(Nc2ccc(F)cc2)nc(-n2cc(C)c(C(C)O)n2)c1]

Step 1[0788]: To a solution of N-((2-((4-fluorophenyl)amino)-6-(3-formyl-4-methyl-1H-pyrazol-1-yl)pyridin-4-yl)methyl)acetamide [0786] (0.15 g, 0.408 mmol) in dry tetrahydrofuran (5 mL) was added methyl magnesium bromide (1.4 M solution in tetrahydrofuran, 0.38 g, 3.266 mmol) drop wise. The resultant reaction mixture was stirred at rt for 24 h. The reaction mixture was quenched with saturated ammonium chloride solution (5 mL) and extracted with ethyl acetate (2×50 mL). The combined organic layer was washed with brine, dried over sodium sulfate, filtered and concentrated under reduced pressure to afford crude product which was purified by column chromatography using 4% methanol in chloroform as solvent to afford N-((2-((4-fluorophenyl)amino)-6-(3-(1-hydroxyethyl)-4-methyl-1H-pyrazol-1-yl)pyridin-4-yl)methyl)acetamide [0788], Compound 167 as off-white solid (0.06 g). MS(M+1)+=384.2, 1H NMR (400 MHz, DMSO-d6) δ 9.26 (s, 1H), 8.49 (t, J=6.1 Hz, 1H), 8.14 (s, 1H), 7.66 (dd, J=8.9, 4.9 Hz, 2H), 7.18 (t, J=8.7 Hz, 2H), 7.06 (s, 1H), 6.51 (s, 1H), 5.11 (d, J=4.6 Hz, 1H), 4.84 (m, 1H), 4.24 (d, J=6.0 Hz, 2H), 2.17 (s, 3H), 1.92 (s, 3H), 1.45 (d, J=6.5 Hz, 3H).

### Example 30

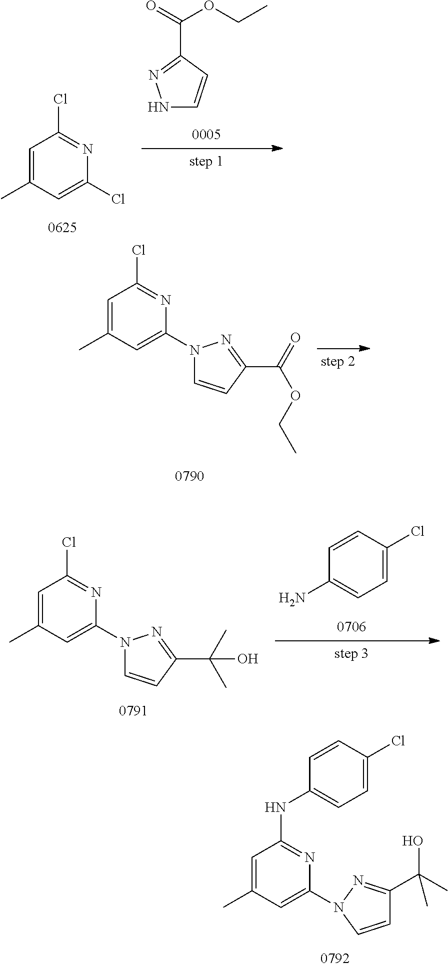

Step 1[0790]: This procedure is similar to step 3[0766] in example 24 (at 100° C.). 0.5 g of 2,6-dichloro-4-methyl pyridine [0625] and 0.52 g of ethyl 1h-pyrazole-3-carboxylate [0005]gave 0.4 g of ethyl 1-(6-chloro-4-methylpyridin-2-yl)-1H-pyrazole-3-carboxylate [0790] as white solid. MS(M+1)+=266.0.

Step 2[0791]: This procedure is similar to step 2[0765] in example 24. 0.5 g of ethyl 1-(6-chloro-4-methylpyridin-2-yl)-1H-pyrazole-3-carboxylate [0790] gave 0.45 g(crude) of 2-(1-(6-chloro-4-methylpyridin-2-yl)-1H-pyrazol-3-yl)propan-2-ol [0791] as yellow oil. MS(M+1)+=252.0.

Step 3[0792]: To the stirred solution of 2-(1-(6-chloro-4-methylpyridin-2-yl)-1H-pyrazol-3-yl)propan-2-ol [0791] (0.5 g, 1.98 mmol) in dioxane were added N-(4-chlorophenyl)formamide (0.61 g, 3.97 mmol), lithium bis(trimethylsilyl)amide (0.99 g, 5.95 mmol), BINAP[rac-2,2′-Bis(diphenylphosphino)-1,1′-binaphthyl] (0.25 g, 0.39 mmol) and the reaction mixture was purged with nitrogen for 5 min. Then tris(dibenzylideneacetone)dipalladium(0) (0.545 g, 0.595 mmol) was added to the reaction mixture and heated at 100° C. for 16 h. The reaction mixture was filtered through celite bed, washed with chloroform, and the filtrate was concentrated under reduced pressure to afford crude product which was purified by column chromatography using 4% methanol in chloroform as solvent to afford 2-(1-(6-((4-chlorophenyl)amino)-4-methylpyridin-2-yl)-1H-pyrazol-3-yl)propan-2-ol, Compound 145 as yellow solid [0792] (0.08 g). MS(M+1)+=343.0, 1H NMR (400 MHz, DMSO-d6) δ 9.30 (s, 1H), 8.34 (d, J=2.5 Hz, 1H), 7.68 (d, J=8.8 Hz, 2H), 7.35 (d, J=8.8 Hz, 2H), 7.08 (s, 1H), 6.51 (d, J=2.3 Hz, 2H), 5.06 (s, 1H), 2.31 (s, 3H), 1.49 (s, 6H).

### Example 31

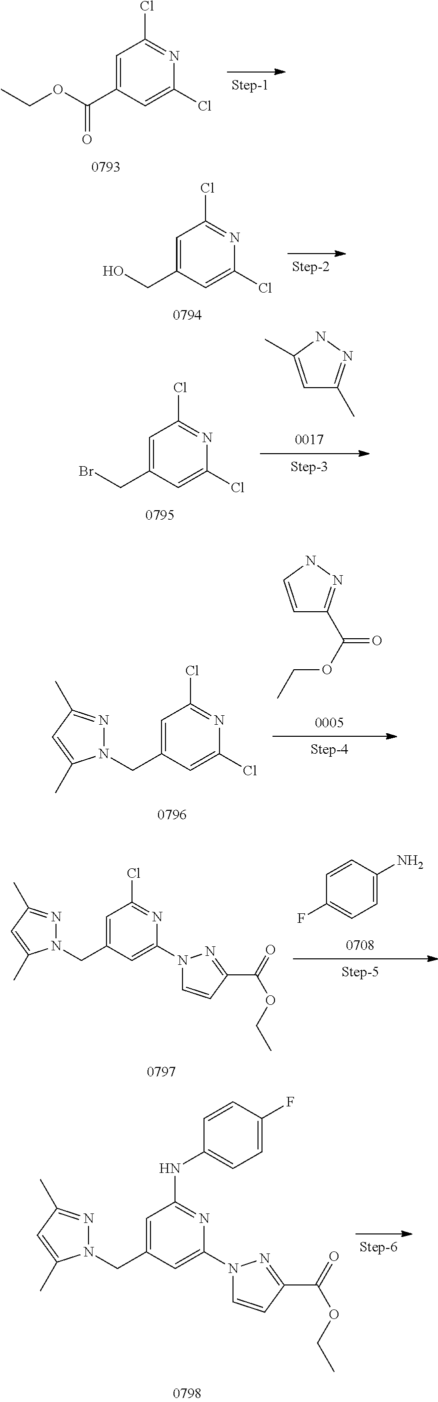

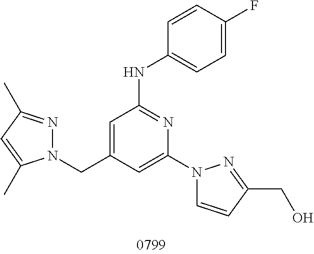 [SMILES: Cc1cc(C)n(Cc2cc(Nc3ccc(F)cc3)nc(-n3ccc(CO)n3)c2)n1]

Step 1[0794]: To a stirred solution of ethyl 2,6-dichloroisonicotinate [0793](14.5 g, 65.8 mmol) in tetrahydrofuran (5 mL) was added lithium borohydride (4.30 g, 197.67 mmol) at 0° C. The reaction mixture was stirred at rt for 1.5 h, quenched with ice and extracted with ethyl acetate (2×15 mL). The combined organic layer was washed with brine (10 mL), dried over anhydrous sodium sulfate, filtered and concentrated under reduced pressure to afford (2,6-dichloropyridin-4-yl)methanol [0794] as white solid (11 g). MS(M+1)+=179.2.

Step 2[0795]: To a solution of (2,6-dichloropyridin-4-yl)methanol [0794] in tetrahydrofuran was added triphenylphosphine and N-bromosuccinimide at 0° C. and the reaction mixture was stirred at same temperature. After 2 h, the reaction mixture was diluted with water and extracted with ethyl acetate (3×150 mL). The combined organic layer was washed with brine (2×100 mL), dried over anhydrous sodium sulfate, filtered and concentrated to afford a crude product which was purified by column chromatography to afford 1.1 g of 4-(bromomethyl)-2,6-dichloropyridine [0795] as colorless liquid. MS(M+1)+=241.8.

Step 3[0796]: The procedure is similar to step 2[0720] in example 2 (at 90° C.). 1.1 g of 4-(bromomethyl)-2,6-dichloropyridine [0795] gave 0.96 g of 2,6-dichloro-4-((3,5-dimethyl-1H-pyrazol-1-yl)methyl)pyridine [0796] as brown solid. MS(M+1)+=257.2.

Step 4[0797]: The procedure is similar to step 2[0720] in example 2 (at 90° C.). 0.96 g of 2,6-dichloro-4-((3,5-dimethyl-1H-pyrazol-1-yl)methyl)pyridine [0005] gave 0.98 g of ethyl 1-(6-chloro-4-((3,5-dimethyl-1H-pyrazol-1-yl)methyl)pyridin-2-yl)-1H-pyrazole-3-carboxylate [0797] as brown solid. MS(M+1)+=360.2.

Step 5[0798]: The procedure is similar to Step 1[0748] in example 15. 0.85 g of ethyl 1-(6-chloro-4-((3,5-dimethyl-1H-pyrazol-1-yl)methyl)pyridin-2-yl)-1H-pyrazole-3-carboxylate [0797] gave 0.64 g of ethyl 1-(4-((3,5-dimethyl-1H-pyrazol-1-yl)methyl)-6-((4-fluorophenyl)amino)pyridin-2-yl)-1H-pyrazole-3-carboxylate [0798] as yellow solid. MS(M+1)+=435.2.

Step 6[0799]: The procedure is similar to step 2 [0771] in example 25. 0.64 g of ethyl 1-(4-((3,5-dimethyl-1H-pyrazol-1-yl)methyl)-6-((4-fluorophenyl)amino)pyridin-2-yl)-1H-pyrazole-3-carboxylate [0798] gave 0.28 g of (1-(4-((3,5-dimethyl-1H-pyrazol-1-yl)methyl)-6-((4-fluorophenyl)amino)pyridin-2-yl)-1H-pyrazol-3-yl)methanol [0799], Compound 169 as off-white solid. MS(M+1)+=493.2, 1H-NMR (400 MHz, DMSO-d6): δ 9.32 (s, 1H), 8.39 (s, 1H), 7.66-7.62 (m, 2H), 7.19-7.15 (m, 2H), 7.03 (s, 1H), 6.51 (s, 1H), 6.24 (s, 1H), 5.92 (s, 1H), 5.24-5.21 (m, 3H), 4.50 (s, 2H), 2.18 (s, 3H), 2.14 (s, 3H).

### Example 32

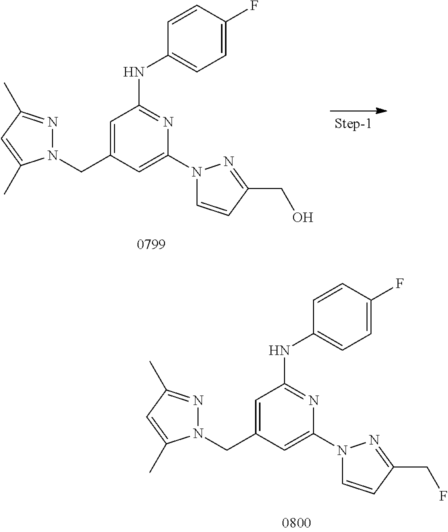 [SMILES: Cc1cc(C)n(Cc2cc(Nc3ccc(F)cc3)nc(-n3ccc(CO)n3)c2)n1 ; Cc1cc(C)n(Cc2cc(Nc3ccc(F)cc3)nc(-n3ccc(CF)n3)c2)n1]

Step 1[0800]: The procedure is similar to step 3[0760] in example 21. 0.24 g of (1-(4-((3,5-dimethyl-1H-pyrazol-1-yl)methyl)-6-((4-fluorophenyl)amino)pyridin-2-yl)-1H-pyrazol-3-yl)methanol [0799] gave 0.045 g of 4-((3,5-dimethyl-1H-pyrazol-1-yl)methyl)-6-(3-(fluoromethyl)-1H-pyrazol-1-yl)-N-(4-fluorophenyl)pyridin-2-amine [0800], Compound 170 as an off-white solid. MS(M+1)+=395.2, 1H-NMR (400 MHz, DMSO-d6): δ 9.37 (s, 1H), 8.47 (d, J=2.44 Hz, 1H), 7.66-7.63 (m, 2H), 7.17 (t, J=8.96 Hz, 2H), 7.08 (s, 1H), 6.71 (s, 1H), 6.28 (s, 1H), 5.93 (s, 1H), 5.45 (d, J_{F}=48 Hz, 2H), 5.26 (s, 2H), 2.19 (s, 3H), 2.14 (s, 3H).

### Example 33

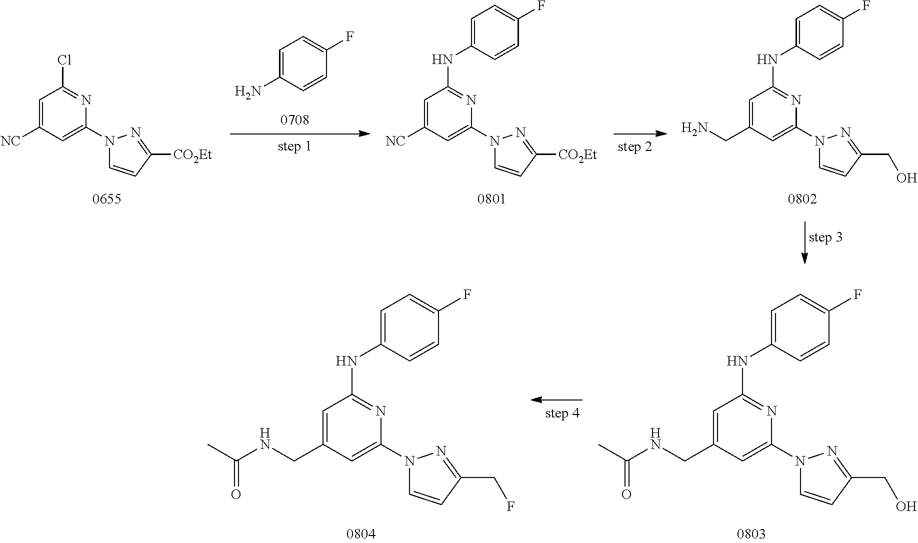 [SMILES: CCOC(=O)c1ccn(-c2cc(C#N)cc(Cl)n2)n1 ; Nc1ccc(F)cc1 ; CCOC(=O)c1ccn(-c2cc(C#N)cc(Nc3ccc(F)cc3)n2)n1 ; NCc1cc(Nc2ccc(F)cc2)nc(-n2ccc(CO)n2)c1 ; CC(=O)NCc1cc(Nc2ccc(F)cc2)nc(-n2ccc(CF)n2)c1 ; CC(=O)NCc1cc(Nc2ccc(F)cc2)nc(-n2ccc(CO)n2)c1]

Step 1[0801]: The procedure is similar to step 1[0748] in example 15. 3.0 g of ethyl 1-(6-chloro-4-cyanopyridin-2-yl)-1H-pyrazole-3-carboxylate [0655] and 4-fluoroaniline (1.2 g, 1.03 mmol) gave 1.4 g of ethyl 1-(4-cyano-6-((4-fluorophenyl)amino)pyridin-2-yl)-1H-pyrazole-3-carboxylate [0801] as a yellow solid. MS(M+1)+=352.0

Step 2[0802]: The procedure is similar to step 2[0771] in example 25. 1.2 g of ethyl 1-(4-cyano-6-((4-fluorophenyl)amino)pyridin-2-yl)-1H-pyrazole-3-carboxylate [0801] gave 0.87 g of (1-(4-(aminomethyl)-6-((4-fluorophenyl)amino)pyridin-2-yl)-1H-pyrazol-3-yl)methanol [0802] as a brownish solid. MS(M+1)+=314.1

Step 3[0803]: The procedure is similar to step 3[0784] in example 27. 0.68 g of (1-(4-(aminomethyl)-6-((4-fluorophenyl)amino)pyridin-2-yl)-1H-pyrazol-3-yl)methanol [0802] gave 0.51 g of N-((2-((4-fluorophenyl)amino)-6-(3-(hydroxymethyl)-1H-pyrazol-1-yl)pyridin-4-yl)methyl)acetamide [0803] as a brown solid. MS(M+1)+=356.2

Step 4[0804]: The procedure is similar to step 3[0760] in example 21. 0.15 g of N-((2-((4-fluorophenyl)amino)-6-(3-(hydroxymethyl)-1H-pyrazol-1-yl)pyridin-4-yl)methyl)acetamide [0803] gave 0.14 g of N-((2-(3-(fluoromethyl)-1H-pyrazol-1-yl)-6-((4-fluorophenyl)amino)pyridin-4-yl)methyl)acetamide [0804], Compound 159 as an off-white solid. MS(M+1)+=358.2, 1H NMR (400 MHz, DMSO-d6) δ 9.36 (s, 1H), 8.51 (t, 1H), 8.47 (d, J=2.44 Hz, 1H), 7.75-7.56 (m, 2H), 7.28-7.12 (m, 3H), 6.76-6.66 (m, 1H), 6.60 (s, 1H), 5.45 (d, J_{F}=48 Hz, 2H), 4.27 (d, J=6.1 Hz, 2H), 1.93 (s, 3H).

### Example 34

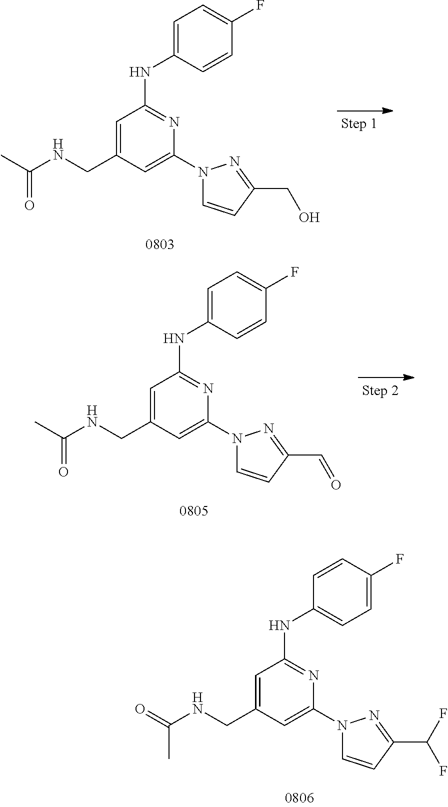 [SMILES: CC(=O)NCc1cc(Nc2ccc(F)cc2)nc(-n2ccc(CO)n2)c1 ; CC(=O)NCc1cc(Nc2ccc(F)cc2)nc(-n2ccc(C=O)n2)c1 ; CC(=O)NCc1cc(Nc2ccc(F)cc2)nc(-n2ccc(C(F)F)n2)c1]

Step 1[0805]: The procedure is similar to step 1[0786] in example 28. 0.5 g of N-((2-((4-fluorophenyl)amino)-6-(3-(hydroxymethyl)-1H-pyrazol-1-yl)pyridin-4-yl)methyl)acetamide [0803] gave 0.31 g of N-((2-((4-fluorophenyl)amino)-6-(3-formyl-1H-pyrazol-1-yl)pyridin-4-yl)methyl)acetamide [0805] as a brown solid. MS(M+1)+=354.35

Step 2 [0806]: The procedure is similar to step 3[0760] in example 21. 0.5 g of N-((2-((4-fluorophenyl)amino)-6-(3-(hydroxymethyl)-1H-pyrazol-1-yl)pyridin-4-yl)methyl)acetamide [0805] gave 0.065 g of N-((2-(3-(difluoromethyl)-1H-pyrazol-1-yl)-6-((4-fluorophenyl)amino)pyridin-4-yl)methyl)acetamide [0806], Compound 160 as an off-white solid. MS(M+1)+=376.2, 1H NMR (400 MHz, DMSO-d6) δ 9.39 (s, 1H), 8.69-8.29 (m, 2H), 7.75-7.65 (m, 2H), 7.25-7.13 (m, 4H), 6.82 (s, 1H), 6.63 (s, 1H), 4.27 (d, J=6.1 Hz, 2H), 1.92 (s, 3H).

### Example 35

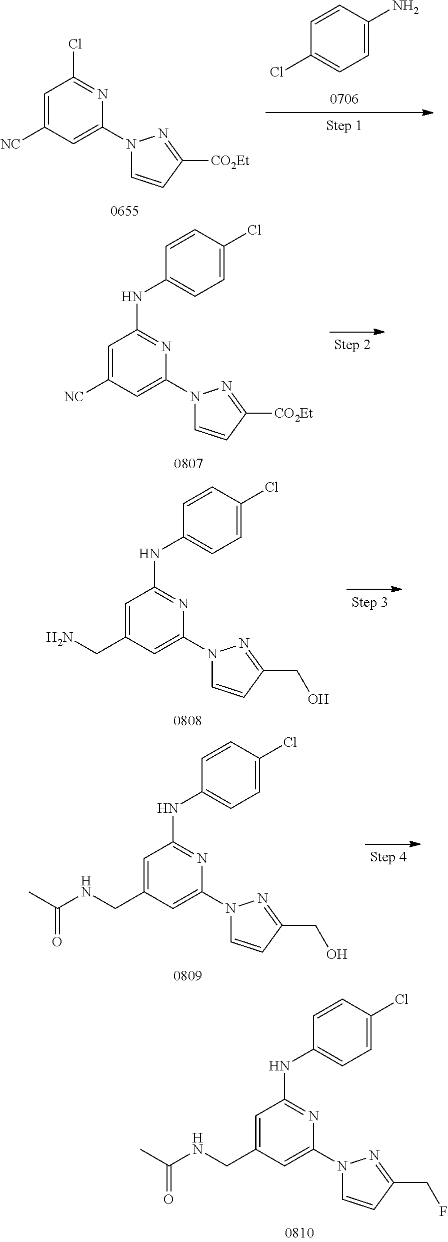

Step 1[0807]: The procedure is similar to step 1[0748] in example 15. 4.6 g of ethyl 1-(6-chloro-4-cyanopyridin-2-yl)-1H-pyrazole-3-carboxylate [0655] and 2.2 g of 4-chloroaniline [0706] gave 3.1 g of ethyl 1-(6-((4-chlorophenyl)amino)-4-cyanopyridin-2-yl)-1H-pyrazole-3-carboxylate [0807] as yellow solid. MS(M+1)+=368.0.

Step 2[0808]: The procedure is similar to step 2[0771] in example 25. 3.1 g of 3.1 g of ethyl 1-(6-((4-chlorophenyl)amino)-4-cyanopyridin-2-yl)-1H-pyrazole-3-carboxylate [0807] gave 2.1 g of (1-(4-(aminomethyl)-6-((4-chlorophenyl)amino)pyridin-2-yl)-1H-pyrazol-3-yl)methanol[0808] as brown solid. MS(M+1)+=330.2

Step 3[0809]: The procedure is similar to step 3[0784] in example 27. 2.1 g of (1-(4-(aminomethyl)-6-((4-chlorophenyl)amino)pyridin-2-yl)-1H-pyrazol-3-yl)methanol [0808] gave 1.2 g of N-((2-((4-chlorophenyl)amino)-6-(3-(hydroxymethyl)-1H-pyrazol-1-yl)pyridin-4-yl)methyl)acetamide [0809] as brown solid. MS(M+1)+=372.2.

Step 4[0810]: The procedure is similar to step 3[0760] in example 21. 0.6 g of N-((2-((4-chlorophenyl)amino)-6-(3-(hydroxymethyl)-1H-pyrazol-1-yl)pyridin-4-yl)methyl)acetamide [0809] gave 0.122 g of N-((2-((4-chlorophenyl)amino)-6-(3-(fluoromethyl)-1H-pyrazol-1-yl)pyridin-4-yl)methyl)acetamide [0810], Compound 164 as off-white solid. MS(M+1)+=374.2, 1H NMR (400 MHz, DMSO-d6) δ 9.49 (s, 1H), 8.62-8.40 (m, 2H), 7.70 (d, J=8.76 Hz, 2H), 7.38 (d, J=8.8 Hz, 2H), 7.20 (s, 1H), 6.71 (s, 1H), 6.64 (s, 1H), 5.45 (d, J_{F}=48 Hz, 2H), 4.28 (d, J=6.1 Hz, 2H), 1.93 (s, 3H).

### Example 36

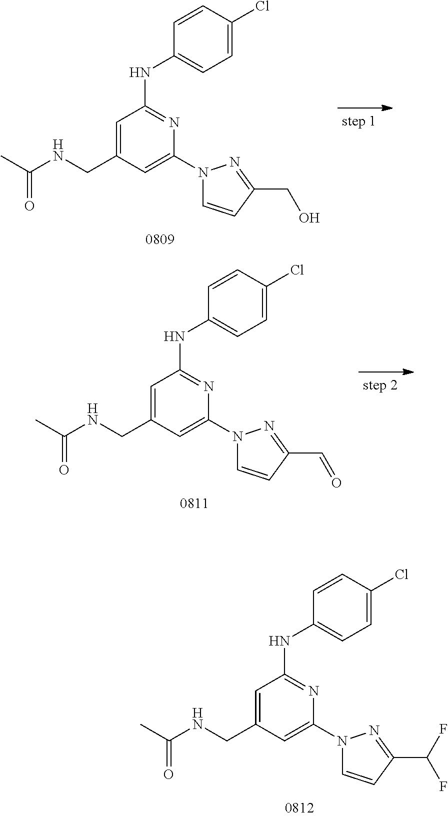 [SMILES: CC(=O)NCc1cc(Nc2ccc(Cl)cc2)nc(-n2ccc(CO)n2)c1 ; CC(=O)NCc1cc(Nc2ccc(Cl)cc2)nc(-n2ccc(C=O)n2)c1 ; CC(=O)NCc1cc(Nc2ccc(Cl)cc2)nc(-n2ccc(C(F)F)n2)c1]

Step 1 [0811]: The procedure is similar to step 1[0786] in example 28. 0.65 g of N-((2-((4-chlorophenyl)amino)-6-(3-(hydroxymethyl)-1H-pyrazol-1-yl)pyridin-4-yl)methyl)acetamide [0809] of gave 0.42 g of N-((2-((4-chlorophenyl)amino)-6-(3-formyl-1H-pyrazol-1-yl)pyridin-4-yl)methyl)acetamide [0811] as an brown solid. MS(M+1)+=370.2.

Step 2[0812]: The procedure is similar to step 3[0760] in example 21. 0.42 g of N-((2-((4-chlorophenyl)amino)-6-(3-formyl-1H-pyrazol-1-yl)pyridin-4-yl)methyl)acetamide [0811] gave 0.045 g of N-((2-((4-chlorophenyl)amino)-6-(3-(difluoromethyl)-1H-pyrazol-1-yl)pyridin-4-yl)methyl)acetamide [0812], Compound 165 as off-white solid. MS(M+1)+=392.2, 1H-NMR (400 MHz, DMSO-d6): δ 9.54 (s, 1H), 8.58 (s, 1H), 8.53 (t, J=5.72 Hz, 1H), 7.70 (d, J=8.80 Hz, 2H), 7.38 (d, J=8.80 Hz, 2H), 7.20 (s, 1H), 7.17 (t, J_{F}=48 Hz, 1H), 6.84 (s, 1H), 6.68 (s, 1H), 4.29 (d, J=5.96 Hz, 2H), 1.93 (s, 3H).

### Example 37

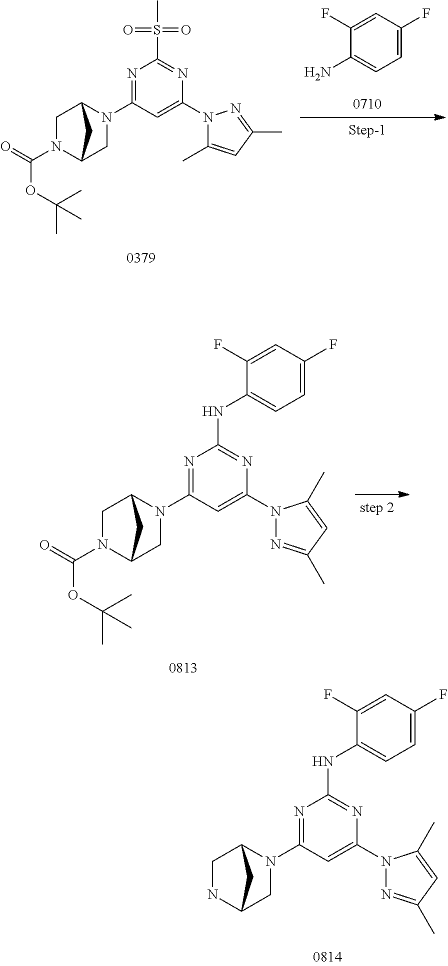 [SMILES: Cc1cc(C)n(-c2cc(N3C[C@H]4C[C@@H]3CN4C(=O)OC(C)(C)C)nc(S(C)(=O)=O)n2)n1 ; Nc1ccc(F)cc1F ; Cc1cc(C)n(-c2cc(N3C[C@H]4C[C@@H]3CN4C(=O)OC(C)(C)C)nc(Nc3ccc(F)cc3F)n2)n1 ; Cc1cc(C)n(-c2cc(N3C[C@H]4C[C@@H]3CN4)nc(Nc3ccc(F)cc3F)n2)n1]

Step 1[0813]: To a solution of t-butyl (1S,4S)-5-(6-(3,5-dimethyl-1H-pyrazol-1-yl)-2-(methylsulfonyl) pyrimidin-4-yl)-2,5-diazabicyclo[2.2.1] heptane-2-carboxylate [0379] (0.2 g, 0.44 mmol) and 2,4-difluoroaniline [0710] (0.05 g, 0.44 mmol) in DMSO (2 mL) was added cesium carbonate (0.29 g, 0.89 mmol) then the reaction mixture was heated at 100° C. in a closed vial for 16 h. The reaction mixture was quenched with ice-cold water and the obtained solid was filtered and washed with hexane to afford tert-butyl (1S,4S)-5-(2-((2,4-difluorophenyl)amino)-6-(3,5-dimethyl-1H-pyrazol-1-yl)pyrimidin-4-yl)-2,5-diazabicyclo [2.2.1]heptane-2-carboxylate [0813] as yellow solid (0.14 g). MS(M+1)+=498.4.

Step 2[0814]: To a cooled solution of tert-butyl (1S,4S)-5-(2-((2,4-difluorophenyl)amino)-6-(3,5-dimethyl-1H-pyrazol-1-yl)pyrimidin-4-yl)-2,5-diazabicyclo [2.2.1]heptane-2-carboxylate [0813] (0.12 g, 0.241 mmol) in dioxane (5 mL) was added hydrogen chloride gas (5 mL) in dioxane. The reaction mixture was stirred at rt for 2 h. The reaction mixture was concentrated under reduced pressure and it was triturated with mixture of solvents (dichloromethane:diethylether) and decanted. The residue was dried under high vacuum to afford 4-((1S,4S)-2,5-diazabicyclo[2.2.1]heptan-2-yl)-N-(2,4-difluorophenyl)-6-(3,5-dimethyl-1H-pyrazol-1-yl)pyrimidin-2-amine[0814], Compound 106 as an off-white solid (0.07 g). MS(M+1)+=398.4. 1H NMR (400 MHz, Methanol-d4) δ 7.88 (d, J=8.4 Hz, 1H), 7.14 (m, 1H), 7.06 (m, 1H), 6.25 (s, 1H), 5.15 (s, 1H), 4.64 (s, 1H), 3.86 (m, 2H), 3.49 (m, 2H), 2.57 (s, 3H), 2.40-2.21 (m, 2H), 2.32 (s, 3H), 2.17 (m, 1H).

### Example 38

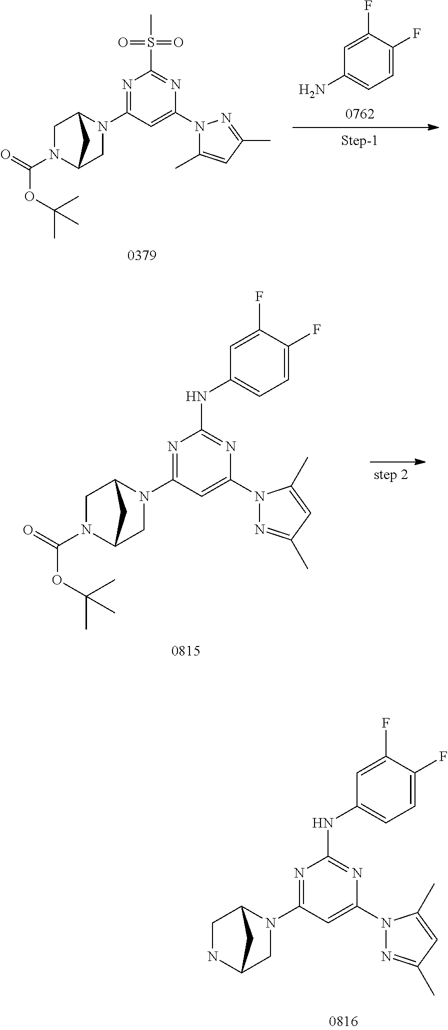 [SMILES: Cc1cc(C)n(-c2cc(N3C[C@H]4C[C@@H]3CN4C(=O)OC(C)(C)C)nc(S(C)(=O)=O)n2)n1 ; Nc1ccc(F)c(F)c1 ; Cc1cc(C)n(-c2cc(N3C[C@H]4C[C@@H]3CN4C(=O)OC(C)(C)C)nc(Nc3ccc(F)c(F)c3)n2)n1 ; Cc1cc(C)n(-c2cc(N3C[C@H]4C[C@@H]3CN4)nc(Nc3ccc(F)c(F)c3)n2)n1]

Step 1[0815]: The procedure is similar to step 1[0813] in Example 37. 0.3 g of t-butyl (1S,4S)-5-(6-(3,5-dimethyl-1H-pyrazol-1-yl)-2-(methylsulfonyl)pyrimidin-4-yl)-2,5-diazabicyclo[2.2. 1]heptane-2-carboxylate [0379] gave 0.22 g of tert-butyl (1S,4S)-5-(2-((3,4-difluorophenyl)amino)-6-(3,5-dimethyl-1H-pyrazol-1-yl)pyrimidin-4-yl)-2,5-diazabicyclo[2.2.1]heptane-2-carboxylate [0815] as off-white solid. MS(M+1)+=498.4.

Step 2[0816]: The procedure is similar to step 2[0814] in Example 37. 0.22 g of tert-butyl (1S,4S)-5-(2-((3,4-difluorophenyl)amino)-6-(3,5-dimethyl-1H-pyrazol-1-yl)pyrimidin-4-yl)-2,5-diazabicyclo [2.2.1]heptane-2-carboxylate [0815] gave 0.21 g of 4-((1S,4S)-2,5-diazabicyclo[2.2.1]heptan-2-yl)-N-(3,4-difluorophenyl)-6-(3,5-dimethyl-1H-pyrazol-1-yl)pyrimidin-2-amine [0816], Compound 109 as off-white. MS(M+1)+=398.2. 1H NMR (400 MHz, DMSO-d6) δ 9.72 (s, 1H), 9.12 (s, 2H), 7.80-7.85 (m, 1H), 7.48-7.37 (m, 1H), 7.37-7.21 (m, 1H), 6.39 (s, 1H), 6.08 (s, 1H), 4.95 (s, 1H), 4.51 (s, 1H), 3.79 (d, J=11.1 Hz, 1H), 3.32 (m, 2H), 2.62 (s, 3H), 2.53 (s, 1H), 2.20 (s, 3H), 2.15 (m, 1H), 2.03 (d, J=11.3 Hz, 1H).

### Example 39

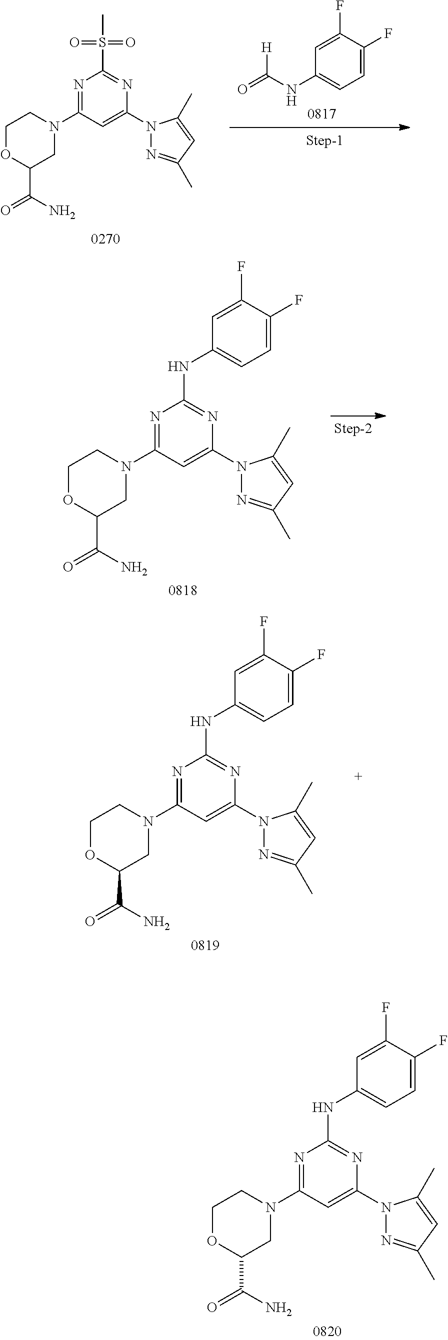 [SMILES: Cc1cc(C)n(-c2cc(N3CCOC(C(N)=O)C3)nc(S(C)(=O)=O)n2)n1 ; O=CNc1ccc(F)c(F)c1 ; Cc1cc(C)n(-c2cc(N3CCOC(C(N)=O)C3)nc(Nc3ccc(F)c(F)c3)n2)n1 ; Cc1cc(C)n(-c2cc(N3CCO[C@H](C(N)=O)C3)nc(Nc3ccc(F)c(F)c3)n2)n1 ; Cc1cc(C)n(-c2cc(N3CCO[C@@H](C(N)=O)C3)nc(Nc3ccc(F)c(F)c3)n2)n1]

Step-1[0818]: The procedure is similar to step Step-1[0748] in example 15. 0.3 g of 4-(6-(3,5-dimethyl-1H-pyrazol-1-yl)-2-(methylsulfonyl) pyrimidin-4-yl)morpholine-2-carboxamide [0270] gave 0.080 g of racemic 4-(2-((3,4-difluorophenyl)amino)-6-(3,5-dimethyl-1H-pyrazol-1-yl)pyrimidin-4-yl)morpholine-2-carboxamide [0818], Compound 129 as an off-white solid, MS(M+1)+=430, 1H NMR (400 MHz, DMSO-d6) δ 9.32 (s, 1H), 7.72 (bs, 1H), 7.52 (s, 1H), 7.42 (m, 1H), 7.37-7.21 (m, 3H), 6.53 (bs, 1H), 6.11 (s, 1H), 4.36 (d, J=12.2 Hz, 1H), 3.94 (d, J=9.8 Hz, 1H), 3.69 (dd, J=11.9, 3.8 Hz, 1H), 3.55-3.35 (m, 3H), 2.62 (s, 3H), 2.19 (s, 3H).

Step-2[0819 and 0820]: 0.08 g of [0818] which was separated by Chiral HPLC to afford (+)-4-(2-((3,4-difluorophenyl)amino)-6-(3,5-dimethyl-1H-pyrazol-1-yl)pyrimidin-4-yl)morpholine-2-carboxamide [0819], Compound 131 as an off-white solid (0.017 g), MS(M+1)+=430, 1H NMR (400 MHz, DMSO-d6) δ 9.32 (s, 1H), 7.72 (bs, 1H), 7.52 (s, 1H), 7.43 (d, J=9.4 Hz, 1H), 7.38-7.19 (m, 2H), 6.53 (bs, 1H), 6.11 (s, 1H), 5.41 (s, 2H), 4.36 (d, J=11.9 Hz, 1H), 3.94 (d, J=9.5 Hz, 1H), 3.69 (dd, J=12.0, 3.9 Hz, 1H), 3.56-3.40 (m, 2H), 2.62 (s, 3H), 2.19 (s, 3H). and (−)-4-(2-((3,4-difluorophenyl)amino)-6-(3,5-dimethyl-1H-pyrazol-1-yl)pyrimidin-4-yl)morpholine-2-carboxamide [0820], Compound 132 as an off-white solid (0.018 g). MS(M+1)+=430, 1H NMR (400 MHz, DMSO-d6) δ 9.32 (s, 1H), 7.72 (bs, 1H), 7.52 (s, 1H), 7.43 (bs, 1H), 7.38-7.19 (m, 2H), 6.53 (bs, 1H), 6.11 (s, 1H), 5.41 (bs, 2H), 4.36 (d, J=11.9 Hz, 1H), 3.94 (d, J=9.5 Hz, 1H), 3.69 (dd, J=12.0, 3.9 Hz, 1H), 3.49 (m, 2H), 2.62 (s, 3H), 2.19 (s, 3H).

### Example 40

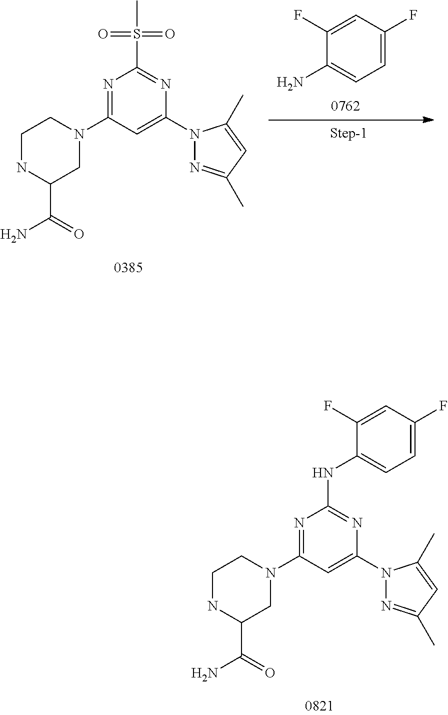 [SMILES: Cc1cc(C)n(-c2cc(N3CCNC(C(N)=O)C3)nc(S(C)(=O)=O)n2)n1 ; Nc1ccc(F)cc1F ; Cc1cc(C)n(-c2cc(N3CCNC(C(N)=O)C3)nc(Nc3ccc(F)cc3F)n2)n1]

Step-1[0821]: The procedure is similar to Step-1 [0813] in example 37. 0.5 g of 4-(6-(3,5-dimethyl-1H-pyrazol-1-yl)-2-(methylsulfonyl)pyrimidin-4-yl)piperazine-2-carboxamide [0385] gave 0.036 g of 4-(2-((2,4-difluorophenyl)amino)-6-(3,5-dimethyl-1H-pyrazol-1-yl)pyrimidin-4-yl)piperazine-2-carboxamide [0821], Compound 110 as an white solid. MS(M+1)+=429. 1H NMR (400 MHz, DMSO-d6) δ 8.69 (s, 1H), 7.58 (m, 1H), 7.37 (bs, 1H), 7.31-7.24 (m, 1H), 7.21 (bs, 1H), 7.10-6.99 (m, 1H), 6.52 (s, 1H), 6.02 (s, 1H), 4.15 (bs, 1H), 3.94 (bs, 1H), 3.21 (dd, J=9.4, 3.5 Hz, 1H), 3.12-2.86 (m, 3H), 2.66 (m, 1H), 2.33 (s, 3H), 2.17 (s, 3H).

### Example 41

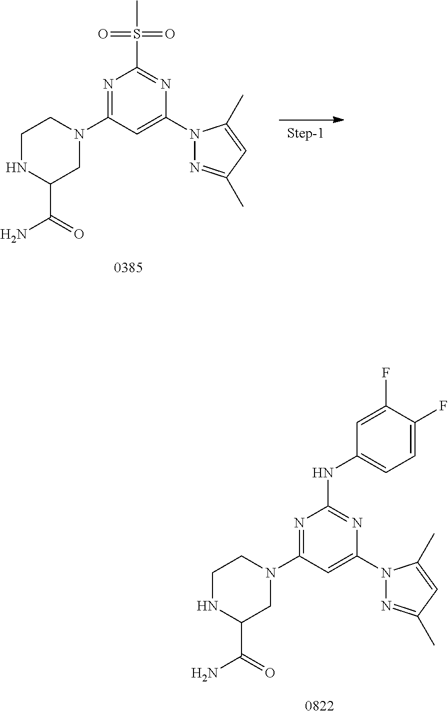 [SMILES: Cc1cc(C)n(-c2cc(N3CCNC(C(N)=O)C3)nc(S(C)(=O)=O)n2)n1 ; Cc1cc(C)n(-c2cc(N3CCNC(C(N)=O)C3)nc(Nc3ccc(F)c(F)c3)n2)n1]

Step-1[0822]: The procedure is similar to Step-1 [0813] in example 37. 0.5 g 4-(6-(3,5-dimethyl-1H-pyrazol-1-yl)-2-(methylsulfonyl)pyrimidin-4-yl)piperazine-2-carboxamide [0385] gave 0.07 g of 4-(2-((3,4-difluorophenyl)amino)-6-(3,5-dimethyl-1H-pyrazol-1-yl)pyrimidin-4-yl)piperazine-2-carboxamide [0822], Compound 116 as an off-white solid. MS(M+1)+=429. 1H-NMR (400 MHz, DMSO-d6): δ 9.32 (s, 1H), 7.85-7.80 (m, 1H), 7.39-7.27 (m, 3H), 7.21 (s, 1H), 6.56 (s, 1H), 6.09 (s, 1H), 4.19 (m, 1H), 4.04-3.95 (m, 1H), 3.28-3.24 (m, 1H), 3.12-3.10 (m, 2H), 2.98-950.00 (m, 1H), 2.75-2.67 (m, 2H), 2.59 (s, 3H), 2.19 (s, 3H).

### Example 42

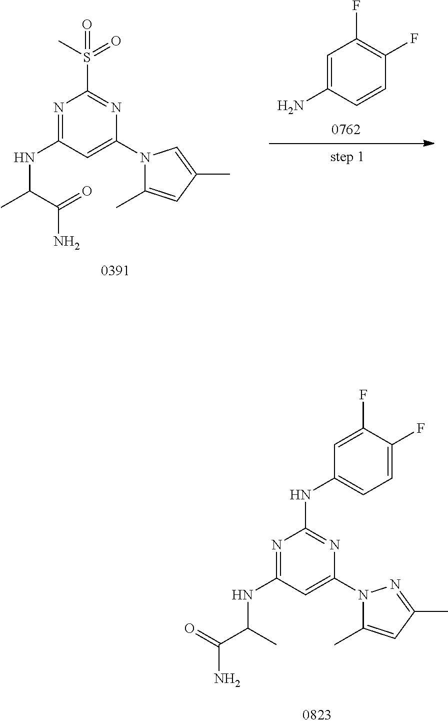 [SMILES: Cc1cc(C)n(-c2cc(NC(C)C(N)=O)nc(S(C)(=O)=O)n2)c1 ; Nc1ccc(F)c(F)c1 ; Cc1cc(C)n(-c2cc(NC(C)C(N)=O)nc(Nc3ccc(F)c(F)c3)n2)n1]

Step 1[0823]: The procedure is similar to Step-1 [0813] in example 37. 0.25 g of 2-((6-(3,5-dimethyl-1H-pyrazol-1-yl)-2-(methylsulfonyl)pyrimidin-4-yl)amino)propanamide [0391] gave 0.02 g of 2-((2-((3,4-difluorophenyl)amino)-6-(3,5-dimethyl-1H-pyrazol-1-yl)pyrimidin-4-yl)amino)propanamide [0823], Compound 113 as an off-white solid. MS(M+1)+=388.3, 1H NMR (400 MHz, DMSO-d6) δ 9.18 (s, 1H), 7.80 (bs, 1H), 7.49 (bs, 2H), 7.34 (bs, 1H), 7.28 (q, J=10.8 Hz, 1H), 7.02 (bs, 1H), 6.47 (bs, 1H), 6.07 (s, 1H), 4.40 (bs, 1H), 2.60 (s, 3H), 2.17 (s, 3H), 1.34 (d, J=7.1 Hz, 3H).

### Example 43

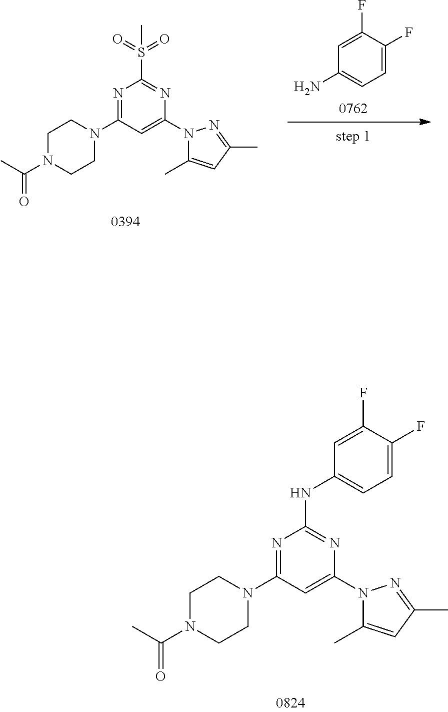 [SMILES: CC(=O)N1CCN(c2cc(-n3nc(C)cc3C)nc(S(C)(=O)=O)n2)CC1 ; Nc1ccc(F)c(F)c1 ; CC(=O)N1CCN(c2cc(-n3nc(C)cc3C)nc(Nc3ccc(F)c(F)c3)n2)CC1]

Step 1[0824]: To a solution of 1-(4-(6-(3,5-dimethyl-1H-pyrazol-1-yl)-2-(methylsulfonyl)pyrimidin-4-yl)piperazin-1-yl)ethan-1-one [0394] (0.4 g, 1.056 mmol) in dimethylsulphoxide (4 mL) was added 3,4-difluoroaniline (0.4 g, 3.17 mmol) and cesium carbonate (1.37 g, 4.22 mmol). The reaction mixture was heated at 100° C. for 18 h. The reaction mixture was quenched with ice. It was then extracted with ethyl acetate (2×20 mL). The combined organic extracts were washed with water (10 mL), followed by brine (10 mL) and dried over anhydrous sodium sulfate to afford 0.32 g of 1-(4-(2-((3,4-difluorophenyl)amino)-6-(3,5-dimethyl-1H-pyrazol-1-yl)pyrimidin-4-yl)piperazin-1-yl)ethan-1-one. This was purified by column chromatography using 1% methanol in chloroform to afford 0.110 g of 1-(4-(2-((3,4-difluorophenyl)amino)-6-(3,5-dimethyl-1H-pyrazol-1-yl)pyrimidin-4-yl)piperazin-1-yl)ethan-1-one [0824], Compound 101 as a white solid. MS(M+1)+=428.2. 1H NMR (400 MHz, DMSO-d6) δ 9.36 (s, 1H), 7.85-7.75 (m, 1H), 7.38-7.33 (m, 2H), 6.58 (s, 1H), 6.11 (s, 1H), 3.71 (bs, 2H), 3.60-3.57 (m, 6H), 2.61 (s, 3H), 2.20 (s, 3H), 2.06 (s, 3H).

### Example 44

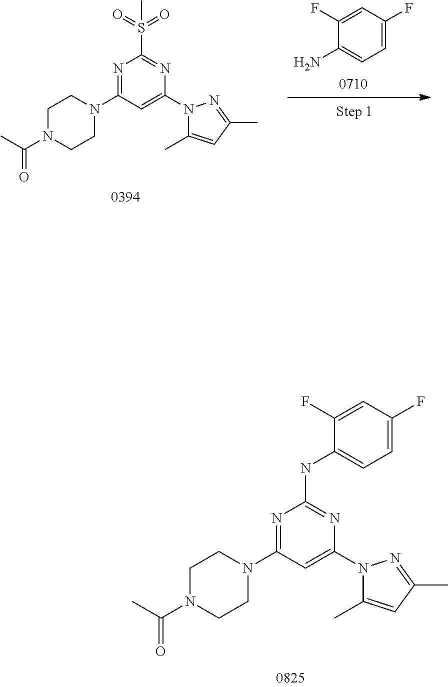 [SMILES: CC(=O)N1CCN(c2cc(-n3nc(C)cc3C)nc(S(C)(=O)=O)n2)CC1 ; Nc1ccc(F)cc1F ; CC(=O)N1CCN(c2cc(-n3nc(C)cc3C)nc(Nc3ccc(F)cc3F)n2)CC1]

Step 1[0825]. The procedure is similar to step 1[0824] in example 43. 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.095 g of 1-(4-(2-((2,4-difluorophenyl)amino)-6-(3,5-dimethyl-1H-pyrazol-1-yl)pyrimidin-4-yl)piperazin-1-yl)ethan-1-one[0825], Compound 104 as a white solid. MS(M+1)+=428.4. 1H NMR (400 MHz, DMSO-d6) δ 8.72 (s, 1H), 7.54 (q, J=9 Hz, 1H), 7.31-7.25 (m, 1H), 7.08-7.04 (m, 1H), 6.53 (s, 1H), 6.03 (s, 1H), 3.65 (bs, 2H), 3.56-3.53 (m, 6H), 2.34 (s, 3H), 2.17 (s, 3H), 2.05 (s, 3H).

### Example 45

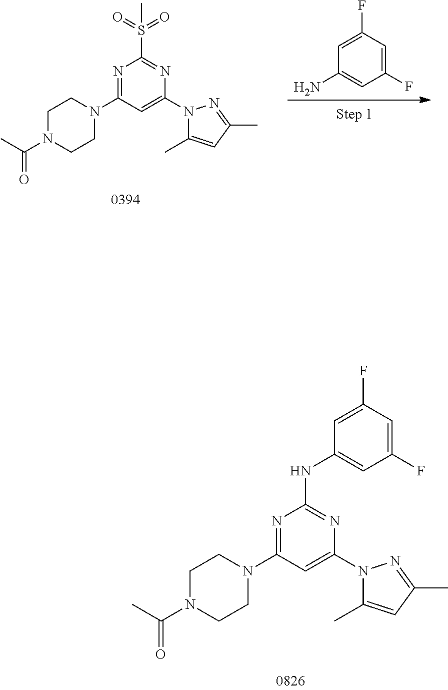 [SMILES: CC(=O)N1CCN(c2cc(-n3nc(C)cc3C)nc(S(C)(=O)=O)n2)CC1 ; Nc1cc(F)cc(F)c1 ; CC(=O)N1CCN(c2cc(-n3nc(C)cc3C)nc(Nc3cc(F)cc(F)c3)n2)CC1]

Step 1[0826]: The procedure is similar to step 1[0824] in example 43. 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.085 g of 1-(4-(2-((3,5-difluorophenyl)amino)-6-(3,5-dimethyl-1H-pyrazol-1-yl)pyrimidin-4-yl)piperazin-1-yl)ethan-1-one [0826], Compound 172 as an off-white solid. MS(M+1)+=428.2. 1H NMR (400 MHz, DMSO-d6) δ 9.56 (s, 1H), 7.51-7.33 (m, 2H), 6.72 (tt, J=9.3, 2.3 Hz, 1H), 6.61 (s, 1H), 6.12 (s, 1H), 3.72 (bs, 2H), 3.66-3.49 (m, 6H), 2.62 (s, 3H), 2.19 (s, 3H), 2.05 (s, 3H).

### Example 46

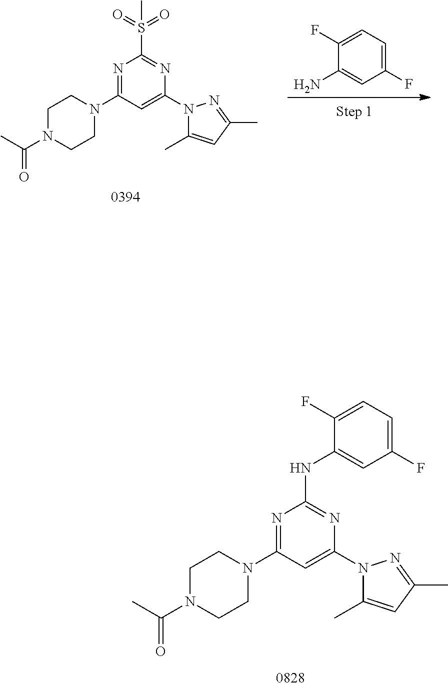 [SMILES: CC(=O)N1CCN(c2cc(-n3nc(C)cc3C)nc(S(C)(=O)=O)n2)CC1 ; Nc1cc(F)ccc1F ; CC(=O)N1CCN(c2cc(-n3nc(C)cc3C)nc(Nc3cc(F)ccc3F)n2)CC1]

Step 1[0828]: The procedure is similar to step 1[0824] in example 43, 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.14 g of 1-(4-(2-((2,5-difluorophenyl)amino)-6-(3,5-dimethyl-1H-pyrazol-1-yl)pyrimidin-4-yl)piperazin-1-yl)ethan-1-one [0828], Compound 176 as a yellow solid. MS(M+1)+=428.2. 1H NMR (400 MHz, DMSO-d6) δ 8.86 (s, 1H), 7.8-7.6 (m, 1H), 7.35-7.2 (m, 1H), 7.0-6.88 (m, 1H), 6.59 (s, 1H), 6.07 (s, 1H), 3.76-3.64 (m, 2H), 3.64-3.50 (m, 6H), 2.47 (s, 3H), 2.18 (s, 3H), 2.06 (s, 3H).

### Example 47

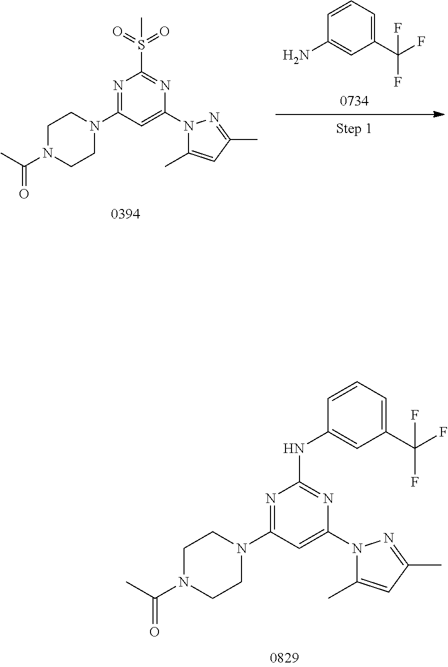 [SMILES: CC(=O)N1CCN(c2cc(-n3nc(C)cc3C)nc(S(C)(=O)=O)n2)CC1 ; Nc1cccc(C(F)(F)F)c1 ; CC(=O)N1CCN(c2cc(-n3nc(C)cc3C)nc(Nc3cccc(C(F)(F)F)c3)n2)CC1]

Step 1[0829]: The procedure is similar to step 1[0824] in example 43. 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.11 g of 1-(4-(6-(3,5-dimethyl-1H-pyrazol-1-yl)-2-((3-(trifluoromethyl)phenyl)amino)pyrimidin-4-yl)piperazin-1-yl)ethan-1-one [0829], Compound 174 as a yellow solid. MS(M+1)+=460.4. 1H NMR (400 MHz, DMSO-d6) δ 9.53 (s, 1H), 8.28 (s, 1H), 7.81 (d, J=8.4 Hz, 1H), 7.51 (t, J=8.0 Hz, 1H), 7.27 (d, J=7.9 Hz, 1H), 6.60 (s, 1H), 6.12 (s, 1H), 3.72 (bs, 2H), 3.65-3.55 (m, 6H), 2.62 (s, 3H), 2.20 (s, 3H), 2.07 (s, 3H).

### Example 48

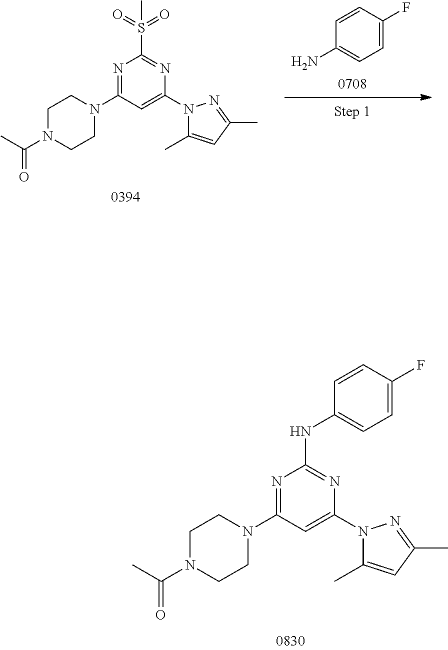 [SMILES: CC(=O)N1CCN(c2cc(-n3nc(C)cc3C)nc(S(C)(=O)=O)n2)CC1 ; Nc1ccc(F)cc1 ; CC(=O)N1CCN(c2cc(-n3nc(C)cc3C)nc(Nc3ccc(F)cc3)n2)CC1]

Step 1[0830]: The procedure is similar to step 1[0824] in example 43. 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.075 g of 1-(4-(6-(3,5-dimethyl-1H-pyrazol-1-yl)-2-((4-fluorophenyl)amino)pyrimidin-4-yl)piperazin-1-yl)ethan-1-one [0830], Compound 103 as a yellow solid. MS(M+1)+=410.2. 1H NMR (400 MHz, DMSO-d6) δ 9.15 (s, 1H), 7.63 (dd=9.0, 5.0 Hz, 2H), 7.11 (t, J=8.8 Hz, 2H), 6.52 (s, 1H), 6.08 (s, 1H), 3.68 (bs, 2H), 3.62-3.46 (m, 6H), 2.57 (s, 3H), 2.18 (s, 3H), 2.05 (s, 3H).

### Example 49

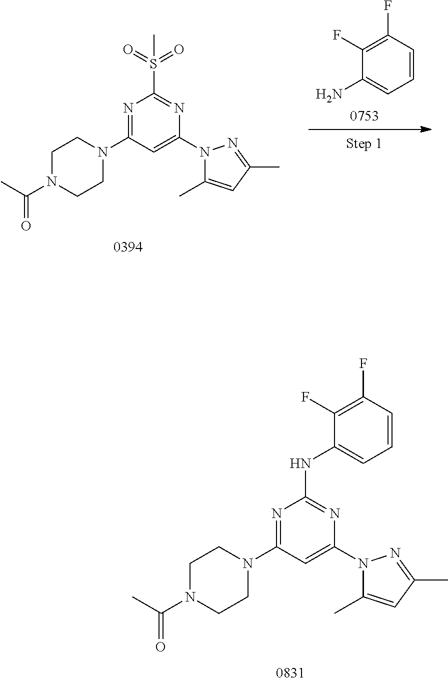

Step 1[0831]: The procedure is similar to step 1[0824] in example 43, 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.08 g of 1-(4-(2-((2,3-difluorophenyl)amino)-6-(3,5-dimethyl-1H-pyrazol-1-yl)pyrimidin-4-yl)piperazin-1-yl)ethan-1-one[0831], Compound 173 as beige solid. MS(M+1)+=428.4. 1H NMR (400 MHz, DMSO-d6) δ 8.96 (s, 1H), 7.42 (s, 1H), 7.21-7.06 (m, 2H), 6.56 (s, 1H), 6.04 (s, 1H), 3.65 (bs, 2H), 3.60-3.47 (m, 6H), 2.39 (s, 3H), 2.17 (s, 3H), 2.04 (s, 3H).

### Example 50

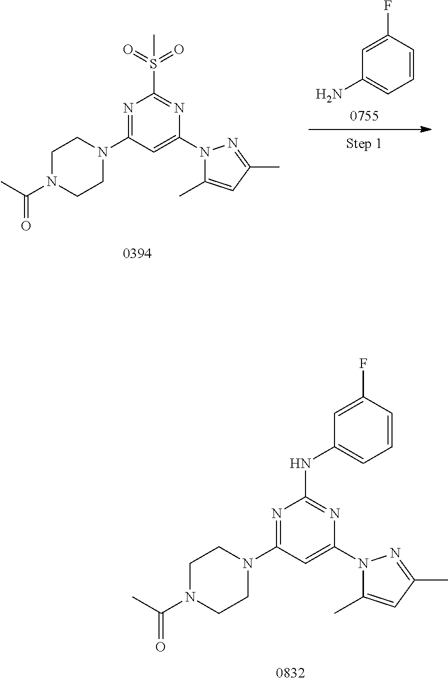

Step 1[0832]: The procedure is similar to step 1[0824] in example 43. 0.4 g of 1-(4-(6-(3,5-dimethyl-1H-pyrazol-1-yl)-2-(methylsulfonyl)pyrimidin-4-yl)piperazin-1-yl)ethan-1-one [0394] gave 0.8 g of 1-(4-(6-(3,5-dimethyl-1H-pyrazol-1-yl)-2-((3-fluorophenyl)amino)pyrimidin-4-yl)piperazin-1-yl)ethan-1-one[0832], Compound 175 as a yellow solid. MS(M+1)+=410.2. 1H-NMR (400 MHz, DMSO-d6): δ 9.39 (s, 1H), 7.66 (d, J=12.32 Hz, 1H), 7.43 (d, J=8.88 Hz, 1H), 7.33-7.27 (m, 1H), 6.76-6.72 (m, 1H), 6.58 (s, 1H), 6.11 (s, 1H), 3.72 (s, 2H), 3.63-3.58 (m, 6H), 2.62 (s, 3H), 2.20 (s, 3H), 2.06 (s, 3H).

### Example 51

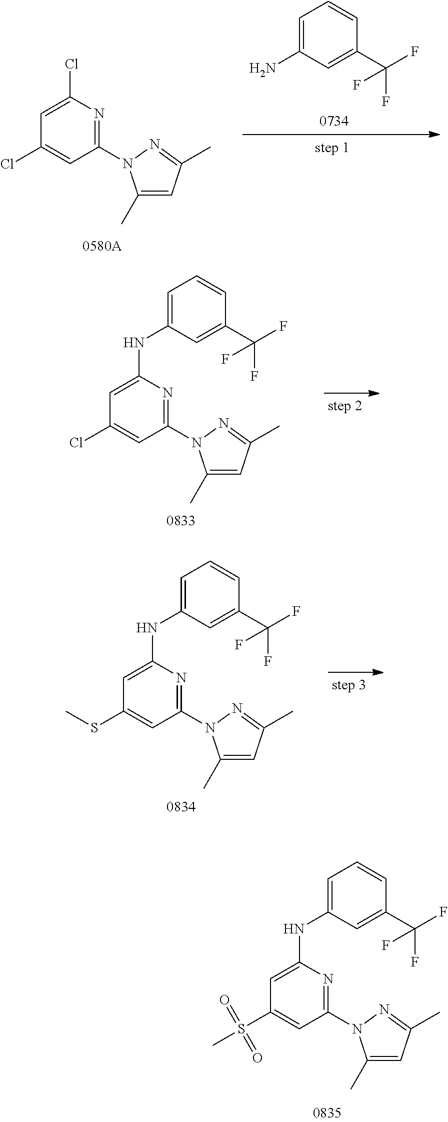 [SMILES: Cc1cc(C)n(-c2cc(Cl)cc(Cl)n2)n1 ; Nc1cccc(C(F)(F)F)c1 ; Cc1cc(C)n(-c2cc(Cl)cc(Nc3cccc(C(F)(F)F)c3)n2)n1 ; CSc1cc(Nc2cccc(C(F)(F)F)c2)nc(-n2nc(C)cc2C)c1 ; Cc1cc(C)n(-c2cc(S(C)(=O)=O)cc(Nc3cccc(C(F)(F)F)c3)n2)n1]

Step 1[0833]: To the stirred solution of 2,4-dichloro-6-(3,5-dimethyl-1H-pyrazol-1-yl)pyridine[0580A] (0.9 g, 3.71 mmol) in dioxane was added 3-aminobenzotrifluoride [0734](0.59 g, 3.71 mmol), Cesium carbonate(2.42 g, 7.43 mmol), 4,5-Bis(diphenylphosphino)-9,9-dimethylxanthene (0.12 g, 0.44 mmol) and the reaction mixture was purged with nitrogen for 5 min. Then Palladium (II) acetate (0.067 g, 0.29 mmol) was added to the reaction mixture and the reaction mixture was irradiated at 100° C. in microwave. After 30 min, the reaction mixture was passed through celite bed, washed with chloroform and the filtrate was concentrated under reduced pressure to afford a brown solid, which was purified in the Reveleris flash system instrument using ethyl acetate in hexane as solvent in 25 g column to afford 0.5 g of 4-chloro-6-(3,5-dimethyl-1H-pyrazol-1-yl)-N-(3-(trifluoromethyl)phenyl)pyridin-2-amine [0833] as brown solid. MS(M+1)+=365.0.

Step 2 [0834]: To a solution of 4-chloro-6-(3,5-dimethyl-1H-pyrazol-1-yl)-N-(3-(trifluoromethyl)phenyl) pyridin-2-amine [0833] (0.8 g, 2.18 mmol) and sodium thiomethoxide (0.305 g, 4.36 mmol) in N,N-dimethylformamide was added cesium carbonate (1.5 g, 3.27 mmol) and the reaction mixture was heated at 80° C. After 16 h, the reaction mixture was cooled to rt, quenched with water and stirred for 10 min. The solid formed was filtered and washed with water to afford 6-(3,5-dimethyl-1H-pyrazol-1-yl)-4-(methylthio)-N-(3-(trifluoromethyl) phenyl)pyridin-2-amine [0834] as a pale yellow solid. (0.8 g). MS(M+1)+=379.4.

Step 3[0835]: To a solution of 6-(3,5-dimethyl-1H-pyrazol-1-yl)-4-(methylthio)-N-(3-(trifluoromethyl)phenyl)pyridin-2-amine [0834] (0.8 g, 2.11 mmol) in dichloromethane (10 mL) was added 3-chloroperbenzoic acid (1.09 g, 6.34 mmol) in portions at 0° C. The reaction mixture was slowly warmed to rt. After 6 h, the reaction mixture was diluted with dichloromethane, washed with saturated sodium thiosulfate solution and followed by 10% sodium bicarbonate solution. The organic layer was dried over anhydrous sodium sulfate, filtered and concentrated and which was purified by column chromatography using 50% ethyl acetate in hexane to afford 6-(3,5-dimethyl-1H-pyrazol-1-yl)-4-(methylsulfonyl)-N-(3-(trifluoromethyl)phenyl)pyridin-2-amine[0835], Compound 196 as brown solid. MS(M+1)+=411.4. 1H NMR (400 MHz, DMSO-d6) δ 10.04 (s, 1H), 8.05 (s, 1H), 7.70 (d, J=8.6 Hz, 1H), 7.63-7.48 (m, 2H), 7.36 (d, J=7.9 Hz, 1H), 7.19 (s, 1H), 6.16 (s, 1H), 3.34 (s, 3H), 2.48 (s, 3H), 2.22 (s, 3H).

### Example 52

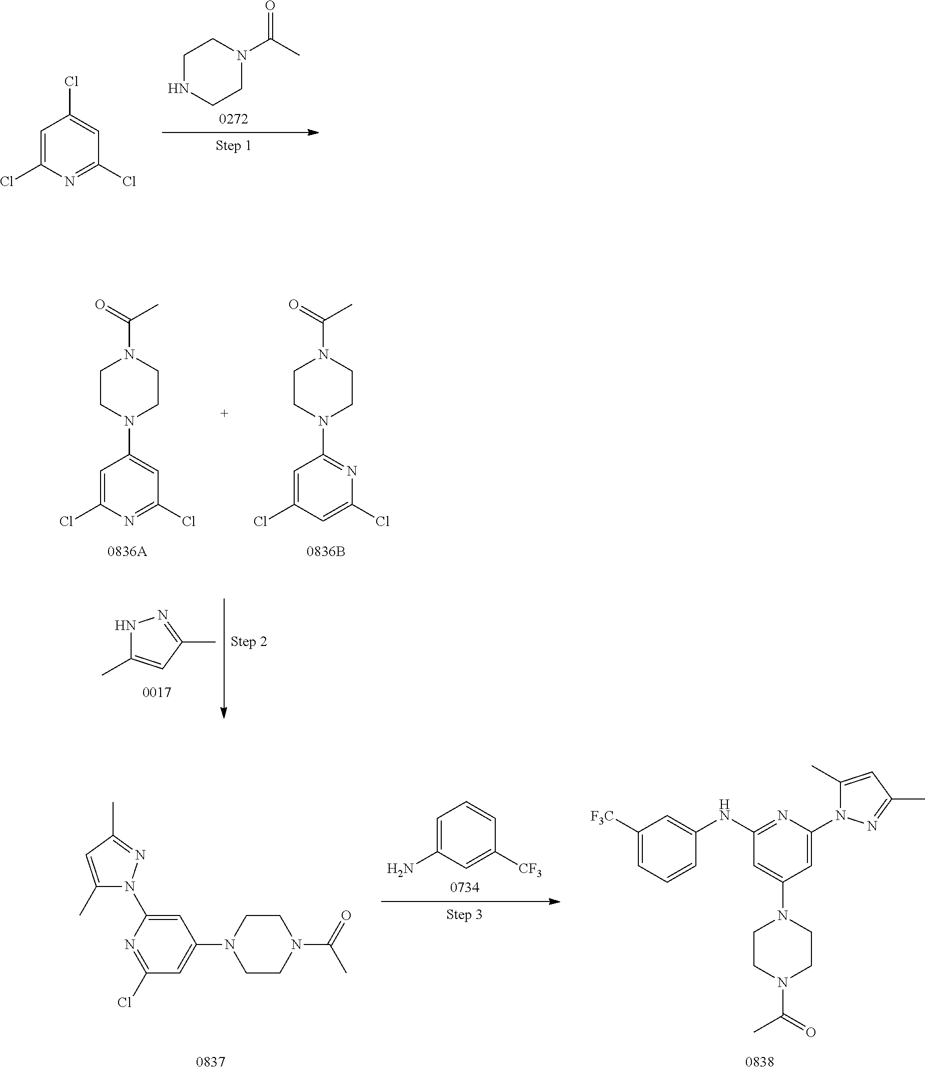

Step 1[0836 and 0836B]: The procedure is similar to step 1[0824] in Example 43. 3 g of 2,4,6-trichloropyridine[0565] gave 1.5 g of 1-(4-(2,6-dichloropyridin-4-yl)piperazin-1-yl)ethan-1-one [0836A] as white solid and 1.5 g of 1-(4-(4,6-dichloropyridin-2-yl)piperazin-1-yl)ethan-1-one[0836B] as white solid MS(M+1)+=275.2.

Step 2[0837]: To a stirred solution of 1-(4-(2,6-dichloropyridin-4-yl)piperazin-1-yl)ethan-1-one [0836A] (1 g, 3.64 mmol) and 3,5-Dimethyl pyrazole [0017] (0.35 g, 3.64 mmol) in Dimethylsulphoxide was added cesium carbonate (1.78 g, 5.47 mmol) and the reaction mixture was heated at 100° C. in sealed tube. After 16 h, the reaction mixture was diluted with water, extracted with ethyl acetate and washed with brine solution. The combined organic layer was dried over sodium sulfate, filtered and concentrated under reduced pressure to afford a colorless gum, which was purified in the Reveleris flash system instrument using ethyl acetate in hexane as solvent in 40 g column to afford 1-(4-(2-chloro-6-(3,5-dimethyl-1H-pyrazol-1-yl)pyridin-4-yl)piperazin-1-yl)ethan-1-one [0837] as colorless gum. (0.33 g, 27% yield). MS(M+1)+=334.2.

Step 3[0838]: The procedure is similar to step 1[0748] in Example 15. 0.3 g of 1-(4-(2-chloro-6-(3,5-dimethyl-1H-pyrazol-1-yl)pyridin-4-yl)piperazin-1-yl)ethan-1-one [0837] gave 0.22 g of 1-(4-(2-(3,5-dimethyl-1H-pyrazol-1-yl)-6-((3-(trifluoromethyl)phenyl)amino)pyridin-4-yl)piperazin-1-yl)ethan-1-one [0838], Compound 182 as yellow solid. MS(M+1)+=459.7. 1H NMR (400 MHz, DMSO-d6) δ 9.29 (s, 1H), 8.13 (d, J=2.0 Hz, 1H), 7.63 (dd, J=7.7, 2.0 Hz, 1H), 7.46 (t, J=8.0 Hz, 1H), 7.18 (d, J=7.8 Hz, 1H), 6.74 (d, J=2.0 Hz, 1H), 6.17 (d, J=2.0 Hz, 1H), 6.06 (s, 1H), 3.60 (m, 4H), 3.40 (m, 2H), 3.32 (m, 2H), 2.46 (s, 3H), 2.19 (s, 3H), 2.06 (s, 3H).

### Example 53

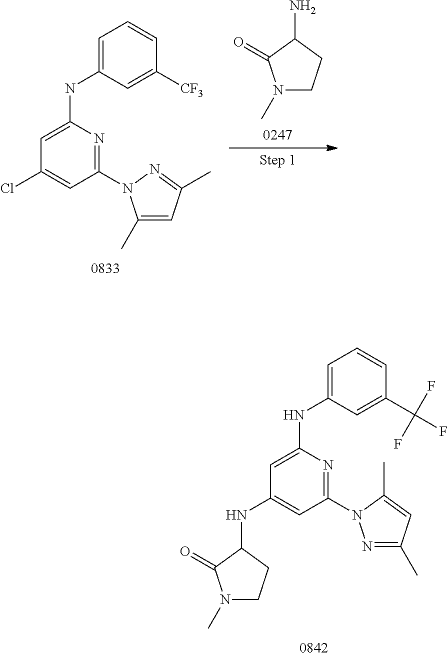

Step 3[0842]: The procedure is similar to step 1[0748] in Example 15. 0.3 g of 4-chloro-6-(3,5-dimethyl-1H-pyrazol-1-yl)-N-(3-(trifluoromethyl)phenyl)pyridin-2-amine[0833] gave 0.025 g 3-((2-(3,5-dimethyl-1H-pyrazol-1-yl)-6-((3-(trifluoromethyl)phenyl)amino)pyridin-4-yl)amino)-1-methylpyrrolidin-2-one [0842], Compound 191 as yellow solid. MS(M+1)+=445.1, 1H NMR (400 MHz, DMSO-d6) δ 9.12 (s, 1H), 8.09 (d, J=2.1 Hz, 1H), 7.63 (d, J=8.0 Hz, 1H), 7.43 (t, J=7.9 Hz, 1H), 7.14 (d, J=7.8 Hz, 1H), 6.86 (d, J=7.3 Hz, 1H), 6.57 (d, J=1.9 Hz, 1H), 6.09-5.91 (m, 2H), 4.18 (q, J=8.4 Hz, 1H), 3.37-3.33 (m, 2H), 2.78 (s, 3H), 2.45 (s, 3H), 2.16 (s, 3H), 1.80 (dd, J=12.5, 9.0 Hz, 2H).

### Example 54

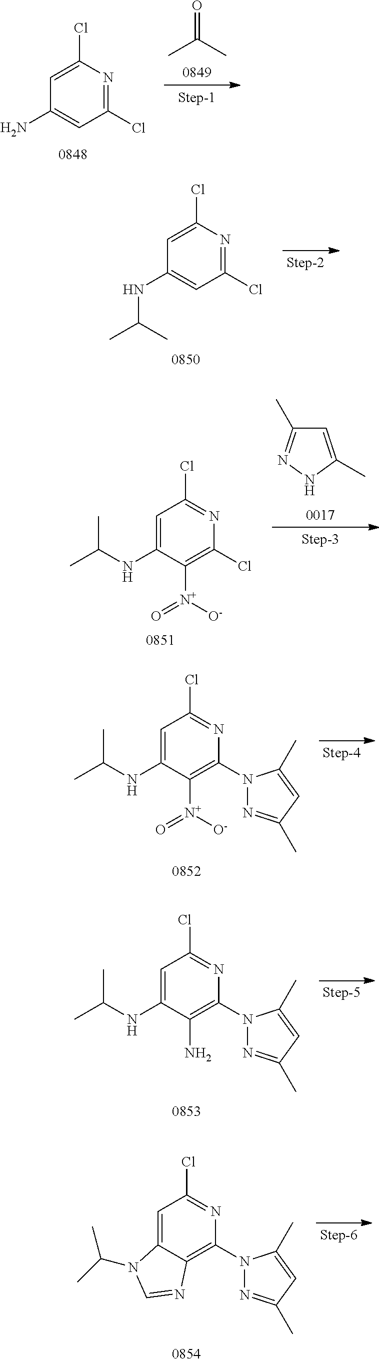

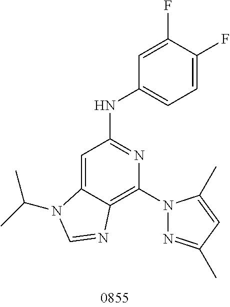 [SMILES: Cc1cc(C)n(-c2nc(Nc3ccc(F)c(F)c3)cc3c2ncn3C(C)C)n1]

Step 1[0850]: To a solution of 4-amino-2,6-dichloropyridine [0848] (6 g, 36.80 mmol) in dichloromethane, acetic acid (60 mL) and acetone (5.7 g, 99.38 mmol) was added borane dimethyl sulfide complex (5.9 g, 73.61 mmol) at 0° C. The reaction mixture was slowly warmed to rt for 2 h. The reaction mixture was basified (pH ˜9) by using liquid ammonia at 0° C. and extracted with dichloromethane, the organic layer was washed with water and brine solution. The combined organic layer was dried over sodium sulfate, filtered and concentrated under reduced pressure to afford crude and which was purified through column using ethyl acetate in hexane as solvent to afford 2,6-dichloro-N-ethylpyridin-4-amine[0850] as a white solid (6.8 g). MS(M+1)+=206.0.

Step 2[0851]: To a solution of 2,6-dichloro-N-ethylpyridin-4-amine [0850] (6.8 g, 33.15 mmol) in concentrated sulfuric acid (55.2 g, 562.8 mmol) was added nitric acid (2.08 g, 33.1 mmol) slowly drop wise at 0° C. and the reaction mixture was stirred at same temperature. After 1 h, the reaction mixture was cooled to 0° C. and quenched with ice and stirred for 10 min. The solid formed was filtered, washed with water and dried under vacuum to afford 2,6-dichloro-N-isopropyl-3-nitropyridin-4-amine [0851] as an yellow solid (7.2 g). MS(M+1)+=251.4.

Step 3[0852]: The procedure is similar to step 1[0719] in Example 2. 5.9 g 2,6-dichloro-N-isopropyl-3-nitropyridin-4-amine [0851] gave 4.5 g of 6-chloro-2-(3,5-dimethyl-1H-pyrazol-1-yl)-N-isopropyl-3-nitropyridin-4-amine [0852] as yellow solid. MS(M+1)+=310.3.

Step 4[0853]: To a suspension of 6-chloro-2-(3,5-dimethyl-1H-pyrazol-1-yl)-N-isopropyl-3-nitropyridin-4-amine[0852] (2 g, 6.45 mmol) in dichloromethane and methanol was added Raney nickel (0.42 g, 3.22 mmol) and the reaction mixture was stirred at rt under hydrogen atmosphere. After 7 h, the reaction mixture was filtered through celite bed, washed with dichloromethane. The filtrate was concentrated under reduced pressure to afford 6-chloro-2-(3,5-dimethyl-1H-pyrazol-1-yl)-N4-isopropylpyridine-3,4-diamine [0853] as an brown solid (2.5 g) and taken as such to next step. MS(M+1)+=280.3

Step 5[0854]: To a solution of 6-chloro-2-(3,5-dimethyl-1H-pyrazol-1-yl)-N4-isopropylpyridine-3,4-diamine [0853] (0.5 g) in formic acid (20 vol) was heated at 80° C. for 6 h. The reaction mixture was concentrated under reduced pressure and the residue was neutralized with sodium bicarbonate solution, extracted with ethyl acetate, washed with water and brine solution. The combined organic layer was concentrated under reduced pressure to afford a purple solid, which was purified in the Reveleris flash system instrument using ethyl acetate in hexane as solvent in 12 g column to afford 6-chloro-4-(3,5-dimethyl-1H-pyrazol-1-yl)-1-isopropyl-1H-imidazo[4,5-c]pyridine [0854] as an brown solid (0.43 g). MS(M+1)+=290.2.

Step 6[0855]: To the stirred solution of 6-chloro-4-(3,5-dimethyl-1H-pyrazol-1-yl)-1-isopropyl-1H-imidazo[4,5-c]pyridine [0854] (0.4 g, 1.38 mmol) in dioxane was added 3,4-Difluoroaniline (0.71 g, 5.52 mmol), Cesium carbonate (0.80 g, 2.48 mmol), 4,5-bis(diphenylphosphino)-9,9-dimethylxanthene (0.319 g, 0.552 mmol) and the reaction mixture was purged with nitrogen for 5 min. Then tris(dibenzylideneacetone)dipalladium (0.37 g, 0.414 mmol) was added to the reaction mixture and the reaction mixture was heated at 90° C. in sealed tube. After 16 h, the reaction mixture was passed through celite bed, washed with chloroform and the filtrate was concentrated under reduced pressure to afford brown oil, which was purified in the Reveleris flash system instrument using methanol in chloroform as solvent in 24 g column, to afford N-(3,4-difluorophenyl)-4-(3,5-dimethyl-1H-pyrazol-1-yl)-1-ethyl-1H-imidazo[4,5-c]pyridin-6-amine [0855], Compound 119 as pale brown solid (0.33 g). MS(M+1)+=383.0, 1H NMR (400 MHz, DMSO-d6) δ 9.27 (s, 1H), 8.33 (s, 1H), 7.84-7.90 (m, 1H), 7.30 (q, J=9.6 Hz, 1H), 7.20-7.18 (m, 1H), 7.01 (s, 1H), 6.10 (s, 1H), 4.69 (p, J=6.8 Hz, 1H), 2.29 (s, 3H), 2.21 (s, 3H), 1.55 (d, J=6.7 Hz, 6H).

### Example 55

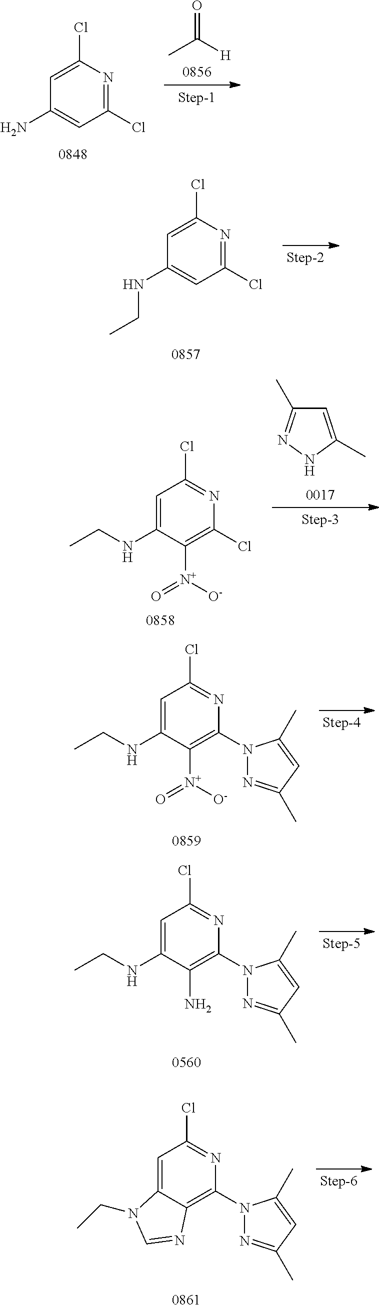

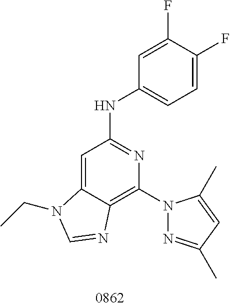 [SMILES: CCn1cnc2c(-n3nc(C)cc3C)nc(Nc3ccc(F)c(F)c3)cc21]

Step 1[0857]: To a solution of 4-amino-2,6-dichloropyridine [0848] (5 g, 30.673 mmol) in dichloromethane (40 mL), acetic acid (10 mL) and acetaldehyde (1.35 g, 1.72 mmol) was added borane dimethyl sulfide complex (3.4 mL, 33.74 mmol) at 0° C. (effervescence occurs). The reaction mixture was slowly warmed to rt for 15 min. The reaction mixture was quenched with liquid ammonia (15 mL) to pH around 9 at 0° C. and extracted with dichloromethane (2×30 mL), washed with water (10 mL) and brine solution (10 mL). The organic layer was dried over sodium sulfate, filtered and concentrated under reduced pressure to afford crude and which was purified by column chromatography using 20% ethyl acetate in pet ether as solvent to afford 2,6-dichloro-N-ethylpyridin-4-amine [0857] as an white solid (4.3 g). MS(M+1)+=192.

Step 2[0858]: To a suspension of 2,6-dichloro-N-ethylpyridin-4-amine [0857] (4.36 g, 22.82 mmol) in concentrated sulfuric acid (40 mL, 750.42 mmol) at 0° C. was added fuming nitric acid (1.15 mL, 22.82 mmol) drop wise (reaction mixture turns clear) and the reaction mixture was stirred at 0° C. for 2 h. The reaction mixture was quenched with ice. The solid formed was filtered to afford 2, 6-dichloro-N-ethyl-3-nitropyridin-4-amine [0858] as a yellow solid (5 g). MS(M+1)+=236.1

Step 3[0859]: To a suspension of sodium hydride (1.25 g, 31.39 mmol) in tetrahydrofuran at 0° C. was added 3,5-dimethyl pyrazole (2.01 g, 20.927 mmol) and stirred at the same temperature for 15 min. After 15 min 2, 6-dichloro-N-ethyl-3-nitropyridin-4-amine [0858] (4.94 g, 20.927 mmol) was added to the reaction mixture at 0° C. (grey suspension turns blood red color) and slowly warmed to rt for 2 h. The reaction mixture was quenched with ice. The solid formed was filtered to afford 6-chloro-2-(3,5-dimethyl-1H-pyrazol-1-yl)-N-ethyl-3-nitropyridin-4-amine [0859] as a yellow solid (5.7 g). MS(M+1)+=296.2.

Step 4[0860]: To a solution of 6-chloro-2-(3,5-dimethyl-1H-pyrazol-1-yl)-N-ethyl-3-nitropyridin-4-amine [0859] (2.8 g, 9.468 mmol) in dichloromethane (10 mL) was added Raney-nickel (0.61 g, 4.734 mmol) in methanol (20 mL) and the reaction mixture was stirred at rt under hydrogen atmosphere (bladder) for 8 h. The reaction mixture was filtered through celite bed, filtrate was concentrated under reduced pressure to afford 6-chloro-2-(3,5-dimethyl-1H-pyrazol-1-yl)-N4-ethylpyridine-3,4-diamine [0860] as a brown solid (2.5 g). MS(M+1)+=266.2.

Step 5[0861]: To a solution of 6-chloro-2-(3,5-dimethyl-1H-pyrazol-1-yl)-N4-ethylpyridine-3,4-diamine [0860] (2.5 g, 9.40 mmol), was added formic acid (25 mL). The reaction mixture was heated at 80° C. for 5 h. The reaction mixture was concentrated under reduced pressure to afford crude and which was purified by column chromatography using 5% methanol in chloroform to afford 6-chloro-4-(3,5-dimethyl-1H-pyrazol-1-yl)-1-ethyl-1H-imidazo[4,5-c]pyridine [0861] as an brown solid (2 g). MS(M+1)+=276.2.

Step 6[0862]: To a stirred solution of 6-chloro-4-(3,5-dimethyl-1H-pyrazol-1-yl)-1-ethyl-1H-imidazo[4,5-c]pyridine [0861] (0.4 g, 1.45 mmol) in dioxane (5 mL) was added 3,4-difluoroaniline (0.74 g, 5.80 mmol), cesium carbonate (0.85 g, 2.611 mmol), 4,5-bis(diphenylphosphino)-9,9-dimethylxanthene (0.335 g, 0.58 mmol) and the reaction mixture was purged with nitrogen for 5 min. Tris(dibenzylideneacetone)dipalladium(0) (0.398 g, 0.435 mmol) was added to the reaction mixture and the reaction mixture was heated at 90° C. in a sealed tube for 48 h. The reaction mixture was diluted with ethyl acetate (20 mL) and it was passed through a celite bed. The filtrate was concentrated under reduced pressure to afford crude and which was purified by preparative HPLC to afford N-(3,4-difluorophenyl)-4-(3,5-dimethyl-1H-pyrazol-1-yl)-1-ethyl-1H-imidazo[4,5-c]pyridin-6-amine of [0862], Compound 120 as a white solid (0.100 g). MS(M+1)+=369.2, 1H NMR (400 MHz, DMSO-d6) δ 9.27 (s, 1H), 8.24 (s, 1H), 7.86 (dddd, J=21.6, 14.1, 7.5, 2.6 Hz, 1H), 7.29 (q, J=10.7, 9.2 Hz, 1H), 7.20-7.18 (m, 1H), 6.97 (s, 1H), 6.09 (s, 1H), 4.23 (q, J=7.3 Hz, 2H), 2.28 (s, 3H), 2.21 (s, 3H), 1.42 (t, J=7.2 Hz, 3H).

### Example 56

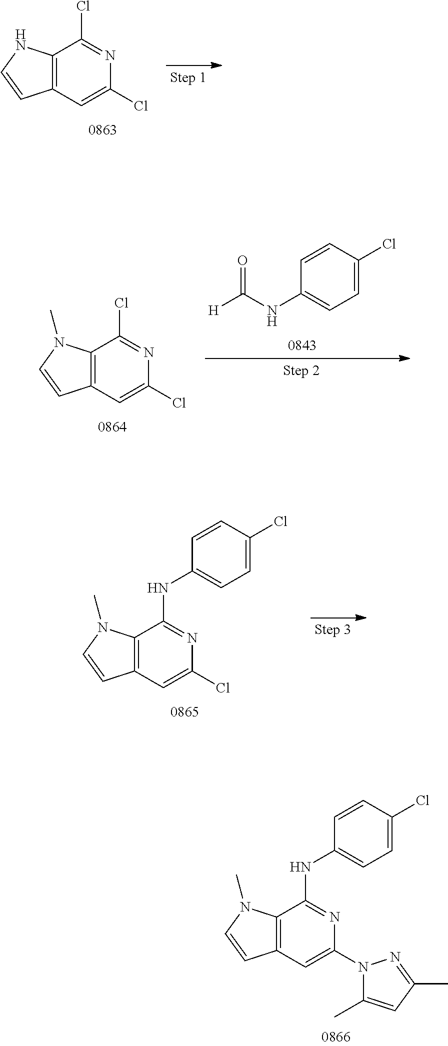 [SMILES: Clc1cc2cc[nH]c2c(Cl)n1 ; Cn1ccc2cc(Cl)nc(Cl)c21 ; O=CNc1ccc(Cl)cc1 ; Cn1ccc2cc(Cl)nc(Nc3ccc(Cl)cc3)c21 ; Cc1cc(C)n(-c2cc3ccn(C)c3c(Nc3ccc(Cl)cc3)n2)n1]

Step 1[0864]: To a suspension of Sodium hydride (0.4 g, 1.38 mmol) in N,N-dimethylformamide was added 5,7-dichloro-1H-pyrrolo[2,3-c]pyridine [0863] (0.4 g, 1.38 mmol) at 0° C. and the reaction mixture was stirred at rt. After 30 min, iodomethane was added to the reaction mixture at 0° C. and reaction mixture was slowly warmed to rt. After 16 h, the reaction mixture was quenched with ice and stirred for 10 min. The solid formed was filtered, washed with water and dried under vacuum to afford 5,7-dichloro-1-methyl-1H-pyrrolo[2,3-c]pyridine [0864], as pale brown solid (1.7 g). MS(M+1)+=202.2.

Step 2[0865]: To a solution of 5,7-dichloro-1-methyl-1H-pyrrolo[2,3-c]pyridine [0864] (1.6 g, 7.95 mmol) and N-(4-chlorophenyl)formamide [0843] (1.2 g, 7.95 mmol) in acetonitrile (15 mL) was added cesium carbonate (3.8 g, 11.9 mmol) and the reaction mixture was irradiated in microwave at 140° C. The reaction mixture was filtered, washed with ethyl acetate and the filtrate was concentrated under reduced pressure to afford a red oil, which was purified in the Reveleris flash system instrument using ethyl acetate in hexane as solvent in 40 g column to afford 5-chloro-N-(4-chlorophenyl)-1-methyl-1H-pyrrolo[2,3-c]pyridin-7-amine [0865] as a pale brown solid (0.75 g). MS(M+1)+=293.1.

Step 3[0866]: The procedure is step 3[0766] in Example 24 (at 150° C.). 0.6 g of 5-chloro-N-(4-chlorophenyl)-1-methyl-1H-pyrrolo[2,3-c]pyridin-7-amine [0865] gave 0.013 g of N-(4-chlorophenyl)-5-(3,5-dimethyl-1H-pyrazol-1-yl)-1-methyl-1H-pyrrolo [2,3-c]pyridin-7-amine[0866], Compound 144 as red solid. MS(M+1)+=352.3, 1H NMR (400 MHz, DMSO-d6) δ 8.46 (s, 1H), 7.50 (d, J=2.9 Hz, 1H), 7.39 (s, 1H), 7.35 (d, J=8.8 Hz, 2H), 7.29 (d, J=8.96 Hz, 2H), 6.49 (d, J=2.9 Hz, 1H), 5.96 (s, 1H), 4.11 (s, 3H), 2.29 (s, 3H), 2.16 (s, 3H).

### Example 57

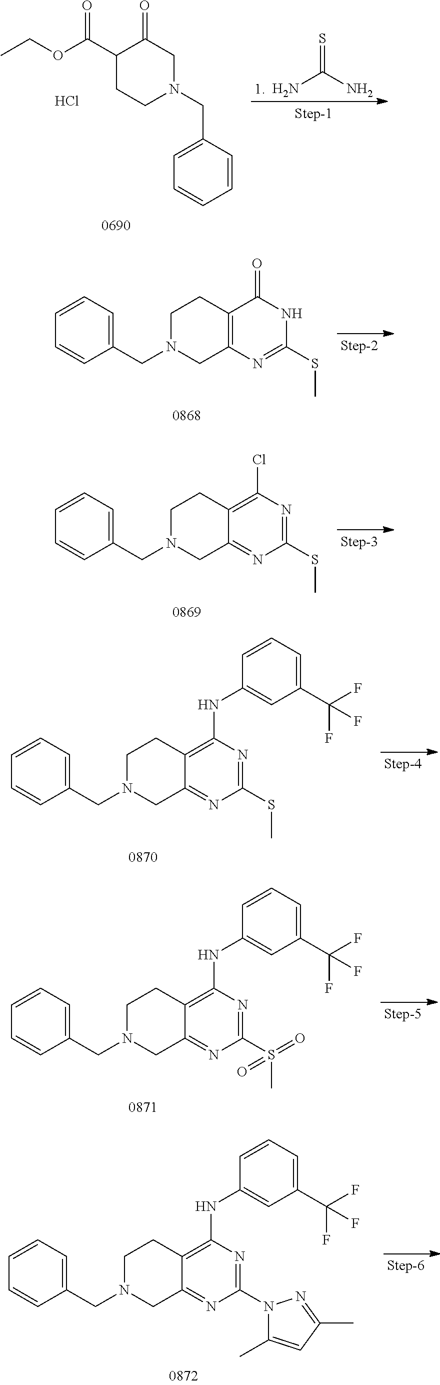

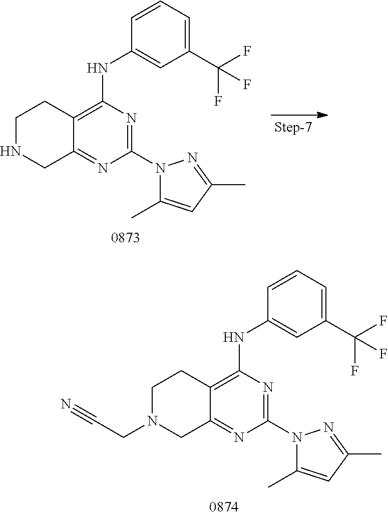 [SMILES: Cc1cc(C)n(-c2nc3c(c(Nc4cccc(C(F)(F)F)c4)n2)CCNC3)n1 ; Cc1cc(C)n(-c2nc3c(c(Nc4cccc(C(F)(F)F)c4)n2)CCN(CC#N)C3)n1]

Step-1[0868]: A suspension of Ethyl 1-benzyl-3-oxo-4-piperidinecarboxylate hydrochloride [0690] (8.9 g, 29.8 mmol) and thiourea [0867] (4.5 g, 59.11 mmol) in the solution of sodium methoxide in methanol (34 mL) was stirred at 100° C. in a sealed tube for 16 h. The reaction mixture was cooled to rt, then added iodomethane (5.1 g, 35.9 mmol) and stirred at rt for 1h. After the completion of the reaction, the reaction mixture was concentrated to remove methanol and the resulting residue was dissolved in water and extracted by chloroform and IPA (3:1, 10×40 mL), the combined organic layer was dried over sodium sulfate and concentrated to afford 7-benzyl-2-(methylthio)-5,6,7,8-tetrahydropyrido[3,4-d]pyrimidin-4(4aH)-one [0868] as an off-white solid (7.5 g), MS(M+1)+=288.

Step-2[0869]: An ice cooled suspension of 7-benzyl-2-(methylthio)-5,6,7,8-tetrahydropyrido[3,4-d]pyrimidin-4(4aH)-one [0868] (7.5 g, 26.0 mmol) in 1,2-dichloroethane (20 mL) was added dimethyl aniline (3.3 g, 27.2 mmol 1.0) and phosphorus oxychloride (32.5 g, 211.9 mmol 8.). Then the reaction mixture was heated at 90° C. After the completion of the reaction, the reaction mixture was cooled to rt and poured into ice cold water, neutralized by solid sodium bicarbonate and extracted with ethyl acetate (3×100 mL), the combined organic layer was dried over sodium sulfate and concentrated to afford 7-benzyl-4-chloro-2-(methylthio)-5,6,7,8-tetrahydropyrido[3,4-d]pyrimidine [0869] as an light brown oil (7 g). MS(M+1)+=306.

Step-3[0870]: To a solution of -benzyl-4-chloro-2-(methylthio)-5,6,7,8-tetrahydropyrido[3,4-d]pyrimidine [0869] (0.5 g, 1.63 mmol) in dioxane (6 mL) was added 3-aminobenzotrifluoride (0.28 g, 1.79 mmol), sodium iodide (0.24 g, 1.63 mmol) and hydroiodic acid (51% aqueous solution) (6 mL), then the reaction mixture was heated at 100° C. in a closed vial for 16h. After the completion of the reaction, the reaction mixture was cooled to rt, the obtained solid was filtered and washed with ethyl acetate and dried under high vacuum to afford 7-benzyl-2-(methylthio)-N-(3-(trifluoromethyl)phenyl)-5,6,7,8-tetrahydropyrido [3,4-d]pyrimidin-4-amine [0870] as an off-white solid (0.4 g). MS(M+1)+=431.

Step-4[0871]: To an ice cooled suspension of 7-benzyl-2-(methylthio)-N-(3-(trifluoromethyl) phenyl)-5,6,7,8-tetrahydropyrido[3,4-d]pyrimidin-4-amine [0870] (0.4 g, 0.929 mmol) in ethanol (8 mL) was added 3-chloroperbenzoic acid (0.48 g, 2.78 mmol). Then the reaction mixture was slowly warmed to rt and stirred for 6 h. After the completion of the reaction, the reaction mixture was quenched with 10% sodium bicarbonate solution and extracted with chloroform:IPA (3:1, 80 mL), and washed saturated sodium thiosulfate solution, dried over sodium sulfate and concentrated to afford 7-benzyl-2-(methylsulfonyl)-N-(3-(trifluoromethyl)phenyl)-5,6,7,8-tetrahydropyrido[3,4-d]pyrimidin-4-amine [0871] as an off-white solid (0.2 g). MS(M+1)+=463.

Step-5[0872]: To a stirred solution of 7-benzyl-2-(methylsulfonyl)cN-(3-(trifluoromethyl) phenyl)-5,6,7,8-tetrahydropyrido[3,4-d]pyrimidin-4-amine [0871] (0.5 g, 1.08 mmol) in dioxane (10 mL) was added 3,5-Dimethyl pyrazole (0.207 g, 2.16 mmol), Cesium carbonate (0.49 g, 1.51 mmol), 4,5-Bis(diphenylphosphino)-9,9-dimethylxanthene (0.187 g, 0.324 mmol) and the reaction mixture was purged with nitrogen for 5 min. Then tris(dibenzylideneacetone)dipalladium(0) (0.19 g, 0.21 mmol0.) was added to the reaction mixture before heating at 90° C. in a closed vial for 16 h. After the completion of the reaction, the reaction mixture was filtered through celite bed to remove catalyst, the organic layer was concentrated to afford as an brownish gum and was which was purified by Prep HPLC to afford 7-benzyl-2-(3,5-dimethyl-1H-pyrazol-1-yl)-N-(3-(trifluoromethyl)phenyl)-5,6,7,8-tetrahydropyrido[3,4-d]pyrimidin-4-amine [0872], Compound 192 as an pink solid (0.6 g). MS(M+1)+=479, 1H NMR (400 MHz, DMSO-d6) δ 8.72 (s, 1H), 8.43 (d, J=10.2 Hz, 1H), 8.02 (t, J=9.2 Hz, 1H), 7.55 (t, J=8.2 Hz, 1H), 7.45-7.24 (m, 6H), 6.02 (d, J=7.8 Hz, 1H), 4.05-3.96 (m, 1H), 3.75 (d, J=13.5 Hz, 1H), 3.20 (dd, J=7.7, 3.5 Hz, 2H), 2.82-2.61 (m, 4H), 2.47-2.40 (m, 3H), 2.18 (s, 3H).

Step6 [0873]: To a solution of 7-benzyl-2-(3,5-dimethyl-1H-pyrazol-1-yl)-N-(3-(trifluoromethyl)phenyl)-5,6,7,8-tetrahydropyrido[3,4-d]pyrimidin-4-amine [0872] (0.25 g, 0.522 mmol) in dichloromethane (10 mL) was added 1-chloroethyl chloroformate (0.14 g, 1.04 mmol) and N,N-diisopropyl ethylamine (0.13 g, 1.04 mmol). The reaction mixture was stirred at rt for 2 h. The reaction mixture was concentrated and purified by Prep HPLC to afford as gummy solid and which was triturated with hexane, the obtained solid was filtered and dried under high vacuum to afford 2-(3,5-dimethyl-1H-pyrazol-1-yl)-N-(3-(trifluoromethyl)phenyl)-5,6,7,8-tetrahydropyrido[3,4-d]pyrimidin-4-amine [0873], Compound 194 as an white solid (0.020 g). MS(M+1)+=389, 1H NMR (400 MHz, DMSO-d6) δ 9.49 (s, 2H), 9.24 (s, 1H), 8.50 (s, 1H), 8.00 (d, J=8.2 Hz, 1H), 7.60 (t, J=8.0 Hz, 1H), 7.46 (d, J=7.8 Hz, 1H), 6.09 (s, 1H), 4.23 (s, 2H), 3.51 (t, J=5.6 Hz, 2H), 2.94 (d, J=6.1 Hz, 2H), 2.43 (s, 3H), 2.19 (s, 3H).

Step-7[0874]: To a solution of 2-(3,5-dimethyl-1H-pyrazol-1-yl)-N-(3-(trifluoromethyl)phenyl)-5,6,7,8-tetrahydropyrido[3,4-d]pyrimidin-4-amine [0873] (0.09 g, 0.231 mmol) in acetonitrile (6 mL) was added bromo acetonitrile (0.057 g, 0.46 mmol) and followed by cesium carbonate (0.151 g, 0.46 mmol). The reaction mixture was stirred at 80° C. for 16 h. After the completion of the reaction, the reaction mixture was filtered and the filtrate was concentrated to afford as a brownish gum which was purified by column of silica gel (60-120 mesh), using 95% ethyl acetate in hexane as eluent to afford 2-(2-(3,5-dimethyl-1H-pyrazol-1-yl)-4-((3-(trifluoromethyl)phenyl)amino)-5,8-dihydropyrido[3,4-d]pyrimidin-7(6H)-yl)acetonitrile [0874], Compound 193 as an Light brown solid (0.018 g). MS(M+1)+=428, 1H NMR (400 MHz, DMSO-d6) δ 8.98 (s, 1H), 8.56 (s, 1H), 8.00 (d, J=8.0 Hz, 1H), 7.67-7.50 (m, 1H), 7.40 (d, J=7.7 Hz, 1H), 6.06 (s, 1H), 3.99 (s, 2H), 3.62 (s, 2H), 2.89 (t, J=5.7 Hz, 2H), 2.78 (d, J=5.7 Hz, 2H), 2.43 (s, 3H), 2.17 (s, 3H).

### Example 58

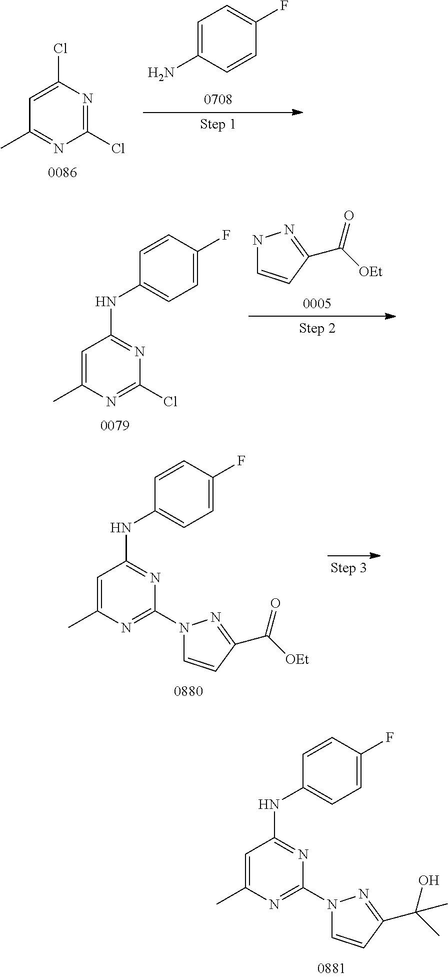

Step 1[0879]: 3.0 g of 2,4-dichloro-6-methylpyrimidine [0086] and 4-fluoroaniline [0708] (2.0 g, 18.4 mmol) gave 1.8 g of 2-chloro-N-(4-fluorophenyl)-6-methylpyrimidin-4-amine [0879] as an off-white solid. (Using DIPEA, ACN, 80° C., 16h). MS(M+1)+=238.8.

Step 2[0880]: The procedure is similar to step 1[0270] in example 98. 1.8 g 2-chloro-N-(4-fluorophenyl)-6-methylpyrimidin-4-amine [0879] and ethyl 1H-pyrazole-3-carboxylate [0005] (1.08 g, 7.57 mmol) gave 1.1 g of ethyl 1-(4-((4-fluorophenyl)amino)-6-methylpyrimidin-2-yl)-1H-pyrazole-3-carboxylate [0880] as an off-white solid. (Using CS₂CO₃, ACN, 90° C., 16h)MS(M+1)+=342.0.

Step 3[0881]: The procedure is similar to step 2[0765] in example 24. 0.25 g of ethyl 1-(4-((4-fluorophenyl)amino)-6-methylpyrimidin-2-yl)-1H-pyrazole-3-carboxylate [0880] gave 0.062 g of 2-(1-(4-((4-fluorophenyl)amino)-6-methylpyrimidin-2-yl)-1H-pyrazol-3-yl)propan-2-ol [0881], Compound 138 as an off-white solid. MS(M+1)+=328.0, 1H NMR (400 MHz, DMSO-d6) δ 9.79 (s, 1H), 8.39 (d, J=2.6 Hz, 1H), 7.93-7.61 (m, 2H), 7.19 (t, J=8.9 Hz, 2H), 6.68-6.36 (m, 2H), 5.08 (s, 1H), 2.35 (s, 3H), 1.49 (s, 6H).

### Example 59

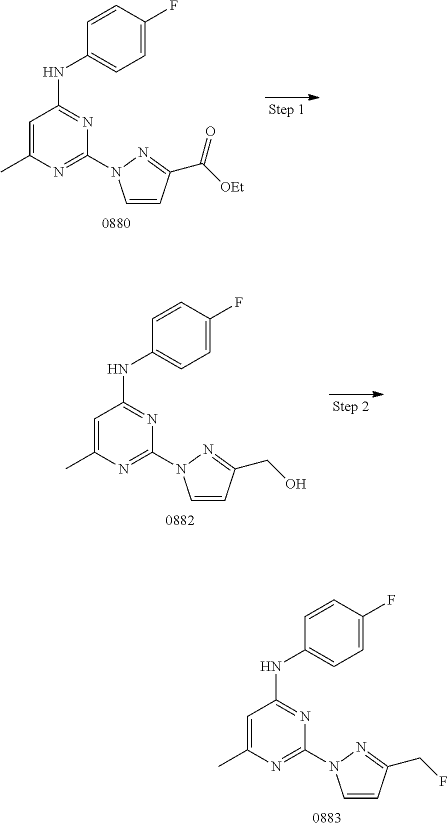 [SMILES: CCOC(=O)c1ccn(-c2nc(C)cc(Nc3ccc(F)cc3)n2)n1 ; Cc1cc(Nc2ccc(F)cc2)nc(-n2ccc(CO)n2)n1 ; Cc1cc(Nc2ccc(F)cc2)nc(-n2ccc(CF)n2)n1]

Step 1[0882]: The procedure is similar to step 2[0771] in example 25. 0.7 g ethyl 1-(4-((4-fluorophenyl)amino)-6-methylpyrimidin-2-yl)-1H-pyrazole-3-carboxylate [0880] gave 0.38 g of (1-(4-((4-fluorophenyl)amino)-6-methylpyrimidin-2-yl)-1H-pyrazol-3-yl)methanol [0882], Compound 137 as an off-white solid. MS(M+1)+=299.8, 1H NMR (400 MHz, DMSO-d6) δ 9.81 (s, 1H), 8.45 (s, 1H), 7.72 (dd, J=9.0, 4.9 Hz, 2H), 7.23 (t, J=8.8 Hz, 2H), 6.50 (d, J=2.9 Hz, 2H), 5.23 (t, J=6.0 Hz, 1H), 4.52 (d, J=5.9 Hz, 2H), 2.35 (s, 3H).

Step 2[0883]: The procedure is similar to step 3[0760] in example 21. 0.15 g (1-(4-((4-fluorophenyl)amino)-6-methylpyrimidin-2-yl)-1H-pyrazol-3-yl)methanol [0882] gave 0.062 g of 2-(3-(fluoromethyl)-1H-pyrazol-1-yl)-N-(4-fluorophenyl)-6-methylpyrimidin-4-amine [0883], Compound 141 as an off-white solid. MS(M+1)+=302.0, 1H NMR (400 MHz, DMSO-d6) δ 9.88 (s, 1H), 8.54 (d, J=2.6 Hz, 1H), 7.72 (dd, J=9.0, 4.9 Hz, 2H), 7.33-7.14 (m, 2H), 6.69 (dd, J=2.7, 1.3 Hz, 1H), 6.54 (s, 1H), 5.45 (d, J_{F}=48 Hz, 2H), 2.37 (s, 3H).

### Example 60

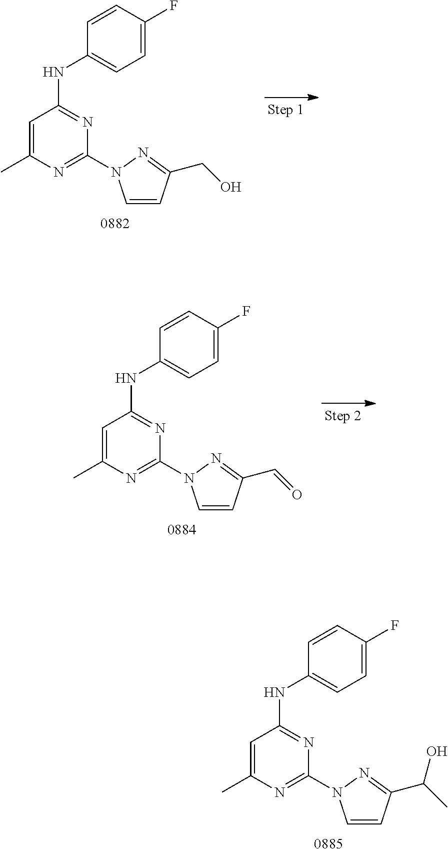 [SMILES: Cc1cc(Nc2ccc(F)cc2)nc(-n2ccc(CO)n2)n1 ; Cc1cc(Nc2ccc(F)cc2)nc(-n2ccc(C=O)n2)n1 ; Cc1cc(Nc2ccc(F)cc2)nc(-n2ccc(C(C)O)n2)n1]

Step 1 [0884]: The procedure is similar to step 1[0786] in example 28. 0.18 g (1-(4-((4-fluorophenyl)amino)-6-methylpyrimidin-2-yl)-1H-pyrazol-3-yl)methanol [0882] gave 0.155 g of 1-(4-((4-fluorophenyl)amino)-6-methylpyrimidin-2-yl)-1H-pyrazole-3-carbaldehyde [0884] as a white solid. MS(M+1)+=297.8.

Step 2[0885]: The procedure is similar to step 2[0765] in example 24. 0.15 g of 1-(4-((4-fluorophenyl)amino)-6-methylpyrimidin-2-yl)-1H-pyrazole-3-carbaldehyde [0884] gave 0.065 g of 1-(1-(4-((4-fluorophenyl)amino)-6-methylpyrimidin-2-yl)-1H-pyrazol-3-yl)ethan-1-ol [0885], Compound 140 as a white solid. MS(M+1)+=314.0, 1H NMR (400 MHz, DMSO-d6) δ 9.80 (s, 1H), 8.43 (d, J=2.6 Hz, 1H), 7.73 (dd, J=8.9, 5.0 Hz, 2H), 7.21 (t, J=8.9 Hz, 2H), 6.55-6.40 (m, 2H), 5.25 (d, J=4.9 Hz, 1H), 4.93-4.73 (m, 1H), 2.35 (s, 3H), 1.42 (d, J=6.5 Hz, 3H).

### Example 61

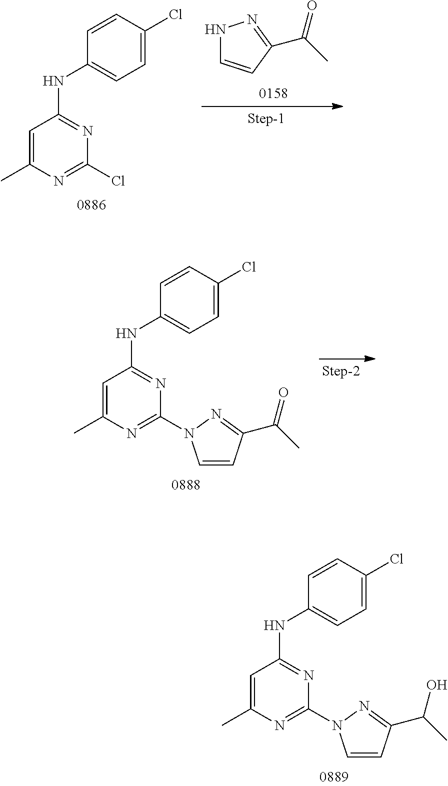

Step 1[0888]: The procedure is similar to Step 3 [0766] in example 24 (at 150° C.). 2 g of 2-chloro-N-(4-chlorophenyl)-6-methylpyrimidin-4-amine [0886] gave 0.5 g of 1-(1-(4-((4-chlorophenyl)amino)-6-methylpyrimidin-2-yl)-1H-pyrazol-3-yl)ethan-1-one[0888], MS(M+1)+=328.2.

Step 2 [0889]: The procedure is similar to Step 4 [0722] in example 2. 0.25 g of 1-(1-(4-((4-chlorophenyl)amino)-6-methylpyrimidin-2-yl)-1H-pyrazol-3-yl)ethan-1-one [0888] gave 1-(1-(4-(4-chlorobenzyl)-6-methylpyrimidin-2-yl)-1H-pyrazol-3-yl)ethan-1-ol [0889], Compound 139. MS(M+1)+=330.2, 1H NMR (400 MHz, DMSO-d6) δ 9.96 (d, J=5.6 Hz, 1H), 8.45 (d, J=2.7 Hz, 1H), 7.90-7.58 (m, 2H), 7.49-7.18 (m, 2H), 6.59-6.36 (m, 2H), 4.81 (q, J=6.5 Hz, 1H), 2.37 (s, 3H), 1.43 (d, J=6.6 Hz, 3H).

### Example 62

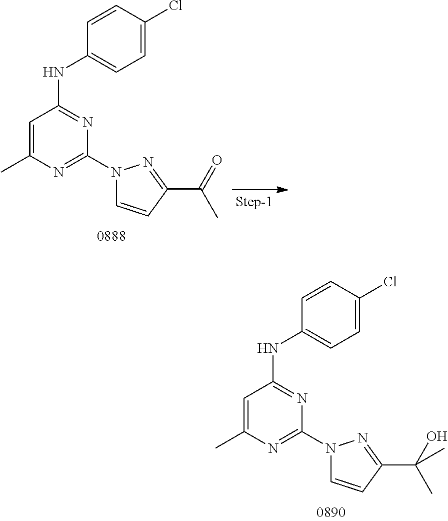 [SMILES: CC(=O)c1ccn(-c2nc(C)cc(Nc3ccc(Cl)cc3)n2)n1 ; Cc1cc(Nc2ccc(Cl)cc2)nc(-n2ccc(C(C)(C)O)n2)n1]

Step 1[0890]: The procedure is similar to Step 2 [0765] in example 24. 0.25 g of 1-(1-(4-((4-chlorophenyl)amino)-6-methylpyrimidin-2-yl)-1H-pyrazol-3-yl)ethan-1-one [0888] gave 0.09 g of 2-(1-(4-((4-chlorophenyl)amino)-6-methylpyrimidin-2-yl)-1H-pyrazol-3-yl)propan-2-ol [0890], Compound 142. MS(M+1)+=344.2, MR=74.1-78.3° C., 1H NMR (400 MHz, DMSO-d6) δ 9.90 (s, 1H), 8.42 (d, J=2.7 Hz, 1H), 7.93-7.74 (m, 2H), 7.46-7.32 (m, 2H), 6.53 (s, 2H), 5.10 (s, 1H), 2.37 (s, 3H), 1.51 (s, 6H).

### Example 63

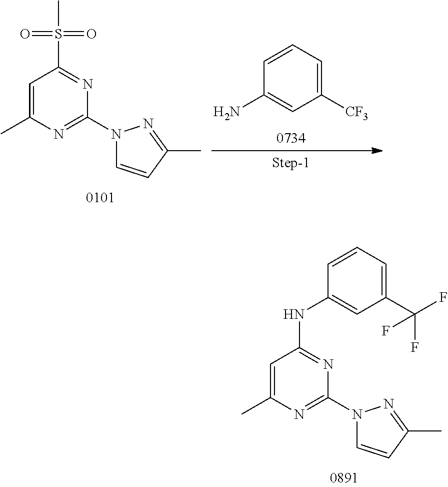

Step-1[0891]: A stirred solution of 4-methyl-2-(3-methyl-1H-pyrazol-1-yl)-6-(methylsulfonyl) pyrimidine [0101] (0.15 g, 0.595 mmol) and 3-aminobenzotrifluoride [0734](0.144 g, 0.892 mmol) in tetrahydrofuran (5 mL) was heated at 100° C. in a sealed tube for 16 h. The reaction mixture was concentrated under reduced pressure to afford crude product which was purified by column chromatography using 35% ethyl acetate in hexane as eluent to afford 6-methyl-2-(3-methyl-1H-pyrazol-1-yl)-N-(3-(trifluoromethyl)phenyl)pyrimidin-4-amine [0891], Compound 198 as an white solid (0.06 g, 31%). MS(M+1)+=334.0, MR=70.1-80.7° C., 1H NMR (400 MHz, DMSO-d6) δ 10.10 (s, 1H), 8.61 (s, 1H), 8.39 (d, J=2.6 Hz, 1H), 7.83 (dd, J=8.4, 2.1 Hz, 1H), 7.58 (t, J=8.0 Hz, 1H), 7.36 (d, J=7.7 Hz, 1H), 6.55 (s, 1H), 6.38 (d, J=2.6 Hz, 1H), 2.38 (s, 3H), 2.28 (s, 3H).

### Example 64

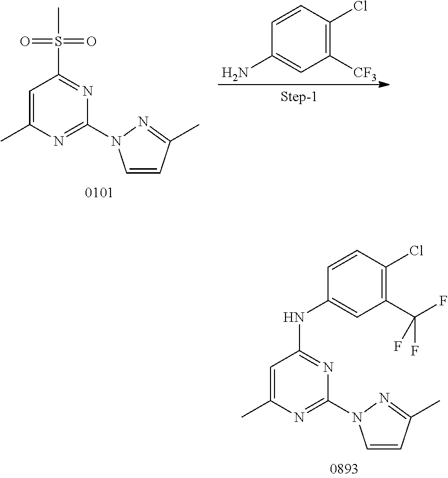

Step-1 [0893]: A stirred solution of 4-methyl-2-(3-methyl-1H-pyrazol-1-yl)-6-(methylsulfonyl)pyrimidine [0101] (0.15 g, 0.595 mmol) and 5-Amino-2-Chlorobenzotrifluoride [0892] (0.232 g, 1.189 mmol) in tetrahydrofuran (5 mL) was heated at 100° C. in a sealed tube for 16 h. The reaction mixture was concentrated under reduced pressure to afford crude product which was purified by column chromatography using 35% ethyl acetate in hexane as eluent to afford N-(4-chloro-3-(trifluoromethyl)phenyl)-6-methyl-2-(3-methyl-1H-pyrazol-1-yl)pyrimidin-4-amine [0893] as white solid (0.08 g), Compound 136. MS(M+1)+=368.0, MR=119.4-124.0° C., 1H NMR (400 MHz, DMSO-d6) δ 10.21 (s, 1H), 8.82 (d, J=2.7 Hz, 1H), 8.41 (d, J=2.5 Hz, 1H), 7.87 (dd, J=8.9, 2.7 Hz, 1H), 7.69 (d, J=8.9 Hz, 1H), 6.55 (s, 1H), 6.39 (d, J=2.5 Hz, 1H), 2.39 (s, 3H), 2.30 (s, 3H).

### Example 64

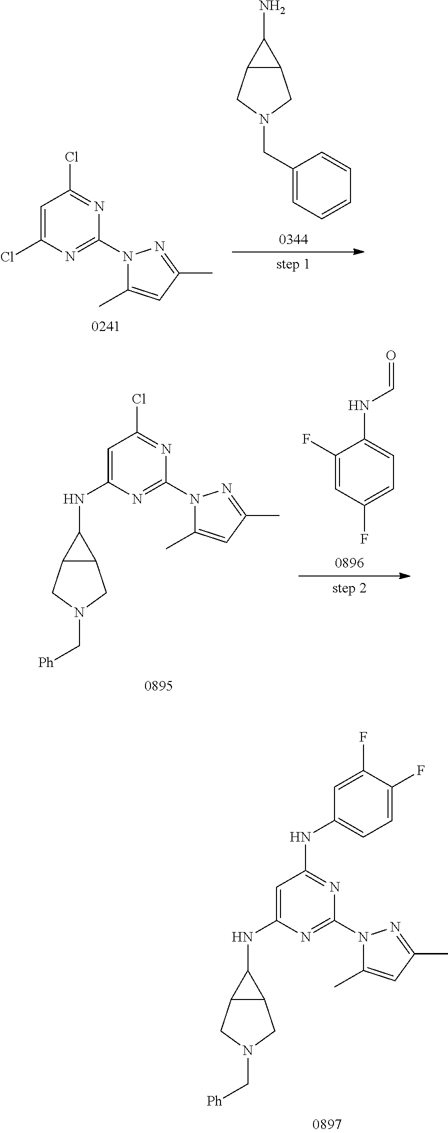 [SMILES: Cc1cc(C)n(-c2nc(Cl)cc(Cl)n2)n1 ; NC1C2CN(Cc3ccccc3)CC12 ; Cc1cc(C)n(-c2nc(Cl)cc(NC3C4CN(Cc5ccccc5)CC43)n2)n1 ; O=CNc1ccc(F)cc1F ; Cc1cc(C)n(-c2nc(Nc3ccc(F)c(F)c3)cc(NC3C4CN(Cc5ccccc5)CC43)n2)n1]

Step 1[0895]: The procedure is similar to step 1[0380] in Example 146 (4h). 0.7 g of 4,6-dichloro-2-(3,5-dimethyl-1H-pyrazol-1-yl)pyrimidine [0241] gave 0.38 g of 3-benzyl-N-(6-chloro-2-(3,5-dimethyl-1H-pyrazol-1-yl)pyrimidin-4-yl)-3-azabicyclo[3.1.0]hexan-6-amine [0895] as white solid. MS(M+1)+=395.7.

Step 2 [0897]: The procedure is similar to step 1 [0748] in Example 15. 0.38 g of 3-benzyl-N-(6-chloro-2-(3,5-dimethyl-1H-pyrazol-1-yl)pyrimidin-4-yl)-3-azabicyclo [3.1.0]hexan-6-amine [0895] gave 0.023 g of N4-(3-benzyl-3-azabicyclo[3.1.0]hexan-6-yl)-N6-(3,4-difluorophenyl)-2-(3,5-dimethyl-1H-pyrazol-1-yl)pyrimidine-4,6-diamine [0897], Compound 134 as yellow solid. MS(M+1)+=488.2. 1H NMR (400 MHz, DMSO-d6) δ 9.46 (bs, 1H), 8.06 (bs, 1H), 7.40-7.18 (m, 8H), 6.02 (s, 1H), 5.77 (bs, 1H), 3.57 (s, 2H), 3.50 (s, 1H), 2.99-3.09 (m, 2H), 2.49 (s, 3H), 2.16 (s, 3H), 1.59 (s, 2H) (angular proton (2H) missing)

### Example 66

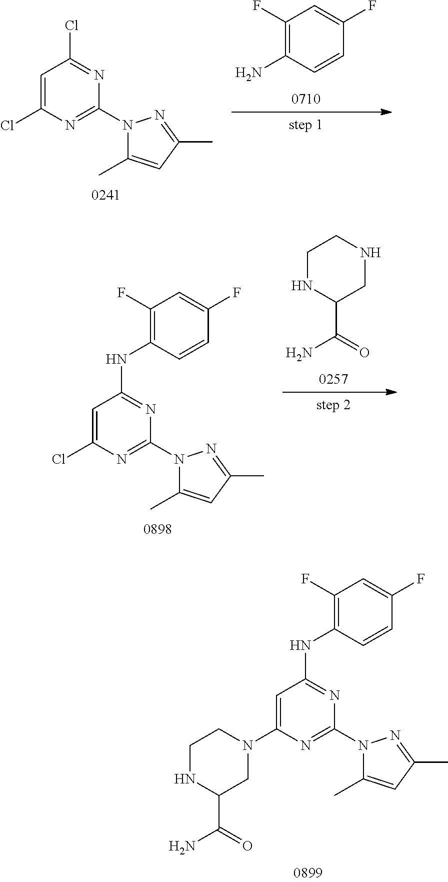 [SMILES: Cc1cc(C)n(-c2nc(Cl)cc(Cl)n2)n1 ; Nc1ccc(F)cc1F ; Cc1cc(C)n(-c2nc(Cl)cc(Nc3ccc(F)cc3F)n2)n1 ; NC(=O)C1CNCCN1 ; Cc1cc(C)n(-c2nc(Nc3ccc(F)cc3F)cc(N3CCNC(C(N)=O)C3)n2)n1]

Step 1[0898]: The procedure is similar to step 1[0742] in Example 12 (at 150° C.). 1 g of 4,6-dichloro-2-(3,5-dimethyl-1H-pyrazol-1-yl)pyrimidine [0241] gave 0.3 g of 6-chloro-N-(2,4-difluorophenyl)-2-(3,5-dimethyl-1H-pyrazol-1-yl)pyrimidin-4-amine [0898] as off-white solid. MS(M+1)+=336.3.

Step 2[0899]: The procedure is similar to step 1[0742] in Example 12 (at 150° C.). 0.3 g of 6-chloro-N-(2,4-difluorophenyl)-2-(3,5-dimethyl-1H-pyrazol-1-yl)pyrimidin-4-amine [0898] gave 0.03 g of 4-(6-((2,4-difluorophenyl)amino)-2-(3,5-dimethyl-1H-pyrazol-1-yl)pyrimidin-4-yl)piperazine-2-carboxamide [0899], Compound 115 as off-white solid. MS(M+1)+=429.5, 1H-NMR (400 MHz, DMSO-d6): δ 9.00 (s, 1H), 7.92-7.86 (m, 1H), 7.38-7.28 (m, 2H), 7.18 (s, 1H), 7.06 (t, J=8.80 Hz, 1H), 6.00 (s, 1H), 5.89 (s, 1H), 4.05-3.83 (m, 1H), 3.81-3.50 (m, 1H), 3.23-3.20 (m, 1H), 3.20-3.04 (m, 3H), 2.92-2.66 (m, 1H), 2.56 (s, 3H), 2.38 (s, 3H), 2.12 (s, 1H).

### Example 67

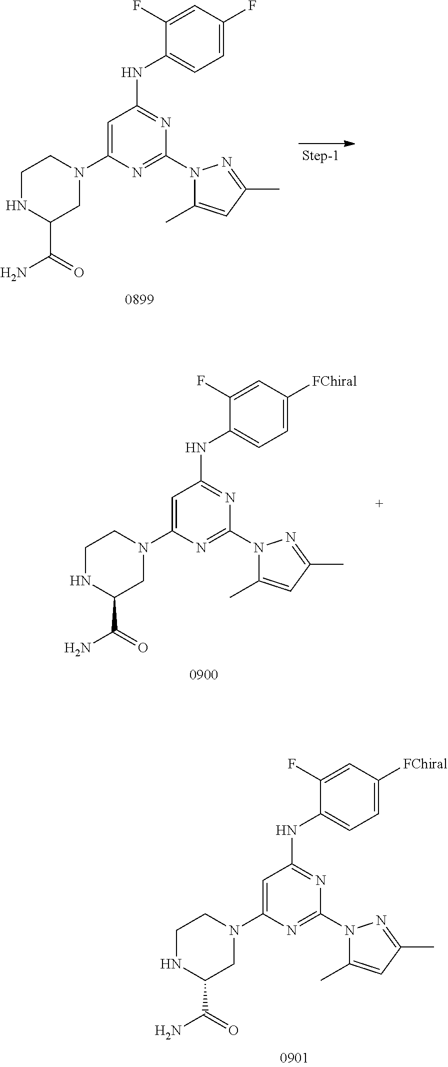

Step-1 [0900 and 0901]: 0.03 g of 4-(6-((2,4-difluorophenyl)amino)-2-(3,5-dimethyl-1H-pyrazol-1-yl)pyrimidin-4-yl)piperazine-2-carboxamide [0899] was separated by chiral HPLC to afford (+)-4-(6-((2,4-difluorophenyl)amino)-2-(3,5-dimethyl-1H-pyrazol-1-yl)pyrimidin-4-yl)piperazine-2-carboxamide [0900], Compound 128 as an off-white solid (8 mg) [MS(M+1)+=429, 1H-NMR (400 MHz, MeOD): δ 7.71-7.68 (m, 1H), 7.66-7.05 (m, 1H), 7.03-6.98 (m, 1H), 6.04 (s, 1H), 5.79 (s, 1H), 4.52 (d, J=Hz, 1H), 4.05 (d, J=Hz, 1H), 3.48 (d, J=3.44 Hz, 1H), 3.25 (s, 3H), 3.15-3.07 (m, 3H), 2.87-2.84 (m, 1H), 2.45 (s, 3H), 2.27 (s, 3H), and (−)-4-(6-((2,4-difluorophenyl)amino)-2-(3,5-dimethyl-1H-pyrazol-1-yl)pyrimidin-4-yl)piperazine-2-carboxamide [0901], Compound 125 as an off-white solid (14 mg) [MS(M+1)+=429, 1H NMR (400 MHz, DMSO-d6) δ 8.95 (s, 1H), 7.52-7.29 (m, 2H), 7.21 (s, 1H), 7.07 (t, J=8.6 Hz, 1H), 6.02 (s, 1H), 5.88 (s, 1H), 4.07 (s, 1H), 3.84 (s, 1H), 3.23 (s, 1H), 3.00 (dd, J=32.4, 12.1 Hz, 3H), 2.56 (s, 3H) 2.16 (s, 3H), 1.25 (d, J=5.7 Hz, 3H).

### Example 68

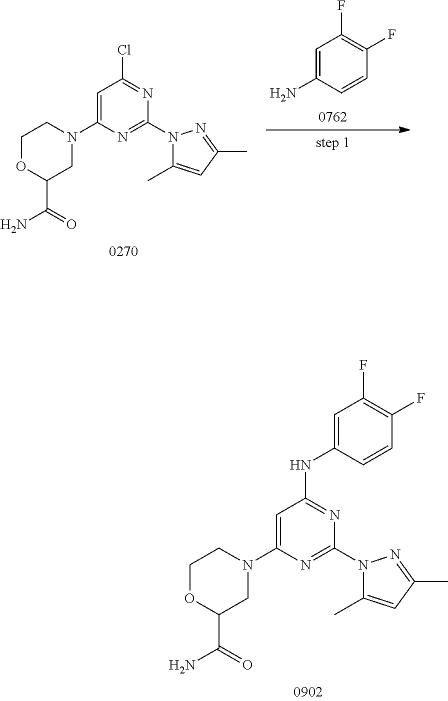 [SMILES: Cc1cc(C)n(-c2nc(Cl)cc(N3CCOC(C(N)=O)C3)n2)n1 ; Nc1ccc(F)c(F)c1 ; Cc1cc(C)n(-c2nc(Nc3ccc(F)c(F)c3)cc(N3CCOC(C(N)=O)C3)n2)n1]

Step 1[0902]: The procedure is similar to step 1[0748] in Example 15. 0.4 g of 4-(6-chloro-2-(3,5-dimethyl-1H-pyrazol-1-yl)pyrimidin-4-yl)morpholine-2-carboxamide [20] gave 0.03 g of 4-(6-((3,4-difluorophenyl)amino)-2-(3,5-dimethyl-1H-pyrazol-1-yl)pyrimidin-4-yl)morpholine-2-carboxamide [21], Compound 127 as off-white solid. MS(M+1)+=430.2, 1H NMR (400 MHz, DMSO-d6) δ 9.58 (s, 1H), 8.23 (dd, J=14.1, 7.4 Hz, 1H), 7.48 (s, 1H), 7.33 (dt, J=10.7, 9.2 Hz, 1H), 7.25-7.10 (m, 2H), 6.07 (s, 1H), 5.79 (s, 1H), 4.31 (d, J=11.6 Hz, 1H), 3.94 (d, J=11.0 Hz, 1H), 3.70-3.33 (m, 1H), 3.55-3.33 (m, 4H), 2.47 (s, 3H), 2.18 (s, 3H).

### Example 69

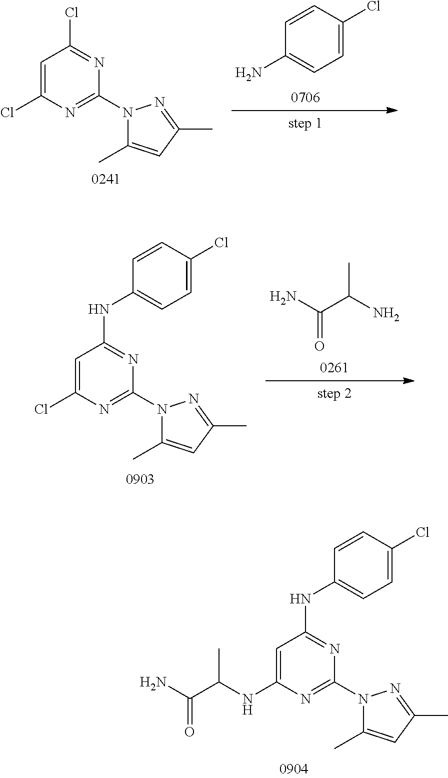

Step 1[0903]: A solution of 4,6-dichloro-2-(3,5-dimethyl-1H-pyrazol-1-yl)pyrimidine [0241] (1.5 g, 6.170 mmol), 4-chloroaniline [0706] (1.574 g, 12.341 mmol) and N,N-diisopropylethyl amine (3.224 mL, 18.511 mmol) in acetonitrile (5 mL) was heated at 150° C. in MW for 5 h. The reaction mixture was concentrated under reduced pressure to afford crude product which was purified by column chromatography using 25% ethyl acetate in hexane to obtain 6-chloro-N-(4-chlorophenyl)-2-(3,5-dimethyl-1H-pyrazol-1-yl)pyrimidin-4-amine[0903] as off-white solid (1.2 g, 58%). MS(M+1)+=334.1.

Step 2 [0904]: A solution of 6-chloro-N-(4-chlorophenyl)-2-(3,5-dimethyl-1H-pyrazol-1-yl)pyrimidin-4-amine [0903] (0.5 g, 1.496 mmol), 2-aminopropanamide [0261] (0.198 g, 2.244 mmol) and N,N-diisopropylethyl amine (0.193 g, 1.496 mmol) in acetonitrile (5 mL) was heated at 170° C. in MW for 5 h. The reaction mixture was concentrated under reduced pressure to afford crude product which was purified by preparative HPLC to afford 2-((6-((4-chlorophenyl)amino)-2-(3,5-dimethyl-1H-pyrazol-1-yl)pyrimidin-4-yl)amino)propanamide [0904], Compound 114 as off-white solid (0.11 g). MS(M+1)+=386.2. 1H NMR (400 MHz, DMSO-d6) δ 9.32 (s, 1H), 7.79-7.50 (m, 2H), 7.39 (s, 1H), 7.38-7.10 (m, 3H), 6.98 (bs, 1H), 6.02 (s, 1H), 5.85 (bs, 1H), 4.38 (s, 1H), 2.56 (s, 3H), 2.16 (s, 3H), 1.31 (d, J=7.1 Hz, 3H).

### Example 70

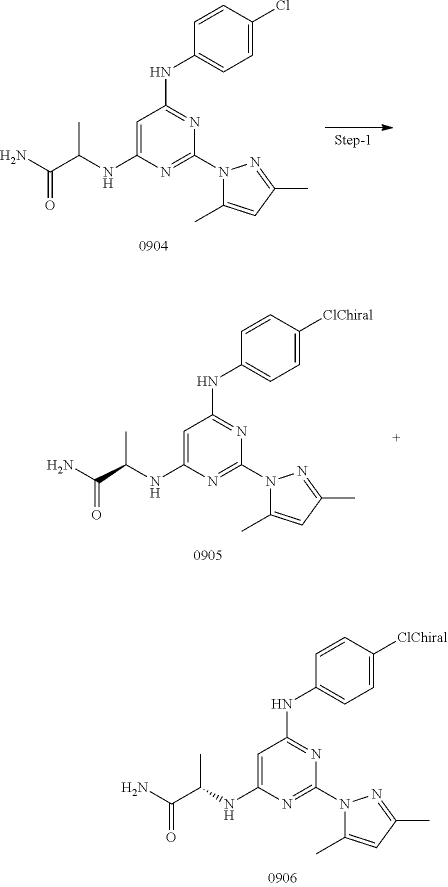

Step-1 [0905 and 0906] 0.11 g of 2-((6-((4-chlorophenyl)amino)-2-(3,5-dimethyl-1H-pyrazol-1-yl)pyrimidin-4-yl)amino)propanamide [0904] was separated by chiral HPLC to afford (+)-2-((6-((4-chlorophenyl)amino)-2-(3,5-dimethyl-1H-pyrazol-1-yl)pyrimidin-4-yl)amino)propanamide [0905], Compound 123 as a white solid (35 mg). [MS(M+1)+=336.1, 1H NMR (400 MHz, Chloroform-d) δ 7.34 (d, J=8.4 Hz, 2H), 7.22 (d, J=8.4 Hz, 2H), 6.88 (s, 1H), 6.56 (s, 1H), 6.00 (s, 1H), 5.61 (s, 1H), 5.49 (s, 1H), 5.26 (s, 1H), 4.36 (s, 1H), 2.61 (s, 3H), 2.32 (s, 3H), 1.51 (s, 3H) and (−)-2-((6-((4-chlorophenyl)amino)-2-(3,5-dimethyl-1H-pyrazol-1-yl)pyrimidin-4-yl)amino)propanamide [0906], Compound 124 as a white solid (35 mg). [MS(M+1)+=336.1, 1H NMR (400 MHz, DMSO-d6) δ 9.33 (s, 1H), 7.61 (d, J=9.0 Hz, 2H), 7.40 (s, 1H), 7.36-7.24 (m, 3H), 6.99 (s, 1H), 6.03 (s, 1H), 5.86 (s, 1H), 4.39 (s, 1H), 2.56 (s, 3H), 2.17 (s, 3H), 1.32 (d, J=7.1 Hz, 3H).

### Example 71

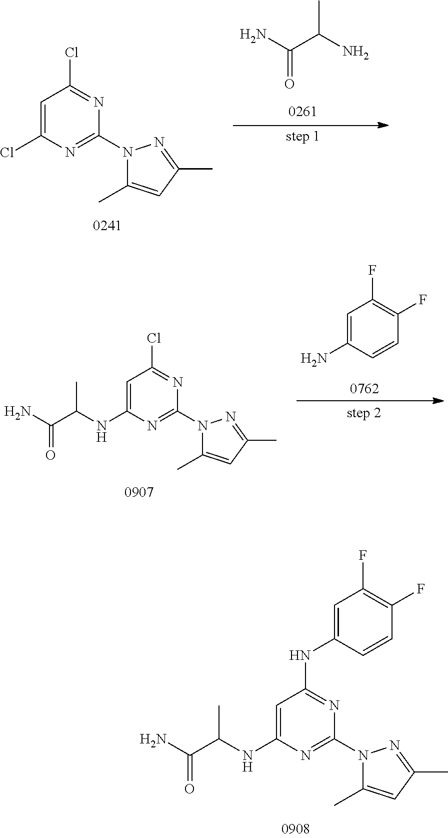

Step 1[0907]: The procedure is similar to step 1[0813] in Example 37 (at 80° C.). 1 g of 4,6-dichloro-2-(3,5-dimethyl-1H-pyrazol-1-yl)pyrimidine [0241] gave 1 g of 2-((6-chloro-2-(3,5-dimethyl-1H-pyrazol-1-yl)pyrimidin-4-yl)amino)propanamide [0907] as white solid. MS(M+1)+=388.0.

Step 2[0908]: The procedure is similar to step 1[0748] in Example 15. 0.5 g of 2-((6-chloro-2-(3,5-dimethyl-1H-pyrazol-1-yl)pyrimidin-4-yl)amino) propanamide [0907] gave 0.05 g of 2-((6-((3,4-difluorophenyl)amino)-2-(3,5-dimethyl-1H-pyrazol-1-yl)pyrimidin-4-yl)amino)propanamide [0908], Compound 126 as white solid. MS(M+1)+=295.2, 1H-NMR (400 MHz, Methanol-d4): δ 7.62-7.56 (m, 1H), 7.18-7.14 (m, 2H), 6.07 (s, 1H), 5.79 (s, 1H), 4.45 (bs, 1H), 2.58 (s, 3H), 2.29 (s, 3H), 1.47 (d, J=7.16 Hz, 3H).

### Example 72

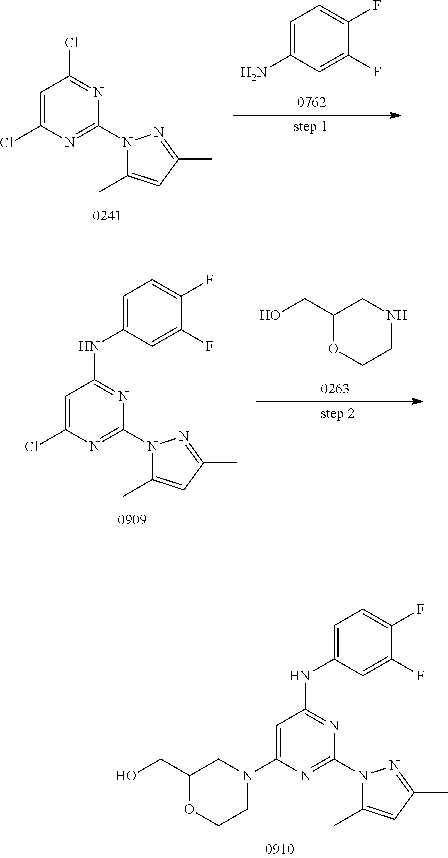 [SMILES: Cc1cc(C)n(-c2nc(Cl)cc(Cl)n2)n1 ; Nc1ccc(F)c(F)c1 ; Cc1cc(C)n(-c2nc(Cl)cc(Nc3ccc(F)c(F)c3)n2)n1 ; OCC1CNCCO1 ; Cc1cc(C)n(-c2nc(Nc3ccc(F)c(F)c3)cc(N3CCOC(CO)C3)n2)n1]

Step 1[0909]: 1 g of 4,6-dichloro-2-(3,5-dimethyl-1H-pyrazol-1-yl)pyrimidine [0241] gave 0.6 g of 6-chloro-N-(3,4-difluorophenyl)-2-(3,5-dimethyl-1H-pyrazol-1-yl)pyrimidin-4-amine [0909] as white solid, Using DIPEA, ACN, 80° C., 24 h. MS(M+1)+=336.2.

Step 2 [0910]: The procedure is similar to step 1[0813] in Example 37 (at 80° C.). 0.15 g of 6-chloro-N-(3,4-difluorophenyl)-2-(3,5-dimethyl-1H-pyrazol-1-yl)pyrimidin-4-amine [0909] gave 0.13 g of (4-(6-((3,4-difluorophenyl)amino)-2-(3,5-dimethyl-1H-pyrazol-1-yl)pyrimidin-4-yl)morpholin-2-yl)methanol[0910], Compound 117 as yellow solid. MS(M+1)+=417.2, 1H NMR (400 MHz, DMSO-d6) δ 9.55 (s, 1H), 8.28-8.09 (m, 1H), 7.34 (q, J=9.6 Hz, 1H), 7.25-7.17 (m, 1H), 6.08 (s, 1H), 5.86 (s, 1H), 4.88 (t, J=5.5 Hz, 1H), 4.19-3.99 (m, 2H), 3.96-3.90 (m, 1H), 3.60-3.38 (m, 4H), 2.99 (td, J=12.6, 3.6 Hz, 1H), 2.75 (dd, J=12.8, 10.0 Hz, 1H), 2.53 (s, 3H), 2.19 (s, 3H).

### Example 73

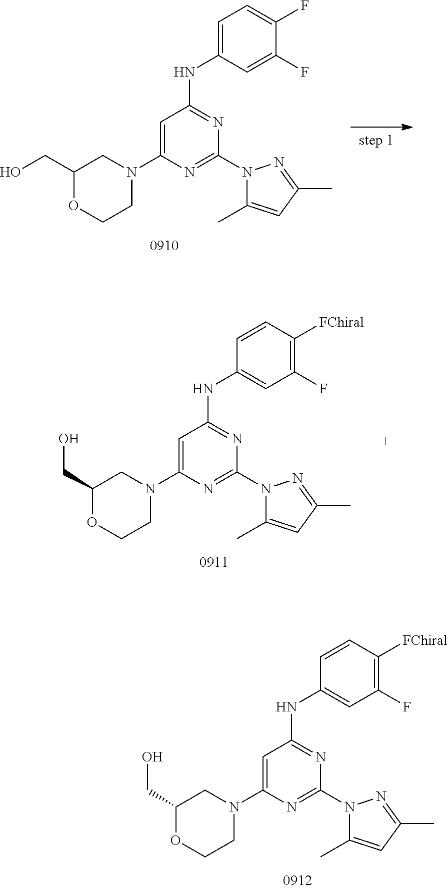

Step 1[0911 and 0912]: 0.1 g of (4-(6-((3,4-difluorophenyl)amino)-2-(3,5-dimethyl-1H-pyrazol-1-yl)pyrimidin-4-yl)morpholin-2-yl)methanol [0910] on chiral separation gave 0.04 g of (+)-(4-(6-((3,4-difluorophenyl)amino)-2-(3,5-dimethyl-1H-pyrazol-1-yl)pyrimidin-4-yl)morpholin-2-yl) methanol [0911], Compound 121 as off-white solid. MS(M+1)+=417.2. SOR: +15.686°, C=0.102, S=Methanol. T=24.0° C. 1H NMR (400 MHz, DMSO-d6) δ 9.56 (s, 1H), 8.28-8.12 (m, 1H), 7.34 (q, J=9.4 Hz, 1H), 7.25-7.15 (m, 1H), 6.08 (s, 1H), 5.86 (s, 1H), 4.88 (t, J=5.5 Hz, 1H), 4.18-4.0 (m, 2H), 3.98 (dd, J=25.4, 11.8 Hz, 1H), 3.63-3.37 (m, 4H), 2.91-3.05 (m, 1H), 2.80-2.71 (m, 1H), 2.53 (s, 3H), 2.19 (d, J=1.6 Hz, 3H). and 0.045 g of (−)-(4-(6-((3,4-difluorophenyl)amino)-2-(3,5-dimethyl-1H-pyrazol-1-yl)pyrimidin-4-yl)morpholin-2-yl)methanol as yellow solid [0912], Compound 122. MS(M+1)+=417.2. SOR: −2.679°, C=0.112, S=Methanol. T=24.2° C. 1H NMR (400 MHz, DMSO-d6) δ 9.56 (s, 1H), 8.28-8.07 (m, 1H), 7.34 (q, J=9.4 Hz, 1H), 7.25-7.15 (m, 1H), 6.08 (s, 1H), 5.86 (s, 1H), 4.88 (t, J=5.5 Hz, 1H), 4.18-4.0 (m, 2H), 3.98 (dd, J=25.4, 11.8 Hz, 1H), 3.62-3.35 (m, 4H), 2.91-3.05 (m, 1H), 2.80-2.71 (m, 1H), 2.53 (s, 3H), 2.19 (d, J=1.6 Hz, 3H).

### Example 74

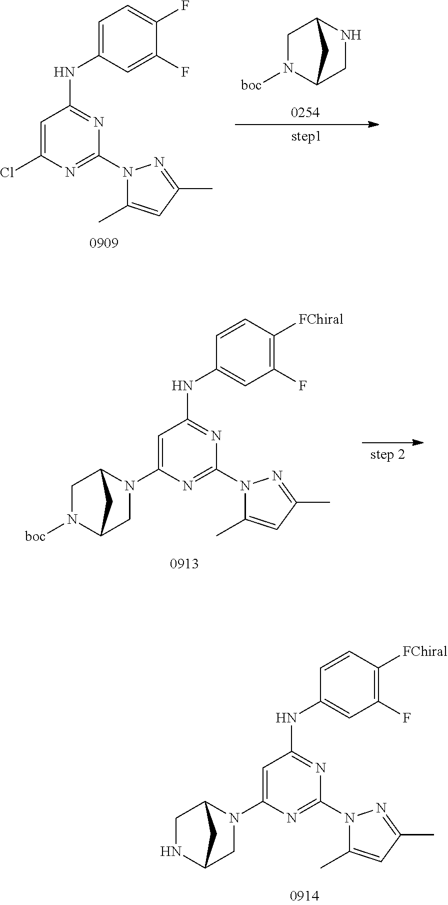

Step 1[0913]: The procedure is similar to step 1[0742] in Example 12 (at 80° C.). 0.14 g of 6-chloro-N-(3,4-difluorophenyl)-2-(3,5-dimethyl-1H-pyrazol-1-yl)pyrimidin-4-amine [0909] gave 0.13 g of tert-butyl (1R,4R)-5-(6-((3,4-difluorophenyl)amino)-2-(3,5-dimethyl-1H-pyrazol-1-yl)pyrimidin-4-yl)-2,5-diazabicyclo[2.2.1]heptane-2-carboxylate [0913] as colorless gum. MS(M+1)+=498.6.

Step 2 [0914] The procedure is similar to step 2[0814] in Example 37. 0.13 g of tert-butyl (1R,4R)-5-(6-((3,4-difluorophenyl)amino)-2-(3,5-dimethyl-1H-pyrazol-1-yl)pyrimidin-4-yl)-2,5-diazabicyclo[2.2.1]heptane-2-carboxylate [0913] gave 0.1 g of N-(3,4-difluorophenyl)-2-(3,5-dimethyl-1H-pyrazol-1-yl)-6-((1R,4R)-5-methyl-2,5-diazabicyclo [2.2.1]heptan-2-yl)pyrimidin-4-amine [0914], Compound 112 as off-white solid. MS(M+1)+=398.2, 1H-NMR (400 MHz, DMSO-d6): δ 9.72 (s, 1H), 9.59 (s, 1H), 9.11 (s, 1H), 8.24-8.19 (m, 1H), 7.39-7.32 (m, 1H), 7.26-7.24 (m, 1H), 6.11 (s, 1H), 5.66 (s, 1H), 4.94 (s, 1H), 4.50 (s, 1H), 3.60-3.58 (m, 1H), 3.31-3.29 (m, 1H), 3.22-3.17 (m, 1H), 2.56-2.51 (m, 3H), 2.20 (s, 3H), 2.14 (d, J=10.40 Hz, 1H), 1.95 (d, J=11.04 Hz, 1H).

### Example 75

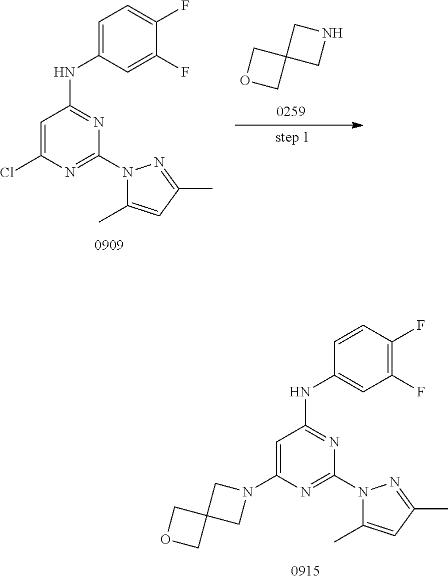 [SMILES: Cc1cc(C)n(-c2nc(Cl)cc(Nc3ccc(F)c(F)c3)n2)n1 ; C1NCC12COC2 ; Cc1cc(C)n(-c2nc(Nc3ccc(F)c(F)c3)cc(N3CC4(COC4)C3)n2)n1]

Step 1[0915]: The procedure is similar to step 1[08132] in Example 37. 0.12 g of 6-chloro-N-(3,4-difluorophenyl)-2-(3,5-dimethyl-1H-pyrazol-1-yl)pyrimidin-4-amine [0909] gave 0.09 g of N-(3,4-difluorophenyl)-2-(3,5-dimethyl-1H-pyrazol-1-yl)-6-(2-oxa-6-azaspiro[3.3]heptan-6-yl)pyrimidin-4-amine [0915], Compound 111 as off-white solid. MS(M+1)+=399.2. 1H NMR (400 MHz, DMSO-d6) δ 9.52 (s, 1H), 8.25-8.09 (m, 1H), 7.32 (q, J=9.5 Hz, 1H), 7.16-7.24 (m, 1H), 6.06 (s, 1H), 5.45 (s, 1H), 4.73 (s, 4H), 4.18 (s, 4H), 2.52 (s, 3H), 2.18 (s, 3H).

### Example 76

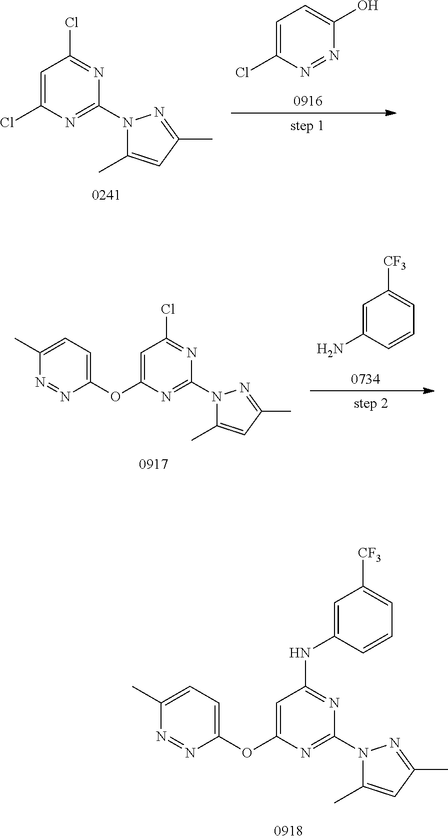

Step 1[0917]: 0.25 g of 4,6-dichloro-2-(3,5-dimethyl-1H-pyrazol-1-yl)pyrimidine [0241] gave 0.26 g of 3-((6-chloro-2-(3,5-dimethyl-1H-pyrazol-1-yl)pyrimidin-4-yl)oxy)-6-methylpyridazine [0917] as off-white solid (Using CS2CO3, DMSO, rt 1 h). MS(M+1)+=317.2.

Step 2 [0918]: The procedure is similar to step 1[0748] in Example 15. 0.2 g of 3-((6-chloro-2-(3,5-dimethyl-1H-pyrazol-1-yl)pyrimidin-4-yl)oxy)-6-methylpyridazine [0917] gave 0.058 g of 2-(3,5-dimethyl-1H-pyrazol-1-yl)-6-((6-methylpyridazin-3-yl)oxy)-N-(3-(trifluoromethyl)phenyl)pyrimidin-4-amine [0918], Compound 181 as off-white solid. MS(M+1)+=442.3, 1H NMR (400 MHz, DMSO-d6) δ 10.46 (s, 1H), 8.70 (s, 1H), 7.78 (d, J=8.4 Hz, 1H), 7.60 (t, J=8.0 Hz, 1H), 7.48 (d, J=9.6 Hz, 1H), 7.42 (d, J=7.8 Hz, 1H), 7.07 (d, J=9.6 Hz, 1H), 7.02 (s, 1H), 6.15 (s, 1H), 2.55 (s, 3H), 2.35 (s, 3H), 2.21 (s, 3H).

### Example 77

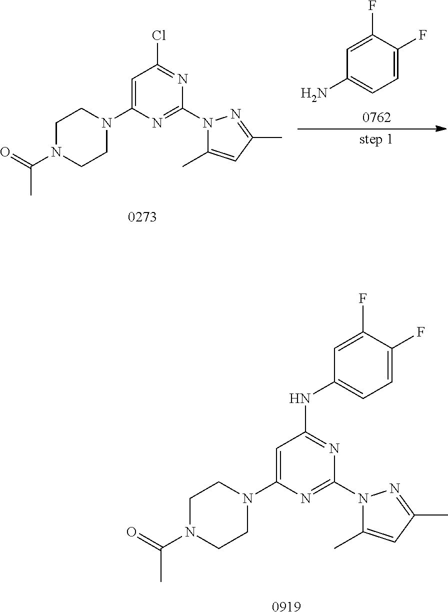 [SMILES: CC(=O)N1CCN(c2cc(Cl)nc(-n3nc(C)cc3C)n2)CC1 ; Nc1ccc(F)c(F)c1 ; CC(=O)N1CCN(c2cc(Nc3ccc(F)c(F)c3)nc(-n3nc(C)cc3C)n2)CC1]

Step 1[0919]: To a stirred degassed suspension of 3,4-difluoroaniline [0762] (0.043 g, 0.329 mmol), 1-(4-(6-chloro-2-(3,5-dimethyl-1H-pyrazol-1-yl)pyrimidin-4-yl)piperazin-1-yl)ethan-1-one [0273] (0.1 g, 0.299 mmol), rac-2,2′-Bis(diphenylphosphino)-1,1′-binaphthyl (0.096 g, 0.149 mmol) and sodium tert-butoxide (0.034 g, 0.358 mmol) in 1,4-dioxane (10 mL) was added tris(dibenzylideneacetone)dipalladium(0) (0.137 g, 0.149 mmol) in a pressure tube. The tube was sealed and the reaction mixture was heated at 100° C. for 20 h. The reaction mixture was concentrated under reduced pressure. The residue was quenched with water (10 mL) and the product was extracted with chloroform (3×100 mL). Combined organic layer was washed with brine (50 mL), dried over anhydrous sodium sulfate, filtered and concentrated under reduced pressure to afford crude product which was purified by column using 4% methanol in chloroform chromatography as eluent to afford 1-(4-(6-((3,4-difluorophenyl)amino)-2-(3,5-dimethyl-1H-pyrazol-1-yl)pyrimidin-4-yl)piperazin-1-yl)ethan-1-one [0919], Compound 100 as off-white solid (0.043 g, 31%). MS(M+1)+=428.4, 1H NMR (400 MHz, DMSO-d6) δ 9.36 (s, 1H), 7.81 (ddd, J=13.9, 7.5, 2.4 Hz, 1H), 7.46-7.23 (m, 2H), 6.58 (s, 1H), 6.11 (s, 1H), 3.71 (bs, 2H), 3.62 (bs, 2H), 3.58 (bs, 4H), 2.61 (s, 3H), 2.20 (s, 3H), 2.06 (s, 3H).

### Example 78

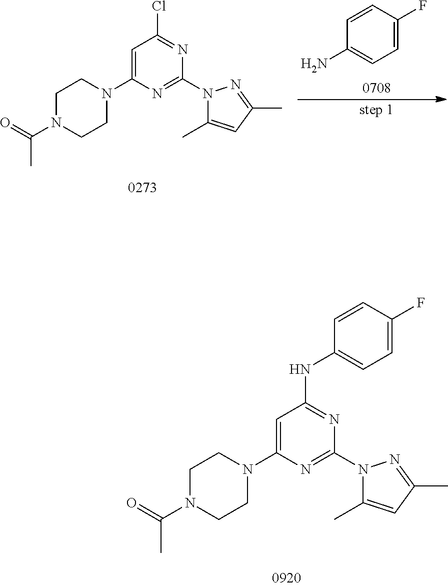 [SMILES: CC(=O)N1CCN(c2cc(Cl)nc(-n3nc(C)cc3C)n2)CC1 ; Nc1ccc(F)cc1 ; CC(=O)N1CCN(c2cc(Nc3ccc(F)cc3)nc(-n3nc(C)cc3C)n2)CC1]

Step 1[0920]: The procedure is similar to step 1[0919] in Example 77. 0.28 g of 1-(4-(6-chloro-2-(3,5-dimethyl-1H-pyrazol-1-yl)pyrimidin-4-yl)piperazin-1-yl)ethan-1-one [0273] gave 0.038 g of 1-(4-(2-(3,5-dimethyl-1H-pyrazol-1-yl)-6-((4-fluorophenyl)amino)pyrimidin-4-yl)piperazin-1-yl)ethan-1-one [0920], Compound 102 as off-white solid. MS(M+1)+=410.3. 1H NMR (400 MHz, DMSO-d6) δ 9.35 (s, 1H), 7.60 (dd, J=5, 4.8 Hz, 2H), 7.14 (t, J=8.8 Hz, 2H), 6.05 (s, 1H), 5.83 (s, 1H), 3.65-3.45 (m, 8H), 2.48 (s, 3H), 2.18 (s, 3H), 2.05 (s, 3H).

### Example 79

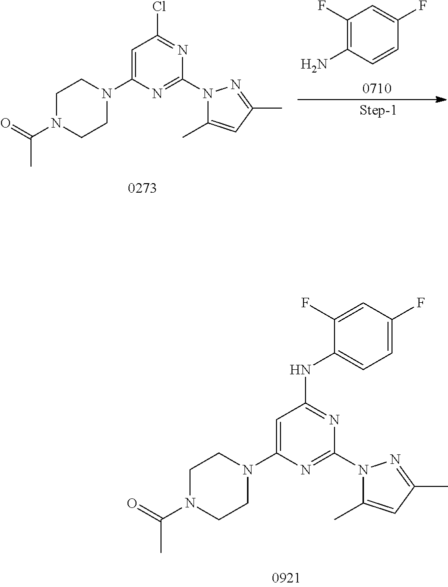 [SMILES: CC(=O)N1CCN(c2cc(Cl)nc(-n3nc(C)cc3C)n2)CC1 ; Nc1ccc(F)cc1F ; CC(=O)N1CCN(c2cc(Nc3ccc(F)cc3F)nc(-n3nc(C)cc3C)n2)CC1]

Step 1[0921]: The procedure is similar to step 1[0919] in example 77. 0.22 g of 1-(4-(6-chloro-2-(3,5-dimethyl-1H-pyrazol-1-yl)pyrimidin-4-yl)piperazin-1-yl)ethan-1-one [0273] gave 0.034 g of 1-(4-(6-((2,4-difluorophenyl)amino)-2-(3,5-dimethyl-1H-pyrazol-1-yl)pyrimidin-4-yl)piperazin-1-yl)ethan-1-one [0921], Compound 105 as off-white solid. MS(M+1)+=428.2, 1H NMR (400 MHz, DMSO-d6) δ 8.99 (s, 1H), 7.84 (q, J=9.24, 6.3 Hz, 1H), 7.38-7.28 (m, 1H), 7.08 (t, J=8.1 Hz, 1H), 6.02 (s, 1H), 5.83 (s, 1H), 3.63-3.45 (m, 8H), 2.40 (s, 3H), 2.16 (d, J=2.9 Hz, 3H), 2.04 (s, 3H).

### Example 80

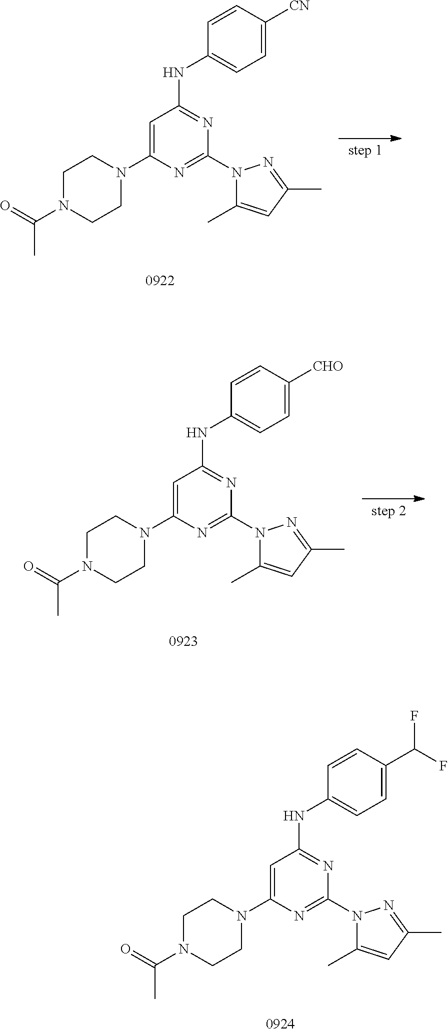 [SMILES: CC(=O)N1CCN(c2cc(Nc3ccc(C#N)cc3)nc(-n3nc(C)cc3C)n2)CC1 ; CC(=O)N1CCN(c2cc(Nc3ccc(C=O)cc3)nc(-n3nc(C)cc3C)n2)CC1 ; CC(=O)N1CCN(c2cc(Nc3ccc(C(F)F)cc3)nc(-n3nc(C)cc3C)n2)CC1]

Step 1[0923]: To a stirred solution of 4-((6-(4-acetylpiperazin-1-yl)-2-(3,5-dimethyl-1H-pyrazol-1-yl)pyrimidin-4-yl)amino)benzonitrile [0922] (0.13 g, 0.299 mmol) in aqueous formic acid (12 mL, 80%) in a pressure tube was added nickel (II) chloride (0.002 g, 0.05 mmol). The tube was sealed and the reaction mixture was heated at 50° C. for 3 h. The reaction mixture was diluted with water (20 mL), filtered through a bed of celite, washed the bed with water (10 mL). The combined filtrate was basified with aq. sodium bicarbonate solution (10%) till pH˜8 and the product was extracted with chloroform (3×75 mL). The combined organic layer was washed with brine (50 mL), dried over anhydrous sodium sulfate, filtered and concentrated under reduced pressure to afford crude which was purified by column chromatography using 30% ethyl acetate in hexane as eluent to afford 4-((6-(4-acetylpiperazin-1-yl)-2-(3,5-dimethyl-1H-pyrazol-1-yl)pyrimidin-4-yl)amino)benzaldehyde [0923] as brownish solid (0.090 g, 75%). MS(M+1)+=420.3.

Step 2[0924]: The procedure is similar to step 3[0760] in Example 21. 0.13 g of 4-((6-(4-acetylpiperazin-1-yl)-2-(3,5-dimethyl-1H-pyrazol-1-yl)pyrimidin-4-yl)amino) benzaldehyde [0923] gave 0.09 g of 1-(4-(6-((4-(difluoromethyl)phenyl)amino)-2-(3,5-dimethyl-1H-pyrazol-1-yl)pyrimidin-4-yl)piperazin-1-yl)ethan-1-one [0924], Compound 203 as brown solid. MS(M+1)+=442.3, 1H NMR (400 MHz, DMSO-d6) δ 9.43 (s, 1H), 7.81 (d, J=8.4 Hz, 2H), 7.49 (d, J=8.3 Hz, 2H), 6.95 (t, J_{F=}56.12 Hz, 1H) 6.58 (s, 1H), 6.11 (s, 1H), 3.72 (bs, 2H), 3.66-3.52 (m, 6H), 2.63 (s, 3H), 2.20 (s, 3H), 2.06 (s, 3H).

### Example 81

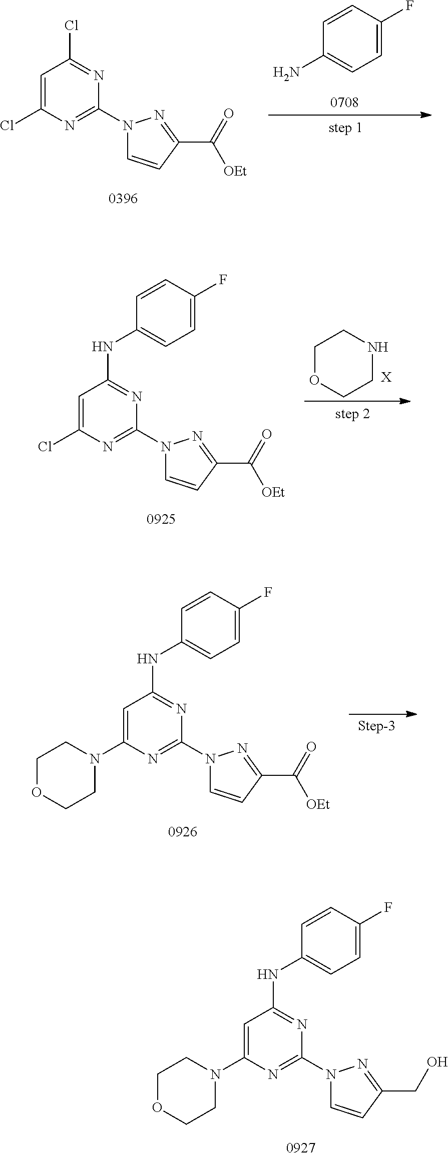

Step 1[0925]: The procedure is similar to step 2[0720] in Example 2. 5 g of ethyl 1-(4,6-dichloropyrimidin-2-yl)-1H-pyrazole-3-carboxylate [0396] gave 7.4 g of ethyl 1-(4-chloro-6-((4-fluorophenyl)amino)pyrimidin-2-yl)-1H-pyrazole-3-carboxylate [0925] as a yellow solid. MS(M+1)+=288.2.

Step 2 [0926]: 4 g of ethyl 1-(4-chloro-6-((4-fluorophenyl)amino)pyrimidin-2-yl)-1H-pyrazole-3-carboxylate [0925] in acetonitrile (26 mL) was added morpholine and the reaction mixture was irradiated in MW at 100° C. for 1.5h to afford 4.1 g of ethyl 1-(4-((4-fluorophenyl)amino)-6-morpholinopyrimidin-2-yl)-1H-pyrazole-3-carboxylate [0926] as an off-white solid. MS(M+1)+=413.2.

Step 3[0927]: The procedure is similar to step 2[0771] in Example 25. 2 g of ethyl 1-(4-((4-fluorophenyl)amino)-6-morpholinopyrimidin-2-yl)-1H-pyrazole-3-carboxylate [0926] gave 0.9 g of (1-(4-((4-fluorophenyl)amino)-6-morpholinopyrimidin-2-yl)-1H-pyrazol-3-yl)methanol [0927], Compound 156 as white solid. MS(M+1)+=371.2, 1H NMR (400 MHz, DMSO-d6) δ 9.40 (s, 1H), 8.46 (s, 1H), 7.63 (dd, J=6.0, 5.2 Hz, 2H), 7.16 (t, 8.8 Hz, 2H), 6.46 (s, 1H), 5.80 (s, 1H), 5.20 (t, 5.6 Hz, 1H), 4.50 (d, 6 Hz, 2H), 3.39 (s, 4H), 3.52 (s, 4H).

### Example 82

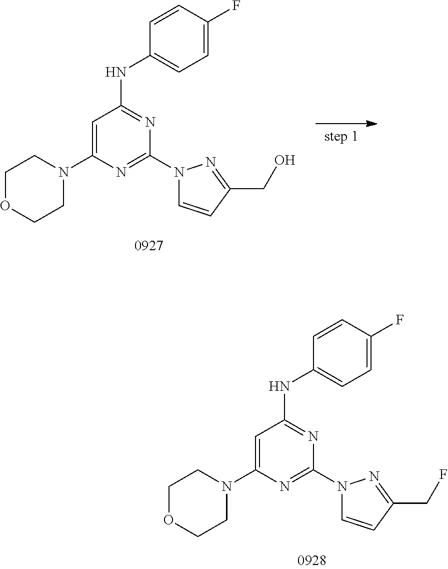 [SMILES: OCc1ccn(-c2nc(Nc3ccc(F)cc3)cc(N3CCOCC3)n2)n1 ; FCc1ccn(-c2nc(Nc3ccc(F)cc3)cc(N3CCOCC3)n2)n1]

Step 1[0928]: The procedure is similar to step 3[0760] in Example 21. 0.2 g of (1-(4-((4-fluorophenyl)amino)-6-morpholinopyrimidin-2-yl)-1H-pyrazol-3-yl)methanol [0927] gave 0.06 g of 2-(3-(fluoromethyl)-1H-pyrazol-1-yl)-N-(4-fluorophenyl)-6-morpholinopyrimidin-4-amine [0928], Compound 157 as off-white solid. MS(M+1)+=373.2, 1H NMR (400 MHz, DMSO-d6) δ 9.44 (s, 1H), 8.56 (s, 1H), 7.78-7.36 (m, 2H), 7.17 (t, J=8.6 Hz, 2H), 6.64 (s, 1H), 5.83-5.51 (d, J_{F}=48 Hz, 2H), 5.39 (s, 1H), 3.69 (t, J=4.7 Hz, 4H), 3.54 (d, J=5.1 Hz, 3H).

### Example 83

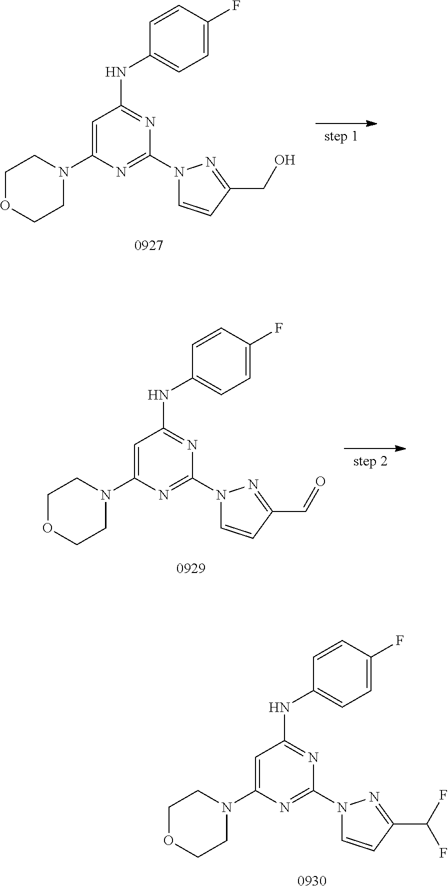 [SMILES: OCc1ccn(-c2nc(Nc3ccc(F)cc3)cc(N3CCOCC3)n2)n1 ; O=Cc1ccn(-c2nc(Nc3ccc(F)cc3)cc(N3CCOCC3)n2)n1 ; Fc1ccc(Nc2cc(N3CCOCC3)nc(-n3ccc(C(F)F)n3)n2)cc1]

Step 1[0929]: The procedure is similar to step 1[0786] in Example 28. 0.4 g of (1-(4-((4-fluorophenyl)amino)-6-morpholinopyrimidin-2-yl)-1H-pyrazol-3-yl)methanol [0927] gave 0.37 g of 1-(4-((4-fluorophenyl)amino)-6-morpholinopyrimidin-2-yl)-1H-pyrazole-3-carbaldehyde [0929] as off-white solid. MS(M+1)+=369.2.

Step 2[0930]: The procedure is similar to step 3[0760] in Example 21. 0.37 g of 1-(4-((4-fluorophenyl)amino)-6-morpholinopyrimidin-2-yl)-1H-pyrazole-3-carbaldehyde [0929] gave 0.165 g of 2-(3-(difluoromethyl)-1H-pyrazol-1-yl)-N-(4-fluorophenyl)-6-morpholinopyrimidin-4-amine [0930], Compound 158 as white solid. MS(M+1)+=391.2, 1H NMR (400 MHz, DMSO-d6) δ 9.48 (s, 1H), 8.64 (d, J=2.7 Hz, 1H), 7.60 (dd, J=8.9, 5.0 Hz, 2H), 7.21 (t, J_{F}=54.4 Hz, 1H), 7.16 (t, J=9 Hz, 2H), 6.77 (d, J=2.7 Hz, 1H), 5.85 (s, 1H), 3.69 (t, J=4.8 Hz, 4H), 3.54 (t, J=4.8 Hz, 4H).

### Example 84

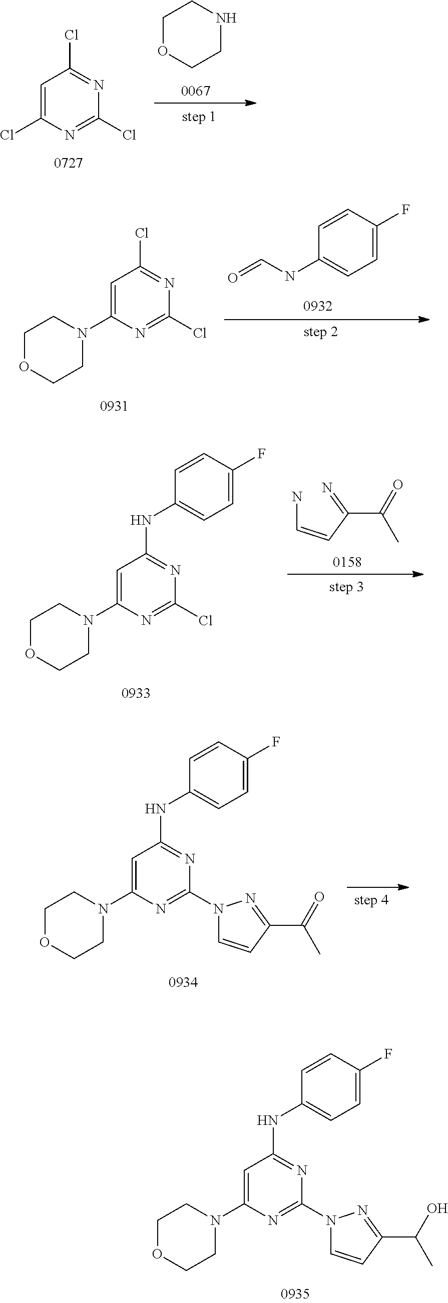

Step 1[0931]: 1 g of 2,4,6-trichloropyrimidine [0727] and 0.47 g of morpholine [0067] gave 0.9 g of 4-(2,6-dichloropyrimidin-4-yl)morpholine [0931] as off-white solid (Using DIPEA, ACN, rt, 5h). MS(M+1)+=235.2.

Step 2[0933]: To a solution of 4-(2,6-dichloropyrimidin-4-yl)morpholine [0931](1.72 g 7.34 mmol) and N-(4-fluorophenyl)formamide [0932] (1.22 g 8.88 mmol) in dioxane was added Sodium iodide (1.1 g 7.34 mmol) and hydroiodic acid (1.7 mL). The reaction mixture was heated at 90° C. After 5 h, the reaction mixture was filtered and the filtrate was extracted with ethyl acetate washed with saturated sodium thiosulfate, water and brine solution. The combined organic layer was dried over sodium sulfate, filtered and concentrated under reduced pressure to afford a brown solid, which was purified using ethyl acetate and hexane as solvent to afford 2-chloro-N-(4-fluorophenyl)-6-morpholinopyrimidin-4-amine [0933] as yellow solid (0.47 g). MS(M+1)+=309.3

Step 3[0934]: The procedure is similar to step 3[0766] in Example 24. 0.36 g of 2-chloro-N-(4-fluorophenyl)-6-morpholinopyrimidin-4-amine [0933] gave 0.35 g of 1-(1-(4-((4-fluorophenyl)amino)-6-morpholinopyrimidin-2-yl)-1H-pyrazol-3-yl)ethan-1-one [0934] as off-white solid. MS(M+1)+=383.4.

Step 4 [0935]: The procedure is similar to step 4[0722] in Example 2. 0.2 g of 1-(1-(4-((4-fluorophenyl)amino)-6-morpholinopyrimidin-2-yl)-1H-pyrazol-3-yl)ethan-1-one [0934]gave 0.03 g of 1-(1-(4-((4-fluorophenyl)amino)-6-morpholinopyrimidin-2-yl)-1H-pyrazol-3-yl)ethan-1-ol [0935], Compound 146 as white solid. MS(M+1)+=385.3, 1H NMR (400 MHz, DMSO-d6) δ 9.32 (s, 1H), 8.40 (d, J=2.6 Hz, 1H), 7.83-7.63 (m, 2H), 7.14 (t, J=8.9 Hz, 2H), 6.57 (s, 1H), 6.53 (d, J=2.6 Hz, 1H), 5.27 (d, J=4.8 Hz, 1H), 4.79 (dd, J=6.6, 5.0 Hz, 1H), 3.70 (d, J=4.6 Hz, 4H), 3.63 (d, J=4.6 Hz, 4H), 1.41 (d, J=6.5 Hz, 3H).

### Example 85

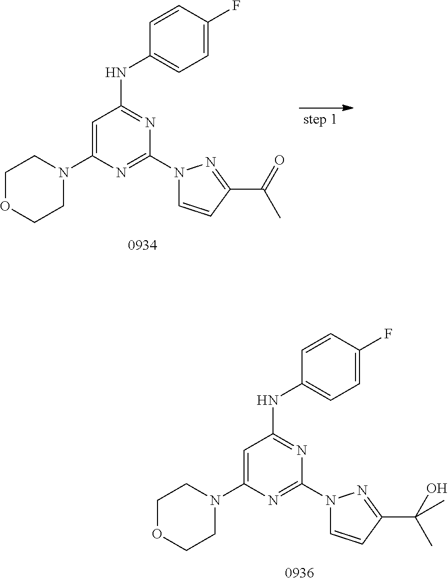 [SMILES: CC(=O)c1ccn(-c2nc(Nc3ccc(F)cc3)cc(N3CCOCC3)n2)n1 ; CC(C)(O)c1ccn(-c2nc(Nc3ccc(F)cc3)cc(N3CCOCC3)n2)n1]

Step-1 [0936]: The procedure is similar to step 2[0765] in Example 24. 0.1 g of 1-(1-(4-((4-fluorophenyl)amino)-6-morpholinopyrimidin-2-yl)-1H-pyrazol-3-yl)ethan-1-one [0934] gave 0.02 g of 2-(1-(4-((4-fluorophenyl)amino)-6-morpholinopyrimidin-2-yl)-1H-pyrazol-3-yl)propan-2-ol [0936], Compound 148 as white solid. MS(M+1)+=399.3, 1H NMR (400 MHz, Chloroform-d) δ 8.40 (s, 1H), 7.54 (dd, J=9.0, 4.8 Hz, 2H), 7.05 (dd, J=8.7 Hz, 4.3 Hz, 2H), 6.81 (s, 1H), 6.66 (s, 1H), 6.39 (s, 1H), 3.83 (t, J=4.9 Hz, 4H), 3.72 (s, 4H), 1.64 (s, 6H).

### Example 86

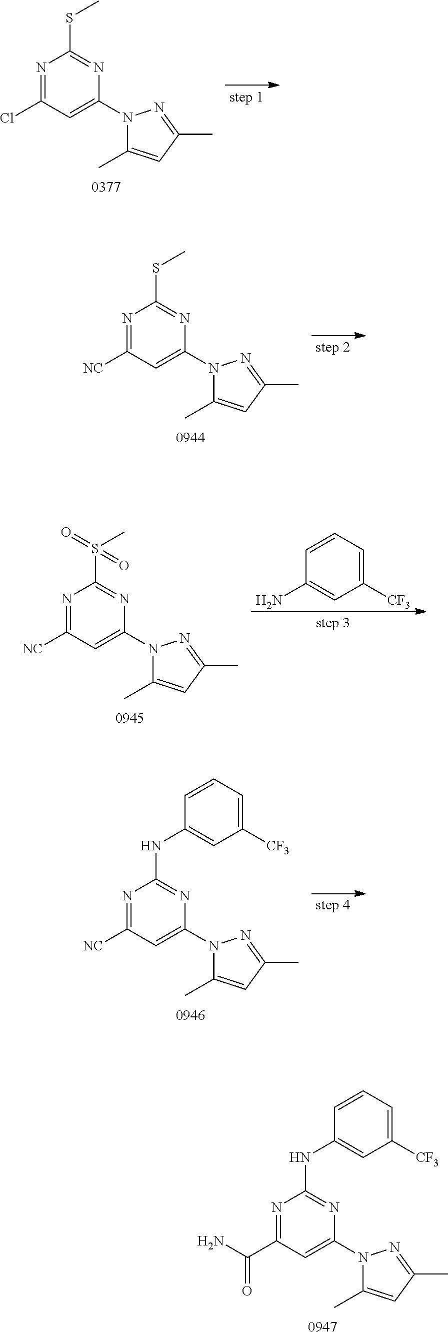

Step 1[0944]: To a degassed stirred mixture of 4-chloro-6-(3,5-dimethyl-1H-pyrazol-1-yl)-2-(methylthio)pyrimidine [0377] (5.5 g, 21.51 mmol), tributyltin chloride (0.421 g, 1.295 mmol), ‘1,1’-bis(diphenylphosphino)ferrocene (1.196 g, 2.159 mmol) and potassium cyanide (2.86 g, 43.18 mmol) in acetonitrile (125 mL) was added tris(dibenzylideneacetone)dipalladium(0) (1.977 g, 2.159 mmol). The mixture was stirred at rt for 30 min, and then heated at 80° C. for 24 h. The reaction mixture was diluted with ethyl acetate (250 mL) and water (100 mL). The Organic layer was separated and washed with water (2×50 mL), brine (50 mL), dried over anhydrous sodium sulfate, filtered and concentrated under reduced pressure to afford crude product which was purified by column chromatography using 12% ethyl acetate in hexane as eluent to afford 6-(3,5-dimethyl-1H-pyrazol-1-yl)-2-(methylthio)pyrimidine-4-carbonitrile [0944] as off-white solid (3.1 g). MS(M+1)+=246.2.

Step 2 [0945]: The procedure is similar to step 3[0835] in example 51. 2.5 g of 6-(3,5-dimethyl-1H-pyrazol-1-yl)-2-(methylthio)pyrimidine-4-carbonitrile [0944] gave 1.9 g of 6-(3,5-dimethyl-1H-pyrazol-1-yl)-2-(methylsulfonyl)pyrimidine-4-carbonitrile [0945] as a brown solid. MS(M+1)+=278.2.

Step 3 [0946]: The procedure is similar to step 1[0748] in example 15. 1.9 g of 6-(3,5-dimethyl-1H-pyrazol-1-yl)-2-(methylsulfonyl)pyrimidine-4-carbonitrile [0945] and 3-(trifluoromethyl)aniline [0734] (1.21 g, 7.536 mmol) gave 1.2 g of 6-(3,5-dimethyl-1H-pyrazol-1-yl)-2-((3-(trifluoromethyl)phenyl)amino)pyrimidine-4-carbonitrile [0946], Compound 184 as a beige solid. MS(M+1)+=358.8, 1H NMR (400 MHz, DMSO-d6) δ 10.28 (s, 1H), 8.85 (s, 1H), 8.41 (s, 1H), 7.89 (d, J=8.1 Hz, 1H), 7.60 (t, J=8.0 Hz, 1H), 7.50 (d, J=7.7 Hz, 1H), 6.14 (s, 1H), 2.38 (s, 3H), 2.18 (s, 3H).

Step 4 [0947]: Potassium hydroxide was added to solution of 6-(3,5-dimethyl-1H-pyrazol-1-yl)-2-((3-(trifluoromethyl)phenyl)amino)pyrimidine-4-carbonitrile (0.1 g, 0.27 m mol) [0946] in a mixture of methanol (5 mL) and water (5 mL) and stirred at rt for 40 h. The reaction mixture was concentrated under reduced pressure, the residue was cooled to 5° C., then pH was made slightly acidic (˜6) by using 1.5 N hydrochloric acid. Then product was extracted with ethyl acetate (3×75 mL), combined organic layer was washed with brine (3×75 mL), dried over anhydrous, sodium sulfate and concentrated to afford crude and which was purified by column chromatography dichloromethane as eluent to afford 0.026 g of 6-(3,5-dimethyl-1H-pyrazol-1-yl)-2-((3-(trifluoromethyl)phenyl)amino) pyrimidine-4-carboxamide [0947], Compound 185 as a white solid. MS(M+1)+=377.0, 1H-NMR (400 MHz, DMSO-d6): δ 10.26 (s, 1H), 8.88 (s, 1H), 8.45 (s, 1H), 8.22 (d, J=8.84 Hz, 1H), 8.09 (d, J=8.84 Hz, 1H), 7.92 (d, J=8.12 Hz, 1H), 7.60 (t, J=8.00 Hz, 1H), 7.51 (d, J=7.72 Hz, 1H), 6.14 (s, 1H), 2.38 (s, 3H), 2.17 (s, 3H).

### Example 87

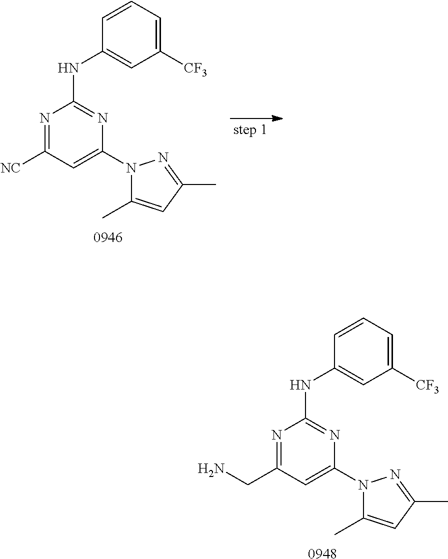

Step 1[0948]: To a degassed solution of 6-(3,5-dimethyl-1H-pyrazol-1-yl)-2-((3-(trifluoromethyl) phenyl)amino) pyrimidine-4-carbonitrile [0946] (0.06 g, 0.167 mmol) in methanol (10 mL) was added palladium on carbon (0.012 g, 10% W/W) in a Parr-shaker bottle. The mixture was hydrogenated under 40 psi hydrogen gas pressure for 6 h. The reaction mixture was filtered through a bed of celite and washed with methanol (20 mL). The combined filtrate was concentrated under reduced pressure to afford crude product which was purified by column chromatography using 2% methanol in chloroform as eluent to afford 4-(aminomethyl)-6-(3,5-dimethyl-1H-pyrazol-1-yl)-N-(3-(trifluoromethyl)phenyl)pyrimidin-2-amine [0948], Compound 190 as an off-white solid (0.018 g). MS(M+1)+=363.2, 1H NMR (400 MHz, Chloroform-d) δ 7.36-7.25 (m, 4H), 7.10-6.98 (m, 3H), 6.09 (s, 1H), 4.52 (bs, 1H), 3.87 (s, 2H), 2.68 (s, 3H), 2.34 (s, 3H).

### Example 88

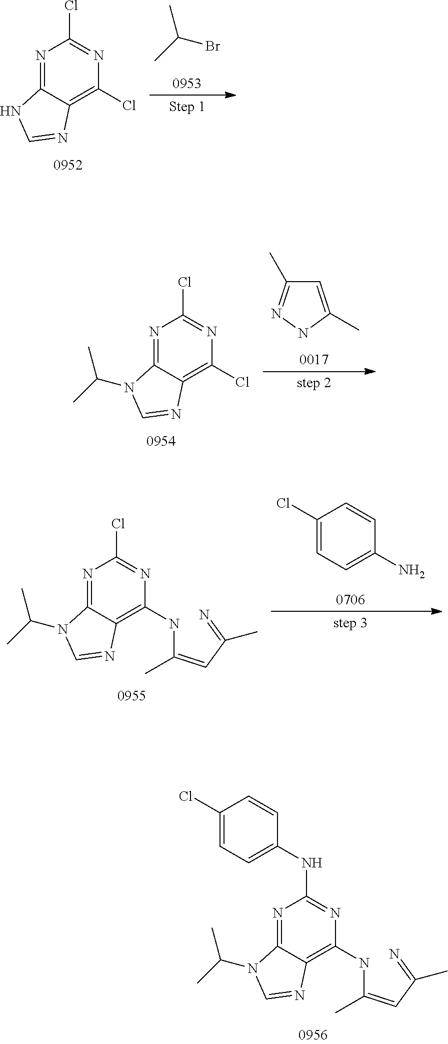

Step 1[0954]: To a stirred solution of 2,6-dichloropurine [0952] (1 g, 5.291 mmol) and 2-bromopropane [0953] (1.952 g, 15.873 mmol) in dimethylsulfoxide (10 mL) was added potassium carbonate (2.194 g, 15.873 mmol). The reaction mixture was stirred at rt for 48 h. The reaction mixture was diluted with ethyl acetate (100 mL) and washed with water (20 mL), brine (20 mL). The organic layer was dried over anhydrous sodium sulfate, filtered and concentrated under reduced pressure to afford crude product which was purified by column chromatography using 2% methanol in chloroform as eluent to afford 2,6-dichloro-9-isopropyl-9H-purine [0954] as off-white solid (0.5 g, 41%). MS(M+1)+=231.1.

Step 2 [0955]: 1.0 g of 2,6-dichloro-9-isopropyl-9H-purine [0954] and 3,5-dimethyl-1H-pyrazole [0017] (0.46 g, 4.327 mmol) gave 0.5 g of 2-chloro-6-(3,5-dimethyl-1H-pyrazol-1-yl)-9-isopropyl-9H-purine [0955] as an off-white solid by using the routine conditions (CS2CO3, ACN, 65° C., 16h). MS(M+1)+=291.2.

Step 3 [0956]: The procedure is similar to step 1[0813] in example 37. 0.25 g of 2-chloro-6-(3,5-dimethyl-1H-pyrazol-1-yl)-9-isopropyl-9H-purine [0955] and 4-chloroaniline [0706] (0.13 g, 1.031 mmol) gave 0.11 g of N-(4-chlorophenyl)-6-(3,5-dimethyl-1H-pyrazol-1-yl)-9-isopropyl-9H-purin-2-amine[0956], Compound 118 as an off-white solid. MS(M+1)+=382.2, 1H NMR (400 MHz, Chloroform-d) δ 8.10 (s, 1H), 7.97 (s, 1H), 7.89-7.66 (m, 2H), 7.45-7.30 (m, 2H), 6.04 (s, 1H), 5.02 (hept, J=6.8 Hz, 1H), 2.59 (s, 3H), 2.37 (s, 3H), 1.64 (d, J=6.8 Hz, 6H).

### Example 89

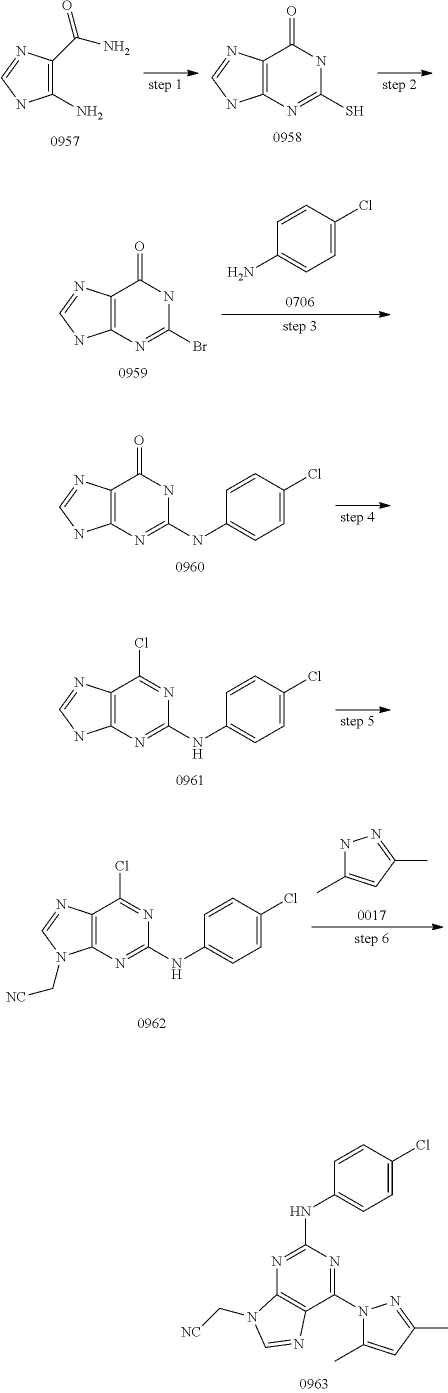

Step 1[0958]: To a stirred solution of 4-aminoimidazole-5-carboxamide hydrochloride [0957] (1 g, 6.151 mmol) in N,N-dimethylformamide (5 mL) was added ethylxanthic acid potassium salt (1.479 g, 9.226) and the mixture was heated at 140° C. for 5 h. The reaction mixture was concentrated under reduced pressure. The residue was triturated with acetonitrile (20 mL). The solid was filtered, washed with acetonitrile (10 mL) and dried under vacuum to afford 2-mercapto-1,9-dihydro-6H-purin-6-one [0958] as a brown solid (0.8 g, 77%). MS(M+1)+=169.0.

Step 2 [0959]: To a stirred suspension of 2-mercapto-1,9-dihydro-6H-purin-6-one [0958] (1 g, 5.946 mmol) in methanol (20 mL) was added bromine (1.44 g, 8.919 mmol) at 0° C. under nitrogen. Then the mixture was stirred with slow warming to rt. After 10 min. the mixture became almost solution and then after 2 h solid was appeared again. After 4 h, the solid was filtered, washed with diethyl ether and dried under vacuum to afford 2-bromo-1,9-dihydro-6H-purin-6-one [0959] as off-white solid (0.8 g, 63%). MS(M+1)+=215.1.

Step 3[0960]: To a stirred suspension of 2-bromo-1,9-dihydro-6H-purin-6-one [0959](0.8 g, 3.720 mmol) and 4-chloroaniline (1.424 g, 11.162 mmol) in a mixture of 2-methoxyethanol (15 mL) and water (5 mL) was heated at 125° C. for 18 h. The solvent was removed under reduced pressure and the residue was triturated with acetonitrile (25 mL) to afford 2-((4-chlorophenyl)amino)-1,9-dihydro-6H-purin-6-one [0960] as off-white solid (0.6 g, 61%). MS(M+1)+=262.2.

Step 4 [0961]: To a stirred suspension of 2-((4-chlorophenyl)amino)-1,9-dihydro-6H-purin-6-one [0960] (0.5 g, 1.911 mmol) in phosphorus oxychloride (2 mL) was added N,N-diisopropyl ethylamine (2 mL). Then the mixture was heated at 90° C. for 16 h and concentrated under reduced pressure to afford crude product which was triturated with chloroform to afford 6-chloro-N-(4-chlorophenyl)-9H-purin-2-amine [0961] as a brown solid (0.4 g, crude). MS(M+1)+=280.1

Step 5[0962]: To a solution of 6-chloro-N-(4-chlorophenyl)-9H-purin-2-amine [0961] (0.4 g, 1.428 mmol) in dimethylsulphoxide (2 mL) were added potassium carbonate (0.395 g, 2.856 mmol) and bromoacetonitrile (0.265 g, 2.142 mmol) at rt under nitrogen. The mixture was then stirred at rt for 18 h. The reaction mixture was diluted with chloroform (50 mL) and washed with water (20 mL), brine (20 mL). The organic layer was dried over anhydrous sodium sulfate, filtered and concentrated under reduced pressure to afford crude product which was triturated with diethyl ether to afford 2-(6-chloro-2-((4-chlorophenyl)amino)-9H-purin-9-yl)acetonitrile [0962] as brownish solid (0.4 g). MS(M+1)+=319.1

Step 6 [0963]: The procedure is similar to step 1[0742] in example 12 (at 160° C.). 0.5 g of 2-(6-chloro-2-((4-chlorophenyl)amino)-9H-purin-9-yl)acetonitrile [0962] and 3,5-dimethyl-1H-pyrazole (0.3 g, 3.133 mmol) gave 0.25 g 2-(2-((4-chlorophenyl)amino)-6-(3,5-dimethyl-1H-pyrazol-1-yl)-9H-purin-9-yl)acetonitrile [0963], Compound 130 as a grey solid. MS(M+1)+=379.3, 1H NMR (400 MHz, DMSO-d6) δ 9.97 (s, 1H), 8.31 (s, 1H), 8.02-7.81 (m, 2H), 7.35 (dd, J=9.3, 2.7 Hz, 2H), 6.21 (s, 1H), 5.51 (d, J=3.1 Hz, 2H), 2.56 (s, 3H), 2.24 (s, 3H).

### Example 90

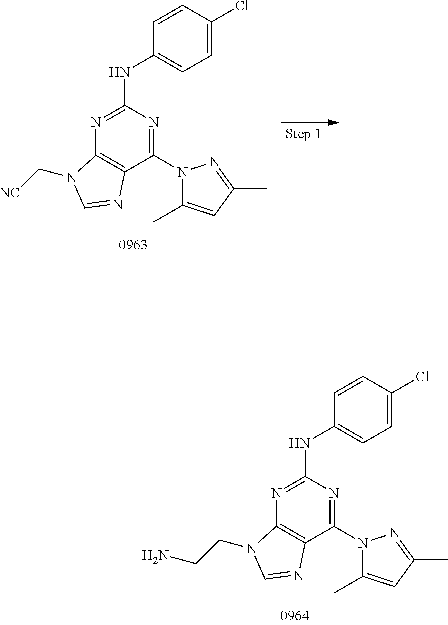 [SMILES: Cc1cc(C)n(-c2nc(Nc3ccc(Cl)cc3)nc3c2ncn3CC#N)n1 ; Cc1cc(C)n(-c2nc(Nc3ccc(Cl)cc3)nc3c2ncn3CCN)n1]

Step 1[0964]: To a stirred suspension of 2-(2-((4-chlorophenyl)amino)-6-(3,5-dimethyl-1H-pyrazol-1-yl)-9H-purin-9-yl)acetonitrile [0963] (0.1 g, 0.264 mmol) in tetrahydrofuran (15 mL) was added a solution of di-isobutylaluminiumhydride in toluene (0.7 mL, IM) at 0° C. under nitrogen. Then the reaction mixture was stirred with slow warming to rt for 18 h. The reaction mixture was quenched with aqueous ammonium chloride solution (10 mL). The product was extracted with chloroform (4×25 mL). The combined organic layer was dried over anhydrous sodium sulfate, filtered and concentrated under reduced pressure to afford crude product which was purified by preparative HPLC to afford 9-(2-aminoethyl)-N-(4-chlorophenyl)-6-(3,5-dimethyl-1H-pyrazol-1-yl)-9H-purin-2-amine [0964], Compound 133 as off-white solid (0.014 g, 14%). MS(M+1)+=383.2, 1H-NMR (400 MHz, DMSO-d6): δ 9.81 (s, 1H), 9.03 (s, 1H), 7.85 (d, J=9.20 Hz, 2H), 7.35 (d, J=9.20 Hz, 2H), 6.17 (s, 1H), 4.17 (t, J=6.80 Hz, 2H), 3.01 (t, J=6.40 Hz, 2H), 2.22 (s, 3H), 1.90 (s, 3H).

### Example 91

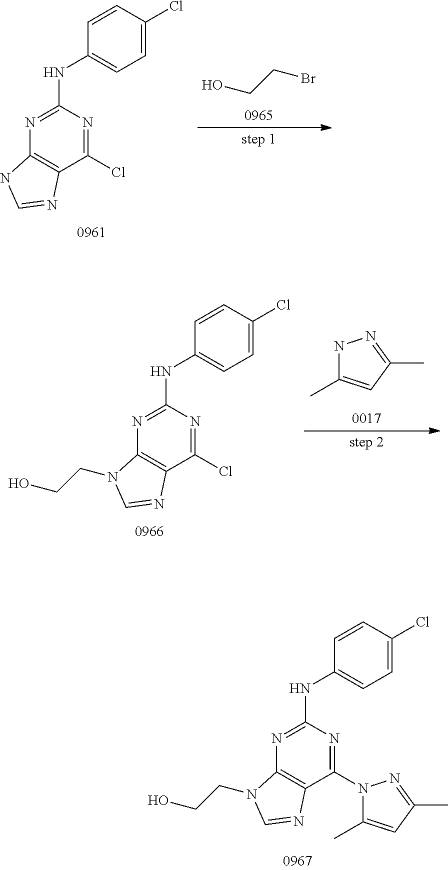

Step 1[0966]: 2.0 g of 2-(6-chloro-2-((4-chlorophenyl)amino)-9H-purin-9-yl)acetonitrile [0961] and 2-bromoethanol [0965] (1.33 g, 10.709 mmol) gave 0.5 g 2-(6-chloro-2-((4-chlorophenyl)amino)-9H-purin-9-yl)ethan-1-ol [0966] as a light brown gum (K2CO3, DMF, rt, 16 h). MS(M+1)+=324.2.

Step 2 [0967]: The procedure is similar to step 1[0742] in example 12 (at 150° C.). 0.5 g of 2-(6-chloro-2-((4-chlorophenyl)amino)-9H-purin-9-yl)ethan-1-ol [0966] and 3,5-dimethyl-1H-pyrazole [0017] (0.22 g, 2.31 mmol) gave 0.035 g 2-(2-((4-chlorophenyl)amino)-6-(3,5-dimethyl-1H-pyrazol-1-yl)-9H-purin-9-yl)ethan-1-ol [0967], Compound 135 as an off-white solid. MS(M+1)+=384.2, 1H NMR (400 MHz, Chloroform-d) δ 7.96 (s, 1H), 7.56 (d, J=8.7 Hz, 2H), 7.44-7.30 (m, 2H), 7.08 (s, 1H), 6.07 (s, 1H), 4.47-4.25 (m, 2H), 4.05 (t, J=4.7 Hz, 2H), 2.63 (t, J=1.4 Hz, 3H), 2.41 (s, 3H).

### Example 92

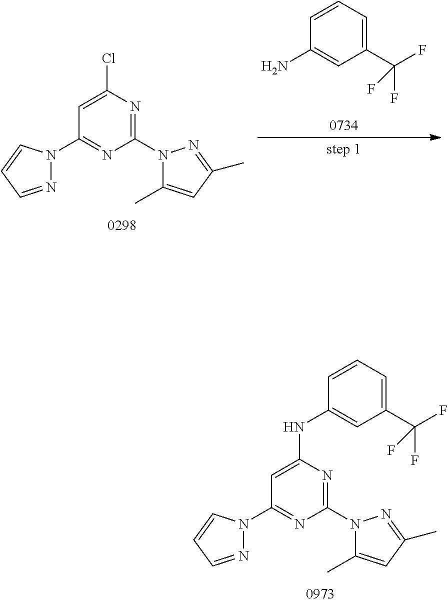 [SMILES: Cc1cc(C)n(-c2nc(Cl)cc(-n3cccn3)n2)n1 ; Nc1cccc(C(F)(F)F)c1 ; Cc1cc(C)n(-c2nc(Nc3cccc(C(F)(F)F)c3)cc(-n3cccn3)n2)n1]

Step 1[0973]: The procedure is similar to Step 1[0748] in example 15. 0.35 g of 4-chloro-2-(3,5-dimethyl-1H-pyrazol-1-yl)-6-(1H-pyrazol-1-yl)pyrimidine [0298] gave 0.120 g of 2-(3,5-dimethyl-1H-pyrazol-1-yl)-6-(1H-pyrazol-1-yl)-N-(3-(trifluoromethyl)phenyl)pyrimidin-4-amine [0973], Compound 183 as an light yellow solid. MS(M+1)+=400, 1H NMR (400 MHz, DMSO-d6) δ 10.42 (s, 1H), 8.88 (s, 1H), 8.57 (d, J=2.6 Hz, 1H), 7.95 (d, J=1.7 Hz, 1H), 7.75 (d, J=8.3 Hz, 1H), 7.59 (t, J=7.9 Hz, 1H), 7.40 (d, J=7.5 Hz, 1H), 7.17 (s, 1H), 6.69 (s, 1H), 6.19 (s, 1H), 2.67 (s, 3H), 2.24 (s, 3H).

### Example 93

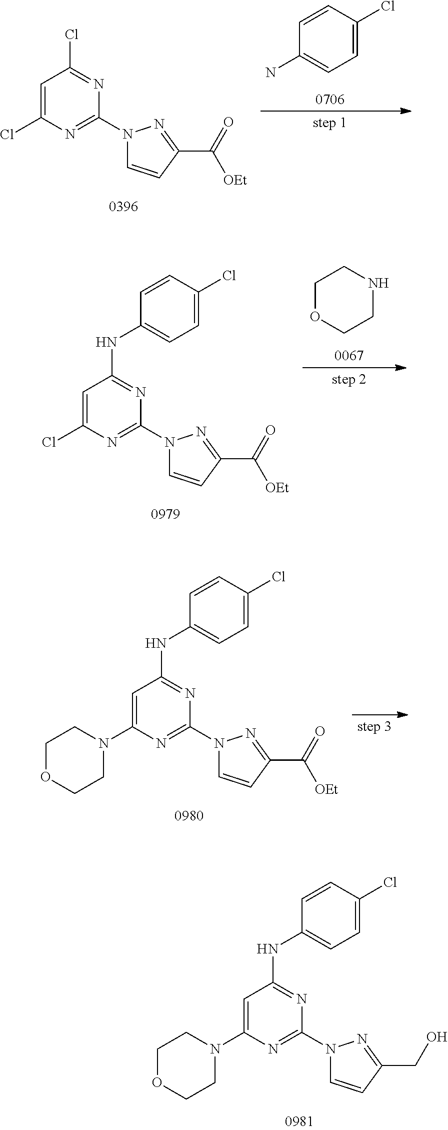 [SMILES: CCOC(=O)c1ccn(-c2nc(Cl)cc(Cl)n2)n1 ; Nc1ccc(Cl)cc1 ; CCOC(=O)c1ccn(-c2nc(Cl)cc(Nc3ccc(Cl)cc3)n2)n1 ; C1COCCN1 ; CCOC(=O)c1ccn(-c2nc(Nc3ccc(Cl)cc3)cc(N3CCOCC3)n2)n1 ; OCc1ccn(-c2nc(Nc3ccc(Cl)cc3)cc(N3CCOCC3)n2)n1]

Step 1[0979]: To a stirred solution of ethyl 1-(4,6-dichloropyrimidin-2-yl)-1H-pyrazole-3-carboxylate [0396] (5 g, 17.415 mmol) in acetonitrile (50 mL), was added 4-chloroaniline [0706] (2.22 g, 17.415 mmol) and N,N-Diisopropyl ethylamine (2.70 g, 20.898 mmol). The reaction mixture was heated at 75° C. for 16h. The reaction mixture was concentrated under reduced pressure and the residue was triturated with water. The solid formed was filtered, dried under vacuum to afford ethyl 1-(4-chloro-6-((4-chloro phenyl)amino)pyrimidin-2-yl)-1H-pyrazole-3-carboxylate [0979] as an off-white solid(4.5 g). MS(M+1)+=296.1

Step 2 [0980]: To a mixture of ethyl 1-(4-chloro-6-((4-chloro phenyl)amino)pyrimidin-2-yl)-1H-pyrazole-3-carboxylate [0979] (0.5 g, 1.322 mmol) and morpholine [0067] (0.230 g, 2.644 mmol) in acetonitrile (8 mL) was added Cesium carbonate(0.510 g, 1.586 mmol). The resultant reaction mixture was irradiated in microwave at 120° C. After 1 h, the reaction mixture was filtered and washed with tetrahydrofuran and the filtrate was concentrated under reduced pressure to afford as a yellow solid, which was purified in the Reveleris flash system instrument using ethyl acetate in hexane as solvent in 40 g column to afford ethyl 1-(4-((4-chlorophenyl)amino)-6-morpholinopyrimidin-2-yl)-1H-pyrazole-3-carboxylate [0980] as yellow solid. 0.28 g, (50% yield). MS(M+1)+=429.2.

Step 3 [0981]: The procedure is similar to step 2[0771] in example 25. 0.28 g of ethyl 1-(4-((4-chlorophenyl)amino)-6-morpholinopyrimidin-2-yl)-1H-pyrazole-3-carboxylate [0980] gave 0.18 g of (1-(4-((4-chlorophenyl)amino)-6-morpholinopyrimidin-2-yl)-1H-pyrazol-3-yl)methanol [0981], Compound 149 as white solid. MS(M+1)+=387.0, 1H NMR (400 MHz, DMSO-d6) δ 9.52 (s, 1H), 8.49 (s, 1H), 7.69 (d, J=8.0 Hz, 2H), 7.46 (d, J=8.8 Hz, 2H), 6.48 (s, 1H), 5.86 (d, J=2.5 Hz, 1H), 5.21 (t, J=5.7 Hz, 1H), 4.51 (d, J=6.0 Hz, 2H), 3.78-3.63 (m, 4H), 3.61-3.47 (m, 4H).

### Example 94

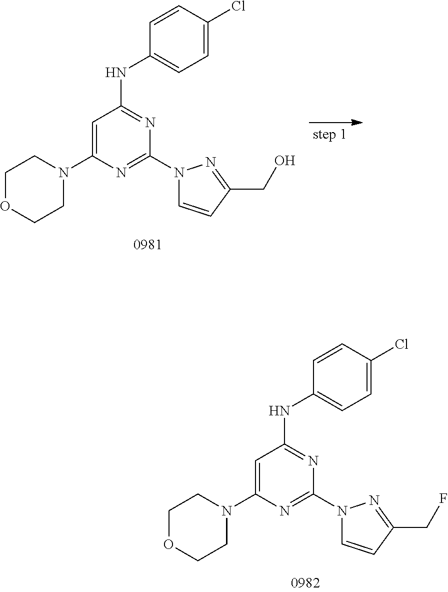 [SMILES: OCc1ccn(-c2nc(Nc3ccc(Cl)cc3)cc(N3CCOCC3)n2)n1 ; FCc1ccn(-c2nc(Nc3ccc(Cl)cc3)cc(N3CCOCC3)n2)n1]

Step 1[0982]: The procedure is similar to step 3[0760] in Example 21. 0.13 g of (1-(4-((4-chloro phenyl)amino)-6-morpholinopyrimidin-2-yl)-1H-pyrazol-3-yl)methanol [0981] gave 0.03 g of N-(4-chlorophenyl)-2-(3-(fluoromethyl)-1H-pyrazol-1-yl)-6-morpholinopyrimidin-4-amine [0982], Compound 151 as a yellow solid. MS(M+1)+=389.4, 1H NMR (400 MHz, DMSO-d6) δ 9.63 (s, 1H), 8.64 (d, J=2.5 Hz, 1H), 7.73 (d, J=8.8 Hz, 2H), 7.43 (d, J=8.8 Hz, 2H), 6.72 (d, J=1.24 Hz, 1H), 5.95 (s, 1H), 5.58 (s, 1H), 5.46 (s, 1H), 3.77 (m, 4H), 3.62 (t, 4H).

### Example 95

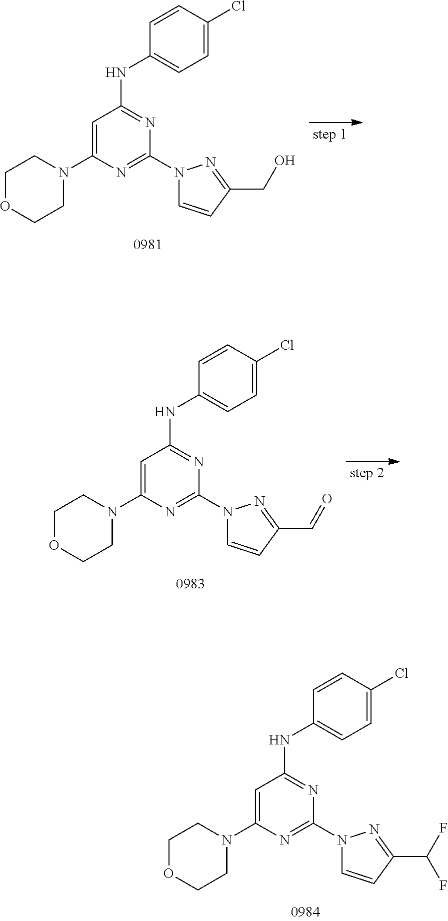 [SMILES: OCc1ccn(-c2nc(Nc3ccc(Cl)cc3)cc(N3CCOCC3)n2)n1 ; O=Cc1ccn(-c2nc(Nc3ccc(Cl)cc3)cc(N3CCOCC3)n2)n1 ; FC(F)c1ccn(-c2nc(Nc3ccc(Cl)cc3)cc(N3CCOCC3)n2)n1]

Step 1[0983]: The procedure is similar to step 1[0786] in Example 28. 0.4 g of (1-(4-((4-chloro phenyl)amino)-6-morpholinopyrimidin-2-yl)-1H-pyrazol-3-yl)methanol [0981] gave 0.3 g of 1-(4-((4-chlorophenyl)amino)-6-morpholinopyrimidin-2-yl)-1H-pyrazole-3-carbaldehyde [0983] as yellow solid. MS(M+1)+=385.2.

Step 2 [0984]: The procedure is similar to step 3[0760] in Example 21. 0.3 g of 1-(4-((4-chlorophenyl)amino)-6-morpholinopyrimidin-2-yl)-1H-pyrazole-3-carbaldehyde [0981] gave 0.055 g of N-(4-chlorophenyl)-2-(3-(difluoromethyl)-1H-pyrazol-1-yl)-6-morpholinopyrimidin-4-amine [0984], Compound 154 as a white solid. MS(M+1)+=407.2. 1H NMR (400 MHz, DMSO-d6) δ 9.61 (s, 1H), 8.67 (d, J=2.7 Hz, 1H), 7.66 (d, J=8.8 Hz, 2H), 7.37 (d, J=8.8 Hz, 2H), 7.14 (t, J_{F}=54.4 Hz, 1H), 6.79 (d, J=2.7 Hz, 1H), 5.92 (s, 1H), 3.71 (m, 4H), 3.57 (m, 4H).

### Example-89

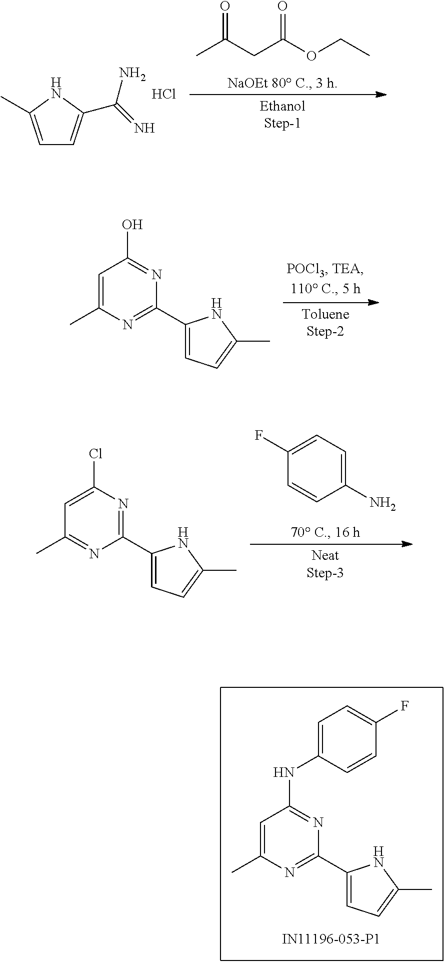

Step 1: The procedure is similar to Step 1[IN10966-057-P2] in Example-48. 2.2 g of 5-methyl-1H-pyrrole-2-carboximidamide gave 6-methyl-2-(5-methyl-1H-pyrrol-2-yl) pyrimidin-4-ol as an off-white solid (2.5 g, crude). MS (M+1)⁺=124.2.

Step 2: The procedure is similar to Step 2[IN10966-057-P2] in Example-48. 0.45 g of 6-methyl-2-(5-methyl-1H-pyrrol-2-yl) pyrimidin-4-ol gave 4-chloro-6-methyl-2-(5-methyl-1H-pyrrol-2-yl) pyrimidine as an off-white solid (0.25 g, crude). MS (M+1)⁺=208.0.

Step 3[IN11196-053-P1]: A solution of 4-chloro-6-methyl-2-(5-methyl-1H-pyrrol-2-yl) pyrimidine in 4-Fluoroaniline was heated at 70° C. for 16h. The reaction mixture was cooled to room temperature and portioned between water (30 mL) and ethyl acetate (2×30 mL). The combined organics were dried over sodium sulfate, filtered and evaporated to afford crude and which was purified by column chromatography using 25% ethyl acetate in hexane as eluent to afford N-(4-fluorophenyl)-6-methyl-2-(5-methyl-1H-pyrrol-2-yl)pyrimidin-4-amine as an off-white solid (0.16 g, 78%). MS (M+1)⁺=283.1; ¹H-NMR (400 MHz, DMSO-d₆): δ 9.30 (s, 1H), 7.76-7.75 (m, 2H), 7.17-7.14 (m, 2H), 6.73 (t, J=2.80 Hz, 1H), 6.29 (s, 1H), 5.85 (t, J=2.40 Hz, 1H), 2.33 (s, 3H), 2.33 (s, 3H).

### Example-90

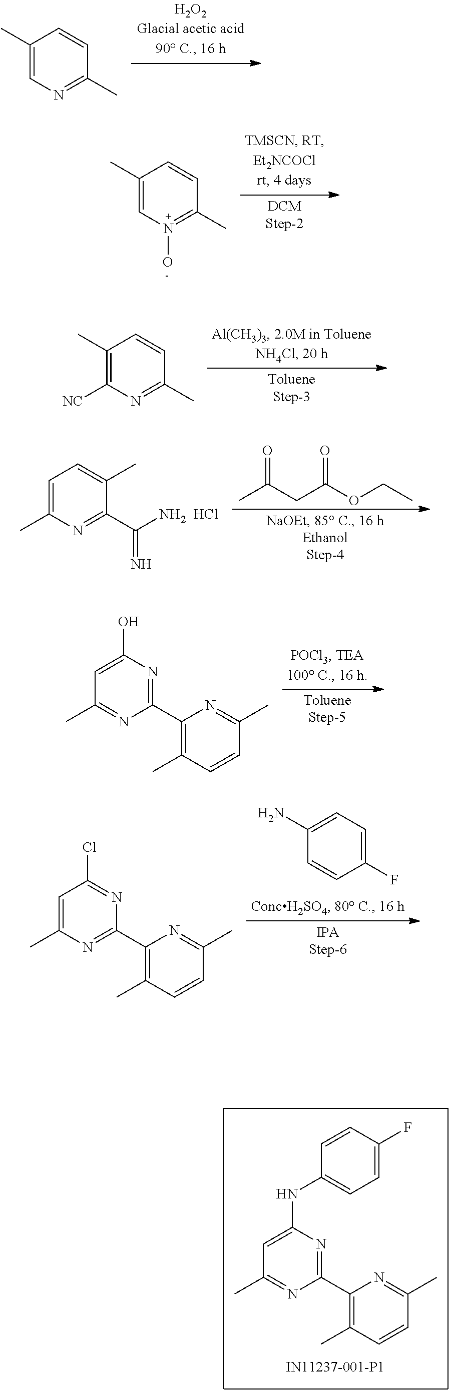

Step 1: To a solution of 2, 5-dimethylpyridine (5 g, 9.88 mmol) in glacial acetic acid (10 mL) was added hydrogen peroxide (12.5 mL). The reaction mixture was heated at 90° C. for 16h. The reaction mixture was neutralized with sat sodium bi-carbonate solution and extracted with DCM (3×50 mL)). The combined organic layer was dried over sodium sulfate and concentrated to afford 2, 5-dimethylpyridine 1-oxide as a yellowish gum (3.4 g, crude). MS (M+1)⁺=124.1.

Step 2: To a solution of 2, 5-dimethylpyridine 1-oxide (3.4 g, 27.60 mmol) in DCM (50 mL) was added trimethyl silyl cyanide (3.79 mL, 30.36 mmol) and stirred at for 30 min. After 30 min, added diethylcarbamic chloride (2.79 mL, 30.36 mmol) and continued to stir at rt for 4 days. Aqueous potassium carbonate solution (100 mL, 10% solution) was added, and the mixture was stirred vigorously for 15 min. The aqueous phase was separated and washed with DCM (3×100 mL). The combined organic layers were washed with water (100 mL), brine (100 mL) dried over sodium sulfate and concentrated under reduced pressure to afford crude and which was purified by column chromatography using 22% ethyl acetate in hexane as eluent to afford 3, 6-dimethylpicolinonitrile as an off-white solid (3 g, 83%). MS (M+1)⁺=133.1.

Step 3: To an ice cooled solution of 3, 6-dimethylpicolinonitrile (2.8 g, 21.18 mmol) in toluene (20 mL) was added Al(CH₃)₃(2.0 M in toluene) (2.28 mL, 42.36 mmol). The reaction mixture was stirred at rt for 30 min. A solution of ammonium chloride (2.26 g, 42.36 mmol) in toluene (10 mL) was added at rt and heated at 80° C. for 16h. The reaction mixture was quenched with methanol (15 mL) the precipitate solid was filtered, washed with methanol and the filtrate was concentrated under reduced pressure to afford crude and which was triturated with diethyl ether (50 mL) and the resulting solid was filtered and dried in vacuum to afford 3, 6-dimethylpicolinimidamide hydrochloride as an off-white solid (2.7 g, crude). MS (M+1)⁺=150.2.

Step 4To a solution of 1.7 g of 3, 6-dimethylpicolinimidamide hydrochloride in ethanol (50 mL) was added ethyl 3-oxobutanoate and sodium ethoxide. The reaction mixture was heated at 80° C. for 16h. The reaction mixture was concentrated under reduced pressure and the resulting residue was diluted with water and washed with ethyl acetate. The aqueous layer was acidified with diluted HCl, pH up to 5, then extracted into ethyl acetate (2×30 mL). The combined organic layer was dried over sodium sulfate and concentrated to afford 6-methyl-2-(4-methylthiazol-2-yl) pyrimidin-4-ol as an off-white solid 2-(3, 6-dimethylpyridin-2-yl)-6-methylpyrimidin-4-ol as an off-white solid (1 g). MS (M+1)⁺=216.2.

Step 5: To a solution of 2-(3, 6-dimethylpyridin-2-yl)-6-methylpyrimidin-4-ol in Phosphorous Oxychloride was added N, N-diethylaniline. The reaction mixture was heated at 95° C. for 2h. The reaction mixture was poured into ice cold water and extracted with ethyl acetate (2×20 mL). The combined organic layer was dried over sodium sulfate and concentrated to afford crude and which was purified by column chromatography using 20% ethyl acetate in hexane as eluent to afford 4-chloro-2-(3, 6-dimethylpyridin-2-yl)-6-methylpyrimidine as an off-white solid (0.44 g, 44%). MS (M+1)⁺=234.1.

Step 6[IN11237-001-P1]: To a suspension of 4-chloro-2-(3, 6-dimethylpyridin-2-yl)-6-methylpyrimidine in Isopropyl alcohol was added 4-Fluoro aniline and Conc Sulphuric acid. The reaction mixture was heated at 80° C. for 16h. The reaction mixture concentrated to remove IPA, the residue was dissolved in ethyl acetate washed with saturated sodium bicarbonate solution (2×25 mL), water (25 mL) and brine (25 mL). The organic layer was dried over sodium sulfate and concentrated under reduced pressure to afford crude and which was purified by column chromatography using 2% methanol in dichloromethane as eluent to afford 2-(3, 6-dimethylpyridin-2-yl)-N-(4-fluorophenyl)-6-methylpyrimidin-4-amine as a pale brown solid (0.35 g, 60%). MS (M+1)⁺=309.2; ¹H-NMR (400 MHz, CDCl₃): δ 7.48 (d, J=8.00 Hz, 1H), 7.28-7.25 (m, 2H), 7.12-7.06 (m, 3H), 6.90 (s, 1H), 6.44 (s, 1H), 2.59 (s, 3H), 2.43 (s, 3H), 2.37 (s, 3H).

### Example-91

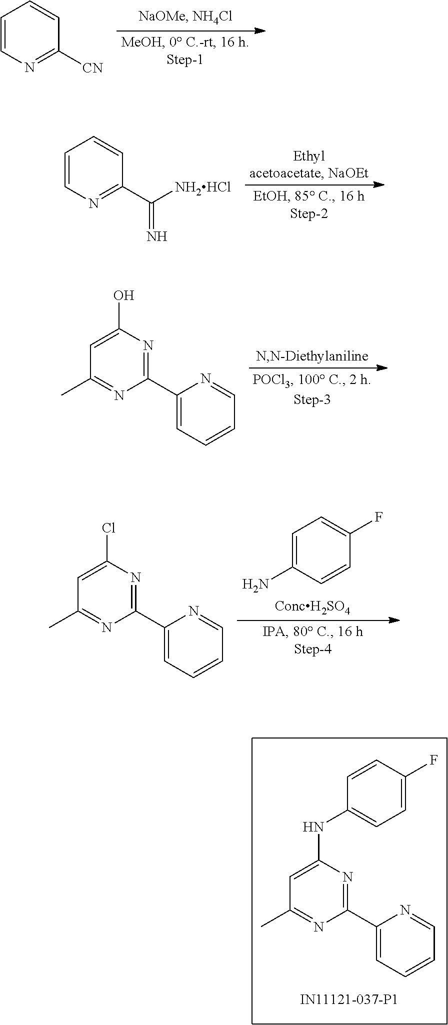

Step 1: To an ice cooled solution of Picolinonitrile (5 g, 48.07 mmol) in methanol (50 mL) was added sodium methoxide (2.85 g, 52.87 mmol) and allowed to stir at rt for 3h. Ammonium chloride (5.09 g, 96.14 mmol) was added to the reaction mixture at rt and the reaction suspension was stirred for about 16h at rt. The reaction mixture was filtered, washed with methanol and the filtrate was concentrated under reduced pressure. The obtained residue was triturated and washed with diethyl ether and the solid were filtered and dried in high vacuum to afford picolinimidamide hydrochloride as a white solid (5 g, 66%). MS (M+1)⁺=122.

Step 2: The procedure is similar to Step 4[IN10966-057-P2] in Example-90. 1 g of picolinimidamide hydrochloride gave 6-methyl-2-(pyridin-2-yl)pyrimidin-4-ol (0.5 g, 42%). MS (M+1)⁺=188.1.

Step 3: The procedure is similar to Step 5[IN10966-057-P2] in Example-90. 0.5 g of 6-methyl-2-(pyridin-2-yl)pyrimidin-4-ol gave 4-chloro-6-methyl-2-(pyridin-2-yl)pyrimidine (0.15 g, 28%). MS (M+1)⁺=206.0.

Step 4[IN11121-037-P1]: To a suspension of 4-chloro-6-methyl-2-(pyridin-2-yl)pyrimidine in Isopropyl alcohol was added 4-Fluoro aniline and Conc Sulphuric acid. The reaction mixture was heated at 80° C. for 16h. The reaction mixture concentrated to remove IPA, the residue was dissolved in ethyl acetate washed with saturated sodium bicarbonate solution (2×25 mL), water (25 mL) and brine (25 mL). The organic layer was dried over sodium sulfate and concentrated under reduced pressure to afford crude and which was purified by column chromatography using 2% methanol in dichloromethane as eluent to afford N-(4-fluorophenyl)-6-methyl-2-(pyridin-2-yl)pyrimidin-4-amine (0.11 g, 55%). MS (M+1)⁺=281.1; ¹H-NMR (400 MHz, DMSO-d₆): δ 9.63 (s, 1H), 8.72 (d, J=4.40 Hz, 1H), 8.14 (d, J=7.60 Hz, 1H), 7.95-7.92 (m, 1H), 7.82-7.79 (m, 2H), 7.50-7.47 (m, 1H), 7.42 (t, J=8.80 Hz, 2H), 6.62 (s, 1H), 2.40 (s, 3H).

### Example-92

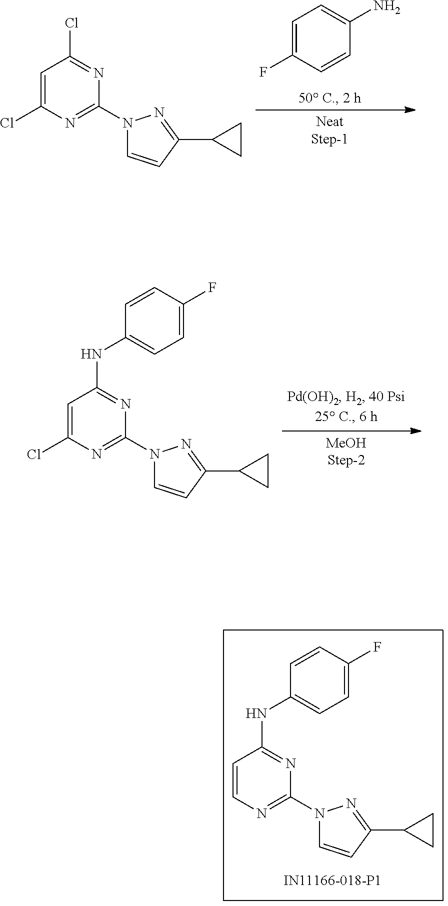 [SMILES: Clc1cc(Cl)nc(-n2ccc(C3CC3)n2)n1 ; Nc1ccc(F)cc1 ; Fc1ccc(Nc2cc(Cl)nc(-n3ccc(C4CC4)n3)n2)cc1 ; Fc1ccc(Nc2ccnc(-n3ccc(C4CC4)n3)n2)cc1]

Step 1: Mixture of 4, 6-dichloro-2-(3-cyclopropyl-1H-pyrazol-1-yl)pyrimidine (0.2 g, 0.787 mmol) and 4-fluoroaniline (1 mL) was heated at 50° C. for 2h, the reaction mixture was quenched with ice cooled water and stirred for 10 min. The solid formed was filtered and dried under vacuum to afford 6-chloro-2-(3-cyclopropyl-1H-pyrazol-1-yl)-N-(4-fluorophenyl) pyrimidin-4-amine as an off-white solid (0.2 g, 77%). MS (M+1)⁺=330.0.

Step 1[IN11166-018-P1]: To a stirred solution of 6-chloro-2-(3-cyclopropyl-1H-pyrazol-1-yl)-N-(4-fluorophenyl) pyrimidin-4-amine in methanol (5 mL) was added Formic acid (0.2 mL) and followed by palladium on carbon (10%, 0.05 g). The reaction mixture was stirred at rt for 16h. The reaction mixture was filtered through celite, filtrate was concentrated under reduced pressure, and residue was quenched with saturated bicarbonate solution and extracted with ethyl acetate (2×50 mL). The combined organic layer was dried over sodium sulfate, filtered and concentrated under reduced pressure to afford 2-(3-cyclopropyl-1H-pyrazol-1-yl)-N-(4-fluorophenyl) pyrimidin-4-amine as an off-white solid (0.045 g, 17%). MS (M+1)⁺=296.0; ¹H-NMR (400 MHz, DMSO-d₆): δ 9.89 (s, 1H), 8.38 (d, J=2.4 Hz, 1H), 8.24 (d, J=5.2 Hz, 1H), 7.78-7.75 (m, 2H), 7.23-7.19 (m, 2H), 6.62 (d, J=6.00 Hz, 1H), 6.28 (d, J=2.4 Hz, 1H), 2.03-1.96 (m, 1H), 0.97-0.89 (m, 2H), 0.82-0.75 (m, 2H).

### Example-93

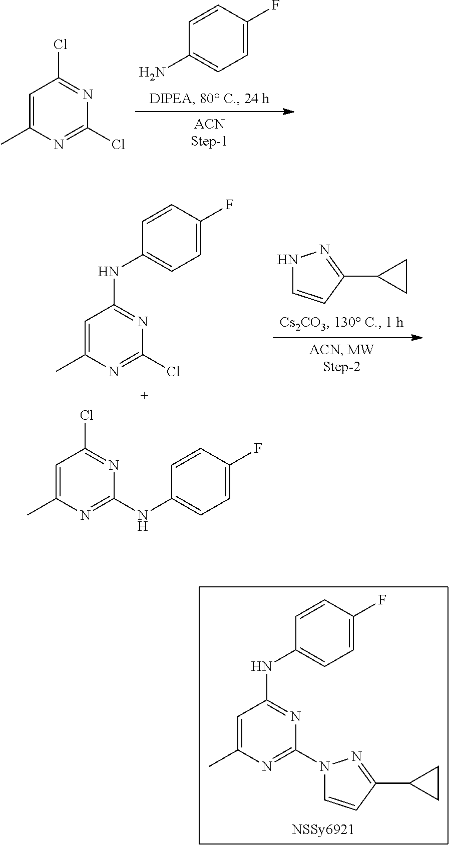

Step 1: To a stirred solution of 4, 6-dichloro-N-(4, -difluorocyclohexyl pyrimidin-2-amine (1 g, 3.54 mmol) in acetonitrile (10 mL) was added 3-fluoro pyrazole (0.36 g, 4.25 mmol) and cesium carbonate (2.30 g, 7.089 mmol). The reaction mixture was heated at 80° C. for 8h. The reaction mixture was filtered and the filtrate was concentrated to afford crude product and which was purified by column chromatography (60-120 mesh) using 22% ethyl acetate in pet ether as solvent to afford 2-chloro-N-(4-fluorophenyl)-6-methylpyrimidin-4-amine as an off-white solid. MS (M+1)⁺=238.0.

Step 2[NSSy6921]: To a solution of 2-chloro-N-(4-fluorophenyl)-6-methylpyrimidin-4-amine and 3-cyclopropyl-1H-pyrazolein acetonitrile (5 mL) was added cesium carbonate and the reaction mixture was irradiated under microwave at 130° C. for 2h. The reaction mixture was filtered and the filtrate was concentrated to afford crude product, which was purified by grace instrument using 80% ethyl acetate in pet-ether to afford 2-(3-cyclopropyl-1H-pyrazol-1-yl)-N-(4-fluorophenyl)-6-methylpyrimidin-4-amine as an off-white solid (0.073 g, 37%). MS (M+1)⁺=310.0; ¹H-NMR (400 MHz, DMSO-d₆): δ 9.77 (s, 1H), 8.37 (s, 1H), 7.73 (m, 2H), 7.23-7.19 (m, 2H), 6.46 (s, 1H), 6.26 (s, 1H), 2.34 (s, 3H), 2.00-1.98 (m, 2H), 0.96-0.94 (m, 2H), 0.77-0.76 (m, 2H).

### Example-94

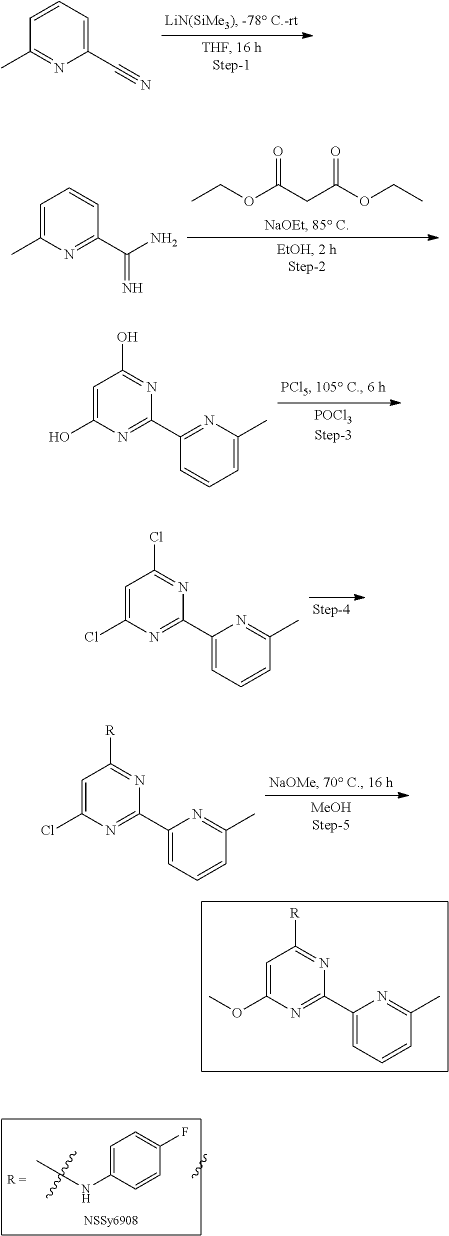

Step 1: To a pre (−78° C.) cooled solution of 6-methyl-2-Pyridinecarbonitrile (5 g, 42.32 mmol) in Tetrahydrofuran (50 mL) was added Lithium bis(trimethylsilyl)amide (14.16 g, 84.64 mmol) and slowly warmed to rt and continued for 16h. After that 1.5 N HCl solution (50 mL) was added to the reaction mixture and stirred for 1 h. Then extracted with ethyl acetate (100 mL), the aqueous layer was basified and extracted with chloroform (3×100 mL). The chloroform was dried over sodium sulfate, filtered and concentrated under reduced pressure to afford 6-methylpicolinimidamide as an off-white solid (3.5 g, 40%). MS (M+1)⁺=136.1.

Step 2: The procedure is similar to Step 4 in Example-90. 3.5 g of 6-methylpicolinimidamide gave 2-(6-methylpyridin-2-yl) pyrimidine-4, 6-diol as red solid (3.5 g, 67%). MS (M+1)⁺=204.1.

Step 3: To a suspension of 2-(6-methylpyridin-2-yl)pyrimidine-4, 6-diol (3.5 g, 17.2 mmol) in Phosphorus oxychloride (16.06 mL, 172.2 mmol) was added Phosphorus Pentachloride (3.58 g, 17.2 mmol) and heated at 105° C. After 6h, the reaction mixture was cooled to room temperature and quenched with ice and basified using saturated sodium bicarbonate solution to pH=7. The reaction mixture was extracted with ethyl acetate and washed with brine solution. The organic layer was dried over sodium sulfate, filtered and concentrated under reduced pressure to afford crude product, which was purified using ethyl acetate in pet-ether as solvent to afford 4, 6-dichloro-2-(6-methylpyridin-2-yl)pyrimidine as yellow solid (1.3 g, 32%). MS (M+1)⁺=242.2.

Step 4:

| R | Condition | Yield (%) | MS (M + 1)⁺ |
|---|---|---|---|
| 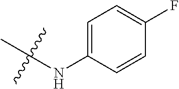 [SMILES: *Nc1ccc(F)cc1] | DIPEA, ACN, 80° C., 16 h, sealed tube. | 86 | 315.1 |

Step 5:

| Compound | | | Yield | MS |
|---|---|---|---|---|
| No | R | Condition | (%) | (M + 1)⁺ |
| NSSy6908 | 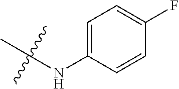 [SMILES: *Nc1ccc(F)cc1] | NaOMe, MeOH, 75° C., 8 h. | 51 | 311.0 |

¹H-NMR (400 MHz, DMSO-d₆): δ 9.61 (s, 1H), 8.13 (d, J=7.36 Hz, 1H), 7.86-7.74 (m, 3H), 7.38 (d, J=7.40 Hz, 1H), 7.19 (s, 1H), 3.96 (s, 3H), 2.59 (s, 3H).

### Example-95

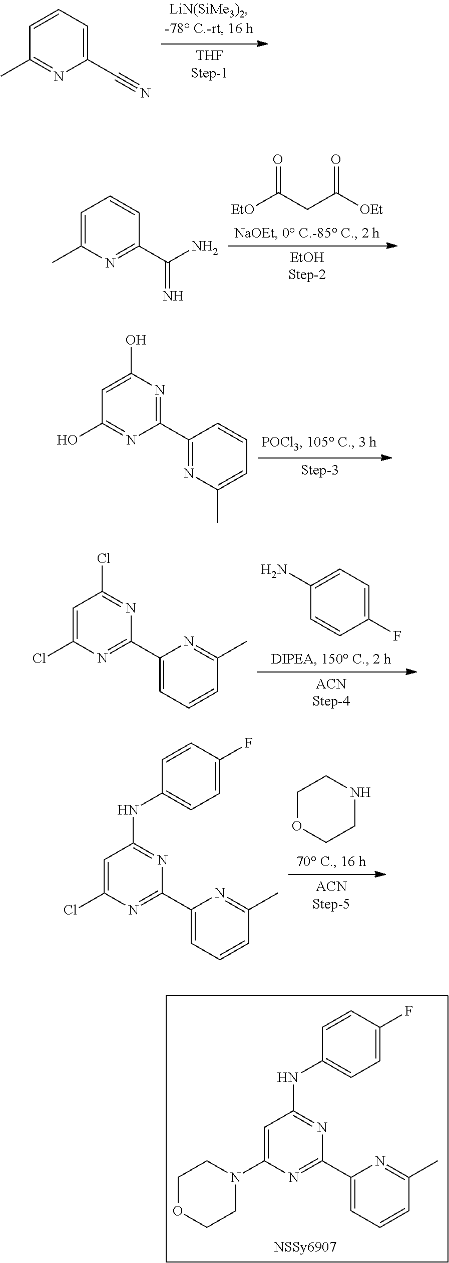

Step 1: The procedure is similar to Step 1 in Example-94. 5.0 g of 6-methylpicolinonitrile gave 6-methylpicolinimidamide as a brownish gum (5.5 g, 94%). MS (M+1)⁺=136.0.

Step 2: The procedure is similar to Step 4 in Example-90. 3.5 g of 6-methylpicolinimidamide gave 2-(6-methylpyridin-2-yl) pyrimidine-4, 6-diol as a brownish gum (3.5 g, 67%). MS (M+1)+=204.1.

Step 3: The procedure is similar to Step 5 in Example-90. 3.5 g of 2-(6-methylpyridin-2-yl) pyrimidine-4, 6-diol gave 4, 6-dichloro-2-(6-methylpyridin-2-yl) pyrimidine as a yellow solid (1.3 g, 31%). MS (M+1)+=240.0.

Step 4: The procedure is similar to Step 1 in Example-93. 0.4 g of 4, 6-dichloro-2-(6-methylpyridin-2-yl) pyrimidine gave 6-chloro-N-(4-fluorophenyl)-2-(6-methylpyridin-2-yl) pyrimidin-4-amine as an off-white solid (0.45 g, 86%). MS (M+1)+=315.1.

Step 5[NSSy6907]: The procedure is similar to Step 5 in Example-95. 0.12 g of 6-chloro-N-(4-fluorophenyl)-2-(6-methylpyridin-2-yl) pyrimidin-4-amine gave N-(4-fluorophenyl)-2-(6-methylpyridin-2-yl)-6-morpholinopyrimidin-4-amine as a white solid (0.05 g, 36%). MS (M+1)+=366.1; 1H-NMR (400 MHz, DMSO-d6): δ 9.29 (s, 1H), 8.07 (d, J=7.60 Hz, 1H), 7.77-7.72 (m, 3H), 7.33 (d, J=7.60 Hz, 1H), 7.15 (t, J=2.40 Hz, 2H), 5.95 (s, 1H), 3.73-3.71 (m, 4H), 3.57-3.54 (m, 4H), 2.56 (s, 3H).

### Example-96

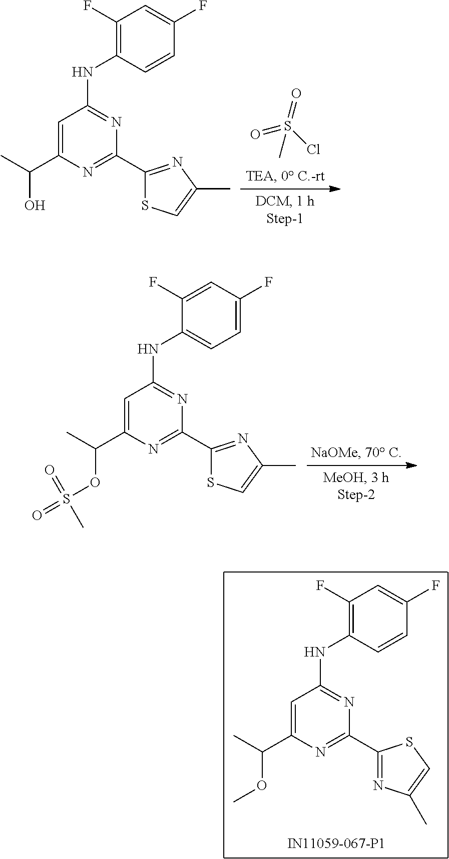

Step 1: To a stirred solution of 1-(6-((2, 4-difluorophenyl) amino)-2-(4-methylthiazol-2-yl) pyrimidin-4-yl) ethan-1-ol in DCM (15 mL) was added trimethylamine followed by methanesulfonyl chloride (0.31 mL, 3.97 mmol) at 0° C. and the reaction mixture was allowed to stir at rt for 1 h. The reaction mixture was diluted DCM (150 mL) and washed with saturated sodium bicarbonate solution, the organic solution was dried over sodium sulfate and concentrated under reduced pressure to afford 1-(6-((2, 4-difluorophenyl) amino)-2-(4-methylthiazol-2-yl) pyrimidin-4-yl) ethyl methanesulfonate as light brownish gum (0.23 g, crude). MS (M+1)⁺=427.

Step 2[IN11059-067-P1]: To a solution of 1-(6-((2, 4-difluorophenyl) amino)-2-(4-methylthiazol-2-yl) pyrimidin-4-yl) ethyl methanesulfonate in methanol (2 mL) was added sodium methoxide. The reaction mixture was heated at 50° C. for 16h. The reaction mixture was concentrated and the resulting residue was dissolved in water, extracted with ethyl acetate (2×20 mL). The combined organic layer was dried over sodium sulfate and concentrated to afford N-(2, 4-difluorophenyl)-6-(1-methoxyethyl)-2-(4-methylthiazol-2-yl) pyrimidin-4-amine as an off-white solid (0.07 g, 50%). MS (M+1)⁺=363.0; ¹H-NMR (400 MHz, DMSO-d₆): δ 9.58 (s, 1H), 7.99 (s, 1H), 7.45-7.35 (m, 2H), 7.08-7.02 (m, 1H), 6.81 (s, 1H), 4.27 (q, J=6.40 Hz, 1H), 3.25 (s, 3H), 2.44 (s, 3H), 1.37 (d, J=6.80 Hz, 3H).

### Example-97

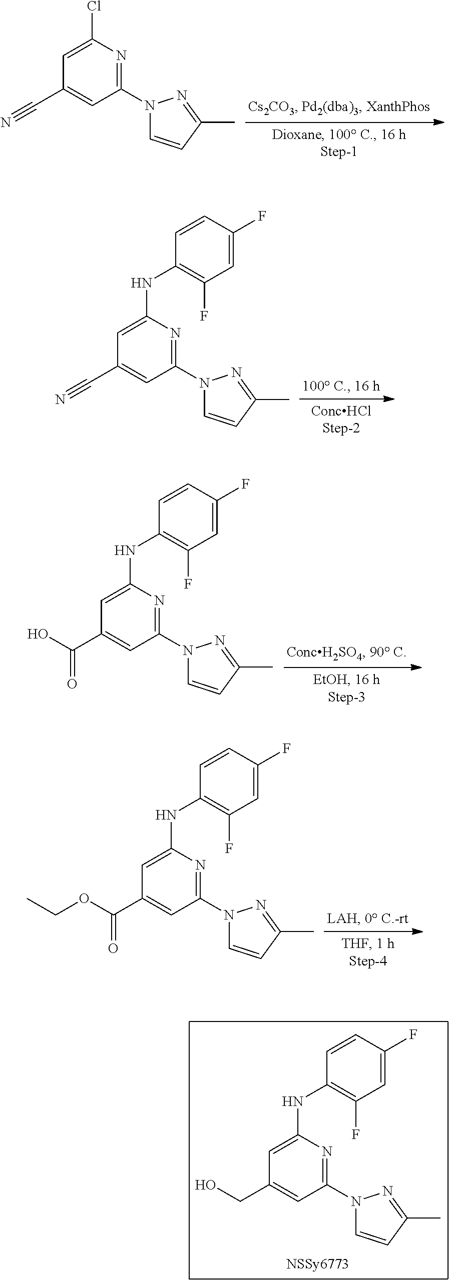 [SMILES: Cc1ccn(-c2cc(C#N)cc(Cl)n2)n1 ; Cc1ccn(-c2cc(C#N)cc(Nc3ccc(F)cc3F)n2)n1 ; Cc1ccn(-c2cc(C(=O)O)cc(Nc3ccc(F)cc3F)n2)n1 ; CCOC(=O)c1cc(Nc2ccc(F)cc2F)nc(-n2ccc(C)n2)c1 ; Cc1ccn(-c2cc(CO)cc(Nc3ccc(F)cc3F)n2)n1]

Step 1: To a solution of 2-chloro-6-(3-methyl-1H-pyrazol-1-yl) isonicotinonitrile (0.3 g, 0.802 mmol) and 3-methylpyrazole in dioxane (10 mL) was added cesium carbonate, followed by 4, 5-Bis(diphenylphosphino)-9, 9-dimethylxanthene and the reaction mixture was purged with N₂ gas for 5 min. Then tris (dibenzylideneacetone) dipalladium (0) was added and the reaction mixture was heated at 90° C. for 24h. The reaction mixture was filtered through celite bed, washed with ethyl acetate and the filtrate was concentrated under reduced pressure to afford crude product, which was purified by flash chromatography using ethyl acetate and pet-ether as solvent system to afford 2-((2, 4-difluorophenyl) amino)-6-(3-methyl-1H-pyrazol-1-yl) isonicotinonitrile as off-white solid (0.2 g, 28%). MS (M+1)⁺=312.0.

Step 2: To a solution of 2-((2, 4-difluorophenyl) amino)-6-(3-methyl-1H-pyrazol-1-yl) isonicotinonitrile in Cone Hydrochloric acid was heated at 100° C. for 16h. The reaction mixture was allowed to cool down, and concentrated under reduced pressure to afford 2-((2, 4-difluorophenyl) amino)-6-(3-methyl-1H-pyrazol-1-yl) isonicotinic acid as yellow solid (0.21 g, 98%). MS (M+1)⁺=331.0.

Step 3: To a stirred solution of 2-((2, 4-difluorophenyl) amino)-6-(3-methyl-1H-pyrazol-1-yl) isonicotinic acid in ethanol (10 mL) was added 0.5 mL Cone sulphuric acid and the reaction mixture was heated at 75° C. for 16h. The reaction mixture was concentrated under reduced pressure and the residue was quenched with saturated bicarbonate solution and extracted with ethyl acetate (2×50 mL). The combined organic layer was dried over sodium sulfate and concentrated to afford ethyl 2-((2, 4-difluorophenyl) amino)-6-(3-methyl-1H-pyrazol-1-yl) isonicotinate as yellow gum (0.22 g, 98%). MS (M+1)⁺=359.0.

Step 4[NSSy6773]: To an ice-cooled solution of ethyl 2-((2, 4-difluorophenyl) amino)-6-(3-methyl-1H-pyrazol-1-yl) isonicotinate in tetrahydrofuran (10 mL) was added Lithium aluminium hydride (2M in THF) and stirred at 0° C. for 1 h. The reaction mixture was quenched with ice cooled water and extracted with ethyl acetate (3×20 mL). The combined organic layer was dried over sodium sulfate and concentrated to afford (2-((2, 4-difluorophenyl) amino)-6-(3-methyl-1H-pyrazol-1-yl) pyridin-4-yl) methanol as an off-white solid (0.08 g, 45%). MS (M+1)⁺=317.0; ¹H-NMR (400 MHz, DMSO-d₆): δ 8.87 (s, 1H), 8.20 (d, J=2.40 Hz, 1H), 7.98-7.92 (m, 1H), 7.35-7.29 (m, 1H), 7.16-7.09 (m, 2H), 6.73 (s, 1H), 6.31 (d, J=2.40 Hz, 1H), 5.45-5.42 (m, 1H), 4.51 (d, J=6.00 Hz, 2H), 2.27 (s, 3H).

### Example-98

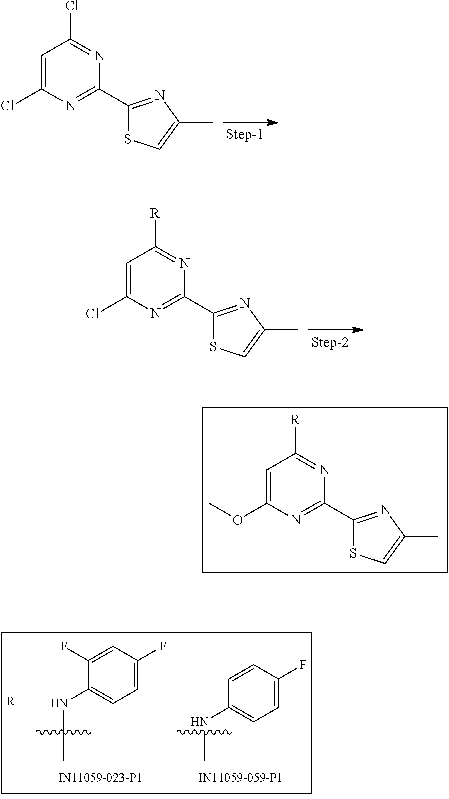

### Step 1:

| R | Condition | Yield (%) | MS (M + 1)⁺ |
|---|---|---|---|
| 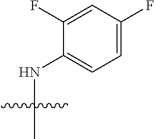 [SMILES: *Nc1ccc(F)cc1F] | H₂SO₄, IPA, 80° C., 16 h | 49 | 339.0 |
| 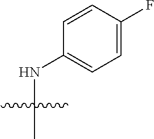 [SMILES: *Nc1ccc(F)cc1] | H₂SO₄, IPA, 80° C., 16 h | 40 | 320.9 |

### Step 2:

| Compound | | | Yield | MS |
|---|---|---|---|---|
| No | R | Condition | (%) | (M + 1)⁺ |
| IN11059- 023-P1 | 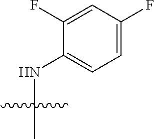 [SMILES: *Nc1ccc(F)cc1F] | NaOMe, 80° C., 16 h, MeOH | 33 | 334.9 |
| IN11059- 059-P1 | 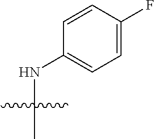 [SMILES: *Nc1ccc(F)cc1] | NaOMe, 80° C., 16 h, MeOH | 45 | 317.0 |

[IN11059-023-P1]: 1H-NMR (400 MHz, DMSO-d6): δ 9.36 (s, 1H), 7.95-7.85 (m, 1H), 7.45 (s, 1H), 7.39-7.34 (m, 1H), 7.18-7.10 (m, 1H), 6.03 (s, 1H), 3.92 (s, 3H), 2.45 (s, 3H).

[IN11059-059-P1]: 1H-NMR (400 MHz, DMSO-d6): δ 9.64 (bs, 1H), 7.67-7.64 (m, 2H), 7.48 (s, 1H), 7.19 (t, J=8.8 Hz, 2H), 6.05 (s, 1H), 3.93 (s, 3H), 2.47 (s, 3H).

### Example-99

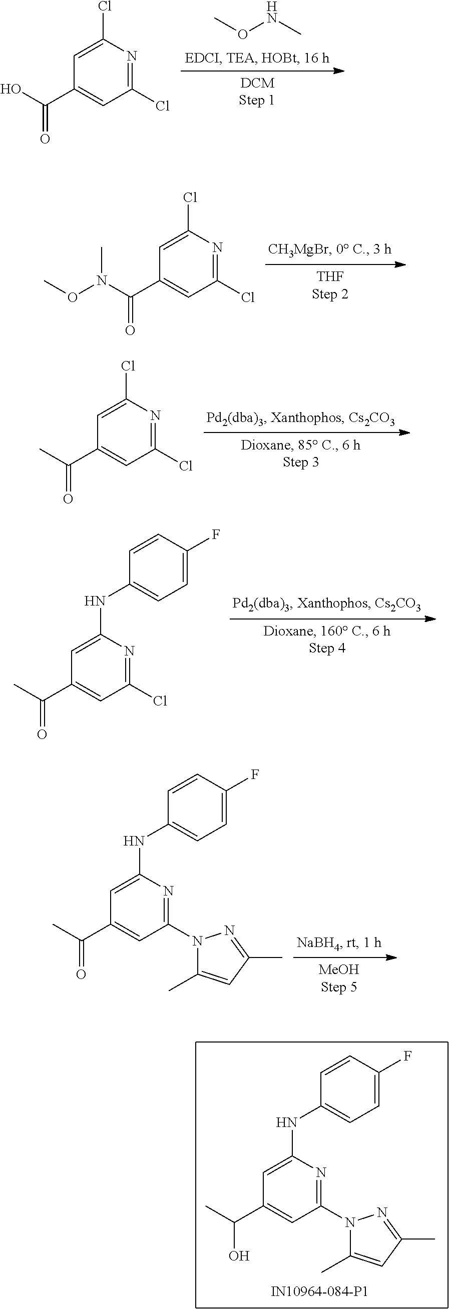

Step 1: To a solution of 2, 6-dichloroisonicotinic acid and N, O-dimethylhydroxylamine in dichloromethane, was added EDCI, HOBt and TEA at room temperature and stirred for 16h. The reaction mixture was diluted with dichloromethane, washed with water and brine solution. The organic layer was dried over sodium sulfate, filtered and concentrated under reduced pressure to afford crude product, which was purified by flash chromatography, using ethyl acetate in hexane as solvent to afford 2, 6-dichloro-N-methoxy-N-methylisonicotinamide as an off-white solid (4 g, 65%). MS (M+1)⁺=235.2.

Step 2: To a pre-cooled (−78° C.) solution of 2, 6-dichloro-N-methoxy-N-methylisonicotinamide in tetrahydrofuran (10 mL) was added methylmagnesium bromide (1.4 M solution in THF:Toluene) and stirred at −78° C. for 2h. The reaction mixture was quenched with saturated aqueous ammonium chloride solution and extracted with ethyl acetate (2×20 mL). The combined organic layer was dried over sodium sulfate and concentrated under reduced pressure to afford crude product, which was purified by preparative HPLC to afford 1-(2, 6-dichloropyridin-4-yl) ethan-1-one as an off-white solid (6 g, 75%). MS (M+1)⁺=191.2.

Step 3: To a solution of 1-(2, 6-dichloropyridin-4-yl) ethan-1-one and 4-fluoroaniline in dioxane (10 mL) was added cesium carbonate, followed by 4, 5-Bis(diphenylphosphino)-9, 9-dimethylxanthene and the reaction mixture was purged with N₂ gas for 5 min. Then tris (dibenzylideneacetone) dipalladium (0) was added and the reaction mixture was heated at 90° C. for 24h. The reaction mixture was filtered through celite bed, washed with ethyl acetate and the filtrate was concentrated under reduced pressure to afford crude product, which was purified by flash chromatography using ethyl acetate and pet-ether as solvent system to afford 1-(2-chloro-6-((4-fluorophenyl) amino) pyridin-4-yl) ethan-1-one as a yellow solid (0.2 g, 29%). MS (M+1)⁺=265.0.

Step 4: The procedure is similar to Step 3 above. 0.2 g of 1-(2-chloro-6-((4-fluorophenyl) amino) pyridin-4-yl) ethan-1-one gave 1-(2-(3, 5-dimethyl-1H-pyrazol-1-yl)-6-((4-fluorophenyl) amino) pyridin-4-yl) ethan-1-one as a yellow solid (0.1 g, crude). MS (M+1)⁺=325.2.

Step 5[IN10964-084-P1]: To an ice cooled solution of 1-(2-(3, 5-dimethyl-1H-pyrazol-1-yl)-6-((4-fluorophenyl) amino) pyridin-4-yl)ethan-1-one in THF (20 mL) was added sodium borohydride and stirred at rt for 5h. The reaction mixture was quenched with water and extracted with ethyl acetate (2×50 mL), the combined organic layer was dried over sodium sulfate and concentrated to afford crude product, which was purified by flash chromatography using 60% ethyl acetate in hexane as eluent to afford 1-(2-(3, 5-dimethyl-1H-pyrazol-1-yl)-6-((4-fluorophenyl)amino)pyridin-4-yl)ethan-1-ol as an off-white solid (0.022 g, 17%). MS (M+1)⁺=327.2; ¹H-NMR (400 MHz, DMSO-d₆): δ 9.09 (s, 1H), 7.54-7.50 (m, 2H), 7.11 (t, J=8.8 Hz, 2H), 7.05 (s, 1H), 6.70 (s, 1H), 6.04 (s, 1H), 5.37 (d, J=4.00 Hz, 1H), 4.70-4.68 (m, 1H), 2.45 (s, 3H), 2.12 (s, 3H), 1.35-1.33 (m, 3H).

### Example-100

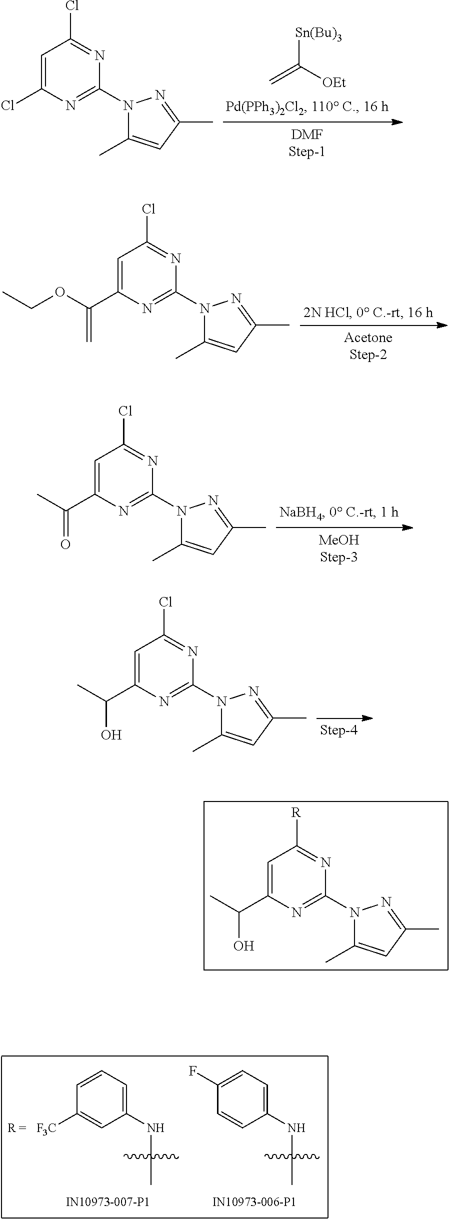

Step 1: To a solution of 4, 6-dichloro-2-(3, 5-dimethyl-1H-pyrazol-1-yl) pyrimidine in toluene (10 mL) was added 4-methyl-2-(tributylstannyl) thiazole. The reaction mixture was purged with N₂ for 5 min, then added bis (triphenylphosphine) Palladium (II) dichloride (0.19 g, 0.28 mmol) and the reaction mixture was heated at 100° C. for 16h. The reaction mixture was filtered through celite bed and the filtrate was concentrated under reduced pressure to afford crude product and which was purified by flash chromatography using ethyl acetate and pet-ether as solvent system to afford 4-chloro-2-(3, 5-dimethyl-1H-pyrazol-1-yl)-6-(1-ethoxyvinyl) pyrimidine as an off-white solid (1.1 g, 53%). MS (M+1)⁺=279.0.

Step 2: To a stirred solution of 4-chloro-2-(3, 5-dimethyl-1H-pyrazol-1-yl)-6-(1-ethoxyvinyl) pyrimidine in acetone (20 mL) was added aqueous hydrochloric acid (2N) (2 mL). The reaction mixture was allowed to stir at rt for 12h. The reaction mixture was concentrated to remove acetone, diluted with ice-cold water, basified with saturated sodium bicarbonate solution and extracted with ethyl acetate (2×25 mL). The combined organic layer was concentrated under reduced pressure to afford crude product and which was purified by column chromatography using ethyl acetate in pet-ether as solvent to afford 1-(6-chloro-2-(3,5-dimethyl-1H-pyrazol-1-yl) pyrimidin-4-yl) ethan-1-one as an off-white solid (0.72 g, 72%). MS (M+1)⁺=251.0.

Step 3: To an ice cooled solution of e-(6-chloro-2-(3, 5-dimethyl-1H-pyrazol-1-yl) pyrimidin-4-yl) ethan-1-one in THF (20 mL) was added Lithium borohydride and stirred at rt for 5h. The reaction mixture was quenched with water and extracted with ethyl acetate (2×50 mL), the combined organic layer was dried over sodium sulfate and concentrated to afford crude product, which was purified by flash chromatography using 60% ethyl acetate in hexane as eluent to afford 1-(6-chloro-2-(3, 5-dimethyl-1H-pyrazol-1-yl) pyrimidin-4-yl) ethan-1-ol as an off-white solid (0.65 g, 92%). MS (M+1)⁺=253.0.

Step 4:

| Compound | | | Yield | MS |
|---|---|---|---|---|
| No | R | Condition | (%) | (M + 1)⁺ |
| IN10973- 007-P1 | 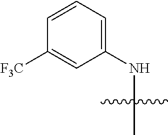 [SMILES: *Nc1cccc(C(F)(F)F)c1] | rt, 16 h | 70 | 378.0 |
| IN10973- 006-P1 | 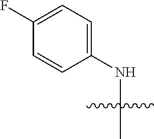 [SMILES: *Nc1ccc(F)cc1] | 70° C., 16 h | 68 | 328.1 |

Step 4[IN10973-007-P1]: ¹H-NMR (400 MHz, DMSO-d₆): δ 8.75 (s, 1H), 7.74 (d, J=8.80 Hz, 1H), 7.53 (t, J=8.00 Hz, 1H), 7.33 (d, J=7.60 Hz, 1H), 6.85 (s, 1H), 6.09 (s, 1H), 5.57 (s, 1H), 4.60-4.55 (m, 1H), 2.52 (s, 3H), 2.18 (s, 3H), 1.38 (d, J=6.40 Hz, 3H).

Step 4[IN10973-006-P1]: ¹H-NMR (400 MHz, DMSO-d₆): δ 9.85 (s, 1H), 7.70 (bs, 2H), 7.19 (t, J=8.8 Hz, 2H), 6.82 (s, 1H), 6.07 (s, 1H), 5.52 (d, J=4.8 Hz, 1H), 4.60-4.52 (m, 1H), 2.48 (s, 3H), 2.19 (m, 3H), 1.38-1.36 (m, 3H).

### Example-101

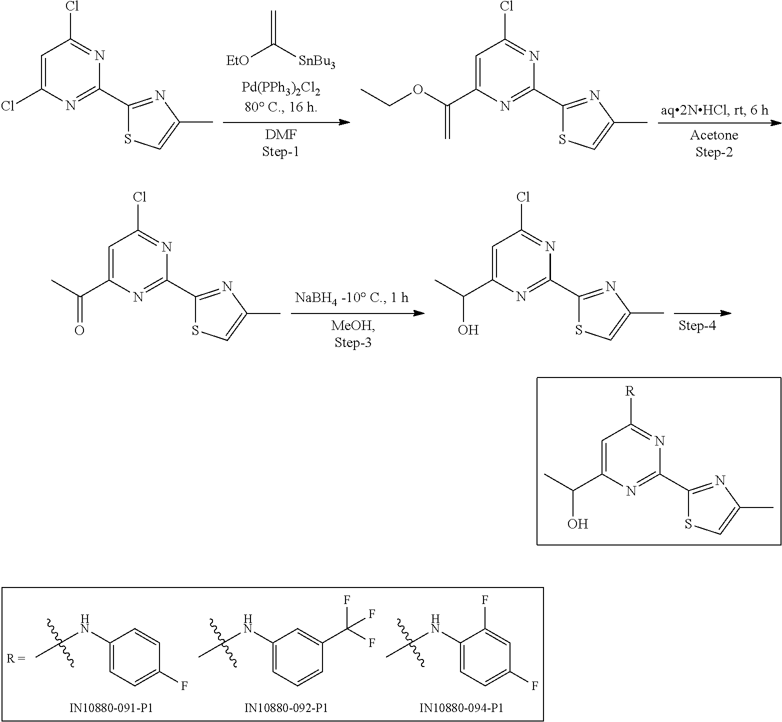

Step 1: The Procedure is similar to Step 1 in Example-100. 0.3 g of 2-(4, 6-dichloropyrimidin-2-yl)-4-methylthiazole gave 2-(4-chloro-6-(1-ethoxyvinyl) pyrimidin-2-yl)-4-methylthiazole as an off-white solid (0.23 g, 67%). MS (M+1)⁺=282.

Step 2: The Procedure is similar to Step 2 in Example-100. 0.23 g of 2-(4-chloro-6-(1-ethoxyvinyl) pyrimidin-2-yl)-4-methylthiazole gave 1-(6-chloro-2-(4-methylthiazol-2-yl) pyrimidin-4-yl) ethan-1-one as an off-white solid (0.17 g, 82%). MS (M+1)⁺=254.

Step 3: The Procedure is similar to Step 3 in Example-100. 0.17 g of 1-(6-chloro-2-(4-methylthiazol-2-yl) pyrimidin-4-yl) ethan-1-one gave 1-(6-chloro-2-(4-methylthiazol-2-yl) pyrimidin-4-yl) ethan-1-ol as an off-white solid (0.16 g, 93%). MS (M+1)⁺=255.9.

Step 4:

| Compound No | R | Condition | Yield (%) |
|---|---|---|---|
| IN10880-091-P1 | 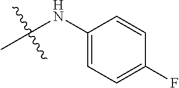 [SMILES: *Nc1ccc(F)cc1] | 4-Fluoroaniline, rt, 16 h, Neat | 68 |
| IN10880-092-P1 | 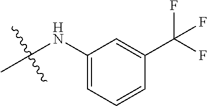 [SMILES: *Nc1cccc(C(F)(F)F)c1] | 3-(Trifluoromethyl) aniline, rt, 16 h, Neat | 66 |
| IN10880-094-P1 | 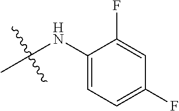 [SMILES: *Nc1ccc(F)cc1F] | 2,4-Difluoroaniline, rt, 16 h, Neat | 68 |

Step 4[IN10880-091-P1]: A solution of 1-(6-chloro-2-(4-methylthiazol-2-yl) pyrimidin-4-yl) ethan-1-ol (0.08 g, 0.313 mmol) in 4-fluoroaniline (0.25 mL) was stirred at rt for 16h. The reaction mixture was diluted with water and extracted with dichloromethane (2×20 mL). The combined organic layer was dried over sodium sulfate and concentrated to afford crude and which was purified by column chromatography using 1% methanol in dichloromethane as eluent to afford 1-(6-((4-fluorophenyl)amino)-2-(4-methylthiazol-2-yl)pyrimidin-4-yl)ethan-1-ol as an brown solid (0.08 g, 68%). MS (M+1)⁺=331.0; ¹H-NMR (400 MHz, DMSO-d₆): δ 9.90 (s, 1H), 7.77 (s, 2H), 7.46 (s, 1H), 7.22 (t, J=9.20 Hz, 2H), 6.94 (s, 1H), 5.55 (d, J=4.40 Hz, 1H), 4.60-4.59 (m, 1H), 2.46 (s, 3H), 1.39 (d, J=6.40 Hz, 3H).

Step 4[IN10880-092-P1]: MS (M+1)⁺=381.0; ¹H-NMR (400 MHz, DMSO-d₆): δ 7.48 (s, 1H), 7.36 (s, 1H), 6.60 (s, 1H), 5.37 (s, 1H), 4.49 (s, 1H), 3.91 (s, 1H), 2.43 (s, 3H), 1.95-1.55 (m, 5H), 1.40-1.10 (m, 8H).

Step 4[IN10880-094-P1]: MS (M+1)⁺=349.0; ¹H-NMR (400 MHz, DMSO-d₆): δ 9.54 (s, 1H), 7.95-7.93 (m, 1H), 7.43-7.37 (m, 2H), 7.18-7.13 (m, 1H), 6.92 (s, 1H), 5.51 (d, J=4.40 Hz, 1H), 4.61-4.58 (m, 1H), 2.51 (s, 3H), 2.43 (s, 3H), 1.37 (d, J=7.20 Hz, 3H).

### Example-102

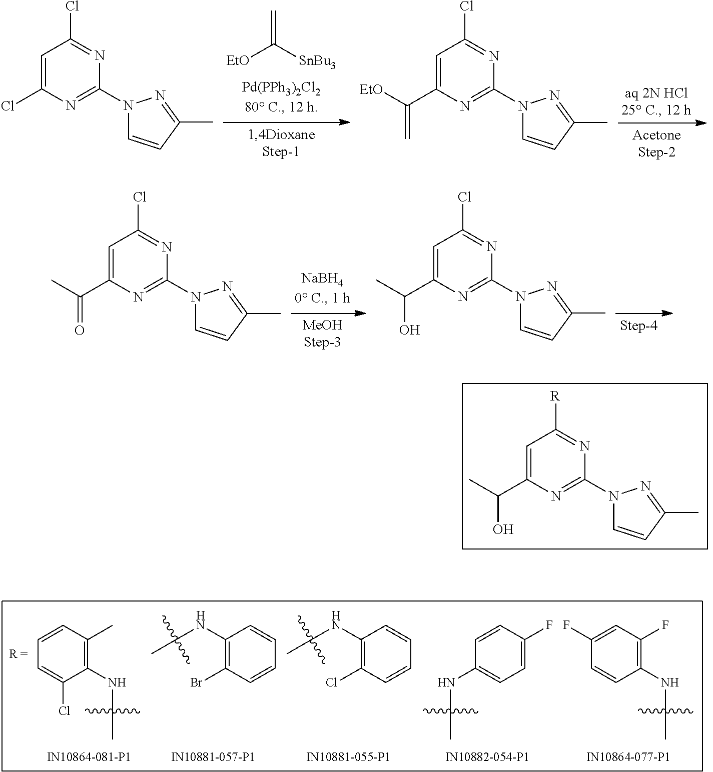

Step 1: The procedure is similar to Step 1 in Example-100. 3.0 g of 4, 6-dichloro-2-(3-methyl-1H-pyrazol-1-yl) pyrimidine gave 4-chloro-6-(1-ethoxyvinyl)-2-(3-methyl-1H-pyrazol-1-yl) pyrimidine as an off-white solid (2.1 g, 60%). MS (M+1)⁺=265.0.

Step 2: The procedure is similar to Step 2 in Example-100. 2.5 g of 4-chloro-6-(1-ethoxyvinyl)-2-(3-methyl-1H-pyrazol-1-yl) pyrimidine gave 1-(6-chloro-2-(3-methyl-1H-pyrazol-1-yl) pyrimidin-4-yl) ethan-1-one as a yellow solid (2.2 g, 66%). MS (M+1)⁺=237.0.

Step 3: The procedure is similar to Step 3 in Example-100. 2.2 g of 1-(6-chloro-2-(3-methyl-1H-pyrazol-1-yl) pyrimidin-4-yl) ethan-1-one gave 1-(6-chloro-2-(3-methyl-1H-pyrazol-1-yl) pyrimidin-4-yl) ethan-1-ol as an off-white solid (1.8 g, 56%). MS (M+1)⁺=239.0.

Step 4:

| Compound No | R | Condition | Yield (%) |
|---|---|---|---|
| IN10882-054-P1 | 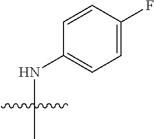 [SMILES: *Nc1ccc(F)cc1] | rt, 12 h | 58 |
| IN10864-077-P1 | 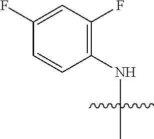 [SMILES: *Nc1ccc(F)cc1F] | 90° C., 24 h | 57 |
| IN10864-081-P1 | 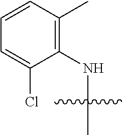 [SMILES: *Nc1c(C)cccc1Cl] | 90° C., 48 h | 55 |
| IN10881-057-P1 | 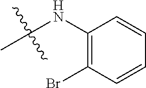 [SMILES: *Nc1ccccc1Br] | 80° C., 12 h | 45 |
| IN10881-055-P1 | 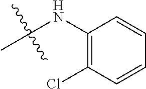 [SMILES: *Nc1ccccc1Cl] | 80° C., 12 h | 45 |

[IN10882-054-P1]: MS (M+1)⁺=314.0; ¹H-NMR (400 MHz, DMSO-d₆): δ 9.87 (s, 1H), 8.42 (d, J=2.40 Hz, 1H), 7.74-7.72 (m, 2H), 7.23-7.19 (m, 2H), 6.80 (s, 1H), 6.35 (d, J=2.40 Hz, 1H), 5.49 (d, J=4.40 Hz, 1H), 4.56-4.53 (m, 1H), 2.29 (s, 3H), 1.38 (d, J=6.80 Hz, 3H).

[IN10864-077-P1]: MS (M+1)⁺=332.0; ¹H-NMR (400 MHz, CD₃OD): δ 9.62 (bs, 1H), 8.31 (d, J=2.0 Hz, 1H), 7.40 (m, 1H), 7.17 (m, 1H), 6.80 (bs, 1H), 6.32 (s, 1H), 5.51 (s, 1H), 4.55 (d, J=6.8 Hz, 1H), 2.26 (s, 3H), 1.37 (d, J=6.8 Hz, 3H).

[IN10864-081-P1]: MS (M+1)⁺=344.0; ¹H-NMR (400 MHz, DMSO-d₆): δ 9.53 (bs, 2H), 8.45 (bs, 1H), 7.97 (bs, 1H), 7.45-7.34 (m, 6H), 6.87 (bs, 1H), 6.33-6.24 (m, 2H), 5.88 (bs, 1H), 5.53-5.35 (m, 2H), 4.49 (bs, 2H), 2.22 (m, 12H), 1.36-1.23 (m, 7H).

[IN10881-057-P1]: MS (M+1)⁺=374.9; ¹H-NMR (400 MHz, DMSO-d₆): δ 9.48 (s, 1H), 8.27 (d, J=2.4 Hz, 1H), 7.75-7.73 (m, 1H), 7.69-7.66 (m, 1H), 7.48-7.44 (m, 1H), 7.23-7.19 (m, 1H), 6.68 (s, 1H), 6.30 (d, J=2.4 Hz, 1H), 5.44 (d, J=4.4 Hz, 1H), 4.56-4.50 (m, 1H), 2.25 (s, 3H), 1.36 (d, J=6.8 Hz, 3H).

[IN10881-055-P1]: MS (M+1)⁺=330.0; ¹H-NMR (400 MHz, DMSO-d₆): δ 9.51 (s, 1H), 8.29 (d, J=2.40 Hz, 1H), 7.78 (d, J=8.00 Hz, 1H), 7.57 (d, J=7.20 Hz, 1H), 7.41 (t, J=7.20 Hz, 1H), 7.27 (t, J=1.20 Hz, 1H), 6.80 (s, 1H), 6.30 (d, J=2.40 Hz, 1H), 5.45 (d, J=5.20 Hz, 1H), 4.53-4.54 (m, 1H), 2.26 (s, 3H), 1.40 (d, J=6.80 Hz, 3H).

### Example-103

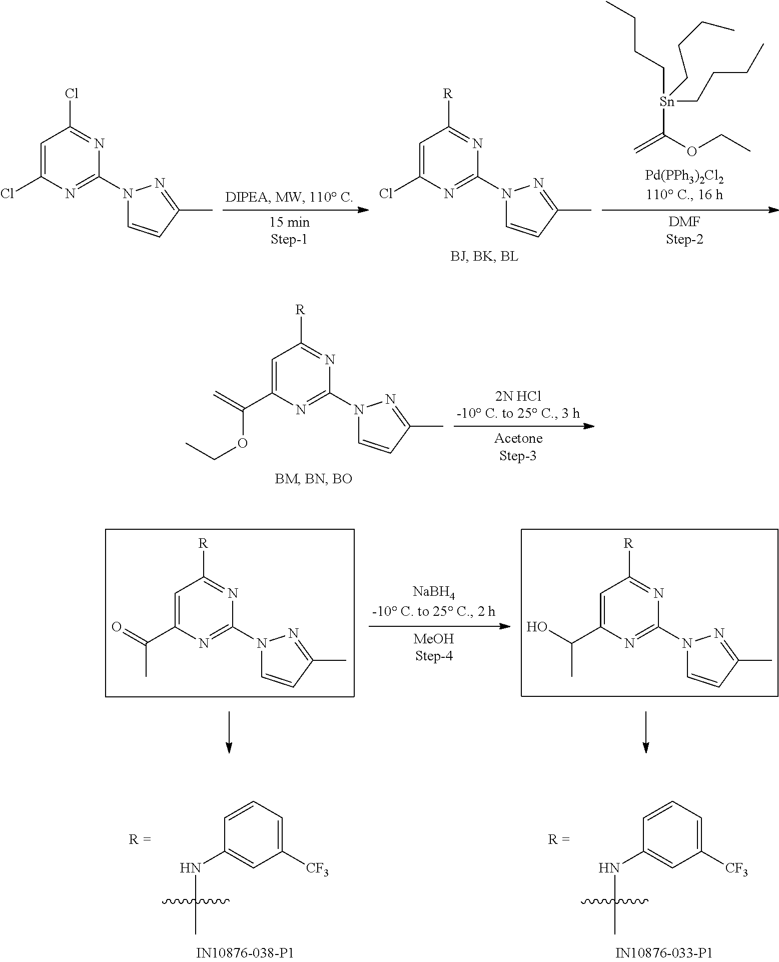

Step 1:

| | | | MS |
|---|---|---|---|
| R | Condition | Yield (%) | (M + 1)⁺ |
| 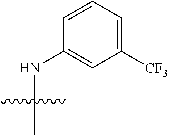 [SMILES: *Nc1cccc(C(F)(F)F)c1] | DIPEA, 110° C., 15 min | 96 | 354.1 |

Step 2:

| R | Condition | Yield (%) | MS (M + 1)⁺ |
|---|---|---|---|
| 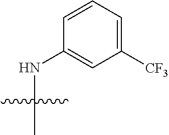 [SMILES: *Nc1cccc(C(F)(F)F)c1] | Pd(PPh₃)₂Cl₂, DMF, 110° C., 16 h | 72 | 390.2 |

Step 3:

| Compound No | R | Condition | Yield (%) | MS (M + 1)⁺ |
|---|---|---|---|---|
| IN10876-038-P1 | 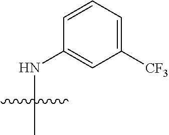 [SMILES: *Nc1cccc(C(F)(F)F)c1] | 2N HCl, acetone, −10° C.-25° C., 3 h | 47 | 362.1 |

Step 3[IN10876-038-P1]: ¹H-NMR (400 MHz, DMSO-d₆): δ 10.45 (s, 1H), 8.73 (s, 1H), 8.54 (d, J=2.80 Hz, 1H), 7.84 (d, J=8.40 Hz, 1H), 7.62 (t, J=7.60 Hz, 1H), 7.43 (d, J=7.60 Hz, 1H), 7.14 (s, 1H), 6.45 (d, J=2.80 Hz, 1H), 2.67 (s, 3H), 2.29 (s, 3H).

Step 4:

| Compound No | R | Condition | Yield (%) | MS (M + 1)⁺ |
|---|---|---|---|---|
| IN10876-033-P1 | 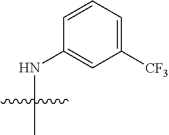 [SMILES: *Nc1cccc(C(F)(F)F)c1] | NaBH₄, MeOH, −10° C.-25° C., 2 h | 70 | 364.1 |

Step 4[IN10876-033-P1]: ¹H-NMR (400 MHz, DMSO-d₆): δ 8.64 (s, 1H), 8.41 (s, 1H), 7.84 (d, J=7.60 Hz, 1H), 7.59 (t, J=8.00 Hz, 1H), 7.37 (d, J=8.00 Hz, 1H), 6.88 (s, 1H), 6.38 (s, 1H), 5.55 (d, J=8.00 Hz, 1H), 4.60-4.56 (m, 1H), 2.29 (s, 3H), 1.36-1.41 (m, 3H).

### Example-104

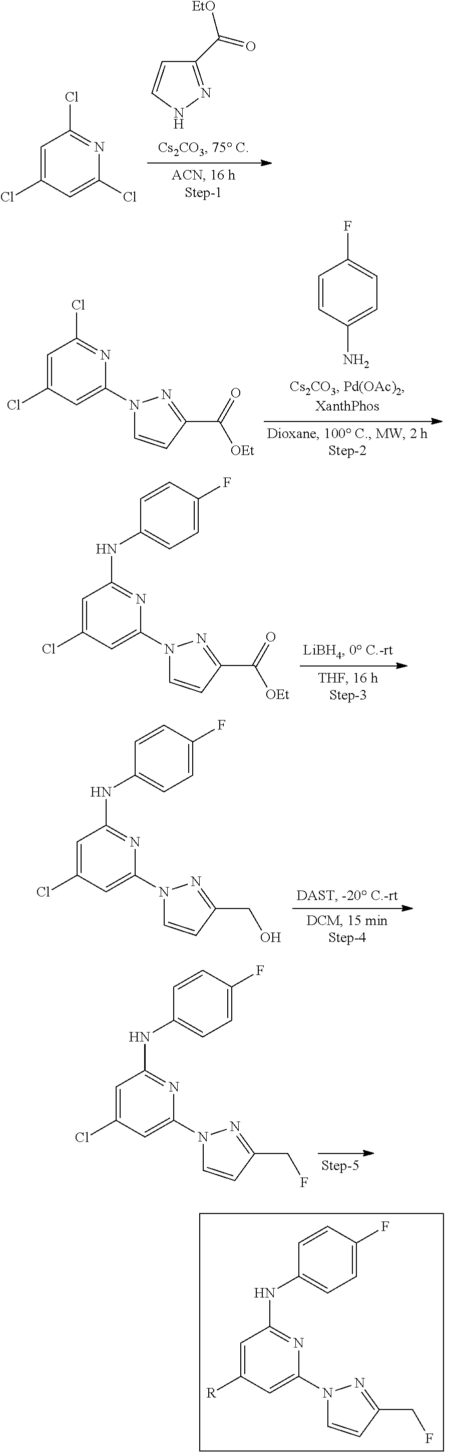

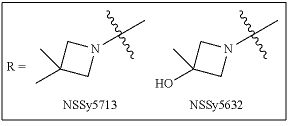

Step 1: To a stirred solution of 2, 4, 6-trichloropyridine (1 g, 3.54 mmol) in acetonitrile was added reagent and cesium carbonate (2.30 g, 7.089 mmol). The reaction mixture was heated at 80° C. for 8h. The reaction mixture was filtered and the filtrate was concentrated to afford crude product and which was purified by column chromatography (60-120 mesh) using 22% ethyl acetate in pet ether as solvent to afford ethyl 1-(4, 6-dichloropyridin-2-yl)-1H-pyrazole-3-carboxylate as white solid (9 g, 29%). MS (M, M+2)⁺=286.0, 288.1.

Step 2: The procedure is similar to Step 1 in Example-97. 1 g of ethyl 1-(4, 6-dichloropyridin-2-yl)-1H-pyrazole-3-carboxylate gave ethyl 1-(4-chloro-6-((4-fluorophenyl) amino) pyridin-2-yl)-1H-pyrazole-3-carboxylate as yellow solid (0.4 g, 23%). MS (M+1)⁺=361.0.

Step 3: The procedure is similar to Step 4 in Example-97. 0.8 g of ethyl 1-(4-chloro-6-((4-fluorophenyl) amino) pyridin-2-yl)-1H-pyrazole-3-carboxylate gave (1-(4-chloro-6-((4-fluorophenyl) amino) pyridin-2-yl)-1H-pyrazol-3-yl) methanol as an off-white solid (0.42 g, 60%). MS (M+1)⁺=319.2.

Step 4: To an ice cooled solution of (1-(4-chloro-6-((4-fluorophenyl) amino) pyridin-2-yl)-1H-pyrazol-3-yl) methanol in DCM was added diethylaminosulphur trifluoride, then the reaction mixture was slowly warmed to rt and stirred for 30 mins. Then the reaction mixture was quenched with 10% sodium bicarbonate solution and extracted with dichloromethane (2×50 mL). The combined organic layer was washed with brine, dried over sodium sulfate, filtered and concentrated under reduced pressure to afford crude product, which was purified by flash chromatography using ethyl acetate and pet-ether as solvent system to afford gave 4-chloro-6-(3-(fluoromethyl)-1H-pyrazol-1-yl)-N-(4-fluorophenyl) pyridin-2-amine as a yellow solid (0.12 g, 23%). MS (M+1)⁺=321.0.

Step 5:

| Compound | | | Yield | MS |
|---|---|---|---|---|
| No | R | Condition | (%) | (M + 1)⁺ |
| NSSy5713 | 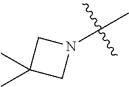 [SMILES: *N1CC(C)(C)C1] | Pd₂(dba)₃, XanthPhos, Cs₂CO₃, Dioxane, 100° C., 16 h | 15 | 385.1 |
| NSSy5632 | 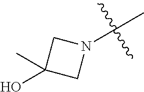 [SMILES: *N1CC(C)(O)C1] | Pd₂(dba)₃, XanthPhos, Cs₂CO₃, Dioxane, 100° C., 16 h | 8 | 372.0 |

Step 5[NSSy5713]: ¹H-NMR (400 MHz, DMSO-d₆): δ 8.78 (s, 1H), 8.47 (d, J=2.40 Hz, 1H), 7.23-7.20 (m, 4H), 6.77 (s, 1H), 6.61 (s, 1H), 5.71 (s, 1H), 5.41 (d, J=48.00 Hz, 2H), 3.99-3.95 (m, 2H), 3.73-3.69 (m, 2H), 3.15-2.01 (m, 1H), 2.11 (s, 6H).

Step 5[NSSy5632]: ¹H-NMR (400 MHz, DMSO-d₆): δ 8.96 (s, 1H), 8.44 (s, 1H), 7.61 (d, J=8.2 Hz, 2H), 7.14 (d, J=8.20 Hz, 2H), 6.66 (s, 1H), 6.39 (s, 1H), 5.71 (s, 2H), 5.65 (s, 1H), 5.51 (s, 1H), 5.39 (s, 1H), 3.88-3.86 (m, 2H), 3.78-3.76 (m, 2H), 1.46 (s, 3H).

### Example-105

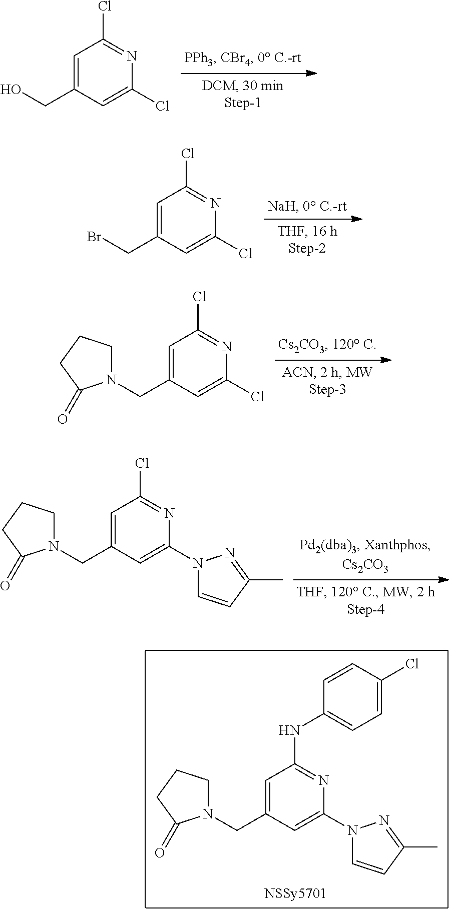

Step 1: To a solution of (2, 6-dichloropyridin-4-yl) methanol (2.5 g, 14.04 mmol) in dichloromethane was added triphenyl phosphine (5.52 g, 21.06 mmol) at 0° C. After 15 min, carbon tetrabromide was added and the reaction mixture was stirred at rt. After 40 min, the reaction mixture was quenched with water and extracted with DCM and washed with brine solution. The organic layer was dried over sodium sulphate, filtered and concentrated under reduced pressure to afford 4-(bromomethyl)-2, 6-dichloropyridine as a brown solid (2.2 g, 66%). MS (M, M+2)⁺=241.0, 243.0.

Step 2: To the suspension of sodium hydride in THF was added 2-Pyrrolidone (1.76 g, 20.75 mmol) at 0° C. under nitrogen atmosphere and stirred at same temperature for 45 mins. The reaction mixture was added slowly to an ice cooled solution of 4-(bromomethyl)-2, 6-dichloropyridine (2.5 g, 10.37 mmol) in THF under nitrogen atmosphere. The reaction mixture was slowly warmed to rt and stirred at rt for 20h. The reaction was quenched with water and extracted in ethyl acetate, washed with water and brine solution. The organic layer was dried over sodium sulphate, filtered and concentrated to afford crude product, which was purified by column chromatography using ethyl acetate in pet-ether as solvent to afford 1-((2, 6-dichloropyridin-4-yl)methyl)pyrrolidin-2-one as yellow solid (1.4 g, 53%). MS (M+1)⁺=247.0.

Step 3: The procedure is similar to Step 2 in Example-93. 1 g of 1-((2, 6-dichloropyridin-4-yl) methyl) pyrrolidin-2-one gave 1-((2-chloro-6-(3-methyl-1H-pyrazol-1-yl) pyridin-4-yl) methyl) pyrrolidin-2-one as yellow solid (0.8 g, 68%). MS (M+1)⁺=291.2.

Step 4 NSSy5701: The procedure is similar to Step 3[NSSy6629] in Example-99. 0.3 g of 1-((2-chloro-6-(3-methyl-1H-pyrazol-1-yl) pyridin-4-yl) methyl) pyrrolidin-2-one gave 1-((2-((4-chlorophenyl) amino)-6-(3-methyl-1H-pyrazol-1-yl) pyridin-4-yl) methyl) pyrrolidin-2-one as yellow solid (0.2 g, 52%). MS (M+1)⁺=382.2; ¹H-NMR (400 MHz, DMSO-d₆): δ 9.43 (s, 1H), 8.36 (d, J=2.16 Hz, 1H), 7.69 (d, J=8.84 Hz, 2H), 7.37 (d, J=8.80 Hz, 2H), 7.10 (s, 1H), 6.52 (s, 1H), 6.37 (d, J=2.12 Hz, 1H), 4.38 (s, 2H), 3.33-3.29 (m, 2H), 2.02-1.94 (m, 5H), 1.94-1.93 (m, 2H).

### Biological Assays

The biological activity of certain compounds were determined as follows. The ionic current through small-conductance Ca²⁺-activated K⁺ channels (SK channels, subtype 2) is recorded using the whole-cell configuration of the patch-clamp technique in a classic patch-clamp set-up using HEK293 tissue culture cells expressing hSK2 channels as described in Kasumu et. al., Chemistry & Biology 19, 1340-1353, Oct. 26, 2012.

The SC₁₀₀ value determined is defined as the Stimulating Concentration required for increasing the baseline current by 100%. The SC₁₀₀ values given in Table 3 and Table 4.

TABLE 3

| Cmpd | SC₁₀₀ |
|---|---|
| No. | uM |
| 100 | +++ |
| 101 | +++ |
| 102 | ++ |
| 103 | +++ |
| 104 | ++ |
| 105 | ++ |
| 106 | ++ |
| 107 | + |
| 108 | ++ |
| 109 | ++ |
| 110 | ++ |
| 111 | +++ |
| 112 | ++ |
| 113 | ++ |
| 114 | ++ |
| 115 | ++ |
| 116 | +++ |
| 117 | +++ |
| 118 | +++ |
| 119 | + |
| 120 | + |
| 121 | +++ |
| 122 | ++ |
| 123 | ++ |
| 124 | ++ |
| 125 | ++ |
| 126 | ++ |
| 127 | + |
| 128 | + |
| 129 | +++ |
| 130 | ++ |
| 131 | +++ |
| 132 | +++ |
| 133 | + |
| 134 | ++ |
| 135 | ++ |
| 136 | +++ |
| 137 | + |
| 138 | + |
| 139 | ++ |
| 140 | + |
| 141 | ++ |
| 142 | ++ |
| 143 | ++ |
| 144 | + |
| 145 | ++ |
| 146 | + |
| 147 | ++ |
| 148 | + |
| 149 | ++ |
| 150 | ++ |
| 151 | ++ |
| 152 | +++ |
| 153 | + |
| 154 | ++ |
| 155 | + |
| 156 | + |
| 157 | ++ |
| 158 | +++ |
| 159 | +++ |
| 160 | + |
| 161 | +++ |
| 162 | ++ |
| 163 | ++ |
| 164 | +++ |
| 165 | ++ |
| 166 | + |
| 167 | ++ |
| 168 | +++ |
| 169 | + |
| 170 | ++ |
| 171 | NT |
| 172 | NT |
| 173 | NT |
| 174 | NT |
| 175 | NT |
| 176 | NT |
| 177 | NT |
| 178 | NT |
| 179 | NT |
| 180 | NT |
| 181 | NT |
| 182 | NT |
| 183 | NT |
| 184 | NT |
| 185 | NT |
| 186 | NT |
| 187 | NT |
| 188 | NT |
| 189 | NT |
| 190 | NT |
| 191 | NT |
| 192 | NT |
| 193 | NT |
| 194 | NT |
| 195 | NT |
| 196 | NT |
| 197 | NT |
| 198 | NT |
| 199 | NT |
| 200 | NT |
| 201 | NT |
| 202 | NT |
| 203 | NT |

+ means >1 uM;
++ means 200 nM-1000 nM;
+++ means <200 nM;
NT means not tested.

TABLE 4

| Compound Ref. | SC₁₀₀ (μM) |
|---|---|
| IN11196-053-P1 | + |
| IN11237-001-P1 | + |
| IN11121-037-P1 | + |
| IN11130-036-P1 | + |
| IN11166-018-P1 | + |
| NSSy6921 | + |
| NSSy6908 | + |
| NSSy6907 | ++ |
| IN11059-067-P1 | + |
| NSSy6773 | ++ |
| IN11059-059-P1 | ++ |
| IN11059-023-P1 | ++ |
| IN10964-084-P1 | + |
| IN10973-007-P1 | +++ |
| IN10973-006-P1 | ++ |
| IN10880-094-P1 | ++ |
| IN10880-092-P1 | ++ |
| IN10880-091-P1 | ++ |
| IN10864-077-P1 | + |
| IN10864-081-P1 | + |
| IN10881-055-P1 | ++ |
| IN10882-054-P1 | ++ |
| IN10876-038-P1 | + |
| IN10876-033-P1 | ++ |
| NSSy5713 | + |
| NSSy5701 | +++ |
| NSSy5632 | +++ |

+ means >1 uM; ++ means 200 nM-1000 nM; +++ means <200 nM; NT means not tested.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural formula I: 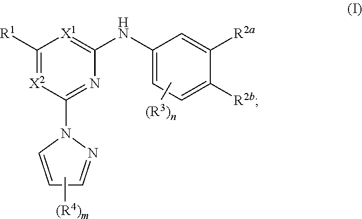
or a pharmaceutically acceptable salt thereof, wherein:
each of X¹ and X² is independently N or C(R⁵), wherein X¹ and X² are not simultaneously N;
R¹ is selected from —CN, —C₁-C₄ alkyl, -heterocyclyl, -heteroaryl, —NH-heterocyclyl, —NH—C₁-C₄ alkyl, —O-heterocyclyl, —O-heteroaryl, —O—C₁-C₄ alkyl, —S—C₁-C₄ alkyl, —S(O)—C₁-C₄ alkyl, —S(O)₂—C₁-C₄ alkyl, and —S(O)₂-heterocyclyl; or
when X¹ or X² is C(R⁵), R¹ is optionally taken together with X¹ or X² and their intervening atoms to form a 5- or 6-membered heteroaryl or heterocyclyl ring comprising 1 or 2 nitrogen atoms;
each of R^{2a} and R^{2b} is independently selected from hydrogen, fluoro, chloro, —CN, —CF₃, —CHF₂, and C₁-C₄ alkyl, wherein at least one of R^{2a} or R^{2b} is fluoro, chloro, —CN, —CF₃, or —CHF₂;
each R³ is independently selected from fluoro, chloro and C₁-C₄ alkyl;
each R⁴ is independently selected from cyano and C₁-C₄ alkyl;
each R⁵ is independently selected from hydrogen and C₁-C₄ alkyl;
n is 0, 1, 2, or 3;
m is 1, 2 or 3;
wherein any alkyl portion of R¹, R^{2a}, R^{2b}, R³, R⁴ or R⁵ and any heterocyclyl or heteroaryl portion of R¹ or the ring formed by taking R¹ together with X¹ or X² is optionally substituted with one or more substituents independently selected from R⁶;
R⁶ is selected from halogen, —CN, —OR^{c}, —NR^{d}R^{e}, —S(O)ₖR^{c}, —NR^{c}S(O)₂R^{c}, —S(O)₂NR^{d}R^{e}, —C(═O)OR^{c}, —OC(═O)OR^{c}, —OC(═O)R^{c}, —OC(═S)OR^{c}, —C(═S)OR^{c}, —OC(═S)R^{c}, —C(═O)NR^{d}R^{e}, —NR^{c}C(═O)R^{c}, —C(═S)NR^{d}R^{e}, —NR^{c}C(═S)R^{c}, —NR^{c}C(═O)OR^{c}, —OC(═O)NR^{d}R^{e}, —NR^{c}(C═S)OR^{c}, —OC(═S)NR^{d}R^{e}, —NR^{c}C(═O)NR^{d}R^{e}, —NR^{c}(C═S)NR^{d}R^{e}, —C(═S)R^{c}, —C(═O)R^{c}, (C₁-C₆)alkyl, cycloalkyl, —(CH₂)₁₋₄-cycloalkyl, heterocyclyl, —(CH₂)₁₋₄-heterocyclyl, aryl, —NHC(═O)-heterocyclyl, —NHC(═O)-cycloalkyl, —(CH₂)₁₋₄-aryl, heteroaryl and —(CH₂)₁₋₄-heteroaryl,
wherein the alkyl, cycloalkyl, heterocyclyl, aryl or heteroaryl portion present in each of said (C₁-C₆)alkyl, cycloalkyl, —(CH₂)₁₋₄-cycloalkyl, heterocyclyl, —(CH₂)₁₋₄-heterocyclyl, aryl, —(CH₂)₁₋₄-aryl, heteroaryl and —(CH₂)₁₋₄-heteroaryl substituent for R⁶ are further optionally substituted with halogen, OR^{c}, —NO₂, —CN, —NR^{c}C(═O)R^{c}, —NR^{d}R^{e}, —S(O)ₖR^{c}, —C(═O)OR^{c}, —C(═O)NR^{d}R^{e}, —C(═O)R^{c}, (C₁-C₃)alkyl, halo(C₁-C₃)alkyl, (C₁-C₃)alkoxy(C₁-C₃)alkyl, (C₁-C₃)alkoxy, or halo(C₁-C₃)alkoxy;
each R^{c} is independently selected from hydrogen and (C₁-C₆)alkyl optionally substituted with 1 to 3 halogen;
each R^{d} and R^{e} is independently selected from hydrogen and (C₁-C₆)alkyl;
k is 0, 1 or 2; and
any alkyl portion of any of R¹, R^{2a}, R^{2b}, R³, or R⁵ and any heterocyclyl or heteroaryl portion of R¹ or the ring formed by taking R¹ together with X¹ or X² is further optionally substituted with ═O;
provided that:
when X² is N; X¹ and R¹ are taken together to form: 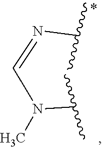 [SMILES: */N=C\N(*)C]
wherein “*” represents a portion of the moiety bound to X¹; n is 0 or 1; and R³, when present, is halo, then the portion of the molecule represented by 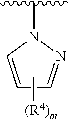
is other than 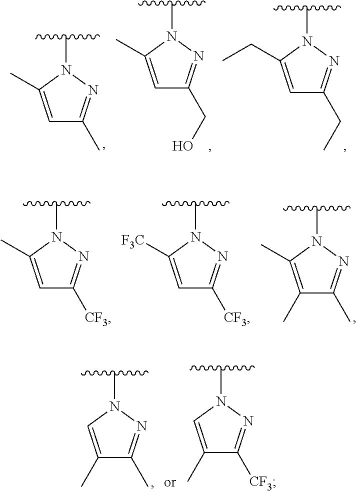
when X² is N; X¹ and R¹ are taken together to form: 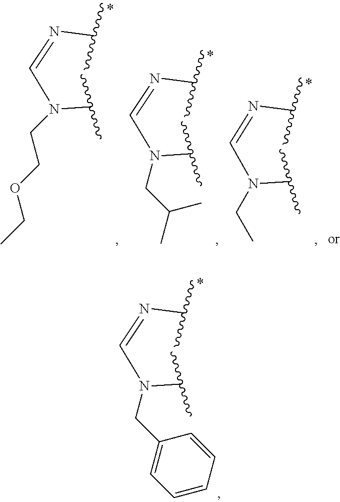
wherein “*” represents a portion of the moiety bound to X¹; and n is 0, then the portion of the molecule represented by 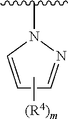
is other than 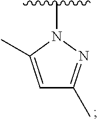
when R¹ is methyl, the portion of the molecule represented by 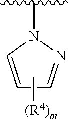
is other than 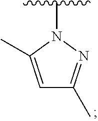
and
the compound is other than 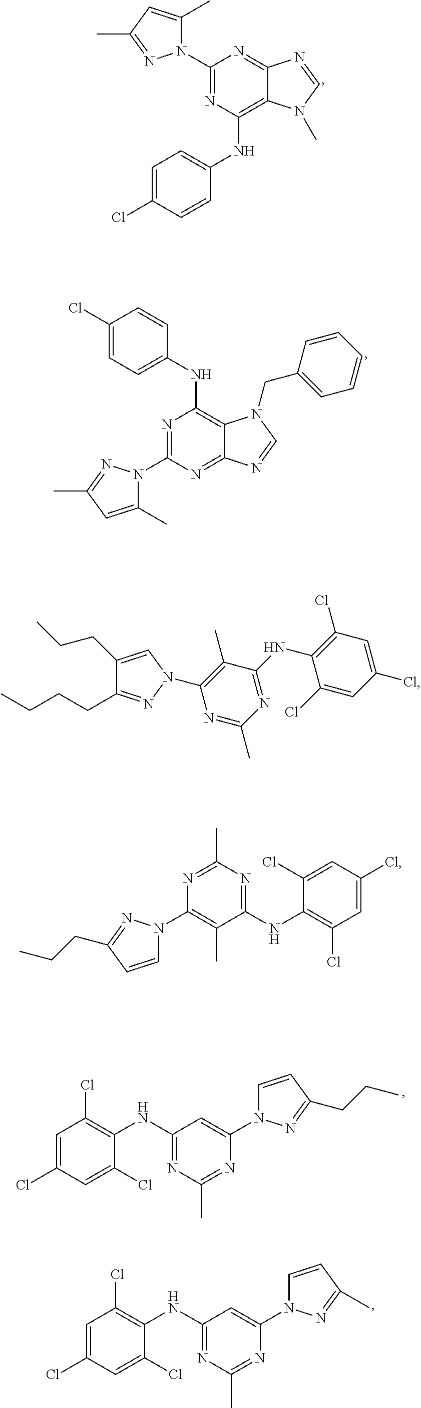 [SMILES: Cc1cc(C)n(-c2nc(Nc3ccc(Cl)cc3)c3c(ncn3C)n2)n1 ; Cc1cc(C)n(-c2nc(Nc3ccc(Cl)cc3)c3c(ncn3Cc3ccccc3)n2)n1 ; CCCCc1nn(-c2nc(C)nc(Nc3c(Cl)cc(Cl)cc3Cl)c2C)cc1CCC ; CCCc1ccn(-c2nc(C)nc(Nc3c(Cl)cc(Cl)cc3Cl)c2C)n1 ; CCCc1ccn(-c2cc(Nc3c(Cl)cc(Cl)cc3Cl)nc(C)n2)n1 ; Cc1ccn(-c2cc(Nc3c(Cl)cc(Cl)cc3Cl)nc(C)n2)n1] 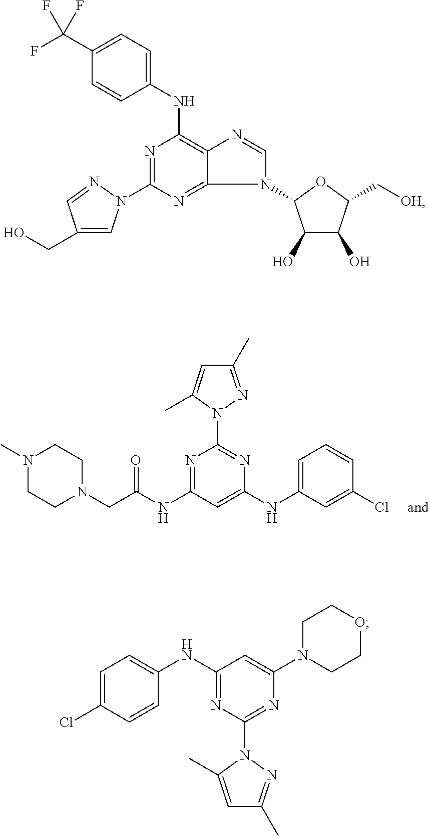 [SMILES: OCc1cnn(-c2nc(Nc3ccc(C(F)(F)F)cc3)c3ncn([C@@H]4O[C@H](CO)[C@@H](O)[C@H]4O)c3n2)c1 ; Cc1cc(C)n(-c2nc(NC(=O)CN3CCN(C)CC3)cc(Nc3cccc(Cl)c3)n2)n1 ; "and" ; Cc1cc(C)n(-c2nc(Nc3ccc(Cl)cc3)cc(N3CCOCC3)n2)n1]
or a pharmaceutically acceptable salt thereof.

2. A compound having the structural formula Ia: 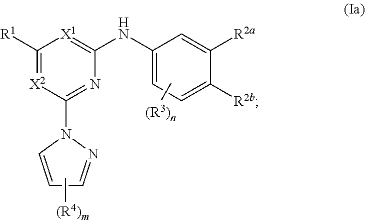
or a pharmaceutically acceptable salt thereof, wherein:
each of X¹ and X² is independently N or C(R⁵), wherein X¹ and X² are not simultaneously N;
R¹ is selected from —CN, —(C₀-C₄ alkylene)-heterocyclyl, —(C₀-C₄ alkylene)-heteroaryl, —C(O)—C₁-C₄ alkyl, —C(O)—O—C₁-C₄ alkyl, —C(O)—NH₂, —C(O)—NH—C₁-C₄ alkyl, —C(O)—N(C₁-C₄ alkyl)₂, —(C₀-C₄ alkylene)-NH-heterocyclyl, —(C₀-C₄ alkylene)-NH—C₁-C₄ alkyl, —(C₀-C₄ alkylene)-NH₂, —(C₀-C₄ alkylene)-O-heterocyclyl, —(C₀-C₄ alkylene)-O-heteroaryl, —(C₀-C₄ alkylene)-O—C₁-C₄ alkyl, —(C₀-C₄ alkylene)-S—C₁-C₄ alkyl, —(C₀-C₄ alkylene)-S(O)—C₁-C₄ alkyl, —(C₀-C₄ alkylene)-S(O)₂—C₁-C₄ alkyl, and —(C₀-C₄ alkylene)-S(O)₂-heterocyclyl;
each of R^{2a} and R^{2b} is independently selected from hydrogen, fluoro, chloro, —CN, —CF₃, —CHF₂, and C₁-C₄ alkyl, wherein at least one of R^{2a} or R^{2b} is fluoro, chloro, —CN, —CF₃, or —CHF₂
each R³ is independently selected from fluoro, chloro and C₁-C₄ alkyl;
each R⁴ is independently selected from cyano and C₁-C₄ alkyl;
each R⁵ is independently selected from hydrogen and C₁-C₄ alkyl;
n is 0, 1, 2, or 3;
m is 1, 2 or 3;
wherein any alkyl portion of R^{2a}, R^{2b}, R³, R⁴ or R⁵ and any alkyl, alkylene, heterocyclyl or heteroaryl portion of R¹ is optionally substituted with one or more substituents independently selected from R⁶;
R⁶ is selected from halogen, —CN, —OR^{c}, —NR^{d}R^{e}, —S(O)ₖR^{c}, —NR^{c}S(O)₂R^{c}, —S(O)₂NR^{d}R^{e}, —C(═O)OR^{c}, —OC(═O)OR^{c}, —OC(═O)R^{c}, —OC(═S)OR^{c}, —C(═S)OR^{c}, —OC(═S)R^{c}, —C(═O)NR^{d}R^{e}, —NR^{c}C(═O)R^{c}, —C(═S)NR^{d}R^{e}, —NR^{c}C(═S)R^{c}, —NR^{c}C(═O)OR^{c}, —OC(═O)NR^{d}R^{e}, —NR^{c}(C═S)OR^{c}, —OC(═S)NR^{d}R^{e}, —NR^{c}C(═O)NR^{d}R^{e}, —NR^{c}(C═S)NR^{d}R^{e}, —C(═S)R^{c}, —C(═O)R^{c}, (C₁-C₆)alkyl, cycloalkyl, —(CH₂)₁₋₄-cycloalkyl, heterocyclyl, —(CH₂)₁₋₄-heterocyclyl, aryl, —NHC(═O)-heterocyclyl, —NHC(═O)-cycloalkyl, —(CH₂)₁₋₄-aryl, heteroaryl and —(CH₂)₁₋₄-heteroaryl,
wherein the alkyl, cycloalkyl, heterocyclyl, aryl or heteroaryl portion present in each of said (C₁-C₆)alkyl, cycloalkyl, —(CH₂)₁₋₄-cycloalkyl, heterocyclyl, —(CH₂)₁₋₄-heterocyclyl, aryl, —(CH₂)₁₋₄-aryl, heteroaryl and —(CH₂)₁₋₄-heteroaryl substituent for R⁶ are further optionally substituted with halogen, OR^{c}, —NO₂, —CN, —NR^{c}C(═O)R^{c}, —NR^{d}R^{e}, —S(O)ₖR^{c}, —C(═O)OR^{c}, —C(═O)NR^{d}R^{e}, —C(═O)R^{c}, (C₁-C₃)alkyl, halo(C₁-C₃)alkyl, (C₁-C₃)alkoxy(C₁-C₃)alkyl, (C₁₋C₃)alkoxy, or halo(C₁-C₃)alkoxy;
each R^{c} is independently selected from hydrogen and (C₁-C₆)alkyl optionally substituted with 1 to 3 halogen;
each R^{d} and R^{e} is independently selected from hydrogen and (C₁-C₆)alkyl;
k is 0, 1 or 2; and
any alkyl portion of any of R^{2a}, R^{2b}, R³, or R⁵ and any alkyl, alkylene, heterocyclyl or heteroaryl portion of R¹ is further optionally substituted with ═O;
provided that the compound is other than 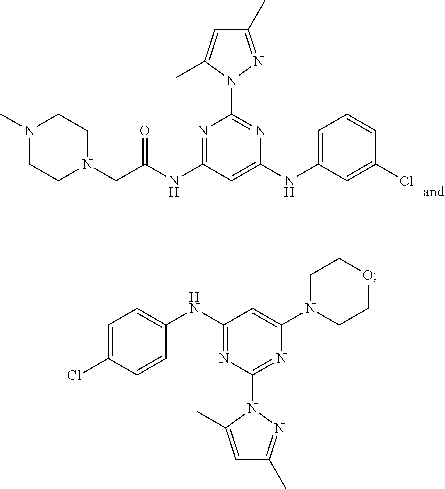 [SMILES: Cc1cc(C)n(-c2nc(NC(=O)CN3CCN(C)CC3)cc(Nc3cccc(Cl)c3)n2)n1 ; Cc1cc(C)n(-c2nc(Nc3ccc(Cl)cc3)cc(N3CCOCC3)n2)n1]
or a pharmaceutically acceptable salt thereof.

3. The compound of claim 1, wherein R¹ is selected from —CN, —C(O)NH₂, heterocyclyl, heteroaryl, —NH-heterocyclyl, —NH—(C₁-C₄ alkylene)-heteroaryl, —O—(C₁-C₄ alkylene)-heteroaryl, —O-heterocyclyl, —O-heteroaryl, —(C₁-C₄ alkylene)-NH—C(O)—C₁-C₄ alkyl, —NH—(C₁-C₄ alkylene)-C(O)—NH₂, —(C₁-C₄ alkylene)-heteroaryl, and —S(O)₂—C₁-C₄ alkyl, wherein each alkyl, alkylene, heterocyclyl and heteroaryl portion of R¹ is optionally substituted with one or more substituents independently selected from R⁶.

4. The compound of claim 1, wherein R¹ is selected from —CN, —C(O)NH₂, heterocyclyl, heteroaryl, —NH-heterocyclyl, —NH—(C₁-C₂ alkylene)-heteroaryl, —O—(C₁-C₂ alkylene)-heteroaryl, —O-heterocyclyl, —O-heteroaryl, —(C₁-C₂ alkylene)-NH—C(O)—C₁-C₄ alkyl, —NH—(C₁-C₂ alkylene)-C(O)—NH₂, —(C₁-C₂ alkylene)-heteroaryl, and —S(O)₂—C₁-C₂ alkyl, wherein each heterocyclyl and heteroaryl portion of R¹ is optionally substituted with one or more substituents independently selected from R⁶.

5. The compound of claim 1, wherein R¹ is selected from piperazinyl, 2,5-diazabicyclo[2.2.1]heptan-2-yl, 2-oxa-6-azaspiro[3.3]heptan-6-yl, morpholinyl, —NH-3-azabicyclo[3.1.0]hexan-6-ylamino, O-oxetanyl, O-pyridazinyl, —(C₁-C₂ alkylene)-pyrazolyl, O-pyridazinyl, —NH—(C₁-C₂ alkylene)-thiazolyl, —NH—(C₁-C₂ alkylene)-oxazolyl, —NH-pyrrolidin-2-one-3-yl, —NH-piperidin-2-one-3-yl, —O—(C₁-C₂ alkylene)-triazolyl, —CN, —C(O)NH₂, —(C₁-C₂ alkylene)-NH—C(O)—C₁-C₄ alkyl, —NH—(C₁-C₂ alkylene)-C(O)—NH₂, and —S(O)₂—C₁-C₂ alkyl, wherein each of said piperazinyl 2,5-diazabicyclo[2.2.1]heptan-2-yl, 2-oxa-6-azaspiro[3.3]heptan-6-yl, morpholinyl, 3-azabicyclo[3.1.0]hexan-6-ylamino, oxetanyl, pyridazinyl, pyrazolyl, thiazolyl, oxazolyl, triazolyl, pyrrolidin-2-one-3-yl, and piperidin-2-one-3-yl are optionally substituted with one or more substituents independently selected from R⁶.

6. The compound of claim 1, wherein R⁶ is selected from —C(═O)R^{c}, —OR^{c}, —NR^{d}R^{e}, —C(═O)NR^{d}R^{e}, —(C₁-C₄)alkyl, —(C₁-C₄)alkyl-OR^{c}, and —(C₁-C₄)alkyl-phenyl; R^{d} and R^{e} are each independently hydrogen or (C₁-C₄)alkyl; and R^{c} is hydrogen or (C₁-C₃)alkyl.

7. The compound of claim 1, wherein R⁶ is selected from —C(═O)R^{c}, —C(═O)NR^{d}R^{e}, —(C₁-C₄)alkyl, —(C₁-C₄)alkyl-OR^{c}, and —(C₁-C₄)alkyl-phenyl; R^{d} and R^{e} are each hydrogen; and R^{c} is hydrogen or (C₁-C₃)alkyl.

8. The compound of claim 1, wherein R¹ is selected from —CN, —C(O)NH₂, —CH₂NH₂, —CH₂NHCOCH₃, —S(O)₂CH₃, —NHCH(C(O)NH₂)CH(CH₃)₂,

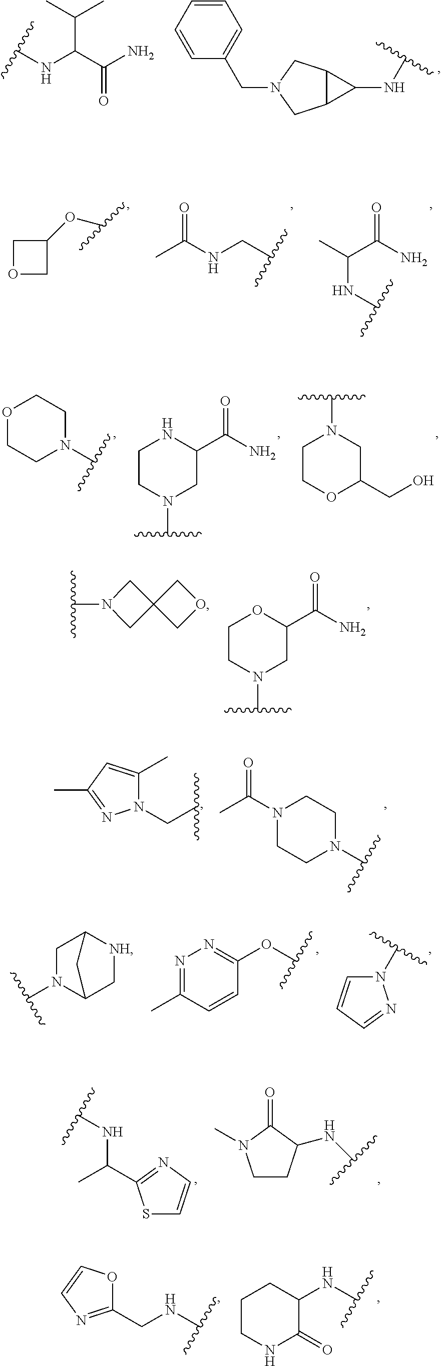

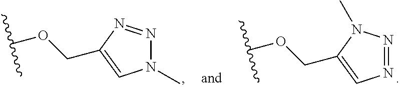

9. The compound of claim 1, wherein R^{2b} is selected from hydrogen, fluoro and chloro; and R^{2a} is selected from hydrogen and —CF₃.

10. The compound of claim 1, wherein n is 0 or 1.

11. The compound of claim 1, wherein R³ is selected from fluoro and CF₃.

12. The compound of claim 1, wherein each R⁴ is independently selected from —CN and C₁-C₄ alkyl optionally substituted hydroxy or one or more with halo.

13. The compound of claim 1, wherein each R⁴ is independently selected from —CN, —CH₃, —CH₂F, —CHF₂, —CH₂OH, —CH(OH)CH₃, —C(CH₃)₂OH, and —CH(CH₃)F.

14. The compound of claim 1, wherein each R⁴ is —CH₃.

15. The compound of claim 1, wherein each R⁵, if present, is independently selected from CH₃ and CH(OH)CH₃.

16. The compound of claim 1, wherein R¹ is taken together with X² to form a ring selected from: 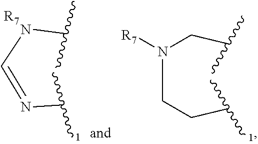
wherein “1” represents a point of attachment to X², and R⁷ is selected from hydrogen and C₁-C₄ alkyl optionally substituted with one or more substituents independently selected from R⁶.

17. The compound of claim 1, wherein R¹ is taken together with X² to form a ring selected from:

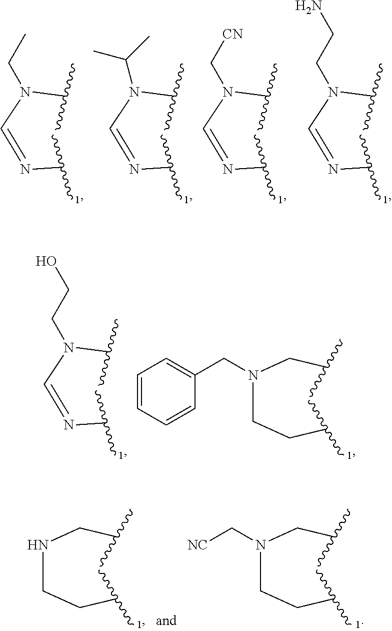

18. The compound of claim 1, wherein R¹ is C₁-C₄ alkyl optionally substituted with halo.

19. The compound of claim 1, wherein R¹ is methyl.

20. The compound of claim 1, wherein the compound is selected from: 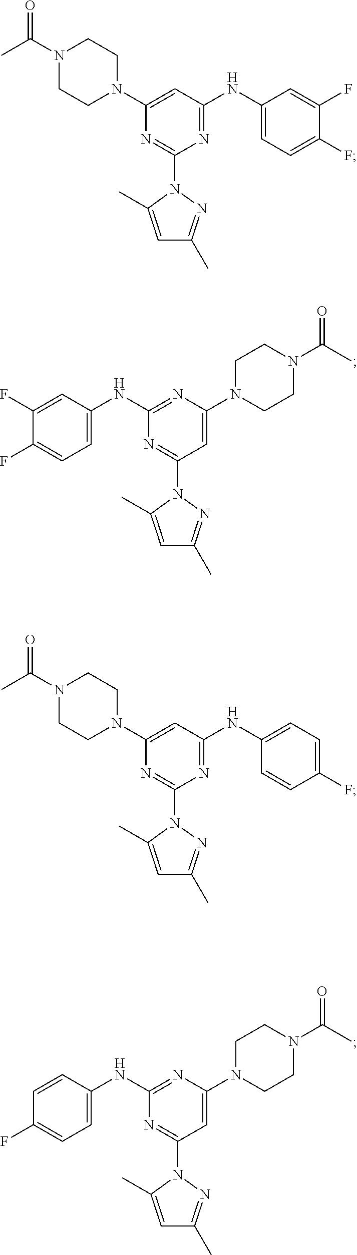 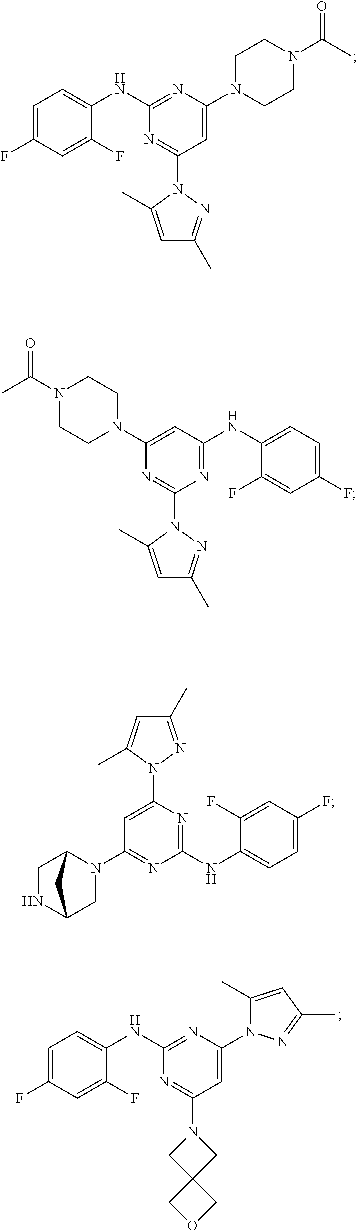 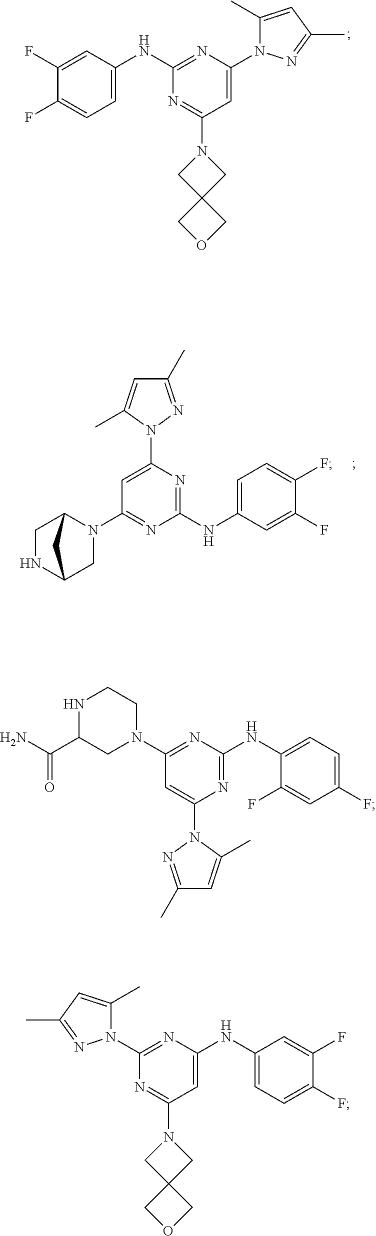 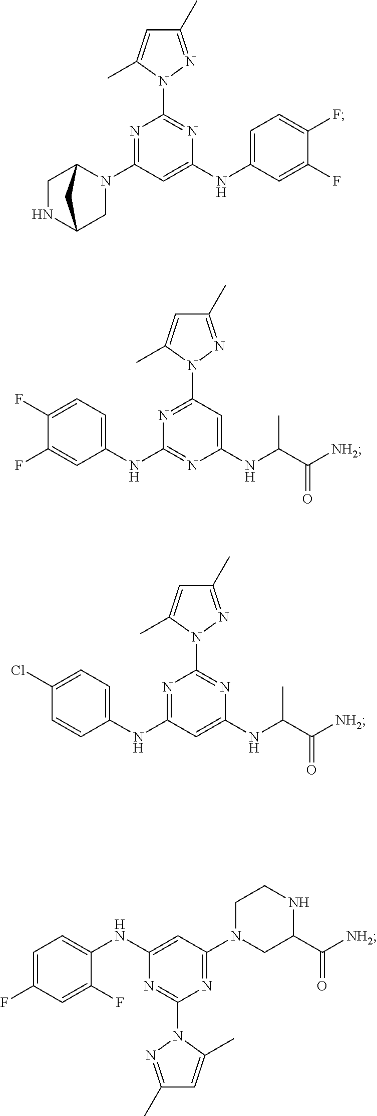 [SMILES: Cc1cc(C)n(-c2nc(Nc3ccc(F)c(F)c3)cc(N3C[C@H]4C[C@@H]3CN4)n2)n1 ; Cc1cc(C)n(-c2cc(NC(C)C(N)=O)nc(Nc3ccc(F)c(F)c3)n2)n1 ; Cc1cc(C)n(-c2nc(Nc3ccc(Cl)cc3)cc(NC(C)C(N)=O)n2)n1 ; Cc1cc(C)n(-c2nc(Nc3ccc(F)cc3F)cc(N3CCNC(C(N)=O)C3)n2)n1] 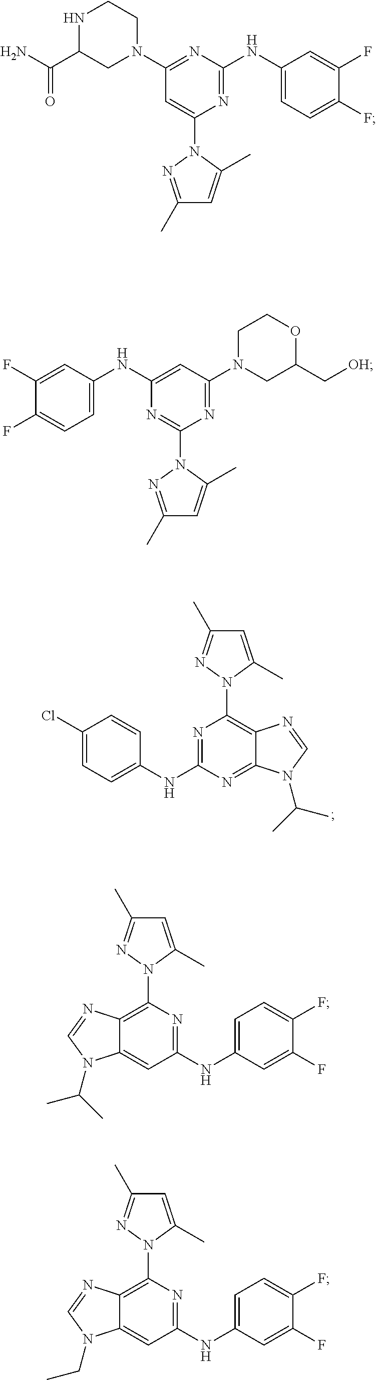 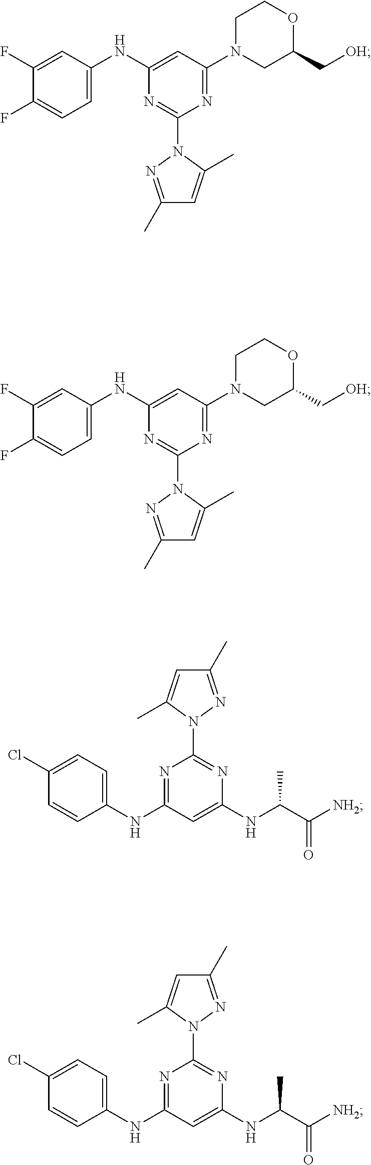 [SMILES: Cc1cc(C)n(-c2nc(Nc3ccc(F)c(F)c3)cc(N3CCO[C@@H](CO)C3)n2)n1 ; Cc1cc(C)n(-c2nc(Nc3ccc(F)c(F)c3)cc(N3CCO[C@H](CO)C3)n2)n1 ; Cc1cc(C)n(-c2nc(Nc3ccc(Cl)cc3)cc(N[C@H](C)C(N)=O)n2)n1 ; Cc1cc(C)n(-c2nc(Nc3ccc(Cl)cc3)cc(N[C@@H](C)C(N)=O)n2)n1] 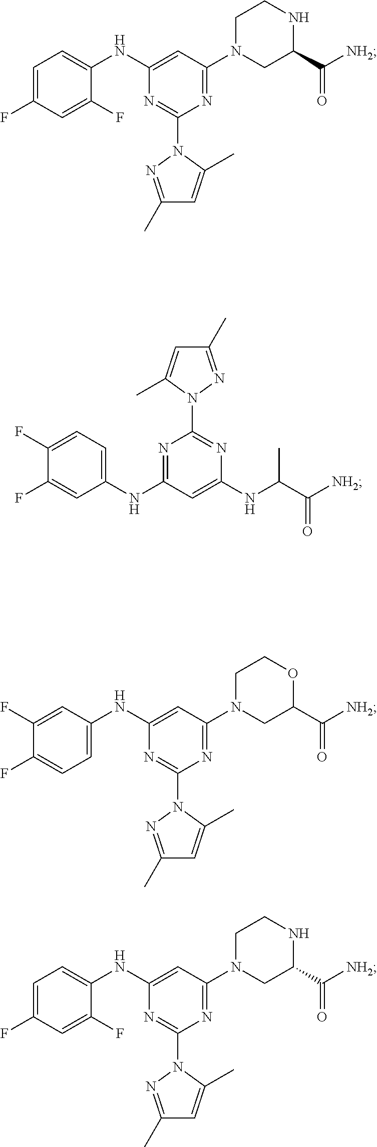 [SMILES: Cc1cc(C)n(-c2nc(Nc3ccc(F)cc3F)cc(N3CCN[C@@H](C(N)=O)C3)n2)n1 ; Cc1cc(C)n(-c2nc(Nc3ccc(F)c(F)c3)cc(NC(C)C(N)=O)n2)n1 ; Cc1cc(C)n(-c2nc(Nc3ccc(F)c(F)c3)cc(N3CCOC(C(N)=O)C3)n2)n1 ; Cc1cc(C)n(-c2nc(Nc3ccc(F)cc3F)cc(N3CCN[C@H](C(N)=O)C3)n2)n1] 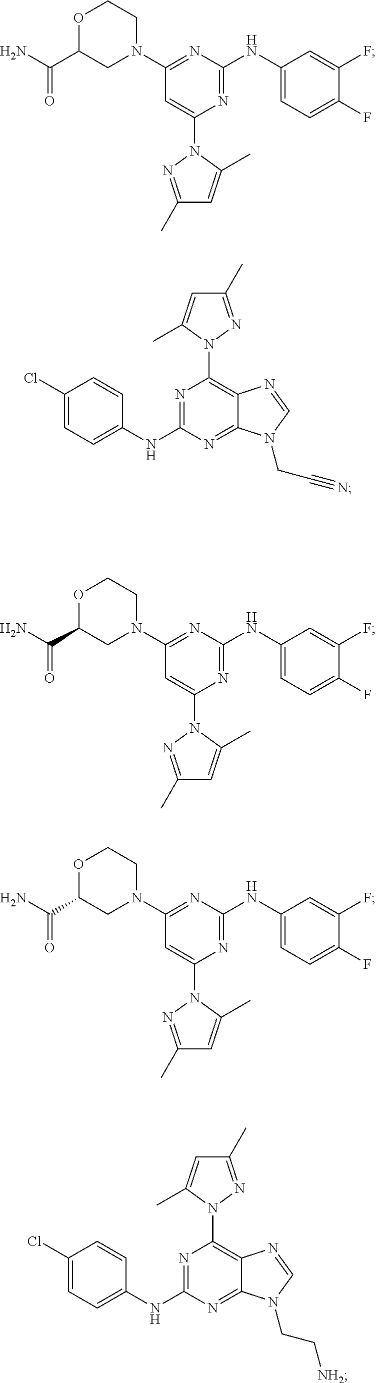 [SMILES: Cc1cc(C)n(-c2cc(N3CCOC(C(N)=O)C3)nc(Nc3ccc(F)c(F)c3)n2)n1 ; Cc1cc(C)n(-c2nc(Nc3ccc(Cl)cc3)nc3c2ncn3CC#N)n1 ; Cc1cc(C)n(-c2cc(N3CCO[C@H](C(N)=O)C3)nc(Nc3ccc(F)c(F)c3)n2)n1 ; Cc1cc(C)n(-c2cc(N3CCO[C@@H](C(N)=O)C3)nc(Nc3ccc(F)c(F)c3)n2)n1 ; Cc1cc(C)n(-c2nc(Nc3ccc(Cl)cc3)nc3c2ncn3CCN)n1] 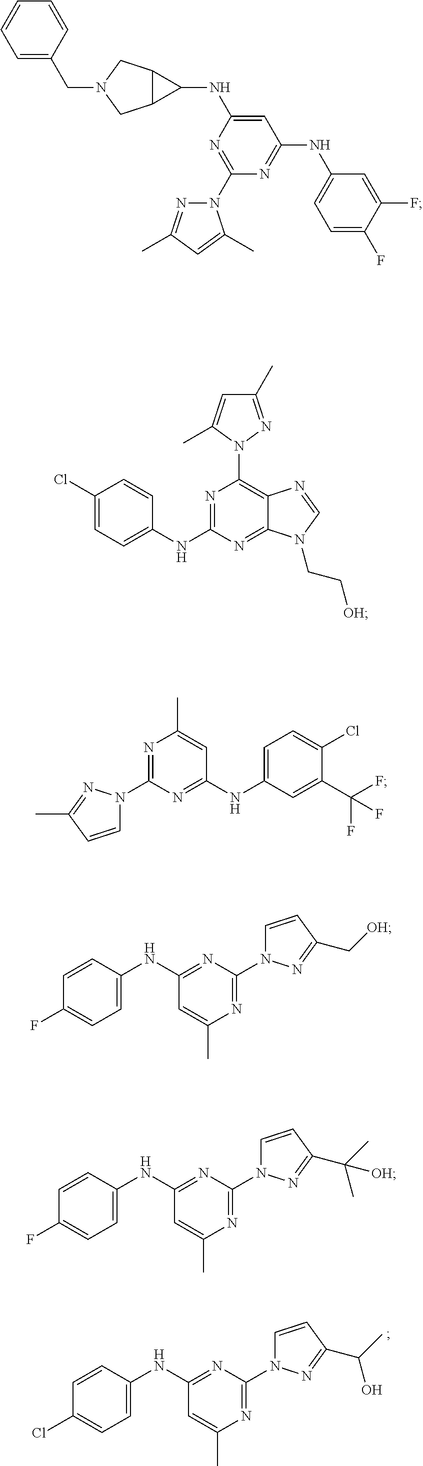 [SMILES: Cc1cc(C)n(-c2nc(Nc3ccc(F)c(F)c3)cc(NC3C4CN(Cc5ccccc5)CC43)n2)n1 ; Cc1cc(C)n(-c2nc(Nc3ccc(Cl)cc3)nc3c2ncn3CCO)n1 ; Cc1cc(Nc2ccc(Cl)c(C(F)(F)F)c2)nc(-n2ccc(C)n2)n1 ; Cc1cc(Nc2ccc(F)cc2)nc(-n2ccc(CO)n2)n1 ; Cc1cc(Nc2ccc(F)cc2)nc(-n2ccc(C(C)(C)O)n2)n1 ; Cc1cc(Nc2ccc(Cl)cc2)nc(-n2ccc(C(C)O)n2)n1] 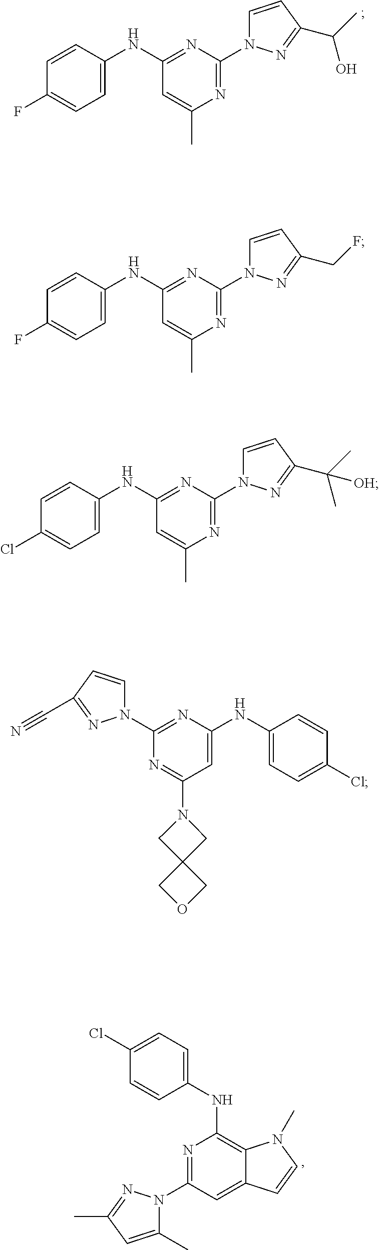 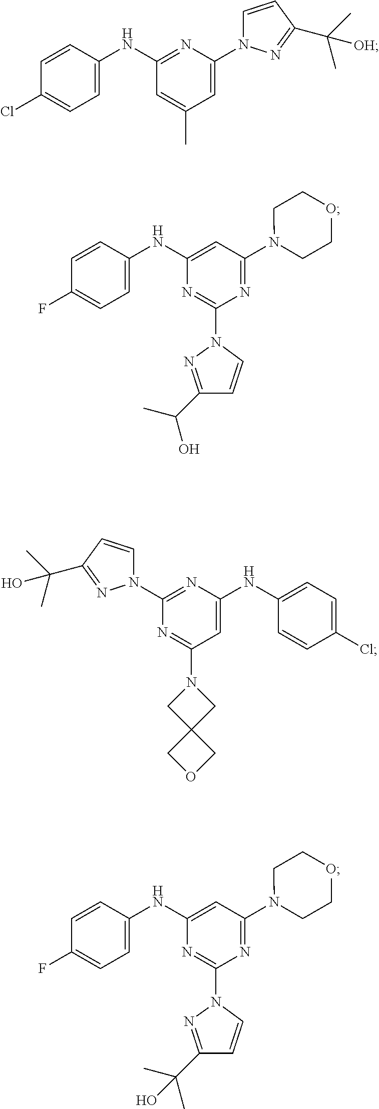 [SMILES: Cc1cc(Nc2ccc(Cl)cc2)nc(-n2ccc(C(C)(C)O)n2)c1 ; CC(O)c1ccn(-c2nc(Nc3ccc(F)cc3)cc(N3CCOCC3)n2)n1 ; CC(C)(O)c1ccn(-c2nc(Nc3ccc(Cl)cc3)cc(N3CC4(COC4)C3)n2)n1 ; CC(C)(O)c1ccn(-c2nc(Nc3ccc(F)cc3)cc(N3CCOCC3)n2)n1] 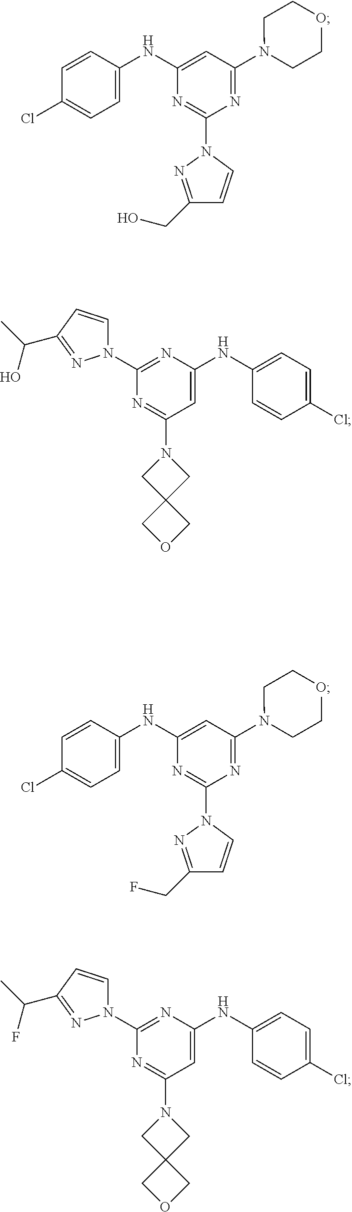 [SMILES: OCc1ccn(-c2nc(Nc3ccc(Cl)cc3)cc(N3CCOCC3)n2)n1 ; CC(O)c1ccn(-c2nc(Nc3ccc(Cl)cc3)cc(N3CC4(COC4)C3)n2)n1 ; FCc1ccn(-c2nc(Nc3ccc(Cl)cc3)cc(N3CCOCC3)n2)n1 ; CC(F)c1ccn(-c2nc(Nc3ccc(Cl)cc3)cc(N3CC4(COC4)C3)n2)n1] 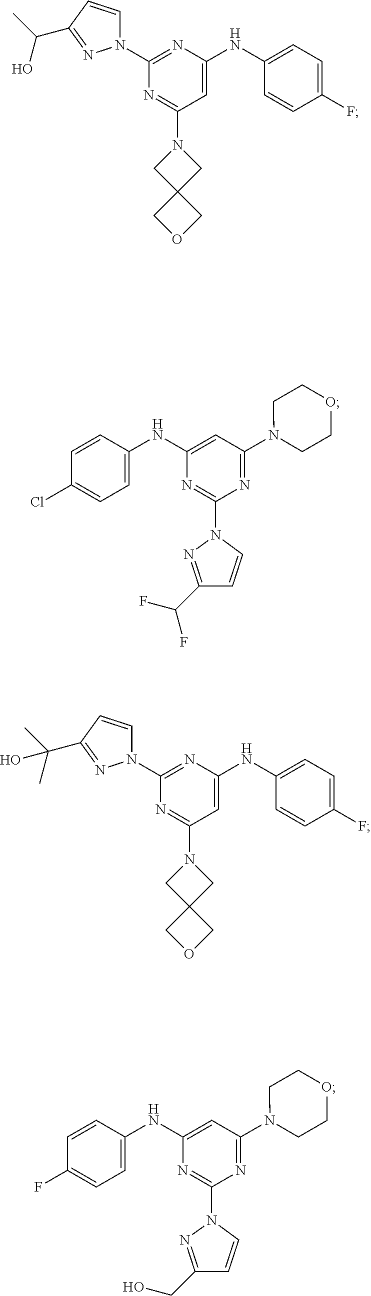 [SMILES: CC(O)c1ccn(-c2nc(Nc3ccc(F)cc3)cc(N3CC4(COC4)C3)n2)n1 ; FC(F)c1ccn(-c2nc(Nc3ccc(Cl)cc3)cc(N3CCOCC3)n2)n1 ; CC(C)(O)c1ccn(-c2nc(Nc3ccc(F)cc3)cc(N3CC4(COC4)C3)n2)n1 ; OCc1ccn(-c2nc(Nc3ccc(F)cc3)cc(N3CCOCC3)n2)n1] 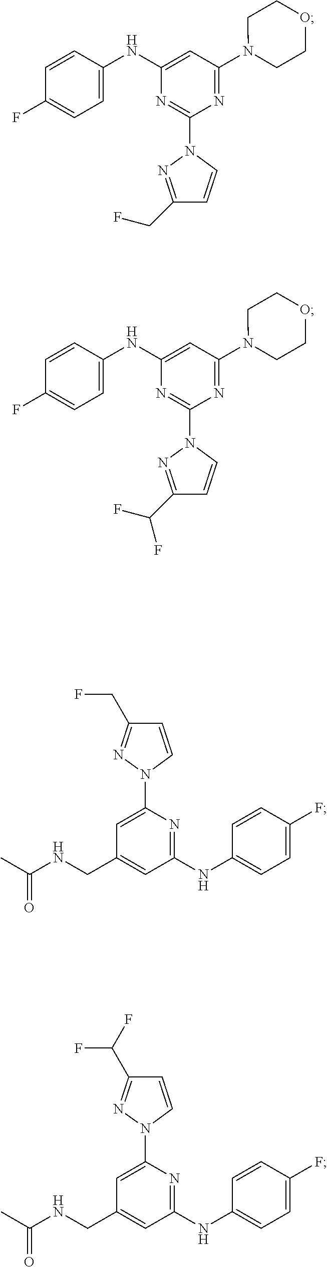 [SMILES: FCc1ccn(-c2nc(Nc3ccc(F)cc3)cc(N3CCOCC3)n2)n1 ; Fc1ccc(Nc2cc(N3CCOCC3)nc(-n3ccc(C(F)F)n3)n2)cc1 ; CC(=O)NCc1cc(Nc2ccc(F)cc2)nc(-n2ccc(CF)n2)c1 ; CC(=O)NCc1cc(Nc2ccc(F)cc2)nc(-n2ccc(C(F)F)n2)c1] 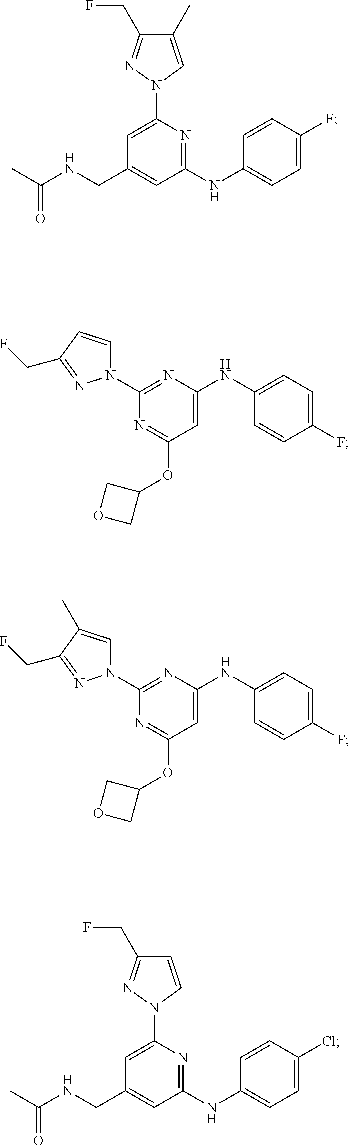 [SMILES: CC(=O)NCc1cc(Nc2ccc(F)cc2)nc(-n2cc(C)c(CF)n2)c1 ; FCc1ccn(-c2nc(Nc3ccc(F)cc3)cc(OC3COC3)n2)n1 ; Cc1cn(-c2nc(Nc3ccc(F)cc3)cc(OC3COC3)n2)nc1CF ; CC(=O)NCc1cc(Nc2ccc(Cl)cc2)nc(-n2ccc(CF)n2)c1] 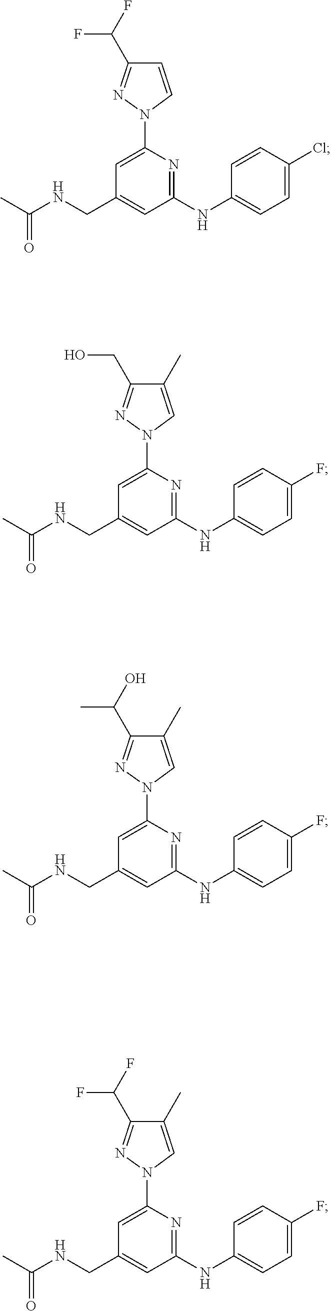 [SMILES: CC(=O)NCc1cc(Nc2ccc(Cl)cc2)nc(-n2ccc(C(F)F)n2)c1 ; CC(=O)NCc1cc(Nc2ccc(F)cc2)nc(-n2cc(C)c(CO)n2)c1 ; CC(=O)NCc1cc(Nc2ccc(F)cc2)nc(-n2cc(C)c(C(C)O)n2)c1 ; CC(=O)NCc1cc(Nc2ccc(F)cc2)nc(-n2cc(C)c(C(F)F)n2)c1] 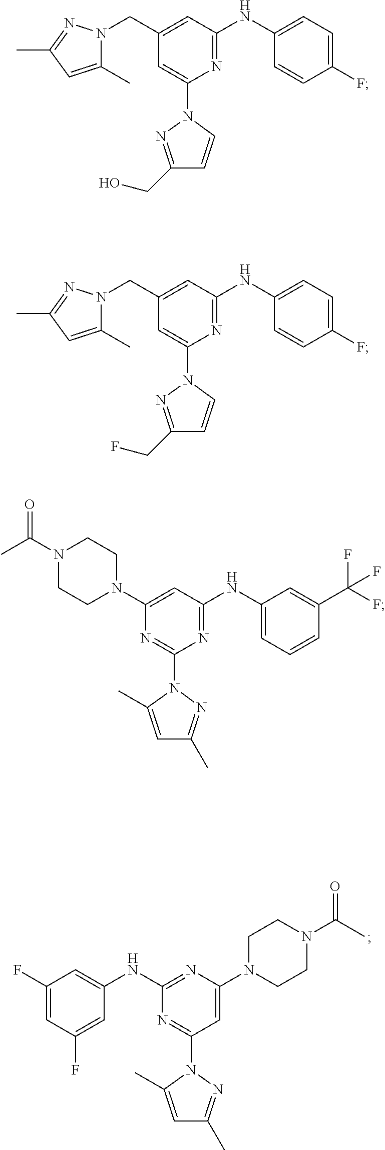 [SMILES: Cc1cc(C)n(Cc2cc(Nc3ccc(F)cc3)nc(-n3ccc(CO)n3)c2)n1 ; Cc1cc(C)n(Cc2cc(Nc3ccc(F)cc3)nc(-n3ccc(CF)n3)c2)n1 ; CC(=O)N1CCN(c2cc(Nc3cccc(C(F)(F)F)c3)nc(-n3nc(C)cc3C)n2)CC1 ; CC(=O)N1CCN(c2cc(-n3nc(C)cc3C)nc(Nc3cc(F)cc(F)c3)n2)CC1] 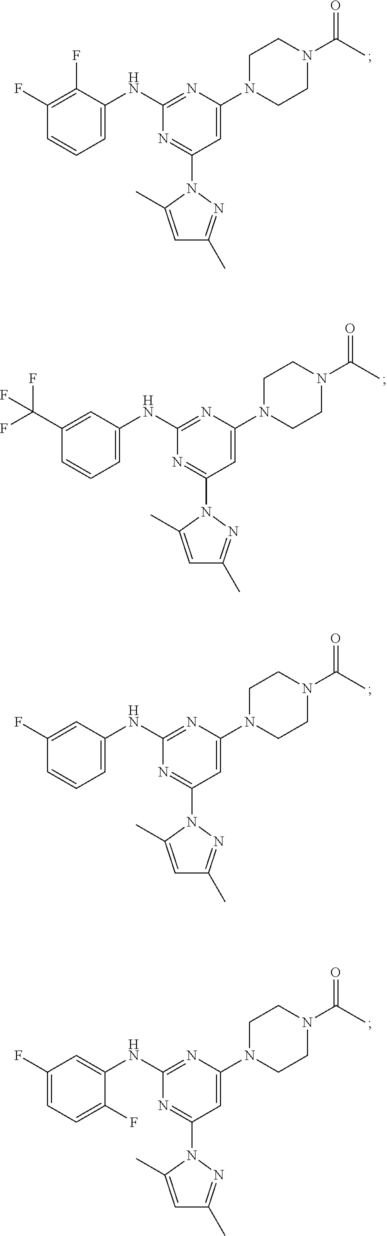 [SMILES: CC(=O)N1CCN(c2cc(-n3nc(C)cc3C)nc(Nc3cccc(F)c3F)n2)CC1 ; CC(=O)N1CCN(c2cc(-n3nc(C)cc3C)nc(Nc3cccc(C(F)(F)F)c3)n2)CC1 ; CC(=O)N1CCN(c2cc(-n3nc(C)cc3C)nc(Nc3cccc(F)c3)n2)CC1 ; CC(=O)N1CCN(c2cc(-n3nc(C)cc3C)nc(Nc3cc(F)ccc3F)n2)CC1] 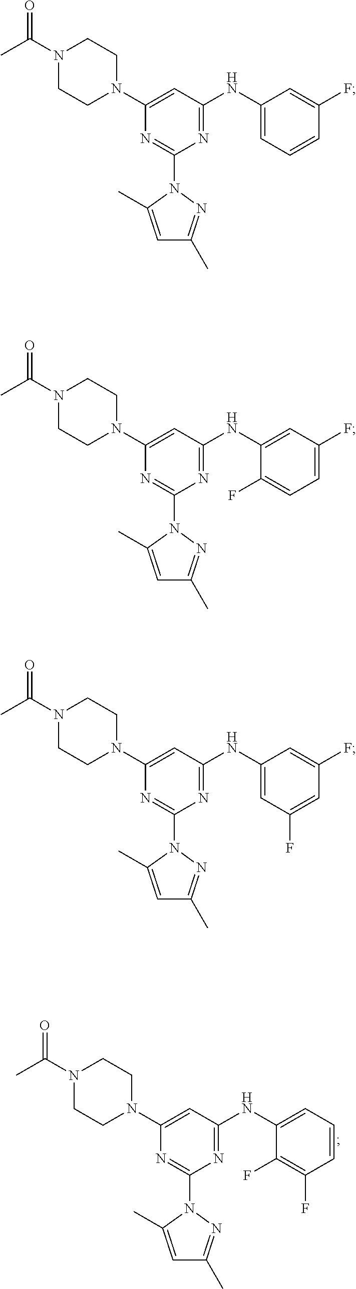 [SMILES: CC(=O)N1CCN(c2cc(Nc3cccc(F)c3)nc(-n3nc(C)cc3C)n2)CC1 ; CC(=O)N1CCN(c2cc(Nc3cc(F)ccc3F)nc(-n3nc(C)cc3C)n2)CC1 ; CC(=O)N1CCN(c2cc(Nc3cc(F)cc(F)c3)nc(-n3nc(C)cc3C)n2)CC1 ; CC(=O)N1CCN(c2cc(Nc3cccc(F)c3F)nc(-n3nc(C)cc3C)n2)CC1] 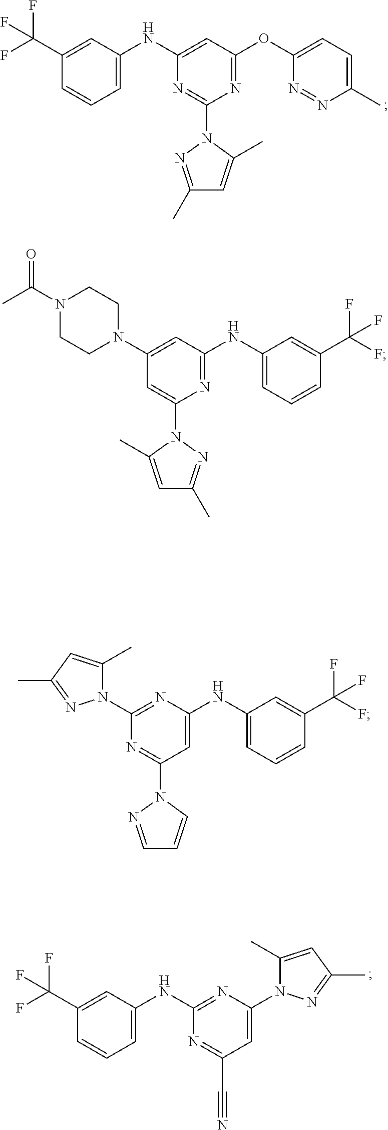 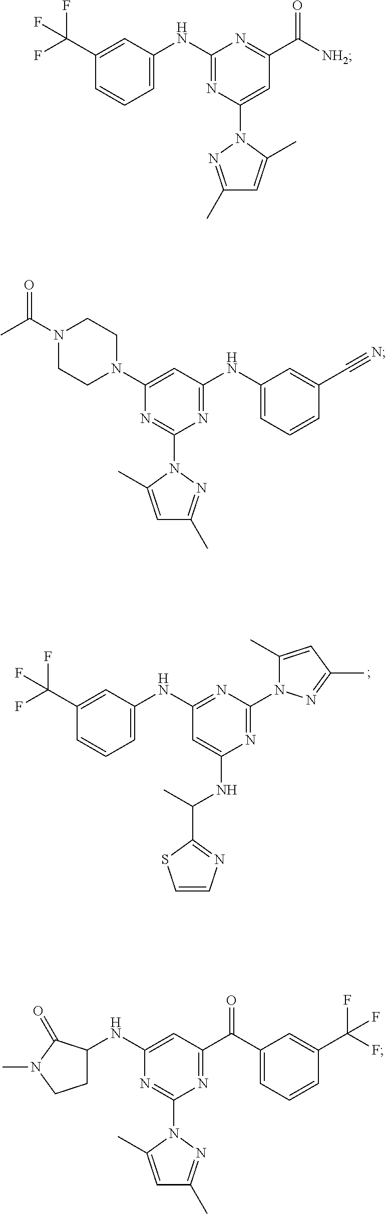 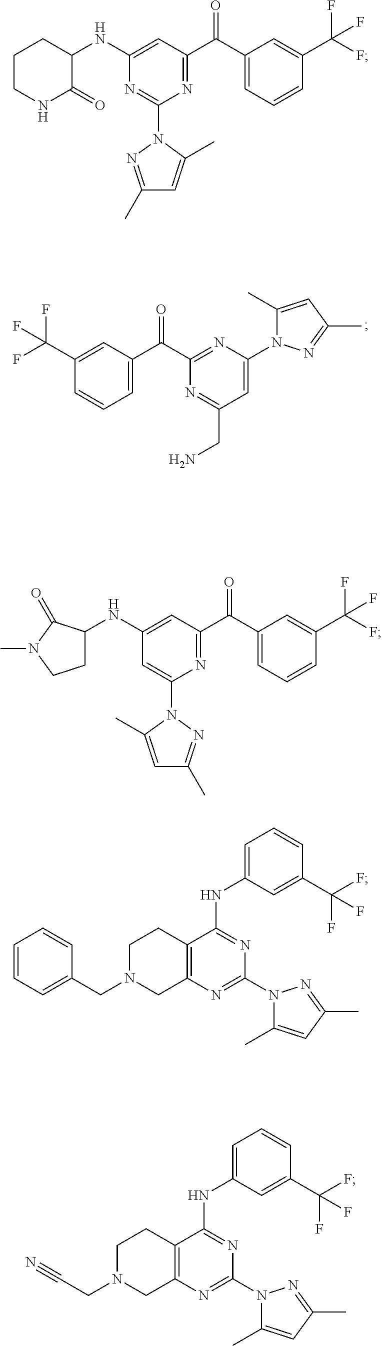 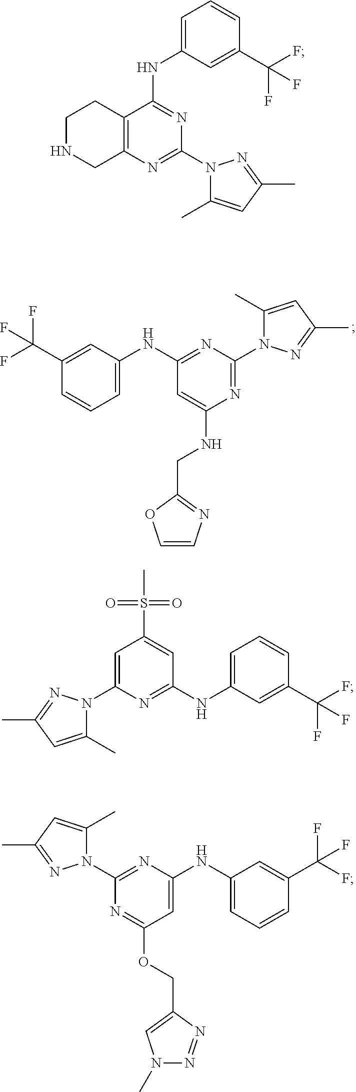 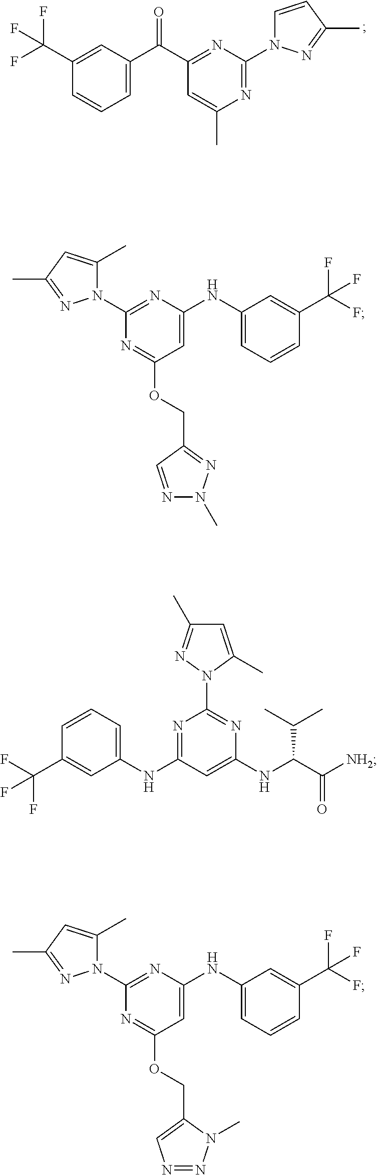 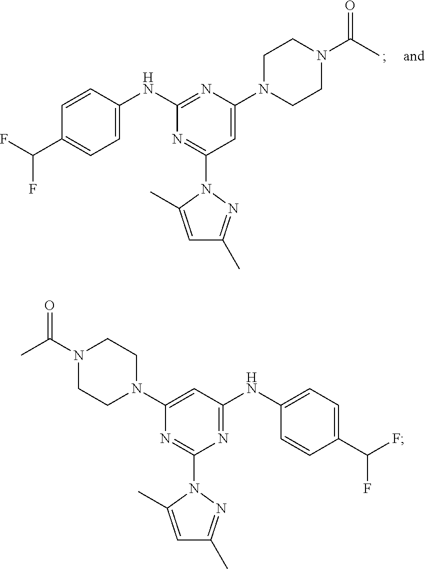
or pharmaceutically acceptable salt thereof.

21. A pharmaceutical composition comprising the compound claim 1, or a pharmaceutically acceptable salt thereof; and a pharmaceutically acceptable carrier.

22. (canceled)

23. A method of treating a disease or condition in a subject selected from a neurodegenerative disease, dementia, heart disease, withdrawal symptoms associated with termination of addiction, metabolic disease, and bladder disease comprising the step of administering the compound of claim 1, or a pharmaceutically acceptable salt thereof, to the subject.

24. The method of claim 23, wherein the disease or condition is selected from ataxia, dystonia, tremors, Parkinson's disease, ischemia, traumatic brain injury, amyotrophic lateral sclerosis, hypertension, atherosclerosis, diabetes, arrhythmia, over-active bladder, and withdrawal symptoms caused by the termination of abuse of alcohol and other drugs of abuse.
